(19) United States
(12) Patent Application Publication
(10) Pub. No.: US 2005/0203082 A1
(43) Pub. Date: Sep. 15, 2005

(54) Combination therapy with inhibitors of inducible nitric oxide synthase and alkylating agents

(76) Inventors: Chung Y. Hsu, Chicago, IL (US); Pamela T. Manning, Labadie, MO (US); Thomas P. Misko, Olivette, MO (US)

Correspondence Address: David M. Gryte;Harness, Dickey & Pierce, P.L.C., Suite 400, 7700 Bonhomme, St. Louis, MO, 63105, US

(21) Appl. No.: 10/918,535

(22) Filed: Aug. 13, 2004

Related U.S. Application Data
(60) Provisional application No. 60/494,917, filed on Aug. 13, 2003.

(51) Int. Cl. A61K 31/55; A61K 31/53

(52) U.S. Cl. 514/217.1; 514/565; 514/341; 514/183; 514/241; 514/492; 514/589

(57) ABSTRACT

A combination therapy comprising administration of a carbamoylating chemotherapeutic agent in conjunction with administration of a selective iNOS inhibitor compound is disclosed. Optionally, resection and radiation therapy are provided with the therapeutic combination. A medicament comprising a carbamoylating chemotherapeutic agent and a selective iNOS inhibitor compound together with a pharmaceutically acceptable carrier is further disclosed. A kit comprising a carbamoylating chemotherapeutic agent and a selective iNOS inhibitor compound is further disclosed.

### PRIORITY CLAIM TO RELATED PATENT APPLICATION

This patent claims priority to U.S. Provisional Patent Application Ser. No. 60/494,917 (filed Aug. 13, 2003). The entire text of U.S. Provisional Patent Application Ser. No. 60/494,917 is incorporated by reference into this patent.

### FIELD OF THE INVENTION

This invention is directed generally to a combination therapy (particularly for the treatment of cancer, and more particularly for the treatment of human cancer) comprising administration of a carbamoylating chemotherapeutic agent in conjunction with administration of a selective iNOS inhibiting compound. This invention also is generally directed to a medicament comprising a carbamoylating chemotherapeutic agent and a selective iNOS inhibiting compound together with a pharmaceutically acceptable carrier. This invention is further generally directed to a kit comprising a carbamoylating chemotherapeutic agent and a selective iNOS inhibiting compound.

### BACKGROUND OF THE INVENTION

Cancer is one of the leading causes of death in industrialized nations, and represents a staggering cost in terms of medical treatment and lost work.

There are currently four principal therapeutic modalities for the treatment of cancer: surgery, radiation therapy, chemotherapy and biologic therapy. Frequently, several different therapeutic treatment modalities are combined to enhance the benefits of each individual therapy, and to decrease the deleterious side effects of an individual therapeutic modality.

Surgery is used to physically remove a neoplasia, and thereby eliminate the tumor cells. Surgery is also beneficial in the diagnosis of cancer, as well as the evaluation of the tumor cell type and characterization of the cancer. The risks associated with surgery are well known, and include adverse reactions with anesthesia, and risk of infection. Surgery alone may not be effective in terminating cancer if the entire neoplasm is not removed, or if the tumor has metastasized. Further, many areas of the body are not accessible to, or are inappropriate for, surgical procedures.

Radiation interacts with molecular oxygen present in tissues and induces the formation of oxygen radical compounds, such as superoxide, hydrogen peroxide, or hydroxyl radicals that damage or break cellular DNA, and thereby kill cells. High linear energy transfer (LET) radiation can induce direct damage to the molecular structure of DNA. Both tumor cells and non-cancerous cells may be affected. The basic unit of ionizing radiation is the gray (Gy), which is equivalent to one hundred rads. Irradiation is commonly fractionated in doses of about 2.0 Gy to the whole organ being treated, with the total dose being dependent on the type of tumor being treated and the sensitivity of the normal organs and tissues within the radiation field. Some body tissues are more susceptible to radiation than others. For example, in bone marrow, the acceptable limiting dose of radiation is about 2.5 Gy, while in the brain, the generally accepted limiting dose of radiation is relatively high, e.g., about 50.0 Gy ionizing radiation. Some tumors, particularly larger tumors, are less affected by irradiation because of the presence of poorly perfused, hypoxic zones that limit the available molecular oxygen required to produce cytotoxic radicals. Radiation therapy may result in a variety of adverse effects such as aplasia of the bone marrow, nephrosclerosis, hepatitis, tissue fibrosis, acute encephalopathy, transient diffuse encephalopathy, early delayed myelopathy, late delayed myelopathy, headache, dementia, cerebral atrophy, radionecrosis, and even radiation-induced tumors.

Chemotherapy may be broadly categorized into two primary types: Cell cycle-specific (CCS) agents (such as antimetabolites, anthracyclines, bleomycin, camptothecins, and plant alkyloids) and cell-cycle non-specific (CCNS) agents (such as alkylating agents, antibiotics, platinum compounds, nitrosoureas, dacarbazine, and L-asparaginase). CCS agents only exert their effects on cycling cells, while CCNS agents have activity against both cycling and, to a lesser extent, non-cycling cells. It is widely accepted that tumor cells grow in accordance with Gompertzian kinetics, with rapid initial cell growth and traversal of complete cell cycle, followed by slowing cell doubling time as the tumor burden increases and more cells remain in a G₀ phase, with cell growth approaching a Malthusian asymptotic limit. Also, in high tumor burden metastatic solid tumors, a significant amount of heterogeneity exists with respect to biologic, kinetic, antigenic, and drug-sensitive cell types present. Therefore, in an individual subject, some tumor cell subpopulations may be more responsive to CCS agent, such as rapidly growing tumor cells, while other tumor cell subpopulations may be more or less unresponsive to the same agent.

Biologic therapy is a relatively recent therapeutic modality. Biological agents (such as cytokines, antibodies, stem cells, and vaccine growth factors) are employed as biologic response modifiers. Here, cellular and humoral immunity is enhanced to recognize and attack cancer cells.

Glioblastoma multiforme (GBM) is among the most frequent and aggressive neoplasias of the central nervous system in adults. Even after the administration of standard therapies, median survival is 9-15 months. The current World Health Organization (WHO) classification of primary brain tumors lists GBM as a Grade IV astrocytoma. GBM is characterized by cellular pleomorphism, numerous mitotic figures, and often multinucleated giant cells. Proliferation of the vascular endothelium is seen, as well as areas of necrosis with circumjacent pseudopalisading of the neoplastic cells. GBM can appear as either a well- circumscribed globular mass or a more diffuse mass lesion. The cut surface reveals necrosis, fatty degeneration, and hemorrhage. Hemorrhages have been found in 40%, with necrosis in up to 52% of the cases. The tumor is usually solid, although cysts may be present. Rarely the tumor consists of a solitary cyst and mural nodule. Variants of the tumor include gliosarcoma, multifocal GBM, or gliomatosis cerebri (in which the entire brain may be infiltrated with tumor cells).

The nitrosoureas carmustine (BCNU) and lomustine (CCNU) are liposoluble alkylating drugs that have constituted the gold standard of first-line chemotherapy for recurrent GBM after resection and radiotherapy, with a response rate of about 30%. Because of their high lipophilicity, BCNU and CCNU are able to cross the blood-brain barrier, and thus reach brain tumors that are inaccessible to other chemotherapeutic agents. BCNU is capable of inhibiting the synthesis of DNA, RNA, and protein, and kills cells in all phases of the cell cycle.

Chemically, BCNU spontaneously degrades to form a carbonium ion and an organic isocyanate group. The organic isocyanate group formed by the decomposition of BCNU attaches a carbamoyl group to a lysine residue of a protein, such as a DNA repair protein, thus deactivating the protein. The carbonium ion formed by the decomposition of BCNU forms a chloroethyl adduct on the O⁶ position of guanine.

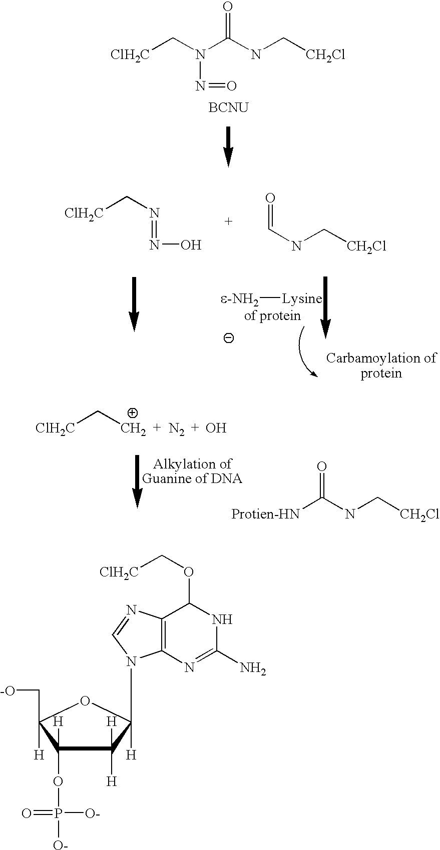

In about 10 to 24 hours, the ethyl chloride group loses the chloride and converts spontaneously into a cyclic N¹—O⁶ ethanol-guanine intermediate that is able to form a covalent bond with the adjacent cytosine. These intrastrand cross-links, 1(N³-cytosine)-2(N¹-guanine), are almost exclusive to BCNU and are the main determinants of BCNU cytotoxicity, because only 10 are needed to provoke cell death by interfering with the processes of DNA duplication and transcription.

Resistance to BCNU is essentially pharmacodynamic and is accomplished by the DNA repair systems through O⁶-alkylguanine-DNA alkyltransferase (AGT), whose mechanism of action is “suicidal”: it binds to DNA, recognizes the alkyl group bound to oxygen at position 6 of guanine, and catalyzes its transfer to a sulfhydryl (—SH) group of a cysteine near its carboxy terminal end (catalytic site); guanine remains intact, while the alkylated protein is no longer active, loses affinity for DNA, and is rapidly degraded. Thus, there is a stoichiometric correspondence between the number of removed alkyl groups and the number of enzyme molecules: at any given moment, a cell may repair only a number of adducts equal to the number of enzyme molecules it possesses, after which it must await the synthesis of new enzyme molecules, which takes over 24 hours.

Another recognized event that leads to the resistance of tumor cells to alkylating agents is the overproduction by the tumor cells of nucleophilic substances, such as glutathione, that can compete with DNA for alkylation.

Several attempts have been made to overcome the acquired resistence to alkylating agents. For example, in WO 02/072008, Welt et al. disclose and claim a combination of immunotherapy and chemotherapy to promote tumor regression by treating a patient in need thereof with a combination of an antibody that binds to A33 antigen and one or more chemotherapeutic agents.

Another approach has been to pre-expose the tumor to other alkylating agents prior to administration of BCNU to saturate the endogenous AGT present in the tumor cells and thereby diminish DNA repair. See Brandes et al., “A multidrug combination designed for reversing resistance to BCNU in glioblastoma multiforme”, Neurology, 58(12), 1759-1764 (2002).

In WO 02/056823, Hoffman discloses and claims oxidation of glutathione, and thus altering the redox state of tumor cells. Nitrogen monoxide (also called “nitric oxide” or “NO”) is an uncharged free radical that serves as a key messenger in immune, cardiovascular, and nervous systems. The physiological activity of what was later identified as NO was initially discovered in the early 1980's when it was found that vascular relaxation caused by acetylcholine is dependent on the presence of the vascular endothelium. The factor derived from the endothelium, then called endothelium-derived relaxing factor (EDRF), that mediates such vascular relaxation is now known to be NO that is generated in the vascular endothelium by one isoform of nitric oxide synthase (NOS). In addition, NO is the active species derived from known nitrovasodilators including amylnitrite, and glyceryltrinitrate. Nitric oxide is also an endogenous stimulator of soluble guanylate cyclase (cGMP), and thus stimulates cGMP production. When NOS is inhibited by N-monomethylarginine (L-NMMA), cGMP formation is completely prevented. In addition to endothelium-dependent relaxation, NO is known to be involved in a number of biological actions, including cytotoxicity of phagocytic cells and cell-to-cell communication in the central nervous system.

The identification of EDRF as NO coincided with the discovery of a biochemical pathway by which NO is synthesized from the amino acid L-arginine by the enzyme NO synthase. There are at least three types of NO synthase:
(i) a constitutive, Ca⁺⁺/calmodulin dependent enzyme, located in the brain, that releases NO in response to receptor or physical stimulation;
(ii) a Ca⁺⁺ independent enzyme (a 130 kD protein) which is induced after activation of vascular smooth muscle, macrophages, endothelial cells, and a number of other cells by endotoxin and cytokines; and
(iii) a constitutive, Ca⁺⁺/calmodulin dependent enzyme, located in the endothelium, that releases NO in response to receptor or physical stimulation.

Once expressed, inducible nitric oxide synthase (hereinafter “iNOS”) generates NO continuously for long periods.

In U.S. patent application Publ. No. 20010038832, Bonavida et al. disclose and claim the use of NO, iNOS, NO donors, and NO mimics in combination with chemotherapeutic agents, to sensitize cells to chemotherapeutic agents. Among the combination of chemotherapeutic agents claimed in that published application is BCNU.

In Int'l Patent Appl. Publ. No. WO 02/056823, Hoffman dislcoses that glutathione (GSH) detoxifies standard chemotherapeutic agents, such as alkylating agents, and indicates that GSH depleting agents, such as oxidizing agents, will convert GSH to glutathione disulfide (GSSG), thereby enhancing the effectiveness of chemotherapeutic agents.

Yin et al., in “Inducible Nitric Oxide Synthase Neutralizes Carbamoylating Potential of 1,3-Bis(2-chloroethyl)-1-nitrosourea in C6 Glioma Cells”, J. Pharmacol. Exp. Therap., Vol. 297, Issue 1, 308-315, (April 2001), note that iNOS-derived NO confers chemoresistance against the carbamoylating potential of chloroethylnitrosoureas in vitro. Inhibition of NO formation by iNOS was achieved by treating C6 cells overexpressing iNOS with L-NAME, a broad-spectrum NOS inhibitor. L-NAME reduced the nitrite levels with corresponding restoration of BCNU toxicity in a concentration-dependent manner.

There is a need in the art for a treatment of cancer (e.g., neoplasia, and in particular glioblastoma multiforme) that is robust and relatively safe, as well as effective for protracted periods of time (e.g., generally not subject to significant resistance by the neoplasias).

### SUMMARY OF THE INVENTION

The present invention is directed, in part, to a combination therapy comprising administration of a selective iNOS inhibitor in combination with a cytotoxic chemotherapeutic agent capable of carbamoylation (e.g., BCNU [1,3-bis(2-chloroethyl)-1-nitrosourea] and CCNU [1-(2-chloroethyl)-3-cyclohexyl-1-nitrosourea]) to treat neoplastic disorders, such as glioblastoma multiforme. It is believed that such a combination therapy will tend to result in an increase in the anti-tumor effects of the carbamoylating chemotherapeutic agent in brain cancer and other human cancers in which carbamoylating cytotoxics (e.g., BCNU) are used as standard of care. Without wishing to be bound by theory, a postulated mechanism of action is the reduction of intracellular levels of nitrosoglutathione, a cytoprotectant generated by the overproduction of nitric oxide and found in many brain tumors; nitric oxide is thus believed to react with intracellular glutathione to produce GSNO (nitrosoglutathione). Again, without wishing to be bound by theory, it is believed that HIF (hypoxia inducible factor) could be important in the mechanism of iNOS induction and tumor neovascularization.

This combination treatment is counterintuitive to the use of NO donors and/or endogenously produced nitric oxide as anti-tumor agents, a concept embodied in the Bonavida et al., U.S. patent application Publ. No. 20010038832.

Further, this combination tends to be safer and more effective than the broad-spectrum iNOS inhibitor disclosed by Yin et al. in “Inducible Nitric Oxide Synthase Neutralizes Carbamoylating Potential of 1,3-Bis(2-chloroethyl)-1-nitrosourea in C6 Glioma Cells”, J. Pharmacol. Exp. Therap., Vol. 297, Issue 1, 308-315, (April 2001). More specifically, non-specific or slightly specific inhibitors of iNOS are known to cause serious side effects in subjects, including hypertension and gastrointestinal distress.

Other agents that have carbamoylating activity and are useful in the present methods and combinations include: methyl-CCNU; CCNU; cyclodisone; PCNU; clomesone; chlorozotocin; CBDCA (carboplatin); mitozolamide; triazinate; and L-cysteine analogue.

In some embodiments, the present invention is directed to, for example, a treatment method comprising the administration of the selective iNOS inhibitor and chemotherapeutic agent in conjunction with radiation therapy. In this regard, no order of therapeutic steps are implied. Thus, for example, the radiation therapy may be administered after the chemotherapeutic agent and iNOS inhibitor. Or, for example, the iNOS inhibitor may be administered before radiation therapy, followed by administration of a chemotherapeutic agent.

In some embodiments, the present invention is directed to, for example, a treatment method comprising adminstration of the selective iNOS inhibitor and chemotherapeutic agent in conjunction with resection of a tumor. For example, the selective iNOS inhibitor may be administered following surgery, and subsequently a chemotherapeutic agent may be administered, or surgery may be performed, followed by administration of a selective iNOS inhibitor and a chemotherapeutic agent, for example.

This invention also is directed, in part, to a treatment method comprising resection of a tumor, radiation therapy, administration of a selective iNOS inhibitor, and administration of a cytotoxic agent for therapeutic intervention in a subject with a neoplastic disorder.

This invention also is directed, in part, to a kit comprising a carbamoylating chemotherapeutic agent and a selective iNOS inhibitor in amounts that, when combined, are therapeutically effective.

This invention also is directed, in part, to a medicament comprising a carbamoylating chemotherapeutic agent and a selective iNOS inhibitor in amounts that, when combined, are therapeutically effective.

The foregoing examples are illustrative only, and not limitative. Further benefits of Applicants' invention will be apparent to one skilled in the art from reading this patent.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this patent, and may be variously modified.

In a broad sense, the present invention is directed to agents and methods for the treatment of cancer. Cancers treatable with the present methods include, without limitation: adrenocortical carcinoma, cerebellar astrocytoma, brain stem glioma, ependymoma, medulloblastoma, supratentorial primitive neuroectodermal and pineal tumors, visual pathway and hypothalamic gliomas, astrocytomas including glioblastoma multiforme, primary central nervous system lymphoma, eye cancers including intraocular melanoma and retinoblastoma, head and neck cancer, neuroblastoma, pituitary tumor, meningioma, primitive neuroectodermal tumor and secondary brain tumor.

The combination therapy of the present invention will result in an increase in the anti-tumor effects of BCNU in brain cancer and other human cancers in which BCNU or other carbamoylating cytotoxics are used as standard of care, or may be therapeutically effective.

Thus, in one embodiment of the present invention, an effective amount of a carbamoylating chemotherapeutic agent is administered in combination with an effective amount of a selective iNOS inhibitor to a subject in need of treatment. In this regard, the carbamoylating chemotherapeutic agent may be administered substantially together with the selective iNOS inhibitor, or may, on the other hand, be administered within a therapeutically effective time of administration of the selective iNOS inhibitor.

Optionally, another treatment modality may be applied in conjunction with the therapeutic combination of carbamoylating chemotherapeutic agent and selective iNOS inhibitor. Therefore, in another embodiment of the present invention, surgery, such as resection of a tumor, may be performed prior to administration of the carbamoylating chemotherapeutic agent and the selective iNOS inhibitor. In addition, in another embodiment of the present invention, surgery, such as resection of a tumor, may be performed subsequent to administration of the carbamoylating chemotherapeutic agent and the selective iNOS inhibitor. Of course, surgery, such as resection of a tumor, may be performed prior to administration of the carbamoylating chemotherapeutic agent and subsequent to administration of the selective iNOS inhibitor, and surgery, such as resection of a tumor, may be performed prior to administration of the selective iNOS inhibitor and subsequent to administration of the carbamoylating chemotherapeutic agent as well.

In still another embodiment of the present invention, radiation therapy may be administered to a subject in conjunction with administration of the selective iNOS inhibitor and the carbamoylating chemotherapeutic agent.

In yet another embodiment of the present invention, radiation therapy may be administered to a subject in conjunction with surgery, such as resection of a tumor, in addition to administration of the selective iNOS inhibitor and the carbamoylating chemotherapeutic agent. No specific order in the administration of the above therapies is implied. Therefore, surgery, radiation therapy, chemotherapy and administration of a selective iNOS inhibitor may be performed in any order.

In another embodiment of the present invention, a medicament comprising a carbamoylating chemotherapeutic agent and a selective iNOS inhibitor is prepared for the treatment of cancer.

Exemplary selective iNOS inhibitors useful in the practice of the present invention include:
a compound having Formula I 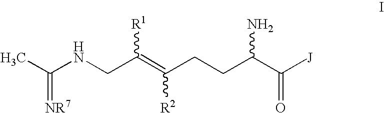
   or a pharmaceutically acceptable salt thereof, wherein:
R¹ is selected from the group consisting of H, halo and alkyl which may be optionally substituted by one or more halo;
R² is selected from the group consisting of H, halo and alkyl which may be optionally substituted by one or more halo;
with the proviso that at least one of R¹ or R² contains a halo;
R⁷ is selected from the group consisting of H and hydroxy;
J is selected from the group consisting of hydroxy, alkoxy, and NR³R⁴ wherein;
R³ is selected from the group consisting of H, lower alkyl, lower alkylenyl and lower alkynyl;
R⁴ is selected from the group consisting of H, and a heterocyclic ring in which at least one member of the ring is carbon and in which 1 to about 4 heteroatoms are independently selected from oxygen, nitrogen and sulfur and the heterocyclic ring may be optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, and acylamino;
a compound having a structure corresponding to Formula II 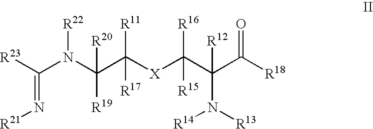
   or a pharmaceutically acceptable salt thereof, wherein X is selected from the group consisting of —S—, —S(O)—, and —S(O)₂—. Preferably, X is —S—. R¹² is selected from the group consisting of C₁-C₆ alkyl, C₂-C₆ alkenyl, C₂-C₆ alkynyl, C₁-C₅ alkoxy-C₁ alkyl, and C₁-C₅ alkylthio-C₁ alkyl wherein each of these groups is optionally substituted by one or more substituent selected from the group consisting of —OH, alkoxy, and halogen. Preferably, R¹² is C₁-C₆ alkyl optionally substituted with a substituent selected from the group consisting of —OH, alkoxy, and halogen. With respect to R¹³ and R¹⁸, R¹⁸ is selected from the group consisting of —OR²⁴ and —N(R²⁵)(R²⁶), and R¹³ is selected from the group consisting of —H, —OH, —C(O)—R²⁷, —C(O)—O—R²⁸, and —C(O)—S—R²⁹; or R¹⁸ is —N(R³⁰)—, and R¹³ is —C(O)—, wherein R¹⁸ and R¹³ together with the atoms to which they are attached form a ring; or R¹⁸ is —O—, and R¹³ is —C(R³¹)(R³²)—, wherein R¹⁸ and R¹³ together with the atoms to which they are attached form a ring. If R¹³ is —C(R3²¹)(R³²)—, then R¹⁴ is —C(O)—O—R³³; otherwise R¹⁴ is —H. R¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-C₁ alkyl. R¹⁹ and R²⁰ independently are selected from the group consisting of —H, C₁-C₆ alkyl, C₂-C₆ alkenyl, C₂-C₆ alkynyl, and C₁-C₅ alkoxy-C₁ alkyl. With respect to R²¹ and R²², R²¹ is selected from the group consisting of —H, —OH, —C(O)—O—R³⁴, and —C(O)—S—R³⁵, and R²² is selected from the group consisting of —H, —OH, —C(O)—O—R³⁶, and —C(O)—S—R³⁷; or R²¹ is —O—, and R²² is —C(O)—, wherein R²¹ and R²² together with the atoms to which they are attached form a ring; or R²¹ is —C(O)—, and R²² is —O—, wherein R²¹ and R²² together with the atoms to which they are attached form a ring. R²³ is C₁ alkyl. R²⁴ is selected from the group consisting of —H and C₁-C₆ alkyl, wherein when R²⁴ is C₁-C₆ alkyl, R²⁴ is optionally substituted by one or more moieties selected from the group consisting of cycloalkyl, heterocyclyl, aryl, and heteroaryl. With respect to R²⁵ and R²⁶, R²⁵ is selected from the group consisting of —H, alkyl, and alkoxy, and R²⁶ is selected from the group consisting of —H, —OH, alkyl, alkoxy, —C(O)—R³⁸, —C(O)—O—R³⁹, and —C(O)—S—R⁴⁰; wherein when R²⁵ and R²⁶ independently are alkyl or alkoxy, R²⁵ and R²⁶ independently are optionally substituted with one or more moieties selected from the group consisting of cycloalkyl, heterocyclyl, aryl, and heteroaryl; or R²⁵ is —H; and R²⁶ is selected from the group consisting of cycloalkyl, heterocyclyl, aryl, and heteroaryl. R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein alkyl is optionally substituted by one or more moieties selected from the group consisting of cycloalkyl, heterocyclyl, aryl, and heteroaryl. When any of R¹¹, R¹², R¹³, R¹⁴, R¹⁵, R¹⁶, R¹⁷, R¹⁸, R19⁹, R²⁰, R²¹, R²², R²³, R²⁴, R²⁵, R²⁶, R²⁷, R²⁸, R²⁹, R³⁰, R³¹, R³², R³³, R³⁴, R³⁵ R³⁶, R³⁷, R³⁸, R³⁹, and R⁴⁰ independently is a moiety selected from the group consisting of alkyl, alkenyl, alkynyl, alkoxy, alkylthio, cycloalkyl, heterocyclyl, aryl, and heteroaryl, then the moiety is optionally substituted by one or more substituent selected from the group consisting of —OH, alkoxy, and halogen;
a compound is represented by Formula III 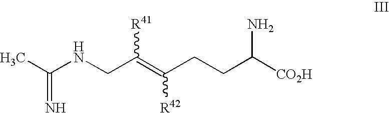
   or a pharmaceutically acceptable salt thereof, wherein:
R⁴¹ is H or methyl; and
R⁴² is H or methyl;
a compound of formula IV 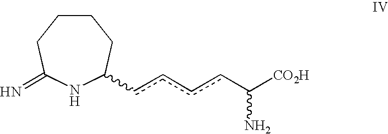
   or a pharmaceutically acceptable salt thereof;
a compound of Formula V: 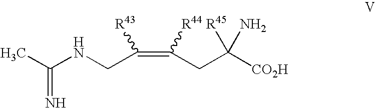
   or a pharmaceutically acceptable salt thereof, wherein:
R⁴³ is selected from the group consisting of hydrogen, halo, C₁-C₅ alkyl and C₁-C₅ alkyl substituted by alkoxy or one or more halo;
R⁴⁴ is selected from the group consisting of hydrogen, halo, C₁-C₅ alkyl and C₁-C₅ alkyl substituted by alkoxy or one or more halo;
R⁴⁵ is C₁-C₅ alkyl or C₁-C₅ alkyl be substituted by alkoxy or one or more halo;
a compound of Formula VI: 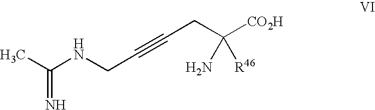
   or a pharmaceutically acceptable salt thereof, wherein:
R⁴⁶ is C₁-C₅ alkyl, the C₁-C₅ alkyl optionally substituted by halo or alkoxy, the alkoxy optionally substituted by one or more halo;
A compound of Formula VII 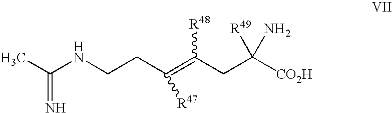
   or a pharmaceutically acceptable salt thereof, wherein:
R⁴⁷ is selected from the group consisting of hydrogen, halo, C₁-C₅ alkyl and C₁-C₅ alkyl substituted by alkoxy or one or more halo;
R⁴⁸ is selected from the group consisting of hydrogen, halo, C₁-C₅ alkyl and C₁-C₅ alkyl substituted by alkoxy or one or more halo;
R⁴⁹ is C₁-C₅ alkyl or C₁-C₅ alkyl be substituted by alkoxy or one or more halo;
a compound of Formula VIII 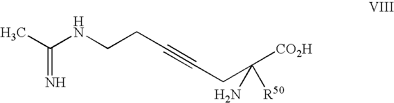
   or a pharmaceutically acceptable salt thereof, wherein:
R⁵⁰ is C₁-C₅ alkyl, the C₁-C₅ alkyl optionally substituted by halo or alkoxy, the alkoxy optionally substituted by one or more halo;
a compound of formula IX 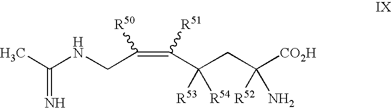
   or a pharmaceutically acceptable salt thereof, wherein:
R⁵⁰ is selected from the group consisting of hydrogen, halo, and C₁-C₅ alkyl, the C₁-C₅ alkyl optionally substituted by halo or alkoxy, the alkoxy optionally substituted by one or more halo;
R⁵¹ is selected from the group consisting of hydrogen, halo, and C₁-C₅ alkyl, the C₁-C₅ alkyl optionally substituted by halo or alkoxy, the alkoxy optionally substituted by one or more halo;
R⁵² is C₁-C₅ alkyl, the C₁-C₅ alkyl optionally substituted by halo or alkoxy, the alkoxy optionally substituted by one or more halo;
R⁵³ is selected from the group consisting of hydrogen, halo, andC₁-C₅ alkyl, the C₁-C₅ alkyl optionally substituted by halo or alkoxy, the alkoxy optionally substituted by one or more halo; and
R⁵⁴ is selected from the group consisting of halo and C₁-C₅ alkyl, the C₁-C₅ alkyl optionally substituted by halo or alkoxy, the alkoxy optionally substituted by one or more halo; and
a compound of formula X 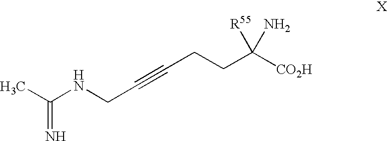
   or a pharmaceutically acceptable salt thereof, wherein:
R⁵⁵ is C₁-C₅ alkyl, the C₁-C₅ alkyl optionally substituted by halo or alkoxy, the alkoxy optionally substituted by one or more halo.

In another exemplary compound, the inducible nitric oxide synthase selective inhibitor is the compound having the formula XI, or a pharmaceutically acceptable thereof. Compound XI has previously been described in International Publication Number WO 00/26195, published May 11, 2000, which is herein incorporated by reference. 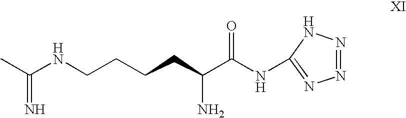
2S-amino-6-[(1-iminoethyl)amino]-N-(1H-tetrazol-5-yl) hexanamide, hydrate, dihydrochloride

The invention also contemplates use of other selective iNOS inhibitors. By way of example, iNOS selective inhibitors also useful in the present invention are described in U.S. Pat. No. 6,355,689, Beswick et al., filed Nov. 29, 2000 and issued Mar. 12, 2002, which describes and claims a selective iNOS inhibitor with the formula XII: 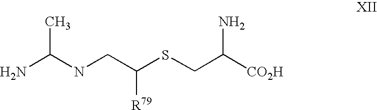
wherein R⁷⁹ is selected from C₁₋₄ alkyl, C₃₋₄ cycloalkyl, C₁₋₄ hydroxyalkyl, and C₁₋₄ haloalkyl. The description of U.S. Pat. No. 6,355,689 states that R⁷⁹ is preferably C₁₋₄ alkyl, and most preferably, methyl. Specific embodiments disclosed in U.S. Pat. No. 6,355,689 and suitable for use in the present methods and compositions include:
S-((R)-2-(1-iminoethylamino)propyl)-L-cysteine;
S-((S)-2-(1-iminoethylamino)propyl)-L-cysteine;
S-((R/S)-2-(1-iminoethylamino)propyl)-L-cysteine;
S-((R)-2-(1-iminoethylamino)propyl)-D-cysteine;
S-((S)-2-(1-iminoethylamino)propyl)-D-cysteine;
S-((R/S)-2-(1-iminoethylamino)propyl)-D-cysteine;
S-((R/S)-2-(1-iminoethylamino)butyl)-L-cysteine;
S-((R/S)-2-(1-iminoethylamino,2-cyclopropyl)ethyl)-L-cysteine; and
S-((R/S)-2-(1-iminoethylamino,3-hydroxy)propyl)-L-cysteine,
or a pharmaceutically acceptable salt, solvate, or physiologically functional derivative thereof.

The above selective iNOS inhibitors are believed to work by competing with arginine as a substrate for the iNOS enzyme. Another strategy for inhibition of iNOS has been described by Arnaiz et al. in international patent application number PCT/US98/03176, publication number WO 98/37079 (Berlex Laboratories, Inc. Richmond, Calif. 94804-0099 and Pharmacopeia, Inc. Princeton, N.J. 08540), published Aug. 27, 1998 (Arnaiz). The Arnaiz application describes inhibitors of iNOS monomer dimerization. The iNOS enzyme is a homodimer; each monomer has a reductase domain, incorporating binding sites for flavin cofactors (FAD and FMN) and for NADPH. The reductase domain supplies electrons to the oxidase domain of the other monomer, where L-arginine is oxidized at the active site, which incorporates a heme group (Fe) cytochrome P-450 domain. Tetrahydrobiopterin (BH4) is required for homodimerization and modulates the heme redox state during electron transfer. iNOS monomers are inactive, and dimerization is required for activity.

Thus, in another embodiment of the present invention, the selective iNOS inhibitor is a dimerization inhibitor represented by a compound of Formula XIII, Formula XIV or Formula XV: 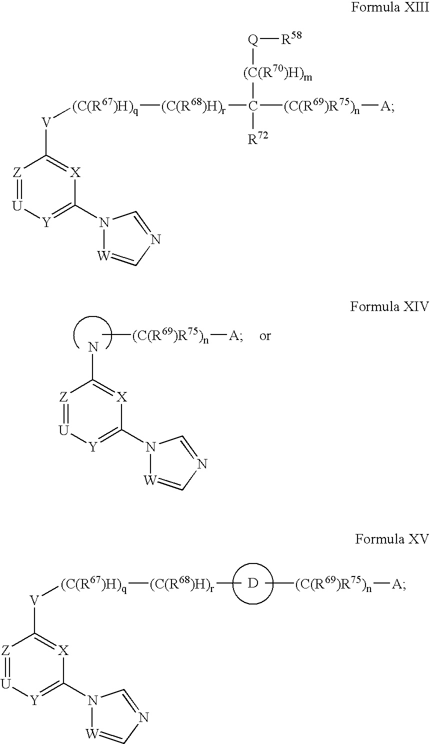
wherein:
A is —R⁵⁶, —OR⁵⁶, C(O)N(R⁵⁶)R⁵⁷, P(O)[N(R⁵⁶)R⁵⁷]₂, —N(R⁵⁶)C(O)R⁵⁷, —N(R⁷⁶)C(O)OR⁵⁶, —N(R⁵⁶)R⁷⁶,
—N(R⁷¹)C(O)N(R⁵⁶)R⁷¹, —S(O)ₜR⁵⁶, —SO₂NHC(O)R⁵⁶, —NHSO₂R⁷⁷, —SO₂NH(R⁵⁶)H, —C(O)NHSO₂R⁷⁷, and —CH═NOR⁵⁶;
each X, Y and Z are independently N or C(R¹⁹);
each U is N or C(R⁶⁰), provided that U is N only when X is N and Z and Y are CR⁷⁴;
V is N(R⁵⁹), S, O or C(R⁵⁹)H;
Each W is N or CH;
Q is chosen from the group consisting of a direct bond, —C(O)—, —O—, —C(═N—R⁵⁶)—, S(O)ₜ, and —N(R⁶¹)—;
m is zero or an integer from 1 to 4;
n is zero or an integer from 1 to 3;
q is zero or one;
r is zero or one, provided that when Q and V are heteroatoms, m, q, and r cannot all be zero;
when A is —OR⁵⁶, N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —S(O)ₜR⁵⁶ (where t is zero), or —NHSO₂R⁷⁷, n, q, and r cannot all be zero; and when Q is a heteroatom and A is —OR⁵⁶, N(R⁵⁶)C(O)R⁵⁷, —N(R⁷¹)C(O)OR⁵⁷, —N(R⁵⁶)R⁷⁶, N(R⁷¹)C(O)N(R⁵⁶)R⁷¹, —S(O)ₜR⁵⁶ (when t is zero), or —NHSO₂R⁷⁷, m and n cannot both be zero;
t is zero, one or two; 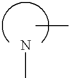
   is an optionally substituted N-heterocyclyl; 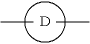
   is an optionally substituted carbocyclyl or optionally substituted N-heterocyclyl;
each R⁵⁶ and R⁵⁷ are independently chosen from the group consisting of hydrogen, optionally substituted C₁-C₂₀ alkyl, optionally substituted cycloalkyl,
—[C₀-C₈ alkyl]-R⁶⁴, —[C₂-C₈ alkenyl]-R⁶⁴, —[C₂-C₈ alkynyl]-R⁶⁴, —[C₂-C₈ alkyl]-R⁶⁵ (optionally substituted by hydroxy), —[C₁-C₈]—R⁶⁶ (optionally substituted by hydroxy), optionally substituted heterocyclyl;
or R⁵⁶ and R⁵⁷ together with the nitrogen atom to which they are attached is an optionally substituted N-heterocyclyl;
R⁵⁸ is chosen from the group consisting of hydrogen, alkyl, cycloalkyl, optionally substituted aryl, haloalkyl, —[C₁-C₈ alkyl]-C(O)N(R⁵⁶)R⁵⁷,
—[C₁-C₈ alkyl]-N(R⁵⁶)R⁵⁷, —[C₁-C₈ alkyl]-R⁶³, —[C₂-C₈ alk2yl]-R⁶⁵,
—[C₁-C₈ alkyl]-R⁶⁶, and heterocyclyl (optionally substituted by one or more substitutents selected from the group consisting of halo, alkyl, alkoxy and imidazolyl);
or when Q is —N(R⁵⁸)— or a direct bond to R⁵⁸, R⁵⁸ may additionally be aminocarbonyl,
alkoxycarbonyl, alkylsulfonyl, monoalkylaminocarbonyl, dialkylaminocarbonyl and —C(═NR⁷³)—NH₂;
or -Q-R⁵⁸ taken together represents —C(O)OH, —C(O)N(R⁵⁶)R⁵⁷ or 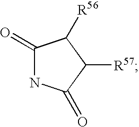
R⁵⁹ is chosen from the group consisting of hydrogen, alkyl, aryl, aralkyl and cycloalkyl;
provided that when A is —R⁵⁶ or —OR⁵⁶, R⁵⁹ cannot be hydrogen, and when V is CH, R⁵⁹ may additionally be hydroxy;
R⁶⁰ is chosen from the group consisting of hydrogen, alkyl, aryl, aralkyl, haloalkyl,
optionally substituted aralkyl, optionally substituted aryl, —OR⁷¹, —S(O)ₜ—R⁷¹, N(R⁷¹)R⁷⁶, N(R⁷¹)C(O)N(R⁵⁶)R⁷¹, N(R⁷¹)C(O)OR⁷¹, N(R⁷¹)C(O) R⁷¹, —[C₀-C₈ alkyl]—C(H)[C(O)R⁷¹]₂ and —[C₀-C₈ alkyl]-C(O)N(R⁵⁶)R⁷¹;
R⁶¹ is chosen from the group consisting of hydrogen, alkyl, cycloalkyl,
—[C₁-C₈ alkyl]-R⁶³, —[C₂-C₈]alkyl]-R⁶⁵, —[C₁-C₈ alkyl]-R⁶⁶, acyl, —C(O)R⁶³,
—C(O)——[C₁-C₈ alkyl]-R⁶³, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl,
monoalkylaminosulfonyl dialkylaminosulfonyl, arylaminosulfonyl, arylsulfonylaminocarbonyl, optionally substituted N-heterocyclyl, —C(═NH)—N(CN)R⁵⁶, —C(O)R⁷⁸—N(R⁵⁶)R⁵⁷, —C(O)—N(R⁵⁶)R⁷⁸—C(O)OR⁵⁶;
each R⁶³ and R⁶⁴ are independently chosen from the group consisting of haloalkyl,
cycloalkyl, (optionally substituted with halo, cyano, alkyl or alkoxy), carbocyclyl (optionally substituted with one or more substituents selected from the group consisting of halo, alkyl and alkoxy) and heterocyclyl (optionally substituted with alkyl, aralkyl or alkoxy);
each R⁶⁵ is independently chosen from the group consisting of halo, alkoxy, optionally
substituted aryloxy, optionally substituted aralkoxy, optionally substituted —S(O)ₜ—R⁷⁷, acylamino, amino, monoalkylamino, dialkylamino, (triphenylmethyl)amino, hydroxy, mercapto, alkylsulfonamido;
each R⁶⁶ is independently chosen from the group consisting of cyano, di(alkoxy)alkyl,
carboxy, alkoxycarbonyl, aminocarbonyl, monoalkylaminocarbonyl and dialkylaminocarbonyl;
each R⁶⁷, R⁶⁸, R⁶⁹, R⁷⁰, R⁷², and R⁷⁵ are independently hydrogen or alkyl;
each R⁷¹ is independently hydrogen, alkyl, optionally substituted aryl, optionally
substituted aralkyl or cycloalkyl;
R⁷³ is hydrogen, NO₂, or toluenesulfonyl;
each R⁷⁴ is independently hydrogen, alkyl (optionally substituted with hydroxy),
cyclopropyl, halo or haloalkyl;
each R⁷⁶ is independently hydrogen, alkyl, cycloalkyl, optionally substituted aryl,
optionally substituted aralkyl, —C(O)R⁷⁷ or —SO₂R⁷⁷;
or R⁷⁶ taken together with R⁵⁶ and the nitrogen to which they are attached is an optionally
substituted N-heterocyclyl;
or R⁷⁶ taken together with R⁷¹ and the nitrogen to which they are attached is an optionally
substituted N-heterocyclyl;
each R⁷⁷ is independently alkyl, cycloalkyl, optionally substituted aryl or optionally
substituted aralkyl; and
R⁷⁸ is an amino acid residue;
as a single stereoisomer or mixture thereof, or a pharmaceutically acceptable salt thereof.

Another iNOS dimerization inhibitor, 3-(2,4-difluorophenyl)-6-{2-[4-(1H-imidazol-1-ylmethyl) phenoxy]ethoxy}-2-phenylpyridine (PPA250) has been described in Ohtsuka et al., J Phamacol Exp Ther Vol. 303, Issue 1, 52-57, October 2002. PPA250 has the structure:

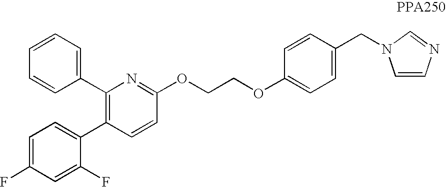

In one illustrative example of a selective iNOS inhibitor, treatment is facilitated through compounds having Formula I: 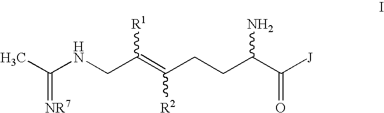
or a pharmaceutically acceptable salt or prodrug thereof, wherein:
R¹ is selected from the group consisting of H, halo and alkyl which may be optionally substituted by one or more halo;
R² is selected from the group consisting of H, halo and alkyl which may be optionally substituted by one or more halo;
with the proviso that at least one of R¹ or R² contains a halo;
R⁷ is selected from the group consisting of H and hydroxy; and
J is selected from the group consisting of hydroxy, alkoxy, and NR³R⁴ wherein;
R³ is selected from the group consisting of H, lower alkyl, lower alkylenyl and lower alkynyl; and R⁴ is selected from the group consisting of H, and a heterocyclic ring in which at least one member of the ring is carbon and in which 1 to about 4 heteroatoms are independently selected from oxygen, nitrogen and sulfur and the heterocyclic ring may be optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, and acylamino.

In another embodiment, the present invention provides treatment utilizing a compound or a salt thereof, the compound having a structure corresponding to Formula II: 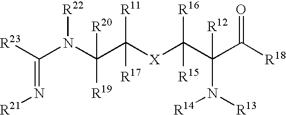
or a pharmaceutically acceptable salt or prodrug thereof.

In the structure of Formula II, X is selected from the group consisting of —S—, —S(O)—, and —S(O)₂—. Preferably, X is —S—. R¹² is selected from the group consisting of C₁-C₆ alkyl, C₂-C₆ alkenyl, C₂-C₆ alkynyl, C₁-C₅ alkoxy-C₁ alkyl, and C₁-C₅ alkylthio-C₁ alkyl wherein each of these groups is optionally substituted by one or more substituent selected from the group consisting of —OH, alkoxy, and halogen. Preferably, R¹² is C₁-C₆ alkyl optionally substituted with a substituent selected from the group consisting of —OH, alkoxy, and halogen. With respect to R¹³ and R¹⁸, R¹⁸ is selected from the group consisting of —OR²⁴ and —N(R²⁵)(R²⁶), and R¹³ is selected from the group consisting of —H, —OH, —C(O)—R²⁷, —C(O)—O—R²⁸, and —C(O)—S—R²⁹; or R¹⁸ is —N(R³⁰)—, and R³ is —C(O)—, wherein R¹⁸ and R¹³ together with the atoms to which they are attached form a ring; or R¹⁸ is —O—, and R¹³ is —C(R³¹)(R³²)—, wherein R¹⁸ and R¹³ together with the atoms to which they are attached form a ring. If R¹³ is —C(R3²¹)(R³²)—, then R¹⁴ is —C(O)—O—R³³; otherwise R^{is —H. R}¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-C₁ alkyl. R¹⁹ and R²⁰ independently are selected from the group consisting of —H, C₁-C₆ alkyl, C₂-C₆ alkenyl, C₂-C₆ alkynyl, and C₁-C₅ alkoxy-C₁ alkyl. With respect to R²¹ and R²², R²¹ is selected from the group consisting of —H, —OH, —C(O)—O—R³⁴, and —C(O)—S—R³⁵, and R²² is selected from the group consisting of —H, —OH, —C(O)—O—R³⁶, and —C(O)—S—R³⁷; or R²¹ is —O—, and R²² is —C(O)—, wherein R²¹ and R²² together with the atoms to which they are attached form a ring; or R²¹ is —C(O)—, and R²² is —O—, wherein R²¹ and R²² together with the atoms to which they are attached form a ring. R²³ is C₁ alkyl. R²⁴ is selected from the group consisting of —H and C₁-C₆ alkyl, wherein when R²⁴ is C₁-C₆ alkyl, R²⁴ is optionally substituted by one or more moieties selected from the group consisting of cycloalkyl, heterocyclyl, aryl, and heteroaryl. With respect to R²⁵ and R²⁶, R²⁵ is selected from the group consisting of —H, alkyl, and alkoxy, and R²⁶ is selected from the group consisting of —H, —OH, alkyl, alkoxy, —C(O)—R³⁸, —C(O)—O—R³⁹, and —C(O)—S—R⁴⁰; wherein when R²⁵ and R²⁶ independently are alkyl or alkoxy, R²⁵ and R²⁶ independently are optionally substituted with one or more moieties selected from the group consisting of cycloalkyl, heterocyclyl, aryl, and heteroaryl; or R²⁵ is —H; and R²⁶ is selected from the group consisting of cycloalkyl, heterocyclyl, aryl, and heteroaryl. R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein alkyl is optionally substituted by one or more moieties selected from the group consisting of cycloalkyl, heterocyclyl, aryl, and heteroaryl. When any of R¹¹, R¹², R¹³, R¹⁴, R¹⁵, R¹⁶, R¹⁷, R¹⁸, R19⁹, R²⁰, R²¹, R²², R²³, R²⁴, R²⁵, R²⁶, R²⁷, R²⁸, R²⁹, R³⁰, R³¹, R³², R³³, R³⁴, R³⁵ R³⁶, R³⁷, R³⁸, R³⁹, and R⁴⁰ independently is a moiety selected from the group consisting of alkyl, alkenyl, alkynyl, alkoxy, alkylthio, cycloalkyl, heterocyclyl, aryl, and heteroaryl, then the moiety is optionally substituted by one or more substituent selected from the group consisting of —OH, alkoxy, and halogen.

In a preferred compound, R¹⁸ is —OH. When R¹⁸ is —OH, preferably X is S. In a further compound, R¹¹, R¹⁵, R¹⁶, R¹⁷, R¹⁹, and R²⁰ independently are selected from the group consisting of —H and C₁-C₃ alkyl. Preferably R¹⁵, R¹⁶, R¹⁷, R¹⁹, R²⁰ each are —H. R²³ can be a variety of groups, for example fluoromethyl or methyl. R¹¹ can be C₁-C₆ alkyl optionally substituted with a substituent selected from the group consisting of —OH and halogen; preferably R¹¹ is C₁ alkyl optionally substituted with halogen; more preferably R¹¹ is selected from the group consisting of fluoromethyl, hydroxymethyl, and methyl. In one important compound, R¹¹ can be methyl. Alternatively, R¹¹ can be fluoromethyl. In another alternative R¹¹ can be hydroxymethyl. In another compound, R¹² is C₁-C₆ alkyl optionally substituted with a substituent selected from the group consisting of —OH, alkoxy, and halogen. In one preferred compound R¹² is C₁ alkyl optionally substituted with halogen. For example, R¹² can be methyl. Alternatively, R¹² can be fluoromethyl. In yet another example, R¹² can be hydroxymethyl. In still another example, R¹² can be methoxymethyl.

In this exemplary compound, it is preferred that R¹³, R¹⁴, R²¹ and R²² each is —H. In this compound, it is further preferred that R¹¹, R¹⁵, R¹⁶, R¹⁷, R¹⁹, and R²⁰ independently are selected from the group consisting of —H and C₁-C₃ alkyl. Preferably R¹⁵, R¹⁶, R¹⁷, R¹⁹, R²⁰ each is —H. In this further compound, R²³ can be, for example, fluoromethyl, or in another example R²³ can be methyl. In preferred compounds of these examples, R¹² is C₁-C₆ alkyl optionally substituted with a substituent selected from the group consisting of —OH, alkoxy, and halogen. Preferably R¹² is C₁ alkyl optionally substituted with halogen. In one such example R¹² is fluoromethyl. In another example R¹² is methyl. Alternatively R¹² can be hydroxymethyl. In another alternative, R¹² can be methoxymethyl.

When R²³ is methyl, R¹¹ can be, for example, —H or C₁-C₆ alkyl optionally substituted with a substituent selected from the group consisting of —OH and halogen. In a preferred compound R¹¹ is —H. Alternatively, R¹¹ can be C₁-C₆ alkyl optionally substituted with a substituent selected from the group consisting of —OH and halogen. For example R¹¹ can be methyl, ethyl, n-propyl, i-propyl, n-butyl, sec-butyl, isobutyl, t-butyl, a pentyl isomer, or a hexyl isomer. For example, R¹¹ can be ethyl. Alternatively, R¹¹ can be C₁ alkyl optionally substituted with a substituent selected from the group consisting of —OH and halogen; for example R¹¹ can be methyl. Alternatively, R¹¹ can be fluoromethyl. In another alternative, R¹¹ can be hydroxymethyl.

In another compound R¹⁸ can be —OR²⁴. R²⁴ can be as defined above. Preferably R²⁴ is C₁-C₆ alkyl optionally substituted by one or more moieties selected from the group consisting of cycloalkyl, heterocyclyl, aryl, and heteroaryl; more preferably R²⁴ is C₁-C₃ alkyl; and more preferably still R²⁴ is methyl. In yet another example of compound II, R¹⁸ can be —N(R²⁵)(R²⁶), wherein R²⁵ and R²⁶ are as defined above. In still another compound, R¹⁸ can be —N(R³⁰)—, and R¹³ can be —C(O)—, wherein R¹⁸ and R¹³ together with the atoms to which they are attached form a ring. In another example still, R¹⁸ can be —O—, and R¹³ can be —C(R³¹)(R³²)—, wherein R¹⁸ and R¹³ together with the atoms to which they are attached form a ring.

In a compound of Formula II, R²¹ can be selected from the group consisting of —OH, —C(O)—O—R³⁴, and —C(O)—S—R³⁵. Preferably R²¹ is —OH. In a further example, R²² is —H when R²¹ is —OH.

However, the present example also provides useful compounds of Formula II in which R²¹ is —O—, and R²² is —C(O)—, wherein R²¹ and R²² together with the atoms to which they are attached form a ring. In another useful compound, R²¹ is —C(O)—, and R²² is —O—, wherein R²¹ and R²² together with the atoms to which they are attached form a ring. Alternatively, R²² can be selected from the group consisting of —OH, —C(O)—O—R³⁶, and —C(O)—S—R³⁷. In this alternative, R²¹ is preferably —H.

In another selective iNOS inhibitor useful in the practice of the present invention, a compound is represented by Formula III: 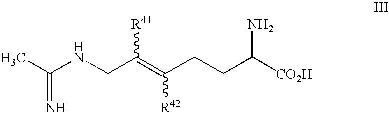
or a pharmaceutically acceptable salt or prodrug thereof, wherein:
R⁴¹ is H or methyl; and
R⁴² is H or methyl.

Another selective iNOS inhibitor useful in the practice of the present invention is represented by a compound of formula IV 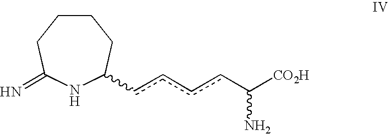
or a pharmaceutically acceptable salt or prodrug thereof.

Another exemplary selective iNOS inhibitor useful in the present invention is represented by Formula V: 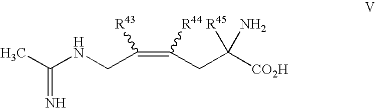
or a pharmaceutically acceptable salt or prodrug thereof, wherein:
R⁴³ is selected from the group consisting of hydrogen, halo, C₁-C₅ alkyl and C₁-C₅ alkyl substituted by alkoxy or one or more halo;
R⁴⁴ is selected from the group consisting of hydrogen, halo, C₁-C₅ alkyl and C₁-C₅ alkyl substituted by alkoxy or one or more halo;
R⁴⁵ is C₁-C₅ alkyl or C₁-C₅ alkyl be substituted by alkoxy or one or more halo.

A further illustrative selective iNOS inhibitor is represented by Formula VI: 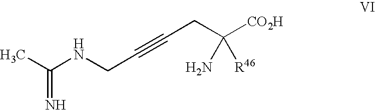
or a pharmaceutically acceptable salt or prodrug thereof, wherein:
R⁴⁶ is C₁-C₅ alkyl, the C₁-C₅ alkyl optionally substituted by halo or alkoxy, the alkoxy optionally substituted by one or more halo.

Another exemplary selective iNOS inhibitor useful in the present invention by Formula VII 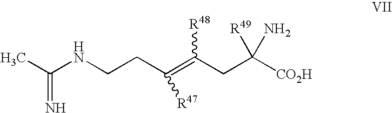
or a pharmaceutically acceptable salt or prodrug thereof, wherein:
R⁴⁷ is selected from the group consisting of hydrogen, halo, C₁-C₅ alkyl and C₁-C₅ alkyl substituted by alkoxy or one or more halo;
R⁴⁸ is selected from the group consisting of hydrogen, halo, C₁-C₅ alkyl and C₁-C₅ alkyl substituted by alkoxy or one or more halo;
R⁴⁹ is C₁-C₅ alkyl or C₁-C₅ alkyl be substituted by alkoxy or one or more halo.

Another exemplary selective iNOS inhibitor useful in the present invention is represented by Formula VIII 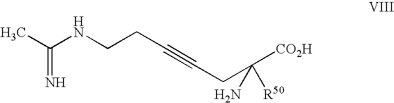
or a pharmaceutically acceptable salt or prodrug thereof, wherein:
R⁵⁰ is C₁-C₅ alkyl, the C₁-C₅ alkyl optionally substituted by halo or alkoxy, the alkoxy optionally substituted by one or more halo.

Another selective iNOS inhibitor useful in the practice of the present invention is represented by a compound of formula IX 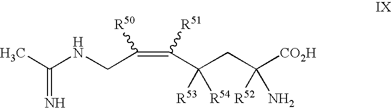
or a pharmaceutically acceptable salt or prodrug thereof, wherein:
R⁵⁰ is selected from the group consisting of hydrogen, halo, and C₁-C₅ alkyl, the C₁-C₅ alkyl optionally substituted by halo or alkoxy, the alkoxy optionally substituted by one or more halo;
R⁵¹ is selected from the group consisting of hydrogen, halo, and C₁-C₅ alkyl, the C₁-C₅ alkyl optionally substituted by halo or alkoxy, the alkoxy optionally substituted by one or more halo;
R⁵² is C₁-C₅ alkyl, the C₁-C₅ alkyl optionally substituted by halo or alkoxy, the alkoxy optionally substituted by one or more halo;
R⁵³ is selected from the group consisting of hydrogen, halo, andC₁-C₅ alkyl, the C₁-C₅ alkyl optionally substituted by halo or alkoxy, the alkoxy optionally substituted by one or more halo; and
R⁵⁴ is selected from the group consisting of halo and C₁-C₅ alkyl, the C₁-C₅ alkyl optionally substituted by halo or alkoxy, the alkoxy optionally substituted by one or more halo.

Yet another selective iNOS inhibitor useful in the practice of the present invention is represented by a compound of formula X 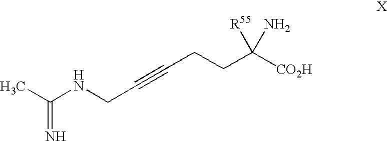
or a pharmaceutically acceptable salt or prodrug thereof, wherein:
R⁵⁵ is C₁-C₅ alkyl, the C₁-C₅ alkyl optionally substituted by halo or alkoxy, the alkoxy optionally substituted by one or more halo.

In another exemplary compound, the inducible nitric oxide synthase selective inhibitor is the compound having the formula XI, or a pharmaceutically acceptable thereof. Compound XI has previously been described in International Publication Number WO 00/26195, published May 11, 2000, which is herein incorporated by reference. 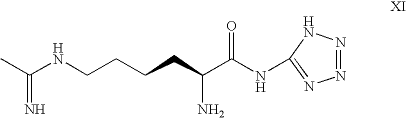
(2S-amino-6-[(1-iminoethyl)amino]-N-(1H-tetrazol-5-yl) hexanamide, hydrate, dihydrochloride).

In another embodiment of the present invention, a selective iNOS inhibitor with the formula XII: 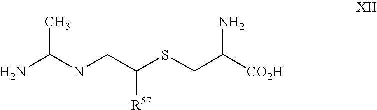
wherein R⁵⁷ is selected from C₁₋₄ alkyl, C₃₋₄ cycloalkyl, C₁₋₄ hydroxyalkyl, and C₁₋₄ haloalkyl may be used in the practice of the present invention. The description of U.S. Pat. No. 6,355,689 states that R⁵⁷ is preferably C₁₋₄ alkyl, and most preferably, methyl. Specific embodiments disclosed in U.S. Pat. No. 6,355,689 and suitable for use in the present methods and compositions include:
S-((R)-2-(1-iminoethylamino)propyl)-L-cysteine;
S-((S)-2-(1-iminoethylamino)propyl)-L-cysteine;
S-((R/S)-2-(1-iminoethylamino)propyl)-L-cysteine;
S-((R)-2-(1-iminoethylamino)propyl)-D-cysteine;
S-((S)-2-(1-iminoethylamino)propyl)-D-cysteine;
S-((R/S)-2-(1-iminoethylamino)propyl)-D-cysteine;
S-((R/S)-2-(1-iminoethylamino)butyl)-L-cysteine;
S-((R/S)-2-(1-iminoethylamino,2-cyclopropyl)ethyl)-L-cysteine; and
S-((R/S)-2-(1-iminoethylamino,3-hydroxy)propyl)-L-cysteine,
or a pharmaceutically acceptable salt, solvate, or physiologically functional derivative thereof is employed as the selective iNOS inhibitor of the present invention.

In yet another embodiment of the present invention, the selective iNOS inhibitor is a dimerization inhibitor represented by a compound of Formula XIII, Formula XIV or Formula XV: 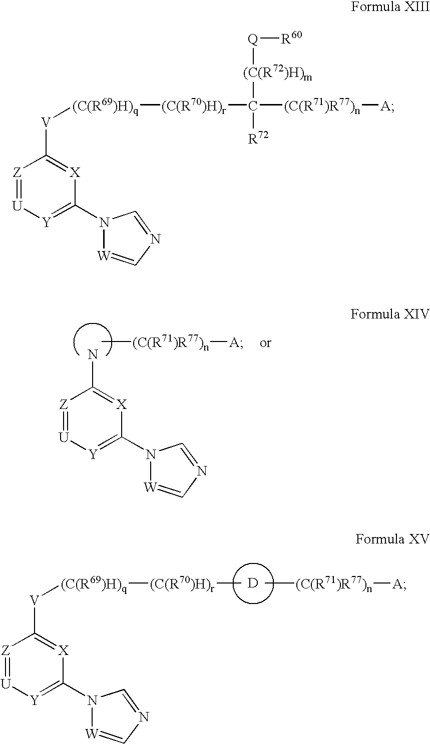
wherein:
A is —R⁵⁸, —OR⁵⁸, C(O)N(R⁵⁸)R⁵⁹, P(O)[N(R⁵⁸)R⁵⁹]₂, —N(R⁵⁸)C(O)R⁵⁹, —N(R⁷⁸)C(O)OR⁵⁸, —N(R⁵⁸)R⁷⁸,
—N(R⁷³)C(O)N(R⁵⁶)R⁷³, —S(O)ₜR⁵⁸, —SO₂NHC(O)R⁵⁸, —NHSO₂R⁷⁹, —SO₂NH(R⁵⁸)H, —C(O)NHSO₂R⁷⁹, and —CH═NOR⁵⁸;
each X, Y and Z are independently N or C(R⁷⁶);
each U is N or C(R⁶²), provided that U is N only when X is N and Z and Y are CR⁷⁶;
V is N(R⁶¹), S, O or C(R⁶¹e)H;
Each W is N or CH;
Q is chosen from the group consisting of a direct bond, —C(O)—, —O—, —C(═N—R⁵⁸)—, S(O)ₜ, and —N(R⁶³)—;
m is zero or an integer from 1 to 4;
n is zero or an integer from 1 to 3;
q is zero or one;
r is zero or one, provided that when Q and V are heteroatoms, m, q, and r cannot all be zero;
when A is —OR⁵⁸, N(R⁵⁸)C(O)R⁵⁹, —N(R⁷³)C(O)OR⁵⁹, —N(R⁵⁸)R⁷⁸, —N(R⁷³)C(O)N(R⁵⁸)R⁷³, —S(O)ₜR⁵⁸ (where t is zero), or —NHSO₂R⁷⁹, n, q, and r cannot all be zero; and when Q is a heteroatom and A is —OR⁵⁸, N(R⁵⁸)C(O)R⁵⁹, —N(R⁷³)C(O)OR⁵⁹, —N(R⁵⁸)R⁷⁸, N(R⁷³ )C(O)N(R⁵⁸)R⁷³, —S(O)ₜR⁵⁸ (when t is zero), or —NHSO₂R⁷⁹, m and n cannot both be zero;
t is zero, one or two; 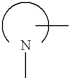
   is an optionally substituted N-heterocyclyl; 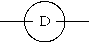
   is an optionally substituted carbocyclyl or optionally substituted N-heterocyclyl;
each R⁵⁸ and R⁵⁹ are independently chosen from the group consisting of hydrogen, optionally substituted C₁-C₂₀ alkyl, optionally substituted cycloalkyl,
—[C₀-C₈ alkyl]-R⁶⁶, —[C₂-C₈ alkenyl]-R⁶⁶, —[C₂-C₈ alkynyl]-R⁶⁶, —[C₂-C₈ alkyl]-R⁶⁷ (optionally substituted by hydroxy), —[C₁-C₈ alkyl]-R⁶⁸ (optionally substituted by hydroxy), optionally substituted heterocyclyl;
or R⁵⁸ and R⁵⁹ together with the nitrogen atom to which they are attached is an optionally substituted N-heterocyclyl;
R⁶⁰ is chosen from the group consisting of hydrogen, alkyl, cycloalkyl, optionally substituted aryl, haloalkyl, —[C₁-C₈ alkyl]-C(O)N(R⁵⁸)R⁵⁹,
—[C₁-C₈ alkyl]-N(R⁵⁸)R⁵⁹, —[C₁-C₈ alkyl]-R⁶⁵, —[C₂-C₈ alkyl]-R⁶⁷,
—[C₁-C₈ alkyl]-R⁶⁸, and heterocyclyl (optionally substituted by one or more substitutents selected from the group consisting of halo, alkyl, alkoxy and imidazolyl);
or when Q is —N(R⁶⁰)— or a direct bond to R⁶⁰, R⁶⁰ may additionally be aminocarbonyl,
alkoxycarbonyl, alkylsulfonyl, monoalkylaminocarbonyl, dialkylaminocarbonyl and —C(═NR⁷⁵)—NH₂;
or -Q-R⁶⁰ taken together represents —C(O)OH, —C(O)N(R⁵⁸)R⁵⁹ or 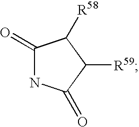
R⁶¹ is chosen from the group consisting of hydrogen, alkyl, aryl, aralkyl and cycloalkyl;
Provided that when A is —R⁵⁸ or —OR⁵⁸, R⁶¹ cannot be hydrogen, and when V is CH, R⁶¹ may additionally be hydroxy;
R⁶² is chosen from the group consisting of hydrogen, alkyl, aryl, aralkyl, haloalkyl,
optionally substituted aralkyl, optionally substituted aryl, —OR⁷³, —S(O)ₜ—R⁷³, N(R⁷³)R⁷⁸, N(R⁷³)C(O)N(R⁵⁸)R⁷³, N(R⁷³)C(O)OR⁷³, N(R⁷³)C(O) R⁷³, —[C₀-C₈ alkyl]-C(H)[C(O)R⁷³]₂ and —[C₀-C₈ alkyl]-C(O)N(R⁵⁸)R⁷³;
R⁶³ is chosen from the group consisting of hydrogen, alkyl, cycloalkyl,
—[C₁-C₈ alkyl]-R⁶⁵, —[C₂-C₈]alkyl]-R⁶⁷, —[C₁-C₈ alkyl]-R⁶⁸, acyl, —C(O)R⁶⁵,
—C(O)——[C₁-C₈ alkyl]-R⁶⁵, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl,
monoalkylaminosulfonyl dialkylaminosulfonyl, arylaminosulfonyl, arylsulfonylaminocarbonyl, optionally substituted N-heterocyclyl, —C(═NH)—N(CN)R⁵⁸, —C(O)R⁸⁰—N(R⁵⁸)R⁵⁹, —C(O)—N(R⁵⁸)R⁸⁰—C(O)OR⁵⁸;
each R⁶⁵ and R⁶⁶ are independently chosen from the group consisting of haloalkyl,
cycloalkyl, (optionally substituted with halo, cyano, alkyl or alkoxy), carbocyclyl (optionally substituted with one or more substituents selected from the group consisting of halo, alkyl and alkoxy) and heterocyclyl (optionally substituted with alkyl, aralkyl or alkoxy);
each R⁶⁷ is independently chosen from the group consisting of halo, alkoxy, optionally
substituted aryloxy, optionally substituted aralkoxy, optionally substituted —S(O)ₜ—R⁷⁹, acylamino, amino, monoalkylamino, dialkylamino, (triphenylmethyl)amino, hydroxy, mercapto, alkylsulfonamido;
each R⁶⁸ is independently chosen from the group consisting of cyano, di(alkoxy)alkyl,
carboxy, alkoxycarbonyl, aminocarbonyl, monoalkylaminocarbonyl and dialkylaminocarbonyl;
each R⁶⁹, R⁷⁰, R⁷¹, R⁷², R⁷⁴, and R⁷⁷ are independently hydrogen or alkyl;
each R⁷³ is independently hydrogen, alkyl, optionally substituted aryl, optionally
substituted aralkyl or cycloalkyl;
R⁷⁵ is hydrogen, NO₂, or toluenesulfonyl;
each R⁷⁶ is independently hydrogen, alkyl (optionally substituted with hydroxy),
cyclopropyl, halo or haloalkyl;
each R⁷⁸ is independently hydrogen, alkyl, cycloalkyl, optionally substituted aryl,
optionally substituted aralkyl, —C(O)R⁷⁹ or —SO₂R⁷⁹;
or R⁷⁵ taken together with R⁵⁸ and the nitrogen to which they are attached is an optionally
substituted N-heterocyclyl;
or R⁷⁸ taken together with R⁷³ and the nitrogen to which they are attached is an optionally
substituted N-heterocyclyl;
each R⁷⁹ is independently alkyl, cycloalkyl, optionally substituted aryl or optionally
substituted aralkyl; and
R⁸⁰ is an amino acid residue;
as a single stereoisomer or mixture thereof, or a pharmaceutically acceptable salt thereof.

In another embodiment of the present invention, the compound PPA250, 3-(2,4-difluorophenyl)-6-{2-[4-(1H-imidazol-1-ylmethyl) phenoxy]ethoxy}-2-phenylpyridine, may be employed as the selective iNOS inhibitor.

Several selective substrate iNOS inhibitors are particularly preferred in the practice of the present invention, as described more fully herein below. Therefore, in another embodiment of the present invention, the selective iNOS inhibitor is selected from the group consisting of: 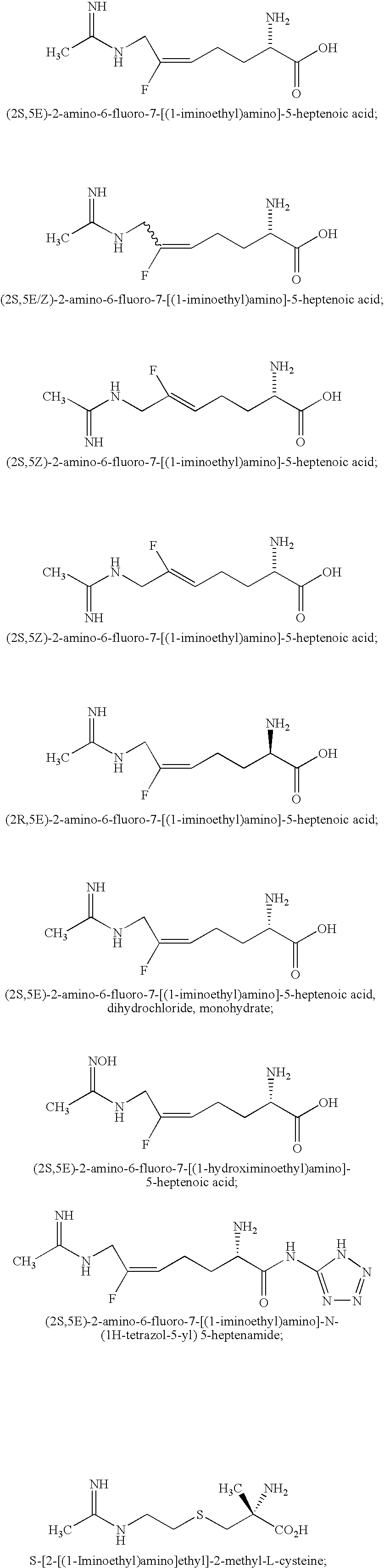 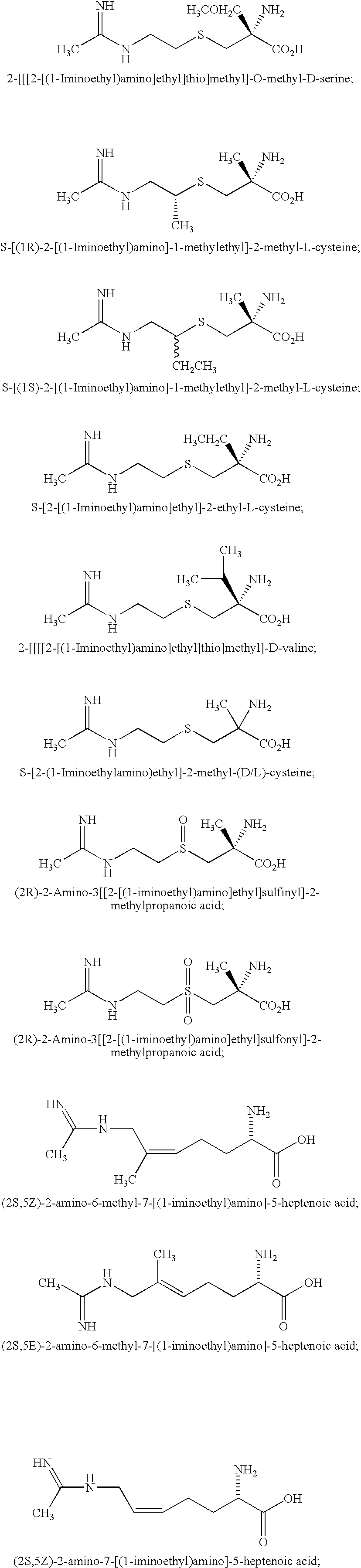 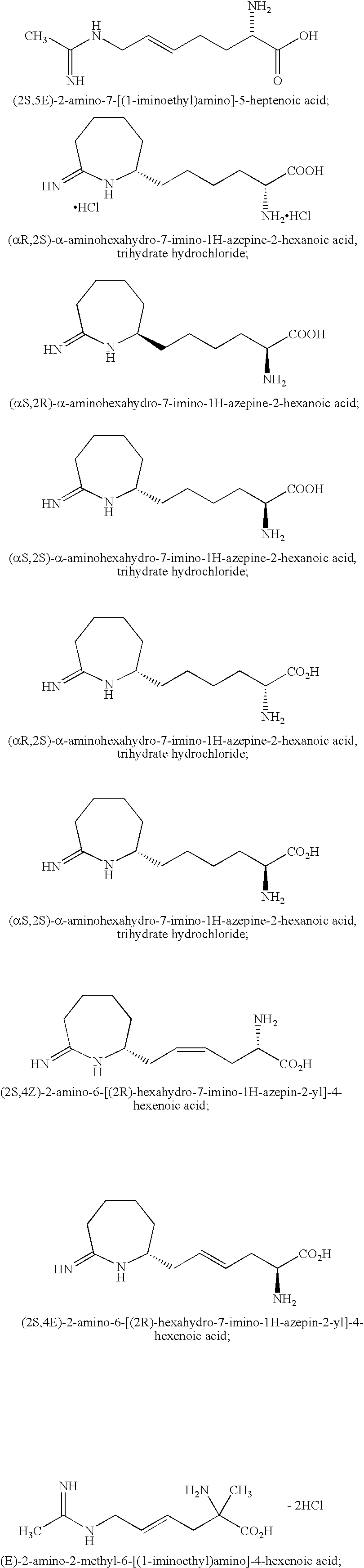 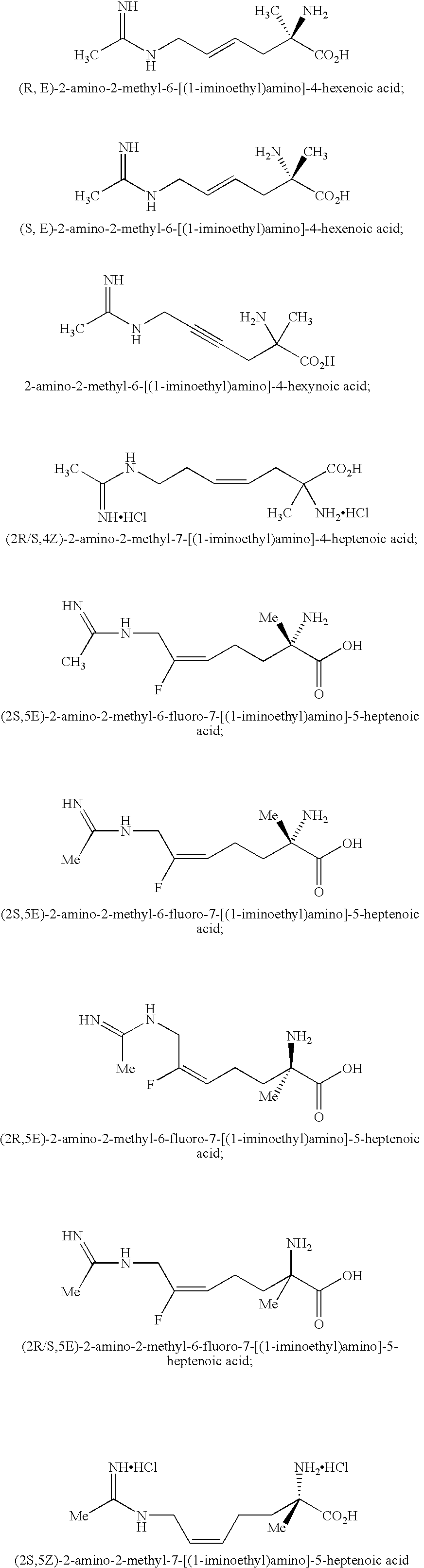 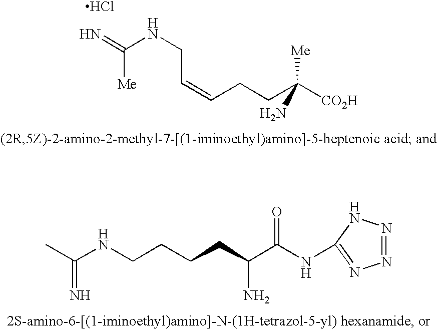
a pharmaceutically acceptable salt thereof.

An especially preferred selective substrate iNOS inhibitor for use in the present invention is 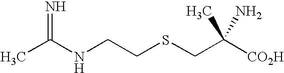
S-[2-[(1-Iminoethyl)amino]ethyl]-2-methyl-L-cysteine, or a pharmaceutically acceptable salt thereof.

Another especially preferred selective substrate iNOS inhibitor for use in the present invention is 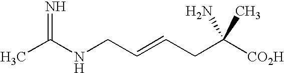
(S, E)-2-amino-2-methyl-6-[(1-iminoethyl)amino]-4-hexenoic acid, or a pharmaceutically acceptable salt thereof.

Still another especially preferred selective substrate iNOS inhibitor useful in the practice of the present invention is 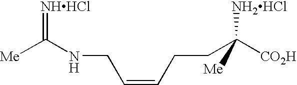
(2S,5Z)-2-amino-2-methyl-7-[(1-iminoethyl)amino]-5-heptenoic acid, or a
pharmaceutically acceptable salt thereof.

Yet another preferred selective substrate iNOS inhibitor useful in the practice of the present invention is 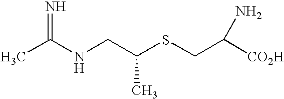
S-[2-(ethanimidoylamino)-1-methylethyl]cysteine (GW-432042), or a
pharmaceutically acceptable salt thereof.

Still another preferred selective selective iNOS inhibitor useful in the practice of the present invention is 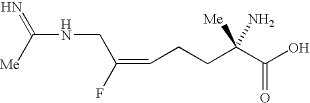
(2S,5E)-2-amino-2-methyl-6-fluoro-7-[(1-iminoethyl)amino]-5-heptenoic acid, or a pharmaceutically acceptable salt thereof.

### DEFINITIONS

The term “compound” means a molecule or salt thereof consisting of a physiologically active ingredient. When a compound may take different or alternative forms in nature, such as a keto-enol tautomer, for example, the alternative forms are intended to be encompassed in the definition of the compound.

The term “composition” means a compound and at least one other ingredient. Examples of such other ingredients are excipients, carriers, adjuvants, surfactants, diluents, fillers and the like. Compositions may be mixtures, solutions, emulsions, suspensions, colloidal dispersions and the like. Compositions may be in the solid phase, liquid phase, gas phase, or combinations thereof.

The term “therapeutic agent” means a compound or a composition that promotes therapy, and acts to modulate a physiological function other than excretion or enzymatic decomposition into inactive components.

The term “chemotherapeutic agent” means a therapeutic agent that acts to modulate a physiological function related to tumor cell growth, maintenance, transformation, metastasis or neovascularization. A chemotherapeutic agent may act either exclusively on tumor cells, or preferentially on tumor cells, or non-preferentially on tumor cells with respect to non-tumor cells.

The term “carbamoylating chemotherapeutic agent” means a compound or composition that acts at least in part by transferring a carbamoyl group to an amino acid residue of a protein, particularly a lysine residue, and thereby modulating the physiological activity of the protein.

The term “alkylating chemotherapeutic agent” means a compound or composition that acts at least in part by transferring an alkyl group to a ribonucleic or deoxyribonucleic acid of RNA or DNA, particularly the guanine of a DNA molecule, and thereby modulating the physiological activity of the DNA molecule.

The term “BCNU” is represented by the chemical structure: 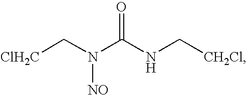
and means 1,3-bis(β-chloroethyl)-1-nitrosourea, CAS Registry Number: 154-93-8, alternatively known as: Urea, N,N′-bis(2-chloroethyl)-N-nitroso-(9CI); Urea, 1,3-bis(2-chloroethyl)-1-nitroso-(8CI); 1,3-bis(2-chlorethyl)-1-nitrosourea; 1,3-bis(2-chloroethyl)-1-nitrosourea; BiCNU; Carmustin; Carmubris Carmustine(USAN); FDA 0345; Nitromon; NCI-C04773; NSC 409962; NSC-409962; SK 27702; SRI 1720; N,N′-bis(2-chloroethyl)-N-nitrosourea; and WLN: ONN2GVM2G.

The term “CCNU” is represented by the chemical structure: 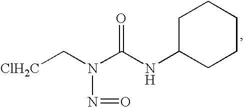
and means urea, N-(2-chloroethyl)-N′ cyclohexyl-N-nitroso-(9CI), CAS Registry Number: 13010474, alternatively known as: Belustine; Cecenu; CeeNU; Chloroethylcyclohexylnitrosourea; CiNu; ICIG 1109; Lomustine(USAN); N-(2-Chloroethyl)-N′-cyclohexyl-N-nitrosourea; NCI-C04740; NSC 79037; SRI 2200; Urea, 1-(2-chloroethyl)-3-cyclohexyl)-1-nitroso; Urea, 1-(2-chloroethyl)-3-cyclohexyl-1-nitroso-(8CI); 1-(2-Chloroethyl)-3-cyclohexyl-1-nitrosourea; 1-(2-Chloroethyl)-3-cyclohexylnitrosourea; 1-Nitrosourea, 1-(2-chloroethyl)-3-cyclohexyl-; and WLN: L6TJ AMVNNO& 2G.

The term “methyl CCNU” is represented by the chemical structure: 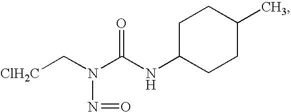
and means urea, N-(2-chloroethyl)-N′-(4-methylcyclohexyl)-N-nitroso-, trans- (9CI), CAS Registry Number: 13909096; alternatively known as NSC-95441; trans-Methyl-CCNU; Lomustine, methyl; MeCCNU; Semustine (USAN); Urea, 1-(2-chloroethyl)-3-(4-methylcyclohexyl)-1-nitroso-, trans-(8CI); 1-(2-Chloroethyl)-3-(trans-4-methylcyclohexane)-1-nitrosourea; and 1-(2-Choroethyl)-3-(4-methylcyclohexyl)-1-nitrosourea.

The term “Cyclodisone” is represented by the chemical structure: 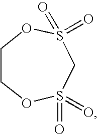
and means 1,5,2,4-Dioxadithiepane, 2,2,4,4-tetraoxide (9C1), CAS Registry Number: 99591738, alternatively known as NSC 348948.

The term “PCNU” is represented by the chemical structure: 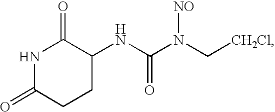
and means urea, N-(2-chloroethyl)-N′-(2,6-dioxo-3-piperidinyl)-N-nitroso-(9CI), CAS Registry Number: 13909-02-9, alternatively known as Urea, 1-(2-chloroethyl)-3-(2,6-dioxo-3-piperidyl)-1-nitroso-(8CI); 1-(2-Chloroethyl)-3-(2,6-dioxo-3-piperidyl)-1-nitrosourea; and NSC 95466.

The term “clomesone” is represented by the chemical structure: 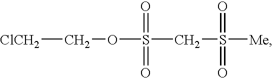
and means methanesulfonic acid, (methylsulfonyl)-, 2-chloroethyl ester (9CI), CAS Registry Number: 88343-72-0, alternatively known as Chlorethyl SOSO; Clomesone; and NSC 338947.

The term “L-cysteine analog” is represented by the chemical structure: 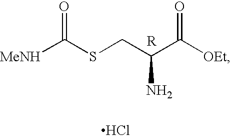
and means L-Cysteine, ethyl ester, methylcarbamate (ester), monohydrochloride (9CI), CAS Registry Number: 51785-99-0, alternatively known as NSC 303861.

The term “triazinate” is represented by the chemical structure: 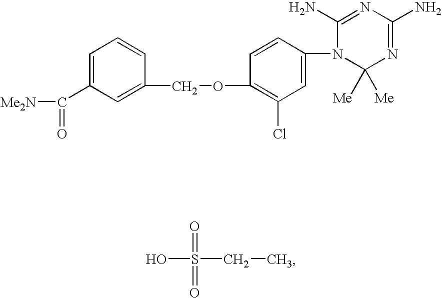
and means ethanesulfonic acid, compd. with 3-[[2-chloro-4-(4,6-diamino-2,2-dimethyl-1,3,5-triazin-1(2H)-yl)phenoxy]methyl]-N,N-dimethylbenzamide (1:1) (9CI), CAS Registry Number 41191-04-2, alternatively known as Benzamide, 3-[[2-chloro-4-(4,6-diamino-2,2-dimethyl-1,3,5-triazin-1(2H)-yl)phenoxy]methyl]-N,N-dimethyl-, monoethanesulfonate (9CI); 1-[3-Chloro-4-(m-dimethylcarbamoylbenzyloxy)phenyl]-4,6-diamino-1,2-dihydro-2,2-dimethyl-s-triazine ethanesulfonate; Baker's Antifol; and NSC 139105.

The term “mitozolomide” is represented by the chemical structure: 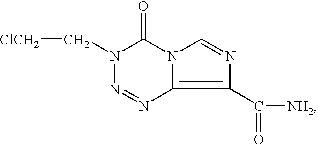
and means midazo[5,1-d]-1,2,3,5-tetrazine-8-carboxamide, 3-(2-chloroethyl)-3,4-dihydro-4-oxo-(9CI), CAS Registry Number: 85622-95-3, alternatively known as Azolastone; CCRG 81010; M and B 39565; and NSC 353451.

The term “carboplatin” is represented by the chemical structure: 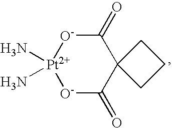
and means platinum, diammine[1,1-cyclobutanedi(carboxylato-_{χ}O)(2-)]-, (SP-4-2)-(9CI), CAS Registry Number: 41575-94-4, alternatively known as 1,1-Cyclobutanedicarboxylic acid, platinum complex; Platinum, diammine[1,1-cyclobutanedicarboxylato(2-)]-, (SP-4-2)-; Carboplatinum; CBDCA; cis-Diammine(1,1-cyclobutanedicarboxylato)platinum; cis-Diammine(1,1-cyclobutanedicarboxylato)platinum(II); cis-Diammineplatinum 1,1-cyclobutanedicarboxylate; cis-Diammine[1,1-cyclobutanedicarboxylato(2-)]platinum; JM 8; NSC 241240; Paraplatin; and Ribocarbo L.

The term “chlorozotocin” is represented by the chemical structure: 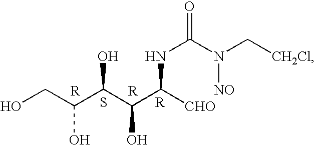
and means D-glucose, 2-[[[(2-chloroethyl)nitrosoamino]carbonyl]amino]-2-deoxy-(9CI) , CAS Registry Number: 54749-90-5, alternatively known as Chlorozotocine; CHLZ; DCNU; and NSC 178248.

The term “treatment” means preventative, palliative or restorative therapeutic methods.

The term “preventative treatment” is used to qualify only prophylactic therapeutic methods.

The term “palliative treatment” is used to qualify only therapeutic methods that relieve symptoms, such as, for example, pain.

The term “restorative treatment” is used to qualify therapeutic methods that halt the progression of, reduce the pathologic manifestations of, or entirely eliminate a cancer condition.

The term “treatment effective amount” means a therapeutically relevant quantity of the indicated therapeutic modality or modalities sufficient to provide the indicated type of treatment. Thus, a “palliative treatment effective amount” will provide a sufficient quantity of therapeutic modality or modalities to relieve symptoms associated with a cancer condition, while a “restorative treatment effective amount will provide a sufficient quantity of therapeutic modality or modalities to halt the progression of, reduce the pathologic manifestations of, or entirely eliminate a cancer condition, for example.

The term “subject” means a human or non-human animal that is susceptible to cancer and treatable with the treatment methods of the present invention, or amenable to clinical investigation.

The term “human subject” is used to qualify the subject to be treated as only a human subject.

The terms “non-human subject”, “animal subject”, and “animal” are used to qualify the subject to be treated or investigated as a non-human animal.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “in conjunction with” means that one first treatment modality is used with at least one, second treatment modality at a period of time conducive to treatment. When a first treatment modality is used in conjunction with a second treatment modality, the treatment modalities may be applied in a substantially simultaneous manner, or the treatment modalities may be applied in a sequential manner. Although the treatment modalities may be referred to as a “first” or “second” treatment modality, the order of application or administration is not necessarily sequential, and the terms “first,” “second” and “third,” when used in the context of the phrase “in conjunction with” are intended only to differentiate the treatment modalities, and do not infer chronological order, unless such treatment modalities are specifically designated as ordered chronologically.

The terms “nitric oxide synthase inhibitor” and “NOS inhibitor” mean a compound that reduces the physiological effect of a nitric oxide synthase enzyme. Such an inhibitor may be selective for a particular isoform of nitric oxide synthase, or may be substantially non-selective, that is, effective to a large extent on two or more isoforms of nitric oxide synthase.

The terms “selective nitric oxide synthase inhibitor” and “selective NOS inhibitor” denote a compound capable of reducing the physiological effect of a particular isoform of nitric oxide synthase preferentially over other isoforms of nitric oxide synthase.

The terms “selective inducible nitric oxide synthase inhibitor”, “selective NOS-2 inhibitor”, and “selective iNOS inhibitor” denote a compound capable of reducing the physiological effect of the calcium ion independent isoform of nitric oxide synthase preferentially over other isoforms of nitric oxide synthase. For purposes of this invention, a selective inducible nitric oxide synthase inhibitor, or selective iNOS inhibitor, acts, at least in part, as either a competitive substrate for the iNOS enzyme (competing with L-arginine at the active site of the iNOS enzyme), or as an inhibitor of dimerization of iNOS monomers.

The term “alkyl”, alone or in combination, means an acyclic alkyl radical, linear or branched, preferably containing from 1 to about 10 carbon atoms and more preferably containing from 1 to about 6 carbon atoms. “Alkyl” also encompasses cyclic alkyl radicals containing from 3 to about 7 carbon atoms, preferably from 3 to 5 carbon atoms. The alkyl radicals can be optionally substituted with groups as defined below. Examples of such radicals include methyl, ethyl, chloroethyl, hydroxyethyl, n-propyl, isopropyl, n-butyl, cyanobutyl, isobutyl, sec-butyl, tert-butyl, pentyl, aminopentyl, iso-amyl, hexyl, octyl and the like.

The term “alkenyl”, alone or in combination, refers to an unsaturated, acyclic hydrocarbon radical, linear or branched, in so much as it contains at least one double bond. Such radicals typically contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1-yl, isobutenyl, penten-1-yl, 2-methylbuten-1-yl, 3-methylbuten-1-yl, hexen-1-yl, 3-hydroxyhexen-1-yl, hepten-1-yl, and octen-1-yl, and the like.

The term “alkynyl”, alone or in combination, refers to an unsaturated, acyclic hydrocarbon radical, linear or branched, in so much as it contains one or more triple bonds. Such radicals typically contain from 2 to about 6 carbon atoms, preferably from 2 to about 4 carbon atoms, more preferably from 2 to about 3 carbon atoms. The alkynyl radicals may be optionally substituted with groups as defined below. Examples of suitable alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “alkoxy”, alone or in combination, embraces linear or branched oxy-containing radicals each having alkyl portions of 1 to about 6 carbon atoms, preferably 1 to about 3 carbon atoms, such as a methoxy radical. The term “alkoxyalkyl”, alone or in combination, also embraces alkyl radicals having one or more alkoxy radicals attached to the alkyl radical, that is, to form monoalkoxyalkyl and dialkoxyalkyl radicals. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylthio”, alone or in combination, embraces radicals containing a linear or branched alkyl radical, of 1 to about 6 carbon atoms, attached to a divalent sulfur atom. An example of “lower alkylthio” is methylthio (CH₃—S—).

The term “alkylthioalkyl”, alone or in combination, embraces alkylthio radicals, attached to an alkyl group. Examples of such radicals include methylthiomethyl.

The term “halo”, alone or in combination, means halogens such as fluorine, chlorine, bromine or iodine atoms.

The term “heterocyclyl”, alone or in combination, means a saturated or unsaturated mono- or multi-ring carbocycle wherein one or more carbon atoms is replaced by N, S, P, or O. This includes, for example, the following structures: 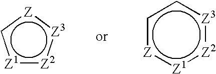
wherein Z, Z¹, Z² or Z³ is C, S, P, O, or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only when each is C. The term “heterocyclyl”, alone or in combination, also includes fully saturated ring structures such as piperazinyl, dioxanyl, tetrahydrofuranyl, oxiranyl, aziridinyl, morpholinyl, pyrrolidinyl, piperidinyl, thiazolidinyl, and others. The term “heterocyclyl”, alone or in combination, also includes partially unsaturated ring structures such as dihydrofuranyl, pyrazolinyl, imidazolinyl, pyrrolinyl, chromanyl, dihydrothiophenyl, and others.

The term “heteroaryl”, alone or in combination, means a fully unsaturated heterocycle.

In either “heterocycle” or “heteroaryl,” the point of attachment to the molecule of interest can be at the heteroatom or elsewhere within the ring.

The term “cycloalkyl”, alone or in combination, means a mono- or multi-ringed carbocycle wherein each ring contains 3 to about 7 carbon atoms, preferably from 3 to about 5 carbon atoms. Examples include cyclopropyl, cyclobutyl, cyclopentyl, cyclohexyl, cycloalkenyl, and cycloheptyl. The term “cycloalkyl”, alone or in combination, additionally encompasses spiro systems wherein the cycloalkyl ring has a carbon ring atom in common with the 7-membered heterocyclic ring of the benzothiepine.

The term “oxo”, alone or in combination, means a double-bonded oxygen.

The term “aryl”, alone or in combination, means a fully unsaturated mono- or multi-ring carbocycle, including, but not limited to, substituted or unsubstituted phenyl, naphthyl, or anthracenyl.

The phrase “optionally substituted” means that the indicated radical may, but need not be substituted for hydrogen. Thus, the phrase “optionally substituted by one or more” means that if a substitution is made at the indicated moiety, more than one substitution is contemplated as well. In this regard, if more than one optional substituent exists, either substituent may be selected, or a combination of substituents may be selected, or more than one of the same substituent may be selected. By way of example, and not limitation, the phrase “C₁-C₅ alkyl optionally substituted by one or more halo or alkoxy” should be taken to mean, for example, that methyl, ethyl, propyl, butyl, or pentyl may have at all substitutable positions: hydrogen, fluorine, chlorine or other halogen, methoxy, ethoxy, propoxy, iso butoxy, tert-butoxy, pentoxy or other alkoxy radicals, and combinations thereof. Non-limiting examples include: propyl, iso-propyl, methoxypropyl, fluoromethyl, fluoropropyl, 1-fluoro-methoxymethyl and the like.

When a compound is described by both a structure and a name, the name is intended to correspond to the indicated structure, and similarly the structure is intended to correspond with the indicated name.

With reference to the use of the words “comprise” or “comprises” or “comprising” in this patent (including the claims), Applicants note that unless the context requires otherwise, those words are to be interpreted inclusively rather than exclusively.

Words and phrases that are not expressly defined herein are to be understood as taking their ordinary and customary meaning, as applied by those of ordinary skill in the art. Reference may be made to a standard dictionary, such as, for example, Webster's Third New International Dictionary of the English Language, Unabridged (1993).

### ILLUSTRATIVE EXAMPLES

The following examples are merely illustrative, and not limiting to the remainder of this disclosure in any way.

### Compound Prepararation

The following synthesis examples are shown for illustrative purposes. Other compounds and salts may be prepared using the methods illustrated in these examples, either alone or in combination with techniques generally known in the art. Where isomers are not defined, utilization of appropriate chromatography methods generally will afford single isomers.

### Example A

### Preparation of (2S,5E)-2-amino-6-fluoro-7-[(1-iminoethyl)amino]-5-heptenoic acid, dihydrochloride, monohydrate

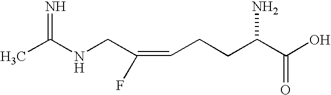

### EX-A-1

### Preparation of

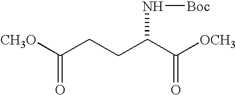 Trimethylsilyl chloride (107.8 g, 1.00 mol) was added dropwise to a cooled solution of L-glutamic acid (30.00 g, 0.20 mol) in 300 mL of methanol at 0° C. The resulting clear, colorless solution was allowed to stir at room temperature. After 18 hr, analysis by thin layer chromatography (30% ethyl acetate in hexane) showed that no starting material remained. The reaction was then cooled to 0° C., triethylamine (134 g, 1.33 mol) was added, and a white precipitate formed. Di-tert-butyldicarbonate (49 g, 0.23 mol) was added, and the mixture was allowed to warm to room temperature. After 3 hr the solvent was removed, and 700 mL of diethyl ether was added. The solution was filtered, and the filter cake was rinsed with an additional 500 mL of diethyl ether. The filtrate was concentrated to 60.8 g (>95%) of a tan oil which was carried onto the next step without further purification. LCMS: m/z=298.1 [M+Na]⁺. HRMS calcd. for C₁₂H₂₁NO₆: 276.1447 [M+H]⁺, found: 276.1462. ¹H NMR (CDCl₃) δ 1.45 (s, 9H), 1.95 (m, 1H), 2.50 (m, 1H), 2.40 (m, 2H), 3.69 (s, 3H), 3.75 (s, 3H), 4.32 (m, 1H), 5.15 (m, 1H).

### EX-A-2

### Preparation of

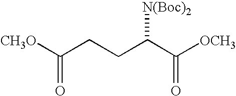 To a solution of the crude product from EX-A-1 (60 g, 0.22 mol) in 300 mL of acetonitrile at room temperature was added 4-dimethylaminopyridine (5.3 g, 0.44 mol) and di-tert-butyldicarbonate (79.2 g, 0.36 mol). The resulting mixture was stirred for 2 days at room temperature, at which time analysis by thin layer chromatography (25% ethyl acetate in hexane) showed that most of the starting material was consumed. The solvent was removed in vacuo affording 85 g of a red oil. The crude material was purified by flash column chromatography on silica gel eluting with 1:10 ethyl acetate in hexane to give 66.4 g (81%) of the desired di-Boc product as a pale-yellow solid. LCMS: m/z=398.2 [M+Na]⁺. HRMS calcd. for C₁₇H₂₉NO₈: 398.1791 [M+Na]⁺, found: 398.1790. ¹H NMR (CDCl₃) δ 1.48 (s, 18H), 2.19 (m, 1H), 2.41 (m, 2H), 2.46 (m, 1H), 3.66 (s, 3H), 3.70 (s, 3H), 4.91 (dd, 1H).

### EX-A-3

### Preparation of

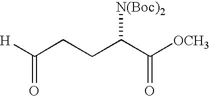 A solution of DIBAL (64 mL of 1.0 M solution in hexanes, 63.9 mmol) was added dropwise to a cold solution of EX-A-2 (20 g, 53.3 mmol) in 400 mL of anhydrous diethyl ether at −78° C. over 30 min. After an additional 30 min at −78° C., the solution was quenched with water (12 mL, 666 mmol) and allowed to warm to room temperature. The cloudy mixture was diluted with 350 mL of ethyl acetate, dried over MgSO₄ and filtered through a pad of celite. The filtrate was concentrated to a yellow oil. The crude material, 18.9 g of yellow oil, was purified by flash column chromatography on silica gel eluting with 1:4 ethyl acetate in hexane to give 13.8 g (75%) of the desired aldehyde product as a clear oil. LCMS: m/z=368.2 [M+Na]⁺. ¹H NMR (CDCl₃) δ 1.48 (s, 18H), 2.19 (m, 1H), 2.41 (m, 2H), 2.46 (m, 1H), 3.70 (s, 3H), 4.91 (dd, 1H), 9.8 (s, 1H).

### EX-A-4

### Preparation of

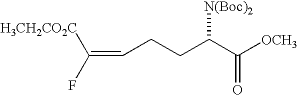 To a cold (−78° C.) solution of triethyl 2-fluorophosphonoacetate (4.67 g, 19.3 mmol) in 20 mL of THF was added n-butyl lithium (10.9 mL of 1.6 M in hexane, 17.5 mmol). This mixture was stirred at −78° C. for 20 min producing a bright yellow solution. A solution of the product from EX-A-3 (6.0 g, 17.5 mmol) in 5 mL of THF was then added via syringe, and the resulting mixture was stirred for 2 hr at −78° C., at which time analysis by thin layer chromatography (30% ethyl acetate in hexane) showed that no starting material remained. The reaction was quenched at −78° C. with sat. aqueous NH₄Cl (30 mL). The organic layer was collected, and the aqueous layer was extracted with diethyl ether (2×50 mL). The combined organics were washed with water (100 mL) and brine (100 mL), dried over MgSO₄, filtered and concentrated. The crude material, 8.6 g of a yellow oil, was purified by flash column chromatography on silica gel eluting with 1:4 ethyl acetate in hexane to give 6.05 g (79%) of the desired fluoro olefin product as a clear oil. ¹H NMR and ¹⁹F NMR indicated that the isolated product had an approximate E:Z ratio of 95:5. LCMS: m/z=456.2 [M+Na]⁺. HRMS calcd. for C₂₀H₃₂NO₈F: 456.2010 [M+Na]⁺, found: 456.2094. ¹H NMR (CDCl₃) δ 1.48 (s, 18H), 2.0 (m, 1H), 2.25 (m, 1H), 2.6 (m, 2H), 3.7 (s, 3H), 4.25 (m, 2H), 4.9 (m, 1H), 5.9 (dt, vinyl, 1H, J=20 Hz), 6.2 (dt, vinyl, 1H, J=30 Hz). ¹⁹F NMR (CDCl₃) δ −129.12 (d, 0.09F, J=31 Hz, 9% Z-isomer), −121.6 (d, 0.91F, J=20 Hz, 91% E-isomer).

### EX-A-5

### Preparation of

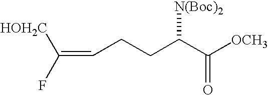 To a solution of EX-A-4 (805 mg, 1.86 mmol) in 20 mL of methanol at room temperature was added solid NaBH₄ (844 mg, 22.3 mmol) in 200 mg portions. The reaction was stirred for 18 hr at ambient temperature, at which time analysis by thin layer chromatography (30% ethyl acetate in hexane) showed that most of the starting material was consumed. The reaction was quenched with 20 mL of sat. aqueous NH₄Cl and extracted with ethyl acetate (2×35 mL). The organic layers were combined, dried over MgSO₄, filtered and concentrated. The crude material, 700 mg of clear oil, was purified by flash column chromatography on silica gel eluting with 1:4 ethyl acetate in hexane to give 353 mg (48%) of the desired allylic alcohol product as a clear oil, that contained primarily the desired E-isomer by ¹⁹F NMR. LCMS: m/z=414.2 [M+Na]⁺. ¹H NMR (CDCl₃) δ 1.48 (s, 18H), 1.95 (m, 1H), 2.1 (m, 1H), 2.2 (m, 1H), 2.35 (t, 1H), 3.7 (s, 3H), 4.25 (m, 2H), 4.8 (m, 1H), 5.15 (dt, 1H, J=20 Hz). ¹⁹F NMR (CDCl₃) δ −119.1 (d, 0.02F, J=37 Hz, 2% Z-isomer), −111.8 (d, 0.98F, J=24 Hz, 98% E-isomer).

### EX-A-6

### Preparation of

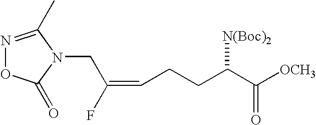 To a mixture of EX-A-5 (1.37 g, 3.5 mmol), polymer-supported triphenylphosphine (3 mmol/g, 1.86 g, 5.6 mmol) and 3-methyl-1,2,4-oxadiazolin-5-one (450 mg, 4.55 mmol) in 50 mL of THF was added dropwise dimethylazodicarboxylate (820 mg, 5.6 mmol). The reaction was stirred for 1 hr at room temperature, at which time analysis by thin layer chromatography (40% ethyl acetate in hexane) showed that no starting material remained. The mixture was filtered through celite, and the filtrate was concentrated. The resulting yellow oil was partitioned between 30 mL of methylene chloride and 30 mL of water. The organic layer was separated, washed with water (1×30 mL) and brine (1×30 mL), dried over MgSO₄, filtered and concentrated. The crude material, 1.8 g of a yellow oil, was purified by flash column chromatography on silica gel eluting with 1:4 ethyl acetate in hexane to give 670 mg (40%) of the desired protected E-allylic amidine product as a clear oil, that contained only the desired E-isomer by ¹⁹F NMR. LCMS: m/z=496.2 [M+Na]⁺. ¹H NMR (CDCl₃) δ 1.48 (s, 18H), 1.85 (m, 1H), 2.2 (m, 3H), 2.25 (s, 3H), 3.64 (s, 3H), 4.25 (m, 2H), 4.8 (m, 1H), 5.3 (dt, 1H, J=20 Hz). ¹⁹F NMR (CDCl₃) δ −110.8 (q, 1F, J=20 Hz).

### EX-A-7

### Preparation of

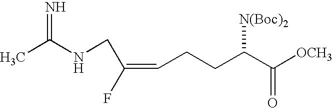 The product from EX-A-6 (670 mg, 1.4 mmol) was dissolved in 25 mL of methanol and 25 mL of 25% acetic acid in water. Zinc dust (830 mg, 12.7 mmol) was added, and the mixture was agitated under sonication for 8 hr, at which time HPLC analysis showed that only 20% of the starting material remained. The Zn dust was filtered from the reaction mixture, and the filtrate was stored at −20 ° C. for 12 hr. The filtrate was warmed to room temperature, additional glacial acetic acid (7 mL) and zinc dust (400 mg, 6.1 mmol) were added, and the mixture was sonicated for 1 hr at room temperature, at which time HPLC analysis showed 96% product. The mixture was filtered through celite, and the filtrate was concentrated. The crude material was purified by reverse-phase HPLC column chromatography on a YMC Combiprep column eluting over 8 min using a gradient of 20-95% A (A: 100% acetonitrile with 0.01% trifluoroacetic acid, B: 100% H₂O with 0.01% trifluoroacetic acid). Fractions containing product were combined and concentrated affording 344 mg (45%) of the desired acetamidine product as a trifluoroacetate salt, that contained only the desired E-isomer by ¹⁹F NMR. LCMS: m/z=432.3 [M+H]⁺. ¹H NMR (CD₃OD) δ 1.52 (s, 18H), 2.9 (m, 1H), 2.2 (m, 3H), 2.27 (s, 3H), 4.2 (d, 1H), 5.4 (dt, vinyl, 1H, J=20 Hz). ¹⁹F NMR (CD₃OD) δ −110.83 (m, 1F, J=20 Hz).

### EX-A-8

### Preparation of

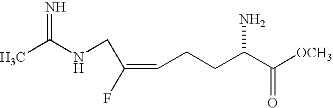 A sample of the product of EX-A-7 is dissolved in glacial acetic acid. To this stirred solution is added 10 equivalents of IN HCl in dioxane. After stirring this solution for 10 min at room temperature, all solvent is removed in vacuo to generate the illustrated methyl ester dihydrochloride salt.

### Ex-A-9

### Preparation of

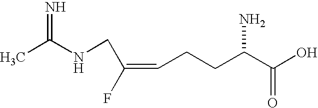 A solution of EX-A-7 (344 mg, 1.4 mmol) in 6 mL of 6.0 N HCl was refluxed for 1 hr. The solvent was removed in vacuo. The resulting solid was dissolved in water and concentrated 3 additional times, followed by 5 subsequent times in 1.0 N HCl to remove any remaining TFA salts. Upon completion, 160 mg (37%) of the desired (2S,5E)-2-amino-6-fluoro-7-[(1-iminoethyl)amino]-5-heptenoic acid, dihydrochloride product was obtained as a white solid, m.p. 51.5-56.3° C., that contained only the desired E-isomer by ¹⁹F NMR. LCMS: m/z=218.1 [M+H]⁺. HRMS calcd. for C₉H₁₆FN₃O₂: 218.1305 [M+H]⁺, found: 218.1325. ¹H NMR (D₂O) δ 1.8 (m, 2H), 2.05 (m, 2H), 2.1 (s, 3H), 3.7 (t, 1H), 4.00 (d, 2H), 5.3 (dt, vinyl, 1H, J=21 Hz). ¹⁹F NMR (D₂O) δ −109.9 (m, 1F, J=20 Hz).

### Example B

### Preparation of (2S,5E/Z)-2-amino-6-fluoro-7-[(1-iminoethyl)amino]-5-heptenoic acid, dihydrochloride

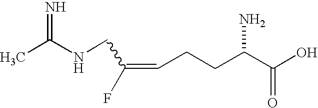

### EX-B-1

### Preparation of

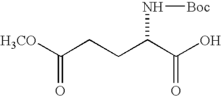 To a cooled (0° C.) solution of L-glutamic acid 5-methyl ester (50.00 g, 0.31 mol) in 400 mL of 1:1 H₂O in dioxane was added triethylamine (38.35 g, 0.38 mol) followed by di-tert-butyldicarbonate (80.00 g, 0.37 mol). The resulting clear, colorless solution was allowed to stir at room temperature. After 18 hr, analysis by thin layer chromatography (30% ethyl acetate in hexane) showed that no starting material remained. The reaction mixture was quenched with 200 mL of 1.0 N aqueous KHSO₄. The organic layer was removed, and the aqueous layer was extracted with ethyl acetate (3×100 mL). The organic layers were combined, dried over MgSO₄, filtered and concentrated to give 72.00 g (89%) of the desired product as a pale yellow oil. LCMS: m/z=284.1 [M+Na]⁺. ¹H NMR (CDCl₃) δ 1.50 (s, 9H), 2.00 (m, 1H), 2.20 (m, 1H), 2.42 (m, 2H), 3.66 (s, 3H), 4.34 (d, 1H), 5.24 (d, 1H).

### EX-B-2

### Preparation of

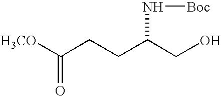 To a solution of the product from EX-B-1 (72.60 g, 0.28 mol) in 300 mL of THF at −10° C. was quickly added 4-methylmorpholine (28.11 g, 0.28 mol) and isobutylchloroformate (37.95 g, 0.28 mol). The clear yellow solution immediately formed a white precipitate. After 4 min, the resulting cloudy yellow mixture was filtered, the filtrate was cooled to −10° C. and a solution of NaBH₄ (15.77 g, 0.42 mol) in 200 mL of H₂O was added dropwise while maintaining a subzero temperature. Once all of the NaBH₄ was added, the ice bath was removed, and the reaction was allowed to stir at room temperature for 1.5 hr. The reaction mixture was quenched with 200 mL of H₂O. The organic layer was separated, and the aqueous layer was extracted with ethyl acetate (3×100 mL). The organic layers were combined, washed with brine, dried over MgSO₄, filtered and concentrated to give 58 g (85%) of the desired product as a yellow oil. LCMS: m/z=270.1 [M+Na]⁺. ¹H NMR (CDCl₃) δ 1.42 (s, 9H), 1.65 (m, 1H), 1.85 (m, 2H), 2.42 (t, 2H), 3.66 (s, 3H), 4.8 (d, 1H).

### EX-B-3

### Preparation of

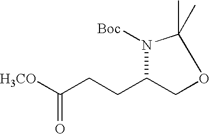 To a solution of EX-B-2 (30.95 g, 0.13 mol) in 100 mL of benzene was added 2,2-dimethoxy propane (65.00 g, 0.63 mol) followed by p-toluenesulfonic acid (2.40 g, 12.5 mmol) and 5 g of 3 Å molecular sieves. The resulting mixture was refluxed for 2 hr, at which time analysis by thin layer chromatography (30% ethyl acetate in hexane) showed complete reaction. The mixture was cooled to room temperature, diluted with diethyl ether (150 mL) and washed with sat. aqueous NaHCO₃ (100 mL) followed by brine (100 mL). The organic layer was dried over MgSO₄, filtered and concentrated. The crude material, 30.5 g of a yellow oil, was purified by flash column chromatography on silica gel eluting with 1:10 ethyl acetate in hexane to give 15.40 g (42%) of the desired product as a pale-yellow oil. LCMS: m/z=310.1 [M+Na]⁺. ¹H NMR (CDCl₃) δ 1.42 (s, 12H), 1.56 (d, 3H), 1.85 (m, 2H), 2.38 (m, 2H), 3.66 (s, 3H), 3.7 (d, 1H), 3.95 (m, 2H).

### EX-B-4

### Preparation of

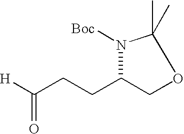 DIBAL (6.0 mL of 1.0 M solution in toluene) was added dropwise to a cold (−78° C.) solution of the product from EX-B-3 (1.00 g, 3.00 mmol) in 10 mL of methylene chloride. After 30 min, the reaction was quenched with 5 mL sat. potassium sodium tartrate (Rochelle salt), then allowed to warm to room temperature. The mixture was then filtered through a pad of celite, dried over MgSO₄, re-filtered and concentrated to give a yellow oil. The crude material, 610 mg of a yellow oil, was purified by flash column chromatography on silica gel eluting with 1:4 ethyl acetate in hexane to give 550 mg (71%) of the desired product as a clear oil. ¹H NMR (CDCl₃) δ 1.50 (s, 12H), 1.58 (d, 3H), 2.00 (m, 2H), 2.5 (m, 2H), 3.7 (d, 1H), 3.95 (m, 2H), 9.8 (s, 1H).

### EX-B-5

### Preparation of

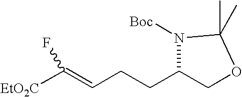 To an ice cold (0° C.) solution of triethyl 2-fluoro-phosphonoacetate (6.70 g, 27.6 mmol) in 100 mL of methylene chloride was added 1,8-diazabicyclo[5.4.0]undec-7-ene (4.70 g, 31.0 mmol). The mixture was stirred at 0° C. for 1 hr resulting in an orange solution. Then, a ice cold (0° C.) solution of the product from EX-B-4 (5.71 g, 22.2 mmol) in 15 mL of methylene chloride was added via syringe, and the resulting mixture was stirred for 18 hr at ambient temperature, at which time analysis by thin layer chromatography (30% ethyl acetate in hexane) showed that no starting material remained. The solvent was removed in vacuo, and the resulting mixture was partitioned between 200 mL of ethyl acetate and 100 mL of water. The organic layer was collected, and the aqueous layer was extracted with ethyl acetate (2×50 mL). The combined organic layers were washed with 1.0 M aqueous KHSO₄ (100 mL), water (100 mL) and brine (100 mL), dried over MgSO₄, filtered and concentrated to give the desired fluoro olefin product as a yellow oil (8.0 g). ¹H NMR and ¹⁹F NMR indicated that the isolated product had an approximate Z:E ratio of 70:30. LCMS: m/z=368.2 [M+Na]⁺. ¹H NMR (CDCl₃) δ 5.9-6.0 (dt, 1H, J=20 Hz), 6.05-6.20 (dt, 1H, J=33 Hz). ¹⁹F NMR (CDCl₃) δ −129.89 (d, 0.7F, J=38 Hz, 70% Z-isomer), −122.05 (d, 0.3F, J=20 Hz, 30% E-isomer). This mixture was carried on crude without further purification.

### EX-B-6

### Preparation of

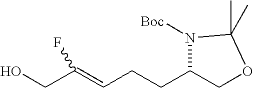 To an ice cold (0° C.) solution of the product from EX-B-5 (8.0 g, 23.0 mmol) in 70 mL of THF was added LiBH₄ (12.7 mL of 2.0 M in THF, 25.0 mmol) via syringe. The reaction mixture was stirred for 18 hr at ambient temperature at which time analysis by thin layer chromatography (30% ethyl acetate in hexane) showed that no starting material remained. The THF was removed, and the resulting mixture was dissolved in methylene chloride. After cooling to 0° C., 1.0 M aqueous KHSO₄ was slowly added to quench the reaction. The mixture was then extracted with ethyl acetate (3×50 mL). The organic layers were combined, dried over MgSO₄, filtered and concentrated. The crude material, 8.0 g of a clear oil, was purified by flash column chromatography on silica gel eluting with 1:4 ethyl acetate in hexane to give 900 mg (13%) of the desired product as a clear oil. LCMS: m/z=326.2 [M+Na]⁺. ¹H NMR (CDCl₃) δ 4.79-4.94 (dm, 1H), 5.10-5.25 (dt, 1H). ¹⁹F NMR (CDCl₃) δ −119.82 (dt, 0.7F, J=38 Hz, 70% Z-isomer), −111.09 (dt, 0.3F, J=27 Hz, 30% E-isomer).

### EX-B-7

### Preparation of

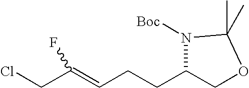 To an ice cold (0° C.) solution of the product from EX-B-6 (950 mg, 3.1 mmol) in 5 mL of pyridine was added methanesulfonyl chloride (390 mg, 3.4 mmol). The reaction was stirred for 5 min at 0° C., then warmed to room temperature and stirred for 3 hr, at which time analysis by thin layer chromatography (30% ethyl acetate in hexane) showed that no starting material remained. The reaction was diluted with diethyl ether (10 mL) and washed with sat. aqueous NaHCO₃ (20 mL) followed by 1.0 M citric acid (20 mL). The organic layer was dried over MgSO₄, filtered and concentrated to give 500 mg (51%) of the desired allylic chloride product as a white solid. This product was carried forward without further purification. LCMS: m/z=344.1 [M+Na]⁺.

### EX-B-8

### Preparation of

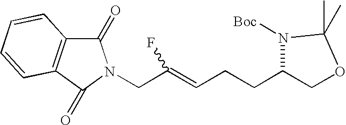 To a stirring solution of the product from EX-B-7 (440 mg, 1.37 mmol) in 10 mL of DMF was added potassium phthalimide (290 mg, 1.57 mmol). The resulting mixture was heated under reflux for 18 hr, at which time analysis by thin layer chromatography (30% ethyl acetate in hexane) showed that no starting material remained. The cooled mixture was diluted with 30 mL of water, extracted twice with ethyl acetate (30 mL), dried over MgSO₄, filtered and concentrated to give 540 mg (91%) of the desired product as a yellow oil. LCMS: m/z=455.2 [M+Na]⁺. HRMS calcd. for: 433.2139 [M+H]⁺, found: 433.2144. ¹H NMR (CDCl₃) δ 1.4 (s, 18H), 1.6 (m, 6H), 2.05 (m, 2H), 3.6-4.42 (m, (4H), 4.9 (dt, vinyl, 1H), 5.2, (m, vinyl, 1H), 7.7 (m, 2H), 7.9 (m, 2H). ¹⁹F NMR (CDCl₃) δ −117.09 (m, 0.7F, J=38 Hz, 70% Z-isomer), −111.61 (m, 0.3F, J=22 Hz, 30% E-isomer).

### EX-B-9

### Preparation of

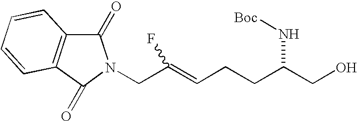 The product from EX-B-8 (600 mg, 1.38 mmol) was dissolved in 8 mL of acetic acid and 2 mL of water. The mixture was stirred at room temperature overnight at which time analysis by thin layer chromatography (30% ethyl acetate in hexane) showed that no starting material remained. The solution was concentrated under a stream of N₂, and the crude product was purified by flash column chromatography on silica gel eluting with 1:2 ethyl acetate in hexane to give 248 mg (63%) of the desired product as a white solid. LCMS: m/z=415.1 [M+Na]⁺. ¹H NMR (CDCl₃) δ 1.41 (s, 9H), 1.56 (m, 2H), 2.15 (m, 1H), 3.64 (m, 2H), 4.35 (d, 2H), 4.9 (dt, vinyl, 1H, J=37 Hz), 7.73 (m, 2H), 7.86 (m, 2H). ¹⁹F NMR (CDCl₃) δ −116.96 (dt, 0.8F, J=37 Hz, 80% Z-isomer), −111.09 (dt, 0.2F, J=22 Hz, 20% E-isomer).

### EX-B-10

### Preparation of

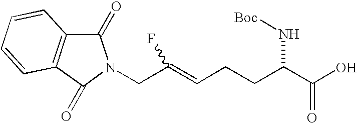 To a stirring solution of the product from EX-B-9 (237 mg, 0.605 mmol) in 6 mL of DMF was added pyridinium dichromate (1.14 g, 3.03 mmol). The solution turned dark orange and was allowed to stir at room temperature for 18 hr, at which time it was poured into 20 mL of H₂O. The mixture was extracted with ethyl acetate (4×25 mL). The combined organic layers were washed with 5% aqueous KHCO₃ (3×25 mL). The aqueous layer was acidified with 1.0 M KHSO₄ to pH=3 followed by extraction with ethyl acetate (3×50 mL). The combined organic layers were concentrated to yield 235 mg (95%) of the desired amino acid product. The resulting white solid was carried on crude without further purification. LCMS: m/z=429.1 [M+Na]⁺.

### EX-B-11

### Preparation of

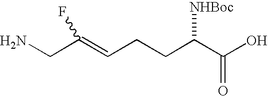 To stirring solution of the product from EX-B-10 (230 mg, 0.56 mmol) in 7 mL of ethanol was added hydrazine hydrate (70 mg, 1.13 mmol), and the resulting solution was refluxed for 2 hr forming a white precipitate. The solvent was removed in vacuo. The resulting white solid was dissolved in 8 mL of water and acidified to pH=4 with glacial acetic acid. It was then cooled in an ice bath and filtered. The filtrate was concentrated to give 136 mg (87%) of the desired allyl amine product as yellow crystals which were carried onto the next step without purification. LCMS: m/z=277.1 [M+H]⁺.

### EX-B-12

### Preparation of

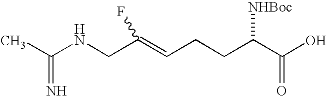 To a stirring solution of the product from EX-B-11 (136 mg, 0.50 mmol) in 6 mL of DMF was added ethyl acetimidate (252 mg, 2.04 mmol) in 3 portions over 1.5 hr intervals. After the addition was complete, the mixture was stirred overnight at room temperature. The pink solution was filtered, and the filter cake was washed with water. The solvent was removed in vacuo, and the resulting yellow oil was purified by reverse-phase HPLC using a YMC Combiprep ODS-A semi-prep column eluting with a 7 min gradient of 1-50% A (A: 100 acetonitrile with 0.05% TFA, B: 100 water with 0.05% TFA). Fractions containing product were combined and concentrated to afford approximately 50 mg of the desired acetamidine product as a trifluoroacetate salt which was carried onto the next step. LCMS: m/z=318.2 [M+H]⁺.

### Ex-B-13

### Preparation of

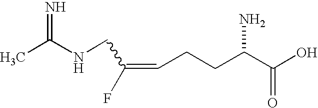 The product from EX-B-12 was dissolved in 6 mL of 6.0 N HCl and stirred for 1 hr at room temperature. The solvent was removed in vacuo. The resulting solid was dissolved in water and concentrated 3 additional times to remove TFA salts. When ¹⁹F NMR indicated that all of the TFA was removed, the product was dried in vacuo to give 30 mg (20%, combined yield over 2 steps) of a 20:80 E:Z mixture containing the desired (2S,5E)-2-amino-6-fluoro-7-[(1-iminoethyl)amino]-5-heptenoic acid, dihydrochloride and (2S,5Z)-2-amino-6-fluoro-7-[(1-iminoethyl)amino]-5-heptenoic acid, dihydrochloride as a foamy clear solid. HRMS calcd. for C₉H₁₆FN₃O₂: 218.1305 [M+H]⁺, found: 218.1309. ¹H NMR (D₂O) δ 2.01 (m, 2H), 2.21 (s, 3H), 2.24 (m, 2H), 3.96 (t, 1H), 4.00 (d, 2H), 5.07 (dt, vinyl, 1H, J=37 Hz), 5.4 (dt, vinyl, 1H, J=37 Hz). ¹⁹F NMR (D₂O) δ −116.8 (m, 0.8F, J=37 Hz, 80% Z-isomer), −109.6 (m, 0.2F, J=21 Hz, 20% E-isomer).

### Example C

### Preparation of (2S,5Z)-2-amino-6-fluoro-7-[(1-iminoethyl)amino]-5-heptenoic acid, dihydrochloride

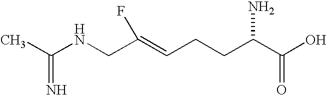

### EX-C-1

### Preparation of

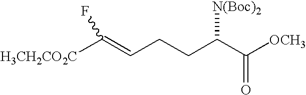 Triethyl 2-fluoro-phosphonoacetate (3.54 g, 14.6 mmol) was dissolved in 20 mL of CH₂Cl₂ at 0° C., and 1,8-diazabicyclo[5.4.0]undec-7-ene (2.4 mL, 16.4 mmol) was added. The mixture was stirred at 0° C. for 20 min producing an orange solution. A solution of the aldehyde product from EX-A-3 (4.04 g, 11.7 mmol) was then added at 0° C., and the resulting brown mixture was stirred overnight at room temperature, at which time LCMS indicated that no starting material remained. The solvent was removed, and the residue was partitioned between water (60 mL) and ethyl acetate (120 mL). The organic layer was collected, and the aqueous layer was extracted with ethyl acetate (2×50 mL). The combined organic layers were washed with water (60 mL) and 10% aqueous KHSO4 (60 mL), dried over MgSO₄, filtered and concentrated. The crude material, 5.7 g of an orange oil, was purified by flash column chromatography on silica gel eluting with 10% ethyl acetate in hexane to give 3.5 g (69%) of the desired fluoro olefin product as a clear oil. ¹H NMR and ¹⁹F NMR indicated that the isolated product had an Z/E ratio of 70:30. HRMS calcd. for C₂₀H₃₂O₈FN: 456.2010 [M+Na]⁺, found 456.2017. ¹H NMR (CDCl₃) δ1.48 (s, 18H), 2.0 (m, 1H), 2.25 (m, 1H), 2.6 (m, 2H), 3.7 (s, 3H), 4.25 (m, 2H), 4.9 (m, 1H), 5.9 (dt, vinyl, 1H, J=21.2 Hz), 6.1 (dt, vinyl, 1H, J=32.4 Hz). ¹⁹F NMR (CDCl₃) δ: −129.4 (d, 0.7F, J=34 Hz, 70% Z isomer), −121.6 (d, 0.3F, J=22 Hz, 30% E isomer).

### EX-C-2

### Preparation of

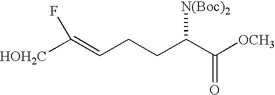 The ester product from EX-C-1 (3.5 g, 8.1 mmol) was dissolved in 80 mL of methanol at room temperature, solid NaBH₄ (3 g, 80 mmol) was then added in portions. The mixture was stirred at room temperature for 18 hr, at which time HPLC analysis indicated that the reaction was >90% complete. The reaction was quenched with sat NH₄Cl. The product was extracted with ethyl acetate and dried over Na₂SO₄. The organic layer was evaporated to give 3.2 g of crude product as a colorless oil, which was purified by Biotage flash column chromatography eluting with 20%-30% ethyl acetate in hexane to give 2.11 g (67%) of a Z/E mixture of the fluoro olefin product as a clear oil along with 0.41 g (13%) of the desired pure (Z:E =97:3 by ¹⁹F NMR) Z-isomer product as a clear oil. HRMS calcd. for C₁₈H₃₀NO₇F: 414.1904 [M+Na]⁺, found 414.1911. ¹H NMR (CDCl₃) δ1.48 (s, 18H), 2.0 (m, 1H), 2.2 (m, 3H), 3.7 (s, 3H), 4.1 (dd, 2H, J=17Hz), 4.8 (dt, 1H, J=39 Hz), 4.9 (m, 1H). ¹⁹FNMR(CDCl₃) δ−119.1 (dt, 1F, J=39Hz,J=17Hz).

### EX-C-3

### Preparation of

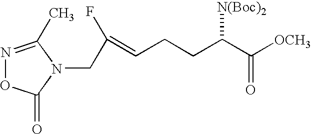 The Z-alcohol product from EX-C-2 (390 mg, 1 mmol) and 3-methyl-1,2,4-oxadiazolin-5-one (130 mg, 1.3 mmol) were dissolved in 20 mL of THF. Then polymer supported-PPh₃ was added into the solution, and the mixture was gently stirred for 10 min. Then diethyl azodicarboxylate was added dropwise, and the mixture was stirred for 1 hr at room temperature, at which time LCMS analysis indicated product formation and that no starting material was present. The polymer was filtered off through a celite pad, and the pad was washed with THF. The filtrate was evaporated to give 1.0 g of crude product which was purified by Biotage flash column chromatography eluting with 20% to 30% ethyl acetate in hexane to give 500 mg of product, contaminated with some hydrazide by-product. This material was further purified by Biotage flash column chromatography eluting with 98:2:0.01 of methylene chloride:methanol:ammon-ium hydroxide to give 180 mg (38%) of the desired protected amidine product as a clear oil, that contained only the desired Z-isomer by ¹⁹F NMR. HRMS calcd. for C₂₁H₃₂N₃O₈F: 491.2517 [M+NH₄]⁺, found 491.2523. ¹H NMR (CDCl₃) δ1.5 (s, 18H), 1.9 (m, 1H), 2.1 (m, 3H), 2.3 (s, 3H), 3.7 (s, 3H), 4.2 (d, 2H), 4.8 (m, 1H), 5.0 (dt, 1H, J=36 Hz). ¹⁹F NMR (CDCl₃) δ−116.5 (dt, 1F, J=38 Hz).

### EX-C4

### Preparation of

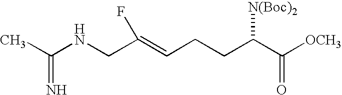 The product from EX-C-3 (88 mg, 0.19 mmol) was dissolved in 4 mL of 25% acetic acid in water containing a few drops of methanol, and then Zn dust (109 mg, 1.67 mmol) was added. The mixture was agitated under sonication for 3 hr. The Zn was filtered off through a celite pad, and the pad was washed with water. The filtrate was evaporated to dryness to give crude product which was purified by reverse-phase HPLC column chromatography on a YMC Combiprep column eluting over 8 min with a gradient of 20-80% A (A: 100% ACN with 0.01% TFA, B: 100% H₂O with 0.01% TFA). The desired product was collected in 2 fractions, and the combined fractions were concentrated. The product was obtained as a colorless oil as a mixture of trifluoroacetate salts that contained only the desired Z-isomer by ¹⁹F NMR: 30% was mono Boc-protected product: HRMS calcd. for C₁₅H₂₆N₃O₄F: 332.1986 [M+H]⁺, found 332.2001, and 70% was di-Boc-protected product: HRMS calcd. for C₂₀H₃₄N₃O₆F: 432.2510 [M+H]⁺, found 432.2503. ¹H NMR of the di-Boc product (D₂O) δ1.3 (s, 18H), 1.8 (m, 1H), 2.1 (m, 3H), 2.1 (s, 3H), 3.6 (s, 3H), 3.9 (d, 2H), 4.9 (dt, vinyl, 1H, J=37 Hz). ¹⁹F NMR (D₂O) δ−117.3 (dt, 1F, J=37 Hz).

### Ex-C-5

### Preparation of

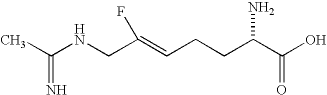 The combined mono- and di-BOC products from EX-C-4 were dissolved in 30 mL of 6N HCl, and the solution was refluxed for 4 hr, at which time LCMS analysis indicated complete reaction. The excess HCl and water was removed in vacuo. Upon completion, 9 mg (40% combined yield for 2 steps) of the desired (2S,5Z)-2-amino-6-fluoro-7-[(1-iminoethyl)amino]-5-heptenoic acid, dihydrochloride product was obtained as a light yellow, very hygroscopic foam, that contained only the desired Z-isomer by ¹⁹F NMR. HRMS calcd. for C₉H₁₆N₃O₂F: 218.1305 [M+H]⁺, found 218.1320. ¹H NMR (D₂O) δ1.3 (s, 18H), 1.9 (m, 2H), 2.1 (m, 2H), 2.1 (s, 3H), 3.8 (t, 1H), 3.9 (d, 2H), 4.9 (dt, vinyl, 1H, J=37 Hz). ¹⁹F NMR (D₂O) δ−117.3 (dt, 1F, J=37 Hz).

### Example D

### Preparation of 2S,5Z)-2-amino-6-fluoro-7-[(1-iminoethyl)amino]-5-heptenoic acid, trihydrochloride, dihydrate

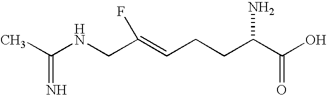

### EX-D-1

### Preparation of

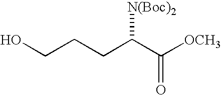 The product from EX-D-2 (3.75 g, 10 mmol) was dissolved in 60 mL of methanol, and solid NaBH₄ (4 g, 106 mmol) was added in portions at room temperature over 10 hr, at which time HPLC analysis indicated approximately 84% reduction. The reaction mixture was quenched with sat. NH₄Cl, and was then extracted with ethyl acetate 3 times. The combined organic layers were dried over MgSO₄, filtered, and evaporated to give 3.2 g of crude product as a yellow oil. HRMS calcd. for C₁₆H₂₉NO₇: 348.2022 [M+H]⁺, found: 348.2034. ¹H NMR (CD₃OD) δ4.9 (q, 1H), 3.7 (s, 3H ), 3.5 (t, 2H), 3.2 (m, 1H), 2.1 (m, 1H), 1.9 (m, 2H), 1.5 (s, 18H).

### EX-D-2

### Preparation of

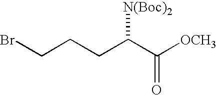 The alcohol product from EX-D-1 (3.2 g, 9.0 mmol) was dissolved in 100 mL of THF and cooled in an ice bath. Carbon tetrabromide (4.27 g, 12.9 mmol) was added, and the resulting solution was stirred at 0° C. for 30 min under N₂. Polymer-supported PPh₃ was added, and the mixture was gently stirred at 0° C. for 1 hr and then overnight at room temperature. The polymer was removed by filtration through celite, and the celite pad was washed with THF. The filtrate was evaporated to give crude product, which was purified by Biotage flash column chromatography eluting with 1:3 ethyl acetate in hexane to give 2.0 g (54%, combined yield over 2 steps) of the desired bromo product as a colorless oil. HRMS calcd. for C₁₆H₂₈NO₆Br: 410.1178 [M+H]⁺, found: 410.1137. ¹H NMR (CDCl₃) δ4.9 (q, 1H), 3.7 (s, 3H ), 3.4 (m, 2H), 2.2 (m, 2H), 1.9 (m, 2H), 1.5 (s, 18H).

### EX-D-3

### Preparation of

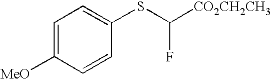 A solution of NaOEt (21% in EtOH, 41.1 mL, 0.11 mol) in 60 mL of ethanol was treated with p-methoxy benzenethiol (14.0 g, 0.1 mol), followed by ethyl chlorofluoroacetate (18.3 g, 0.13 mol). The mixture was stirred at room temperature for 2 hr and diluted with 250 mL of 1:1 hexane in ethyl acetate. The organic layer was washed with water 3 times, and dried over Na₂SO₄. The dried organic layer was evaporated to give 25 g of crude product which was carried forward without further purification. LCMS for C₁₁H₁₃O₃SF: m/z=267.10 [M+Na]⁺. ¹H NMR (CDCl₃) δ7.5 (d, 2H), 6.9 (d, 2H), 6.0 (d, 1H, J=51.9 Hz), 4.2 (q, 2H), 3.8 (s, 3H ), 1.2 (t, 3H). ¹⁹F NMR (CDCl₃) δ−146.2 (d, 1F, J=53.6 Hz).

### EX-D-4

### Preparation of

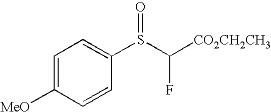 A solution of the crude product from EX-D-3 (24 g, 0.1 mol) in 200 mL of methylene chloride was cooled to −78° C. and treated with 3-chloroperbenzoic acid (27 g, 0.12 mol) in 200 mL of methylene chloride. The reaction mixture was slowly warmed to room temperature and stirred overnight, at which time LCMS analysis indicated product formation and that no starting material remained. The solid was filtered off, and the filtrate was washed with sat. NaHCO₃ and NH₄Cl. The organic layer was dried over MgSO₄ and evaporated to give 30 g of an orange oil, which was purified by Biotage flash column chromatography eluting with 2:1 hexane in ethyl acetate to give 17.5 g (70%) of the desired sulfoxide product as an off-white oil. HRMS calcd. for C₁₁H₁₃O₄FS: 261.0597 [M+H]⁺, found: 261.0598. ¹H NMR (CDCl₃) δ7.6 (m, 2H), 7.0 (m, 2H), 5.6 (d, 1H, J=50 Hz major diastereomer), 5.4 (d, 1H, J=49 Hz minor diastereomer), 4.2 (q, 2H), 3.8 (s, 3H ), 1.2 (t, 3H). ¹⁹F NMR (CDCl₃) δ−194.3 (d, 1F, J=53.6 Hz major diastereomer), −191.7 (d, 1F, J=50.4 Hz minor diastereomer).

### EX-D-5

### Preparation of

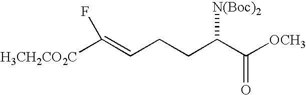 A suspension of NaH (60% in mineral oil, 212 mg, 5.3 mmol) in 6 mL of dried DMF was cooled to 0° C. under N₂ and treated with a solution of the sulfoxide product from EX-D-4 (1.25 g, 4.8 mmol) in 2 mL of DMF. After stirring at room temperature for 20 min, the mixture was cooled to 5° C., and the bromo product from EX-D-2 (2.17 g, 5.3 mmol) was added in one portion. The reaction was stirred at room temperature for 3 hr, then heated at reflux at 95° C. for 1 hr, at which time LCMS analysis indicated product formation. The mixture was poured into an ice/aqueous NH₄Cl mixture. The product was extracted with 1:1 hexane in ethyl acetate. The organic layer was dried over Na₂SO₄ and evaporated to give 3.17 g of a crude yellow oil, which was purified by Biotage flash column chromatography eluting with 10% ethyl acetate in hexane to give 1.05 g (50%) of the desired fluoro olefin ester product as a colorless oil. ¹⁹F NMR indicated that the isolated product contained 95:5 the desired Z-isomer. HRMS calcd. for C₂₀H₃₂O₈FN: 456.2010 [M+Na]⁺, found: 456.2017. ¹H NMR (CDCl₃) δ1.5 (s, 18H), 2.0 (m, 1H), 2.3 (m, 4H), 3.7 (s, 3H), 4.3 (m, 2H ), 4.9 (m, 1H), 6.1 (dt, vinyl, 1H, J=32.4 Hz, Z isomer). ¹⁹F NMR (CDCl₃) δ−129.4 (d, 0.95F, J=34.8 Hz, 95% Z isomer), −121.6 (d, 0.05F, J=21.6 Hz, 5% E isomer).

### EX-D-6

### Preparation of

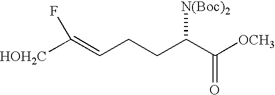 The ester product from EX-D-5 (1.05 g, 2.4 mmol) was dissolved in methanol at room temperature, and solid NaBH₄ was added in portions. The mixture was stirred at room temperature for 18 hr, then 2 mL of water was added, and the mixture was stirred for an additional 3 hr, at which time HPLC analysis indicated the reaction was >95% complete. The reaction was quenched with sat NH₄Cl. The product was extracted with ethyl acetate, and the organic layer was dried over Na₂SO₄ and evaporated to give 0.95 g of crude product as colorless oil. ¹⁹F NMR indicated that the isolated crude product contained only the desired Z-isomer. HRMS calcd. for C₁₈H₃₀NO₇F: 414.1904 [M+Na]⁺, found: 414.1949. ¹H NMR (CDCl₃) δ1.48 (s, 18H), 2.0 (m, 1H), 2.2 (m, 3H), 3.7 (s, 3H), 4.1 (dd, 2H, J=17 Hz), 4.8 (dt, 1H, J=36 Hz), 4.9 (m, 1H). ¹⁹F NMR (CDCl₃) δ−119.1 (dt, 1F, J=38 Hz, J=17 Hz).

### EX-D-7

### Preparation of

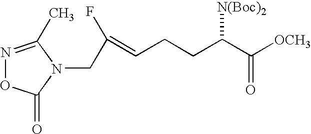 The alcohol product from EX-D-6 (0.95 g, 2.4 mmol) and 3-methyl-1,2,4-oxadiazolin-5-one (290 mg, 2.9 mmol) were dissolved in 60 mL of THF. Polymer-bound triphenyl phosphine was added, and the mixture was gently stirred for 10 min. Then dimethyl azodicarboxylate was added dropwise, and the mixture was stirred for 1 hr at room temperature, at which time LCMS analysis indicated product formation and that no starting material remained. The polymer was filtered off through a celite pad, and the pad was washed with THF. The filtrate was evaporated to give a residue which was partitioned between methylene chloride and water. The organic layer was washed with water twice, dried over MgSO₄, and evaporated to give 1.3 g of crude product which was purified by Biotage flash column chromatography eluting with 20% to 30% ethyl acetate in hexane to give 390 mg (34%, combined yield over 2 steps) of the desired protected amidine product as a colorless oil. ¹⁹F NMR indicated that the isolated product contained only the desired Z-isomer. HRMS calcd. for C₂₁H₃₂N₃O₈F: 491.2517 [M+NH₄]⁺, found: 491.2523. ¹H NMR (CDCl₃) δ1.5 (s, 18H), 1.9 (m, 1H), 2.1 (m, 3H), 2.3 (s, 3H), 3.7 (s, 3H), 4.2 (d, 2H), 4.8 (m, 1H), 5.0 (dt, 1H, J=36 Hz). ¹⁹F NMR (CDCl₃) δ−116.5 (dt,1F,J=38 Hz).

### EX-D-8

### Preparation of

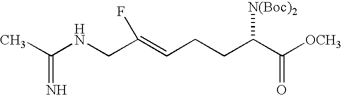 The product from EX-D-7 (390 mg, 0.82 mmol) was dissolved in 20 mL of 25% HOAc in water containing 4 mL of methanol, and Zn dust (482 mg, 7.42 mmol) was added in 2 portions. The mixture was agitated under sonication for 3 hr. The Zn was filtered off through a celite pad, and the pad was washed with water. The filtrate was evaporated to dryness to give crude product which was purified by reverse-phase-HPLC. Fractions containing the desired products were collected, combined and concentrated. The products were obtained as colorless oils as a mixture of trifluoroacetate salts, that contained only the desired Z-isomer by ¹⁹F NMR: 30% was mono-Boc protected product: HRMS calcd. for C₁₅H₂₆N₃O₄F: 332.1986 [M+H]⁺, found 332.2001; 70% was diBoc protected product: HRMS calcd. for C₂₀H₃₄N₃O₆F: 432.2510 [M+H]⁺, 432.2503. ¹H NMR of diBoc product (D₂O) δ1.3 (s, 18H), 1.8 (m, 1H), 2.1 (m, 3H), 2.1 (s, 3H), 3.6 (s, 3H), 3.9 (d, 2H), 4.9 (dt, vinyl, 1H, J=37 Hz). ¹⁹F NMR (D₂O) δ−117.3 (dt, 1F, J=37 Hz).

### Ex-D-9

### Preparation of

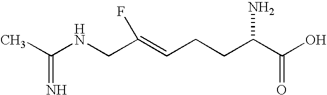 The mono and diBOC products from EX-D-8 were dissolved in 80 mL of 6N HCl and the solution was heated at reflux for 1 hr, at which time LCMS analysis indicated complete reaction. The excess HCl and water was removed in vacuo to give 150 mg (50% combined yield over 2 steps) of the desired (2S,5Z)-2-amino-6-fluoro-7-[(1-iminoethyl)amino]-5-heptenoic acid, trihydrochloride, dihydrate product as a light yellow very hygroscopic foam. HRMS calcd. for C₉H₁₆N₃O₂F: 218.1305 [M+H]⁺, found 218.1290. ¹H NMR (D₂O) δ1.3 (s, 18H), 1.9 (m, 2H), 2.1 (m, 2H), 2.1 (s, 3H), 3.8 (t, 1H), 3.9 (d, 2H), 4.9 (dt, vinyl, 1H, J=37 Hz). ¹⁹F NMR (D₂O) δ−117.3 (dt, 1F, J=37 Hz). Anal. Calcd. for C₉H₁₆N₃O₂F.3HCl.2H₂O: C, 29.81; H, 6.39; N, 11.59; found C, 29.80; H, 6.11; N, 11.20.

### Example E

### Preparation of (2R,5E)-2-amino-6-fluoro-7-[(1-iminoethyl)amino]-5-heptenoic acid, dihydrochloride, monohydrate

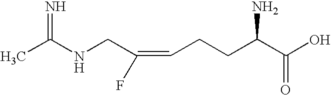

### EX-E-1

### Preparation of

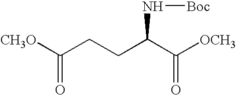 Trimethylsilyl chloride is added dropwise to a cooled solution of D-glutamic acid in methanol at 0° C. The resulting clear, colorless solution is allowed to stir at room temperature until analysis by thin layer chromatography shows that no starting material remains. The reaction is then cooled to 0° C., triethylamine is added, and a white precipitate forms. Di-tert-butyldicarbonate is added, and the mixture is allowed to warm to room temperature. After 3 hr the solvent is removed, and diethyl ether is added. The solution is filtered, and the filter cake is rinsed with additional diethyl ether. The filtrate is concentrated to give the desired mono-Boc diester product which is carried onto the next step without further purification.

### EX-E-2

### Preparation of

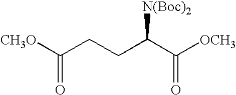 To a solution of the crude product from EX-E-1 in acetonitrile at room temperature is added 4-dimethylaminopyridine and di-tert-butyldicarbonate. The resulting mixture is stirred at room temperature, until analysis by thin layer chromatography shows that most of the starting material is consumed. The solvent is removed in vacuo, and the resulting residue is purified by flash column chromatography on silica gel to give the desired di-Boc protected diester product.

### EX-E-3

### Preparation of

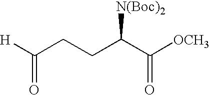 A solution of DIBAL is added dropwise to a cold solution of EX-E-2 in anhydrous diethyl ether at −78° C. After 30 min at −78° C., the solution is quenched with water and allowed to warm to room temperature. The resulting cloudy mixture is diluted with ethyl acetate, dried over MgSO₄ and filtered through a pad of celite. The filtrate is concentrated, and the resulting residue is purified by flash column chromatography on silica gel to give the desired aldehyde product

### EX-E-4

### Preparation of

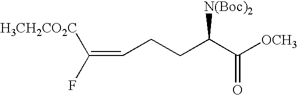 To a cold (−78° C.) solution of triethyl 2-fluorophosphonoacetate in THF is added n-butyl lithium. This mixture is stirred at −78° C. producing a bright yellow solution. A solution of the product from EX-E-3 in THF is then added via syringe, and the resulting mixture is stirred at −78° C., until analysis by thin layer chromatography shows that no starting material remains. The reaction is quenched at −78° C. with sat. aqueous NH₄Cl. The organic layer is collected, and the aqueous layer is extracted with diethyl ether. The combined organics are washed with water and brine, dried over MgSO₄, filtered and concentrated. The crude material is then purified by flash column chromatography on silica gel to give the desired fluoro olefin product.

### EX-E-5

### Preparation of

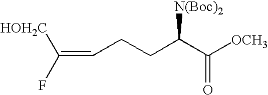 To a solution of EX-E-4 in methanol at room temperature is added solid NaBH₄ in portions. The reaction is stirred at ambient temperature until analysis by thin layer chromatography shows that most of the starting material is consumed. The reaction is quenched with sat. aqueous NH₄Cl and extracted with ethyl acetate. The organic layers are combined, dried over MgSO₄, filtered and concentrated. The crude material is purified by flash column chromatography on silica gel to give the desired allylic alcohol product.

### EX-E-6

### Preparation of

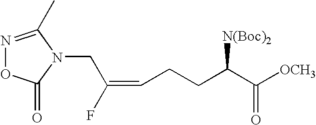 To a mixture of EX-E-5, polymer-supported triphenylphosphine and 3-methyl-1,2,4-oxadiazolin-5-one in THF is added dropwise dimethylazodicarboxylate. The reaction mixture is stirred at room temperature until analysis by thin layer chromatography shows that no starting material remains. The mixture is filtered through celite, and the filtrate is concentrated. The resulting yellow oil is partitioned between methylene chloride and water. The organic layer is separated, washed with water and brine, dried over MgSO₄, filtered and concentrated. The crude material is purified by flash column chromatography on silica gel to give the desired protected E-allylic amidine product.

### EX-E-7

### Preparation of

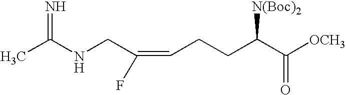 The product from EX-E-6 is dissolved in methanol and acetic acid in water. Zinc dust is added, and the mixture is agitated under sonication until HPLC analysis shows that little of the starting material remains. The Zn dust is filtered through celite from the reaction mixture, and the filtrate is concentrated. The crude material is purified by reverse-phase HPLC column chromatography. Fractions containing product are combined and concentrated affording the desired acetamidine product as a trifluoroacetate salt.

### Ex-E-8

### Preparation of

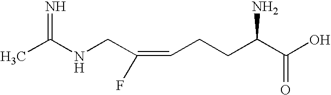 A solution of EX-E-7 in 6.0 N HCl is refluxed for 1 hr. The solvent is removed in vacuo. The resulting solid is dissolved in water and concentrated repeatedly from 1.0 N HCl to remove any remaining TFA salts to give the desired (2R,5E)-2-amino-6-fluoro-7-[(1-iminoethyl)amino]-5-heptenoic acid, dihydrochloride product.

### Example F

### Preparation of (2S,5E)-2-amino-6-fluoro-7-[(1-iminoethyl)amino]-5-heptenoic acid, dihydrochloride, monohydrate

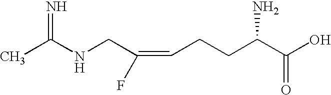

### EX-F-1

### Preparation of

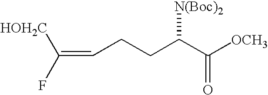 To a THF (45ml) solution of the product of EX-A-3 (5.0g, 11.5mmol) under N₂ was added dropwise a solution of Red-Al (5.22 ml, 17.4 mmol) in 5.6 mL THF over 30 min. The internal temperature was kept below −10° C. After 5 min, the reaction was quenched with 33.7 ml of 1.3M Na.K tartrate. Toluene (11 mL) was added to the mixture to improve separation. The organic layer was washed with 33.7 ml of 1.3M Na.K tartrate followed by brine (40 mL). The organic layers were combined, dried over MgSO4, filtered and concentrated. The crude material, 3.8 g (84%) of light yellow oil, was carried on directly into the next step. LCMS: m/z=414.2 [M+Na]⁺. ¹H NMR (CDCl₃) δ1.48 (s, 18H), 1.95 (m, 1H), 2.1 (m, 1H), 2.2 (m, 1H), 2.35 (t, 1H), 3.7 (s, 3H), 4.25 (m, 2H), 4.8 (m, 1H), 5.15 (dt, 1H, J=20 Hz). ¹⁹F NMR (CDCl₃) δ−119.1 (d, 0.02F, J=37 Hz, 2% Z-isomer), −111.8 (d, 0.98F, J=24 Hz, 98% E-isomer).

### EX-F-2

### Preparation of

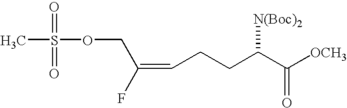 To a solution of the product of EX-F-1 (50.0 g, 0.128 mol) in 500 mL of methylene chloride at −10° C. was added triethylamine (18.0 g, 0.179 mol). A solution of methanesulfonyl chloride (17.5 g, 0.153 mol) in 50 mL methylene chloride was added slowly to maintain temperature at −10° C. The reaction was stirred for 45 min at −10° C., at which time analysis by thin layer chromatography (50% ethyl acetate in hexane) and LCMS showed that most of the starting material was consumed. The reaction was quenched with 600 mL of 1.0 M citric acid and extracted with ethyl acetate (2×400 mL). The organic layers were combined, dried over MgSO₄, filtered and concentrated. The crude material, 70 g of yellow oil, was carried directly into the next step. LCMS: m/z=492.2 [M+Na].

### EX-F-3

### Preparation of

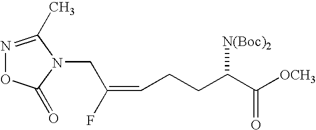 To a solution of the product of EX-F-2 (70.0 g, 0.128 mol) in 400 mL of dimethyl formamide at room temperature was added potassium 3-methyl-1,2,4-oxadiazolin-5-onate (28.7 g, 0.192 mol). The reaction was stirred for 2.5 hr at room temperature, at which time analysis by thin layer chromatography (30% ethyl acetate in hexane) and LCMS showed that the starting material was consumed. The reaction was diluted with 400 mL of water and extracted with ethyl acetate (5×400 mL). The organic layers were combined, washed with 400 mL water, 400 mL brine, dried over MgSO₄, filtered and concentrated. The crude material, 70 g of yellow oil, was purified by flash column chromatography on silica gel eluting with 1:4 ethyl acetate in hexane to give 38 g (63%) of a slightly yellow oil.

EX-F-4. A combination of product of several duplicate preparations of EX-F-3 was purified by HPLC column chromatography on Merk silica gel MODCOL column at a flow of 500 mL/min isocratic at 60:40 MtBE:heptane. A second purification on the 63 g recovered was a chiral HPLC column chromatography on a Chiral Pak-AD column running at a flow of 550 mL/min isocratic at 10:90 A:B (A: 100% ethanol, B: 100% heptane). Fractions containing product were combined and concentrated affording 41 g (68%) of the desired protected L,E-allylic amidine product as a clear oil, that contained only the desired L and E-isomer by ¹⁹F NMR and chiral chromatography. LCMS: m/z=496.2 [M+Na]⁺. [M+NH₄]⁺. HRMS calcd. for C₂₁H₃₂FN₃O₈: 491.2507 [M+NH₄]⁺, found: 491.2517. ¹H NMR (CDCl₃) δ1.48 (s, 18H), 1.85 (m, 1H), 2.2 (m, 3H), 2.25 (s, 3H), 3.64 (s, 3H), 4.25 (m, 2H), 4.8 (m, 1H), 5.3 (dt, 1H, J=20 Hz). ¹⁹F NMR (CDCl₃) δ−110.8 (q, 1F, J=20 Hz).

### EX-F-5

### Preparation of

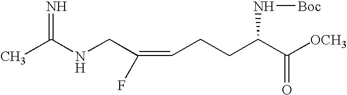 The product from EX-F-4 (22.5 g, 0.047 mol) was dissolved in 112 mL of methanol. Vigorous stirring was begun and 225 mL of 40% acetic acid in water followed by zinc dust (11.5 g, 0.177 mmol) was added. The stirring reaction was placed under reflux (approx. 60° C.) for 2.5 hr, at which time HPLC analysis showed that most of the starting material had been consumed. The reaction was cooled and the Zn was filtered from the reaction mixture through celite, washing the celite well with additional methanol. The filtrate and methanol washings were combined and concentrated. The resulting oily-white solid was washed with methylene chloride (2×500 mL) and filtered through a celite pad, an additional 500 mL methylene chloride wash was performed. The filtrates were combined and concentrated to provide a light yellow oil. The crude material, 39 g of a light-yellow oil, was purified by plug filtration on 200 mL silica gel eluting with 80:19:1 methanol:methylene chloride:acetic acid to give 13 g (83%) of the desired product. LCMS: m/z=432.3 [M+H]⁺. 1 [M+H]⁺. HRMS calcd. for C₁₅H₂₆FN₃O₄: 332.1986 [M+H]⁺, found: 332.1982. ¹H NMR (CD₃OD) δ1.42 (s, 9H), 1.7 (m, 1H), 1.9 (m, 1H), 2.17 (m, 2H), 2.22 (s, 3H), 3.3 (m, 1H), 3.7 (s, 3H), 4.2 (d, 2H), 5.1 (dt, vinyl, 1H, J=21 Hz). ¹⁹F NMR (CD₃OD) δ−110.83 (m, 1F, J=21 Hz).

### Ex-F-6

### Preparation of

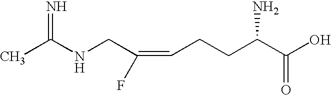 A solution of the product of EX-F-5 (22 g, 0.066 mol) in 750 mL of 6.0 N HCl was refluxed for 45 min. The solvent was removed in vacuo. The resulting solid was dissolved in water and concentrated 3 additional times. The crude material was purified by reverse-phase HPLC column chromatography on a YMC ODS-AQ column eluting over 60 min pumping 100% isocratic B for 30 min followed by a gradient of 0-100% A for 10 min and a 100% A wash for 20 min (A: 100% acetonitrile, B: 100% H₂O with 0.0025% acetic acid). Fractions containing product were combined and concentrated affording 3.5 g (68%) of the desired acetamidine product as a dihydorchloride salt, that contained only the desired (2S,5E)-2-amino-6-fluoro-7-[(1-iminoethyl)amino]-5-heptenoic acid, dihydrochloride product was obtained as a white solid, m.p. 51.5-56.3° C., that contained only the desired E-isomer by ¹⁹F NMR. LCMS: m/z=218.1 [M+H]⁺. HRMS calcd. for C₉H₁₆FN₃O₂: 218.1305 [M+H]⁺, found: 218.1325. ¹H NMR (D₂O) δ1.8 (m, 2H), 2.05 (m, 2H), 2.1 (s, 3H), 3.7 (t, 1H), 4.00 (d, 2H), 5.3 (dt, vinyl, 1H, J=21 Hz). ¹⁹F NMR (D₂O) δ−109.9 (m, 1F, J=20 Hz). [α]₅₈₉=+15.3 (C, 0.334, (H₂O);). [α]₃₆₅=+52.8 (C, 0.334, (H₂O)

### Example G

### Preparation of (2S,5E)-2-amino-6-fluoro-7-[(1-hydroximinoethyl)amino]-5-heptenoic acid

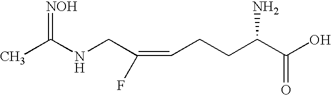

### EX-G-1

### Preparation of

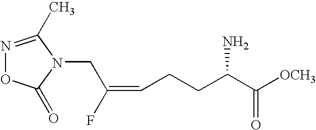 Gaseous HCl was bubbled for 5 min through a stirring cold (0° C.) solution of the product of EX-F-3 (14 g, 30.0 mmol) in 100 mL of methanol. The resulting dark yellow solution was stirred an additional 30 min, at which time HPLC indicated complete consumption of starting material. The resulting mixture was neutralized with saturated NaHCO₃ to pH=8, and the product was extracted out with EtOAc. The organic layer was dried over MgSO₄ and concentrated to give the desired amino ester product as a dark yellow oil that was carried on crude to the next step. LCMS: m/z=274 [M+Na]⁺. ¹H NMR (CDCl₃) δ1.8 (m, 4H), 2.25 (s, 3H), 3.42 (bm, 1H), 3.80 (s, 3H), 4.4 (dd, 2H), 5.40 (dt, vinyl, 1H, J=21 Hz). ¹⁹F NMR (CDCl₃) δ−110.38 (m, 1F, J=21 Hz).

### Ex-G-2

### Preparation of

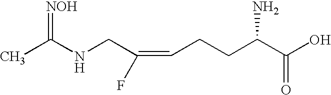 A solution of the product of EX-G-1 (8 g, 30 mmol) in 70 mL of 2.5N NaOH was stirred for 10 min, at which time HPLC analysis indicated the complete consumption of starting material. The resulting solution was neutralized with 12N HCl (approximately 50 mL) to pH=7-8 and concentrated. The resulting slurry was washed with methanol, filtered to remove salts and concentrated to a brownish oil. The crude material was purified by reverse-phase HPLC column chromatography on a YMC ODS-AQ column eluting over 60 min pumping 100% isocratic B for 30 min followed by a gradient of 0-100% A for 10 min and a 100% A wash for 20 min (A: 100% acetonitrile, B: 100%). Fractions containing product were combined and concentrated affording 1.0 g (14%) of the desired product as a white solid. The product was recrystallized from hot water and isopropyl alcohol and collected by filtration to afford pure (2S,5E)-2-amino-6-fluoro-7-[(1-hydroximinoethyl)amino]-5-heptenoic acid as a white crystalline solid. Melting point: 198.00-200.00° C. LCMS: m/z=234.1 [M+H]⁺. ¹H NMR (D₂O) 0 1.8 (m, 4H), 2.05 (m, 2H), 3.6 (t, ₁H), 3.9 (d, 2H), 5.2 (dt, vinyl, 1H, J=21 Hz). ¹⁹F NMR (D₂O) δ−112.1 (m, 1F, J=20 Hz). ). Anal. calcd. for C₉H₁₆FN₃O₃: C, 46.35; H, 6.91; N, 18.02; O, 20.58. Found: C, 46.44; H, 6.95; N, 17.94; O, 20.78. Chiral analysis>97.7%: CrownPak CR(+) at 0.8 mL/min isocratic with 100% A (A: aqueous HClO₄, pH=1.5).

### Example H

### Preparation of (2S,5E)-2-amino-6-fluoro-7-[(1-iminoethyl)amino]-N-(1H-tetrazol-5-yl) 5-heptenamide, dihydrochloride

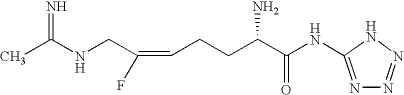

### EX-H-1

### Preparation of

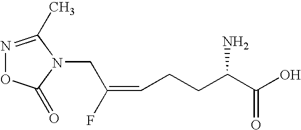 The product from EX-F-3 (6.1 g, 0.013 mol) was dissolved in 4 mL of methanol. Vigorous stirring was begun and 10 mL of 6N HCl was added. The stirring reaction was placed under reflux (approx. 60° C.) for 18 hr, at which time HPLC analysis showed that most of the starting material had been consumed. The reaction was cooled and concentrated to 3.3 g (100%) of orange oil. LCMS: m/z=282 [M+Na]⁺.

### EX-H-2

### Preparation of

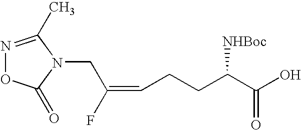 The product from EX-H-1 (3.3 g, 0.013 mol) was dissolved in 12 mL of 1:1 H₂O:dioxane. Stirring was begun and triethylamine (1.95 g, 0.019 mol) was added. The reaction was cooled to 0° C. and di-tert-butyldicarbonate (3.4 g, 0.016 mol) was added. The reaction was allowed to warm to room temperature at which time acetonitrile (4 mL) was added to dissolve solids. The reaction was stirred at room temperature for 18 hr at which time HPLC analysis showed that most of the starting material had been consumed. The reaction was quenched with 1.0N KHSO₄ (25 mL), extracted with ethyl acetate (3×50 mL) and the organic layers dried over MgSO₄ and concentrated. The crude material, 3.5 g of a dark oil, was purified by flash chromatography eluting with 4:95:1 methanol:methylene chloride:acetic acid to give 2.4 g (52%) of desired product as a light-yellow oil. LCMS: m/z=382 [M+Na]⁺.

### EX-H-3

### Preparation of

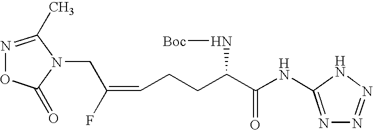 The product from EX-H-2 (2.4 g, 0.007 mol) was dissolved in 13 mL THF. Stirring was begun and 5-aminotetrazole monohydrate (0.83 g, 0.008 mol) was added followed by 1,3-diisopropylcarbodiimide (1.0 g, 0.008 mol). The resulting mixture was allowed to stir at room temperature for 3 hr at which time HPLC showed that most of the starting material had been consumed. To the reaction was added 12 mL water and the THF was removed by vaccum distillation. Ethanol (30 mL) was added and the reaction was heated to reflux. After 15 min at reflux, the reaction was cooled to −10° C. at which time the desired product precipitated from solution. The product was collected by filtration to afford 1.25 g (50%) of a white solid. LCMS: m/z=449 [M+Na]⁺.

### EX-H-4

### Preparation of

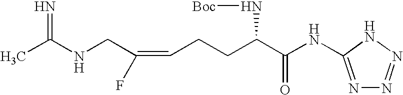 The product from EX-H-3 (1.0 g, 0.0023 mol) was dissolved in 5 mL of methanol. Vigorous stirring was begun and 10 mL of 40% acetic acid in water followed by zinc dust (0.5 g, 0.008 mol) was added. The stirring reaction was placed under reflux (approx. 60° C.) for 1.5 hr, at which time HPLC analysis showed that most of the starting material had been consumed. The reaction was cooled and the Zn was filtered from the reaction mixture through celite, washing the celite well with additional methanol. The filtrate and methanol washings were combined and concentrated. The resulting oily-white solid was purified by reverse-phase HPLC column chromatography on a YMC ODS-AQ column eluting over 60 min pumping 100% isocratic B for 30 min followed by a gradient of 0- 100% A for 10 min and a 100% A wash for 20 min (A: 100% acetonitrile, B: 100% H₂O with 0.0025% acetic acid). Fractions containing product were combined and concentrated affording 0.390 g (44%) of the desired acetamidine product as a white solid. LCMS: m/z=407.3 [M+Na].

### Ex-H-5

### Preparation of

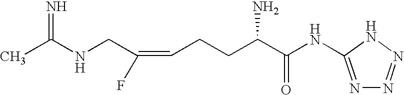 The product from EX-H-4 (0.30 g, 0.780 mmol) was dissolved in 5 mL of conc HOAc. To this was added 1 mL of 4N HCl in dioxane. The reaction was stirred 5 min. at room temperature. The solvent was removed in vacuo. The resulting solid was dissolved in water and concentrated 3 additional times. HPLC indicated amounts of starting material. The solid was dissolved in 1N HCl and stirred 3 h at which time HPLC indicated that most of the starting material had been consumed. The solution was concentrated affording 290 mg (98%) of the desired acetamidine product as a dihydorchloride salt. LCMS: m/z=285.1 [M+H].

### Example I

### Preparation of S-[2-[(1-iminoethyl)amino]ethyl]-2-methyl-L-cysteine, dihydrochloride

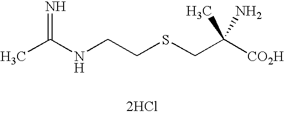

Ex-I-1. Preparation of (2R,4R)-methyl-2-tert-butyl-1,3-thiazoline-3-formyl-4-carboxylate. See Jeanguenat and Seebach, J. Chem. Soc. Perkin Trans. 1, 2291 (1991) and Pattenden et al. Tetrahedron, 49, 2131 (1993): (R)-cysteine methyl ester hydrochloride (8.58 g, 50 mmol), pivalaldehyde (8.61 g, 100 mmol), and triethylamine (5.57 g, 55 mmol) were refluxed in pentane (800 ml) with continuous removal of water using a Dean-Stark trap. The mixture was filtered and evaporated. The resultant thiazolidine (9.15 g, 45 mmol) and sodium formate (3.37 g, 49.5 mmol) were stirred in formic acid (68 ml) and treated with acetic anhydride (13 mL, 138 mmol), dropwise over 1 hr at 0-5° C. The solution was allowed to warm to room temperature and stir overnight. The solvents were evaporated and the residue was neutralized with aqueous 5% NaHCO₃ and extracted with ether (3×). The combined organic layers were dried (anhy. MgSO₄), filtered, and evaporated to give the title compound which was crystallized from hexane-ether as white crystals (8.65 g) (80% overall, 8:1 mixture of conformers). ¹H NMR 5 (CDCl₃) δ major conformer: 1.04 (s, 9H), 3.29 (d, 1H), 3.31 (d, 1H), 3.78 (s, 3H), 4.75 (s, 1H), 4.90 (t, 1H), 8.36 (s, 1H). MS m/z (electrospray) 232 (M+H)⁺ (100%), 204 (10) 164 (24).

Ex-1-2. Preparation of (2R,4R)-Methyl-2-tert-butyl-1,3-thiazoline3-formyl-4-methyl-4-carboxylate. To a solution of the product of Ex-1-1, (2R,4R)-Methyl-2-tert-butyl-1,3-thiazoline-3-formyl-4-carboxylate (8.65 g, 37.4 mmol), in anhydrous tetrahydrofuran (130 mL) under N₂at −78° C. was added DMPU (25 mL) and the mixture stirred for 5 min. Lithium bis(trimethyisilyl)amide, 1 M in tetrahydrofuran, (37.5 mL), was added, and the mixture stirred for 30 min. After methyl iodide (5.84 g, 41.1 mmol) was added, the mixture was held at −78° C. for 4 hr and then warmed to room temperature with continuous stirring. The solvents were evaporated in vacuo and brine and ethyl acetate was added. The aqueous phase was extracted 3× EtOAc, and the combined organic layers were washed with 10% KHSO₄, water, and brine. They were then dried (anhy. MgSO₄), filtered, and stripped of all solvent under reduced pressure. Chromatography of the residual oil on silica with 1-10% EtOAc/hexane yielded the title compound (5.78 g, 63%, 2.4:1 mixture of conformers). ¹H NMR (CDCl₃) δ major conformer, 1.08 (s, 9H), 1.77 (s, 3H), 2.72 (d, 1H), 3.31 (d, 1H), 3.77 (s, 3H), 4.63 (s, 1H), 8.27 (s, 1H); minor conformer, 0.97 (s, 9H), 1.79 (s, 3H), 2.84 (d, 1H), 3.63 (d, 1 H), 3.81 (s, 3H), 5.29 (s, 1H), 8.40 (s, 1H); MS mr/z (electrospray) 246 (M+H)+(100%), 188 (55) 160 (95). Retention time of 16.5 min on a Daicel Chemical Industries Chiracel OAS column, 10-40% IPA/hexane 0-25 min, >95% ee.

Ex-I-3. Preparation of (2R) 2-Methyl-L-cysteine hydrochloride. The product of Ex-I-2, (2R,4R)-Methyl-2-tert-butyl-1,3-thiazoline-3-formyl-4-methyl-4-carboxylate, (5.7 g, 23.2 mmol) was stirred with 6N HCl (100 mL) under N₂ and held at vigorous reflux for 2 days. The solution was cooled, washed with EtOAc and evaporated to yield the product (2R) 2-methyl-cysteine hydrochloride (3.79 g, 95%) as a light yellow powder. ¹H NMR (DMSO-d₆) δ1.48 (s, 3H,) 2.82 (t, 1H), 2.96 (bs, 2H), 8.48 (s, 3H). MS m/z (electrospray) 136 [M+H⁺].

Ex-I-4. Preparation of S-[2-[[(1,1-dimethylethoxy)carbonyl]amino]ethyl]-2-methyl-L-cysteine trifluoroacetate. Sodium hydride (2.6 g, 60% in mineral oil, 65 mmol) was added to an oven-dried, vacuum-cooled RB flask, containing oxygen-free 1-methyl-2-pyrrolidinone (5 mL). The mixture was cooled to −10° C. and stirred under N₂. The product of Ex-I-3, 2-Methyl-L-cysteine hydrochloride, (3.6 g, 21.0 mmol) dissolved in oxygen-free 1-methyl-2-pyrrolidinone (25 ml), was added in portions. After all H₂ evolution ceased, 2-[(1,1-dimethylethoxycarbonyl)-amino]ethyl bromide (4.94 g, 21 mmol) in oxygen-free 1-methyl-2-pyrrolidinone (15 mL) was added at −10° C. The reaction was then stirred for 4 hr allowing warming to room temperature. The solution was neutralized with 1 N HCl and the 1-methyl-2-pyrrolidinone was removed by evaporation in vacuo. Reverse-phase chromatography with 1-20% acetonitrile in 0.05% aqueous trifluoro acetic acid solution yielded the title compound (5.9 g), recovered by freeze-drying appropriate fractions. ¹H NMR (DMSO-d₆/D₂O) δ1.31 (s, 9H), 1.39 (s, 3H), 2.55 (m, 2H), 2.78 (d, 1H), 3.04 (d, 1H), 3.06 (t, 2H). HRMS calc. for C₁₁H₂₂N₂O₄S: 279.1375 (M+H⁺), found 279.1379.

Ex-I-5. Preparation of S-(2-aminoethyl)-2-methyl-L-cysteine hydrochloride. The product of Ex-I-4, S-[2-[[(1,1-dimethylethoxy)carbonyl]amino]ethyl]-2-methyl-L-cysteine trifluoroacetate, (5.5 g, 14.0 mmol) was dissolved in 1 N HCl (100 mL) and stirred at room temperature under N₂ overnight. The solution was removed by freeze-drying to give the title S-(2-aminoethyl)-2-methyl-L-cysteine hydrochloride, ¹H NMR (DMSO-d₆/D₂O) δ1.43 (s, 3H), 2.72 (m, 2H), 2.85 (d, 1 H), 2.95 (t, 2H), 3.07 (d, 1H). m/z [M+H⁺] 179.

Ex-I-6. Preparation of S-[2-[(1-Iminoethyl)amino]ethyl]-2-methyl-L-cysteine, dihydrochloride. The product of Ex-I-5, was dissolved in H₂O, the pH adjusted to 10 with 1 N NaOH, and ethyl acetimidate hydrochloride (1.73 g, 14.0 mmol) was added. The reaction was stirred 15-30 min, the pH was raised to 10, and this process repeated 3 times. The pH was adjusted to 3 with HCl and the solution loaded onto a washed DOWEX 50WX4-200 column. The column was washed with H₂O and 0.25 M NH₄OH, followed by 0.5 M NH₄OH. Fractions from the 0.5 M NH₄OH wash were immediately frozen, combined and freeze-dried to give an oil that was dissolved in 1N HCl and evaporated to give the title compound as a white solid (2.7 g). ¹H NMR (DMSO-d₆/D₂O) δ1.17 (s, 3H), 2.08 (s, 3H), 2.52 (d, 1H), 2.68 (m, 2H), 2.94 (d, 1H), 3.23 (t, 2H). HRMS calc. for C₈H₁₈N₃O₂S: 220.1120 [M+H⁺], found 220.1133.

### Example J

### Preparation of 2-[[[2-[(1-Iminoethyl)amino]ethyl]thiolmethyl]-O-methyl-D-serine, dihydrochloride

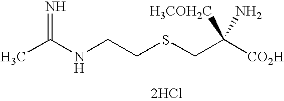 The procedures and methods utilized in this example were identical to those of Example I except that in step Ex-I-2 methoxymethyl iodide was used instead of methyl iodide. These procedures yielded the title product as a white solid (2.7 g). ¹H NMR (D₂O) δ2.06 (s, 3H), 2.70 (m, 3H), 3.05 (d, 1H), 3.23 (s, 3H), 3.32 (t, 2H), 3.46 (d, 1H), 3.62 (d, 1H). HRMS calc. for C₉H₂₀N₃O₃S: 250.1225 [M+H⁺], found 250.1228.

### Example K

### Preparation of S-[(1R)-2-[(1-Iminoethyl)amino]-1-methylethyl]-2-methyl-L-cysteine, dihydrochloride

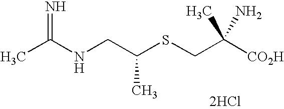

Ex-K-1. Preparation of (S)-1-[(benzyloxycarbonyl)amino]-2-propanol. To a solution of (S)-1-amino-2-propanol (9.76 g, 130 mmol)in anhydrous benzene (60 mL) at 0° C. was added benzyl chloroformate (10.23 g, 60 mmol) in anhydrous benzene (120 mL) slowly, in portions, over a period of 20 min while vigorously stirring under an N₂ atmosphere. The mixture was stirred for 1 hr at 0° C., then allowed to warm to room temperature and stirred for a further 2 hr. The mixture was washed with water (2×) and brine (2×) before the organic layer was dried over anhydrous MgSO₄. Evaporation of all solvent gave the title product as an oil. ¹H NMR (CDCl₃) δ1.22 (d, 3H,) 2.40 (bs, 1H), 3.07 (m, 1H), 3.37 (m, 1H) ), 3.94 (m, 1H), 5.16 (s, 2H), 5.27 (m, 1H), 7.38 (m, 5H). MS m/z (electrospray) 232 [M+23]⁺(100%), 166 (96).

Ex-K-2. Preparation of (S)-1-[(benzyloxycarbonyl)amino]-2-propanol tosylate. To a solution of the product of Ex-K-1, (S)-1-[(benzyloxycarbonyl)amino]-2-propanol, (9.74 g, 46.7 mmol) and triethylamine 7.27 g, 72 mmol) in methylene chloride (60 mL) at 0° C. was added toluene sulfonyl chloride (9.15 g, 48 mmol) in methylene chloride (18 mL) slowly, in portions, over a period of 20 min while vigorously stirring under N₂. The mixture allowed to warm to room temperature and stirred for a further 36 hr under N₂. The organic layer was washed with 1N HCl, water, 5% NaHCO₃ solution, water and brine before it was dried over anhydrous MgSO₄. Evaporation of all solvent gave a white solid which was passed though a silica plug with ethyl acetate/hexane (1:4) to remove excess toluene sulfonyl chloride and then with ethyl acetate/hexane (1:3) to give the title product as white crystals. This material was recrystallized from ethyl acetate/hexane to give white needles (10.8 g). ¹H NMR (CDCl₃) δ1.22 (d, 3H,) 2.39 (s, 3H), 3.20 (m, 1H), 3.43 (dd, 1H) ), 4.66 (m, 1H), 5.02 (m, 1H), 5.04 (ABq, 2H), 7.34 (m, 7H), 7.77 (d, 2H). MS m/z (electrospray) 386 [M+23]⁺(100%), 320 (66). The product was examined on a Regis Technologies Inc. Perkle Covalent (R,R) β-GEM1 HPLC column using mobile phase of isopropanol/hexane and a gradient of 10% isopropanol for 5 min, then 10 to 40% isopropanol over a period of 25 min, and using both UV and Laser Polarimetry detectors. Retention time major peak: 22.2 min, >98% ee.

Ex-K-3. Preparation of S-[(1R)-2-(benzyloxycarbonylamino)-1-methylethyl]-2-methyl-L-cysteine trifluoroacetate. The product of Ex-I-3, 2-methyl-L-cysteine hydrochloride, (1 g, 6.5 mmol) was added to an oven dried, N₂ flushed RB flask, dissolved in oxygen-free 1-methyl-2-pyrrolidinone (5 mL), and the system was cooled to 0° C. Sodium hydride (0.86 g, 60% in mineral oil, 20.1 mmol) was added and the mixture was stirred at 0° C. for 15 min. A solution of the product of Ex-K-2, (2S)-1-[(N-benzyloxycarbonyl)amino]-2-propanol tosylate (2.5 g, 7 mmol) dissolved in oxygen-free 1-methyl-2-pyrrolidinone (10 mL) was added over 10 min. After 15 min at 0° C., the reaction mixture was stirred at room temperature for 4.5 hr. The solution was then acidified to pH 4 with 1N HCl and 1-methyl-2-pyrrolidinone was removed by evaporation in vacuo. Reverse phase chromatography with 20-40% acetonitrile in 0.05% aqueous trifluoro acetic acid solution yielded the title compound in (0.57g), recovered by freeze-drying. ¹H NMR (H₂O, 400 MHz) δ1.0 (d, 3H), 1.4 (s, 3H), 2.6 (m, 2H), 2.8 (m, 1H), 3.1 (m, 2H), 3.6 (s, 1H), 5.0 (ABq, 2H), 7.3 (m, 5H). MS m/z (electrospray): 327 [M+H⁺] (100%), 238 (20), 224 (10), and 100 (25).

Ex-K-4. Preparation of S-[(1R)-2-Amino-1-methylethyl]-2-methyl-L-cysteine hydrochloride. The product of Ex-K-3, S-[(1R)-2-(Benzyloxycarbonylamino)-1-methylethyl]-2-methyl-L-cysteine trifluoroacetate, (0.5 g, 1.14 mmol) was dissolved in 6N HCl and refluxed for 1.5 hr. The mixture was then cooled to room temperature and extracted with EtOAc. The aqueous layer was concentrated in vacuo to give the title product, (2R, 5R)-S-(1-amino-2-propyl)-2-methyl-cysteine hydrochloride (0.29 g), which was used without further purification. ¹H NMR (H₂O, 400 MHz) 5 1.2 (m, 3H), 1.4 (m, 3H), 2.7 (m, 1H), 2.8-3.2 (m, 2H), 3.4 (m, 1H). (some doubling of peaks due to rotameric forms). MS m/z (electrospray): 193 [M+H⁺] (61%), 176 (53), 142 (34), 134 (100), and 102 (10).

Ex-K-5. Preparation of S-[(1R)-2-[(1-Iminoethyl)amino]-1-methylethyl]-2-methyl-L-cysteine, dihydrochloride. The product of Ex-K-4, S-[(1R)-2-Amino-1-methylethyl]-2-methyl-L-cysteine hydrochloride, (0.2 g, 0.76 mmol) was dissolved in 2 mL of H₂O, the pH was adjusted to 10.0 with 1N NaOH, and ethyl acetimidate hydrochloride (0.38 g, 3 mmol) was added in 4 portions over 10 min, adjusting the pH to 10.0 with 1N NaOH as necessary. After 1 h, the pH was adjusted to 3 with 1N HCl. The solution was loaded onto a water-washed DOWEX 50WX4-200 column. The column was washed with H₂O and 0.5N NH₄OH. The basic fractions were pooled and concentrated to dryness in vacuo. The residue was acidified with 1N HCl and concentrated to the title product, (49 mg). ¹H NMR (H₂O, 400 MHz) δ1.3-1.0 (m, 3H), 1.5 (m, 3H), 2.1-1.8 (m, 3H), 3.4-2.6 (m, 5H), 3.6 (m, 1H) (rotamers observed). MS m/z (electrospray): 234 [M+H⁺] (100%), 176 (10), and 134 (10).

### Example L

### Preparation of S-[(1S)-2-[(1-Iminoethyl)amino]-1-methylethyl]-2-methyl-L-cysteine, dihydrochloride

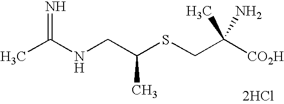 The procedures and methods employed here were identical to those of Example K, except that in step Ex-K-1 (R)-1-amino-2-propanol was used instead of (S)-1-amino-2-propanol to give the title material, S-[(1S)-2-[(1-Iminoethyl)amino]-1-methylethyl]-2-methyl-L-cysteine hydrochloride. ¹H NMR (H₂O, 400 MHz) δ3.6 (m, 1H), 3.4-2.6 (m, 5H), 2.1-1.8 (m, 3H), 1.5 (m, 3H), and 1.3-1.0 (m, 3H). HRMS calc for C₉H₁₉N₃O₂S [M+H⁺]: 234.1276. Found: 234.1286.

### Example M

### Preparation of S-[2-[(1-Iminoethyl)amino]ethyl]-2-ethyl-L-cysteine, dihydrochloride

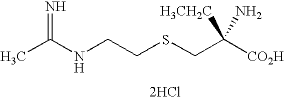 The procedures and methods used in this synthesis were the same as those used in Example I except that ethyl triflate was used in Ex-I-2 instead of methyl iodide. Reverse phase chromatography, using a gradient of 10-40% acetonitrile in water, was used to purify the title product (20% yield). ¹H NMR (D₂O) δ0.83 (t, 3H), 1.80 (m, 2H), 2.08 (s, 3H), 2.68 (m, 1H), 2.78 (m, 1H), 2.83 (m, 1H), 3.11 (m, 1H), 3.36 (t, 2H). HRMS calc. for C₉H₂₀N₃O₂S: 234.1276 [M+H⁺], found 234.1284.

### Example N

### Preparation of 2-[[[[2-(1-iminoethyl)amino]ethyl]thiol methyl]-D-valine, dihydrochloride

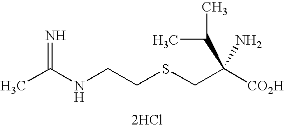

Ex-N-1. Preparation of isopropyl triflate. Silver triflate (25.25 g, 98.3 mmol) stirred in diethyl ether (300 mL) under N₂ was treated with isopropyl iodide (16.54 g, 98.5 mmol) in ether (200 mL) over 15 min. The mixture was stirred for 10 min and then filtered. The filtrate was distilled at reduced pressure. The distillate was redistilled at atmospheric pressure to remove the majority of the diethyl ether, leaving a mixture of the title isopropyl triflate-diethyl ether (84:16 by weight) (15.64 g, 70% corrected) as a colorless liquid. ¹H NMR (CDCl₃, 400 MHz) δ1.52 (d, 6H), 5.21 (septet, 1H).

Ex-N-2. Preparation of 2-[[[[2-(1-iminoethyl)amino]ethyl]thio]methyl]-D-valine, dihydrochloride. The procedures and methods utilized here were the same as those used in Example I, except that isopropyl triflate replaced methyl iodide in Ex-I-2. The crude title product was purified by reversed phase chromatography using a gradient elution of 10-40% acetonitrile in water. ¹H NMR (H₂O, 400 MHz) δ0.94 (dd, 6H), 2.04 (septet, 1H), 2.10 (s, 3H), 2.65, 2.80 (d m, 2H), 2.85, 3.10 (dd, 2H), 3.37 (t, 2H). HRMS calc. for C₁₀H₂₂N₃O₂S: 248.1433 [M+H⁺], found 248.1450.

### Example O

### Preparation of S-[2-(1-Iminoethylamino)ethyl]-2-methyl-(D/L)-cysteine, bistrifluoroacetate

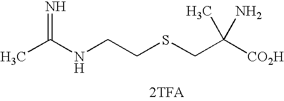

Ex-O-1. Preparation of S-(2-aminoethyl)-L-cysteine, methyl ester. A 10 g (50 mmol) sample of S-(2-aminoethyl)-L-cysteine was dissolved in 400 mL of methanol. Into this cooled solution was bubbled in anhydrous HCl for 30 min. After stirring at room temperature overnight, the solution was concentrated to afford 12.7 g of the title compound.

Ex-O-2. Preparation of N-{4-chlorophenyl)methylene]-S-[2-[[(4-chlorophenyl)methylene]amino]ethyl]-L-cysteine, methyl ester. A 12.7 g (50 mmol) sample of the product of Ex-O-1, S-(2-aminoethyl)-L-cysteine methyl ester, was dissolved in acetonitrile. To this solution was added 12.2 g (100 mmol) of anhydrous MgSO₄, 14 g (100 mmol) of 4-chlorobenzaldehyde and 100 mmol of triethylamine. This mixture was stirred for 12 hr, concentrated to a small volume and diluted with 500 mL of ethyl acetate. The organic solution was washed successively with (0.1%) NaHCO₃, (2N) NaOH, and brine solution. The organic was dried (anhy. MgSO₄), filtered and concentrated to afford 7.5 g of the title compound. [M+H⁺]=179.

Ex-O-3. Preparation of N-[4-chlorophenyl)methylene]-S-[2-[[(4-chlorophenyl)methylene]amino]ethyl]-2-methyl-D/L-cysteine methyl ester. A sample of the product of Ex-O-2, N-{4-chlorophenyl)methylene]-S-[2-[[(4-chlorophenyl)methylene]amino]ethyl]-L-cysteine methyl ester (7.5 g, 17 mmol), in anhydrous THF was treated with 17 mmol of sodium bis(trimethylsilyl)amide at −78° C. under N₂, followed by 2.4 g (17 mmol) of methyl iodide. The solution was held at −78° C. for 4 hr and then warmed to room temperature with continuous stirring. The solvents were evaporated in vacuo and brine and ethyl acetate was added. The aqueous phase was extracted 3× EtOAc, and the combined organic layers were washed with 10% KHSO₄, water, and brine before it was dried (anhy. MgSO₄), filtered, and evaporated to afford the title compound.

Ex-O-4. Preparation of S-(2-aminoethyl)-2-methyl-D/L-cysteine, hydrochloride. A sample of the product of Ex-O-3, N-[4-chlorophenyl)methylene]-S-[2-[[(4-chlorophenyl)methylene]amino]ethyl]-2-methyl-D/L-cysteine methyl ester (4.37 g, 10 mmol), was stirred and heated (60° C.) with 2N HCl overnight and the solution washed (3×) with ethyl acetate. The aqueous solution was freeze-dried to give the title compound.

Ex-O-5. Preparation of S-[2-(1-Iminoethylamino)ethyl]-2-methyl-(D/L)-cysteine, bistrifluoroacetate. A sample of the product of Ex-O-4, S-(2-aminoethyl)-2-methyl-D/L-cysteine dihydrochloride (2.5 g (10 mmol), was dissolved in H₂O and the pH was adjusted to 10 with 1 N NaOH. Ethyl acetimidate hydrochloride (1.24 g, 10.0 mmol) was then added to the reaction mixture. The reaction was stirred 15-30 min, the pH was raised to 10, and this process repeated 3 times. The pH was reduced to 4 with HCl solution and the solution evaporated. The residue was purified on reverse phase HPLC with H₂O containing 0.05% trifluoroacetic acid as the mobile phase to afford the Example 0 title product. M+H=220.

### Example P

### Preparation of (2R)-2-Amino-3[[2-[(1-iminoethyl)amino]ethyl]sulfinyl]-2-methylpropanoic acid, dihydrochloride

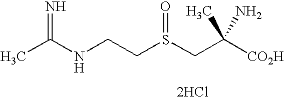 A solution of S-[2-[(1-iminoethyl)amino]ethyl]-2-methyl-L-cysteine, dihydrochloride (Example I, 0.2 g, 0.73 mmol) in 3 mL of water was stirred and cooled to 0° C. and a solution of 3% H₂O₂ (0.8 mL, 0.73 mmol) in formic acid (0.4 mL, 0.73 mmol) was added in 0.3 mL portions. The cold bath was removed and the reaction mixture was stirred at room temperature for 48 hr. The solution was concentrated in vacuo, diluted with of water (10 mL) and concentrated again to give the crude sulfone. This residue was chromatographed (C-18 reverse phase, with mobile phase H₂O containing 0.05% trifluoroacetic acid) to give the pure sulfone. The sulfone was treated with 1M HCl (10 mL) and concentrated in vacuo to give 140 mg of a mixture of 2 diastereomers of the title compound as a colorless oil of the HCl salts. ¹H NMR (300 MHz, D₂O) δ1.5 (s, 2H), 1.6 (s, 1H), 2.0 (s, 3H), 3.1 (m, 2H), 3.3 (m, 2H) 3.6 (m, 2H). HRMS calc. for C₈H₁₈N₃O₃S: 236.1069 [M+H⁺], found: 236.1024.

### Example Q

### Preparation of(2R)-2-Amino-3[[2-[(1-iminoethyl)aminolethyl]sulfonyl]-2-methylpropanoic acid dihydrochloride

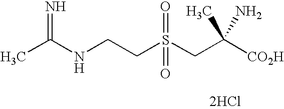 A solution of S-[2-[(1-Iminoethyl)amino]ethyl]-2-methyl-L-cysteine dihydrochloride, the product of Example I, (0.15 g, 0.54 mmol) in 2 mL of water was cooled to 0° C. and a solution of 3% H₂O₂ (1.6 mL, 1.46 mmol) in formic acid (0.8mL, 14.6 mmol) was added. The cold bath was removed and the reaction mixture was stirred at room temperature for 18 hr. The solution was concentrated in vacuo, diluted with 10 mL of water and concentrated again to give the crude sulfoxide. The residue was diluted with 4 mL of water and was adjusted to pH 9 with 2.5 N NaOH. Acetone (5 mL) was added, followed by Boc₂O (0.2 g), and the reaction was stirred for 48 hr at room temperature. The reaction mixture was adjusted to pH 6 with 1M HCl and was concentrated in vacuo. This residue was chromatographed (C-18 reverse phase; 40 to 50% ACN: H₂0, 0.05% TFA) to give the. pure Boc protected material. The fractions were concentrated in vacuo and the residue was treated with 1N HCl (3 mL) for 1 h. The solution was concentrated to give 30 mg of the title compound as colorless oil. ¹H NMR (400 MHz, D₂O) 6 4.0 (d, 1H), 3.7 (d, 1H), 3.6 (t, 2H), 3.5 (t, 2H), 2.1 (s, 3H), and 1.5 (s, 3H) ppm. HRMS calc. for C₈H₁₈N₃O₄S: 252.1018 [M+H⁺], found: 252.0992.

### Example R

### Preparation of (2S,5Z)-2-amino-6-methyl-7-[(1-iminoethyl)amino]-5-heptenoic acid, dihydrochloride

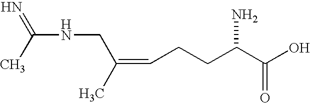

### Ex-R-1

### Preparation of

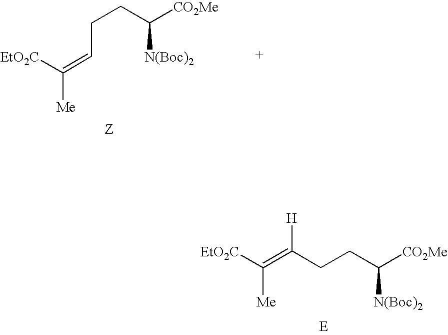 A solution of triethyl-2-phosphonopropionate (6.5 mg, 27.1 mmol) in toluene (60 ML) was treated with 0.5 M potassium bis(trimethylsilyl) amide (50.0 ML, in toluene) and the resulting anion was condensed with the aldehyde product of Ex-U-3 by the method of Ex-U-4 (see Example U infra). This produced, after chromatography, 8 g of a 3:7 mixture respectively of the desired Z and E diesters. (¹H)NMR (300 MHz, CDCl₃) 6.7-6.8 ppm (m,1H), 5.9 ppm (m,1H), 4.9 ppm (m, 1H), 4.2 ppm (q, 2H), 3.7 ppm (s, 3H), 2.5 ppm (m, 1H), 2.2-2.3 ppm (m, 2H), 2.0 ppm (m, 1H), 1.9 ppm (s, 3H), 1.8 ppm (s, 3H), 1.5 ppm (s, 18H), 1.3 ppm (t, 3H).

### Ex-R-2

### Preparation of

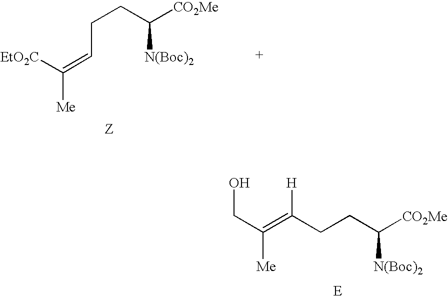 The product mixture of Ex-R-1 (850 mg, 2.0 mmol) in Et₂O (30 mL) was reduced over a period of 20 min with diisobutyl aluminum/hydride (DIBAL) by the method of Ex-U-5 to produce the crude illustrated desired mixture of E-alcohol and unreduced Z-ester. This mixture was chromatographed on silica gel eluting with n-hexane:EtOAc (9:1) to n-hexane:EtOAc (1:1) providing samples of the Z-ester (530 mg) and the E-alcohol desired materials. Z-ester: (¹H)NMR (300 MHz, CDCl₃) 5.9 ppm (m,1H), 4.9 ppm (m, 1H), 4.2 ppm (q, 2H), 3.7 ppm (s, 3H), 2.5 ppm (m, 1H), 2.2-2.3 ppm (m, 2H), 1.9 ppm (s, 3H), 1.5 ppm (s, 18H), 1.3 ppm (t, 3H). E-alcohol: (¹H)NMR (300 MHz, CDCl₃) 5.35 ppm (m,1H), 4.9 ppm (m, 1H), 3.95 ppm (s, 1H), 3.7 ppm (s, 3H), 1.8-2.2 ppm (m, 6H), 1.6 ppm (s, 3H), 1.5 ppm (s, 18H).

### Ex-R-3

### Preparation of

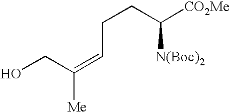 The product Z-ester of Ex-R-2 (510 mg, 1.2 mmol) in Et₂O (30 ML) was reduced over a period of 2 hr with diisobutyl aluminum/hydride (DIBAL) by the method of Ex-U-5 to produce the crude illustrated desired Z-alcohol. This material was chromatographed on silica gel eluting with n-hexane:EtOAc (9:1) to n-hexane:EtOAc (8:2) to yield 340 mg of the desired Z-alcohol product. (¹H)NMR (300 MHz, CDCl₃) δ5.3 ppm (m,1H), 4.9 ppm (m, 1H), 4.2 ppm (d, 1H), 4.0 ppm (d, 1H), 2.2 ppm (m, 3H), 1.95 ppm (m, 1H), 1.8 ppm (s, 3H), 1.5 ppm (s, 18H).

### Ex-R-4

### Preparation of

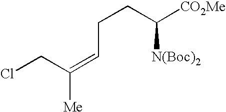 A CH₂Cl₂ solution (5 ML) of the product alcohol of Ex-R-3 (340 mg, 0.9 mmol) was treated with triethylamine (151 mg, 1.5 mmol). To this solution cooled in an ice bath was added a CH₂Cl₂ solution (1.5 ML) of methanesulfonyl chloride. After 15 min the ice bath was removed and the reaction was stirred at ambient temperature for 20 hr. The reaction mixture was then washed with 10% KHSO₄, dried over Na₂SO₄, and stripped of all solvent under reduced pressure to produce 350 mg of the desired Z-allylic chloride. (¹H)NMR (300 MHz, CDCl₃) δ5.4 ppm (m,1H), 4.9 ppm (m, 1H), 4.1 ppm (d, 1H), 4.0 ppm (d, 1H), 2.1 ppm (m, 3H), 1.95 ppm (m, 1H), 1.8 ppm (s, 3H), 1.5 ppm (s, 18H).

### Ex-R-5

### Preparation of

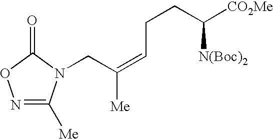 A suspension of potassium 3-methyl-1,2,4-oxa-diazoline-5-one in DMF is reacted with a DMF solution of the product of Ex-R-4 by the method of Ex-S-2 infra to produce the material.

### Ex-R-6

### Preparation of

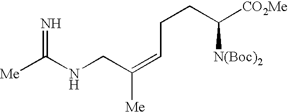 The product of Ex-R-5 is reacted with zinc in HOAc by the method of Ex-U-7 to yield the amidine.

### Ex-R-7

### Preparation of

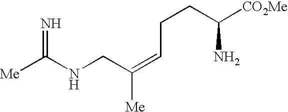 The product of Ex-R-6 was reacted with 4NHCl in dioxane in glacial HOAc to yield the amidine.

### Ex-R-8

### Preparation of

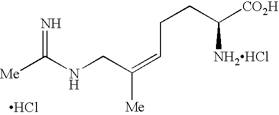 The product of Ex-R-7 is deprotected to yield the amino acid, dihydrochloride.

### Example S

### Preparation of (2S,5E)-2-amino-6-methyl-7-[(1-iminoethyl)amino]-5-heptenoic acid, dihydrochloride

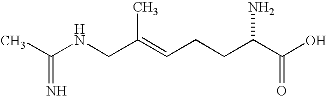

### Ex-S-1

### Preparation of

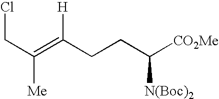 The E-alcohol product of Ex-R-2 (1.3 g, 3.3 mmol) was reacted with triethylamine (525 mg, 5.2 mmol) and methanesulfonyl chloride (560 mg, 5.2 mmol) by the method of Ex-R-4 to yield 1.4 g of the desired E-allylic chloride. (¹H)NMR (400 MHz, CDCl₃) 5.5 ppm (m,1H), 4.9 ppm (m, 1H), 4.0 ppm (s, 2H), 3.7 ppm (s, 3H), 2.1-2.3 ppm (m, 3H), 1.9 ppm (m, 1H), 1.7 ppm (s, 3H), 1.5 ppm (s, 18H).

### Ex-S-2

### Preparation of

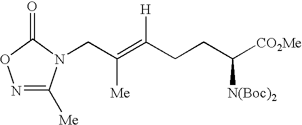 A suspension of potassium 3-methyl-1,2,4-oxa-diazoline-5-one (460 mg, 3.35 mmol) in 5 mL of DMF was treated with a DMF (15 mL) solution of the product of Ex-S-1. This reaction mixture was stirred at 50° C. for 17 hr before an additional 50 mg (0.04 mmol) of the diazoline-5-one salt was added. Heating of the stirred reaction was continued for an additional 3 hr before it was cooled to room temperature and diluted with 180 mL of water. This mixture was extracted with EtOAc and the extracts were diluted with 120 mL of n-hexane, washed with water, dried over Na₂SO₄ and stripped of all solvent under reduced pressure to yield 1.3 g of the material. (¹H)NMR (400 MHz, CDCl₃) 5.5 ppm (m,1H), 4.9 ppm (m, 1H), 4.2 ppm (s, 3H),3.7 ppm (s, 3H), 2.2 ppm (m, 3H), 1.95 ppm (m, 1H), 1.8 ppm (s, 3H), 1.5 ppm (s, 18H).

### Ex-S-3

### Preparation of

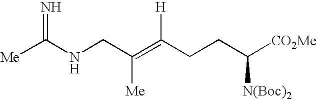 The product of Ex-S-2 (460 mg, 1.0 mmol) was reacted with zinc in HOAc by the method of Ex-U-7 (see Example U infra) to yield 312 mg of the desired amidine after HPLC purification.

### Ex-S-4

### Preparation of

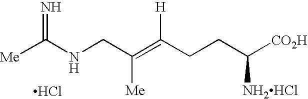 The product of Ex-S-3 (77 mg, 0.2 mmol) was deprotected with 2N HCl by the method of Example U to yield 63 mg the E-amino acid, dihydrochloride.

### Example T

### Preparation of (2S,5Z)-2-amino-7-[(1-iminoethyl)amino]-5-heptenoic acid, dihydrochloride

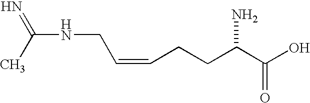

### Ex-T-1

### Preparation of

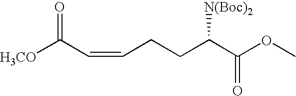 Methyl bis(trifluoroethyl)phosphonoacetate (4.77 g, 15 mmol) and 23.7 g (90 mmol) of 18-crown-6 were dissolved in 80 mL of anhydrous THF and cooled to −78° C. To this soution was added 30 mL (15 mmol) of potassium bis(trimethylsilyl) amide, followed by 5.1 g (14.7 mmol) of N,N-diBoc glutamic aldehyde methyl ester from Ex-U-3 (see Example U infra). After stirring for 30 min at −78° C., the reacion was quenched with aqueous KHSO₄. Extraction of the reaction mixture with EtOAc and concentration afforded 2.95 g (49%) of the desired compound. Mass spectra M+H=402.

### Ex-T-2

### Preparation of

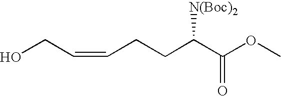 The product from Ex-T-1 was reduced by the method of Ex-U-5 to afford the desired compound.

### Ex-T-3

### Preparation of

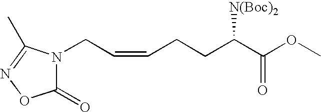 The product from Ex-T-2 was allowed to react with 3-methyl-1,2,4-oxadiazolin-5-one by the method of Ex-U-6 to afford the desired compound.

### Ex-T-4

### Preparation of

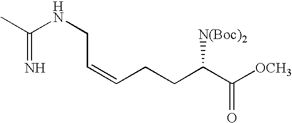 The product from Ex-T-3 was deprotected by the method of Ex-U-7 to afford the desired compound.

### Ex-T-5

### Preparation of

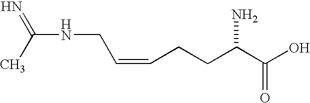

The product from Ex-T-4 was dissolved in 2 N HCl and heated at reflux. The reaction mixture was cooled and concentrated to afford 0.12 g of the desired product. H¹-NMR 1.8-2.0 (m, 2H); 2.05 (s, 3H); 2.15 (q, 2H); 3.75 (d, 2H); 3.9 (t, 1H); 5.45 (m, 1H); 5.6 (m, 1H)

### Example U

### Preparation of (2S,5E)-2-amino-7-1(1-iminoethyl)amino]-5-heptenoic acid, dihydrochloride

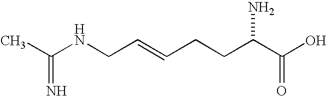

### Ex-U-1

### Preparation of

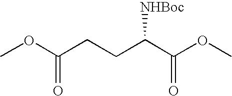 L-glutamic acid (6.0 g, 40.78 mmol) was dissolved in methanol (100 mL). To the reaction mixture trimethylsilyl chloride (22.9 mL, 180 mmol) was added at 0° C. under N₂ and allowed to stir overnight. To the reaction mixture at 0° C. under N₂ triethylamine (37 mL, 256 mmol) and di-tert-butyldicarbonate (9.8 g, 44.9 mmol) was added and stirred 2 hr. The solvent was removed and the residue was triturated with ether (200 mL). The triturated mixture was filtered. The filtrate was evaporated to an oil and chromatographed on silica, eluting with ethyl acetate and hexane, to give the mono boc L-glutamic diester (10.99 g, 98%).

### Ex-U-2

### Preparation of

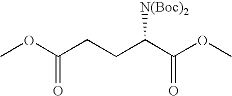 Mono boc L-glutamic acid (10.95 g, 39.8 mmol) was dissolved in acetonitrile (130 mL). To the reaction mixture 4-dimethylaminopyridine (450 mg, 3.68 mmol) and di-tert-butyldicarbonate (14.45 g, 66.2 mmol) was added and stirred for 20 hr. The solvent was evaporated and the residue chromatographed on silica and eluting with ethyl acetate and hexane to give the di-boc-L-glutamic diester (14.63 g, 98%).

### Ex-U-3

### Preparation of

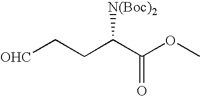 The product from Ex-U-2 (10.79 g, 28.7 mmol) was dissolved in diethyl ether (200 mL) and cooled in a dry ice bath to −80 C. To the reaction mixture Diisobutylaluminum hydride (32.0 mL, 32.0 mmol) was added and stirred 25 min. The reaction mixture was removed from the dry ice bath and water (7.0 mL) was added. Ethyl acetate (200 mL) was added to the reaction mixture and stirred 20 min. Magnesium sulfate (10 g) was added to the reaction mixture and stirred 10 min. The reaction mixture was filtered through celite and concentrated to give a clear yellow oil (11.19 g). The yellow oil was chromatographed on silica and eluting with ethyl acetate and hexane. The product (8.61, 87%) was a clear light yellow oil. Mass Spectrometry: M+H 346, M+Na 378. (¹H)NMR (400 MHz, CDCl₃) 9.74 ppm (s, 1H), 4.85 ppm (m, 1H), 3.69 ppm (s, 3H), 2.49 ppm (m, 3H), 2.08 ppm (m, 1H), 1.48 ppm (s, 18H).

### Ex-U-4

### Preparation of

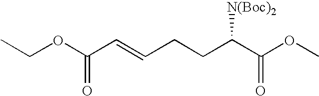 Triethyl phosphonoacetate (6.2 mL, 31.2 mmol) was dissolved in toluene (30 mL) and placed in an ice bath under N₂ and cooled to 0° C. To the reaction mixture, potassium bis(trimethylsilyl) amide (70 mL, 34.9 mmol) was added and stirred 90 min. To the reaction mixture the product from Ex-U-3 (8.51 g, 24.6 mmol) dissolved in toluene (20 mL) was added and stirred 1 hr. The reaction mixture was warmed to room temperature. To the reaction mixture Potassium hydrogen sulfate (25 mL, 25 mmol) was added and stirred 20 min. The mixture was extracted with ethyl acetate (3×100 mL), dried over Magnesium sulfate and concentrated to give a cloudy brownish yellow oil (12.11 g). The oil was chromatographed on silica, eluted with ethyl acetate and toluene to give a light yellow oil (7.21 g, 70%). Mass Spectrometry: M+H 416, M+NH₄⁻433,-boc 316,-2 boc, 216. (¹H)NMR (400 MHz, CDCl₃) 6.88 ppm (m, 1H), 5.82 ppm (d, 1H), 4.81 ppm (m, 1H), 5.76 ppm (s, 3H), 2.50ppm (m, 3H), 2.21 ppm (m, 1H), 1.45 ppm (s, 18H).

### Ex-U-5

### Preparation of

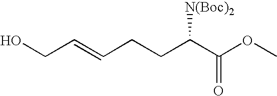 The product from Ex-U-4 (5.0 g, 12.03 mmol) was dissolved in diethyl ether (100 mL) and placed in a dry ice bath and cooled to −80° C. To the reaction mixture was added diisobutylaluminum hydride (21.0 mL, 21.0 mmol). The mixture was then stirred 30 min. To the reaction mixture water (10 mL) was added, removed from dry ice bath, and stirred 60 min. To the reaction mixture magnesium sulfate (10 g) was added and stirred 10 min. The reaction mixture was filtered over celite and concentrated to give a yellow oil (5.0 g). The oil was chromatographed on silica, eluted with ethyl acetate and hexane, to give a light yellow oil (2.14 g, 47%). Mass Spectrometry: M+H 374, M+NH₄ 391. (¹H)NMR (400 MHz, CDCl₃) 5.63 ppm (m, 2H), 4.88 ppm (m, 1H), 4.02 ppm (s, 2H), 3.68 ppm (s, 3H), 2.12 ppm (m, 4H), 1.47 ppm (s, 18H).

### Ex-U-6

### Preparation of

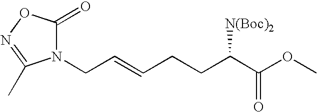 The product from Ex-U-5 was dissolved in tetrahydrofuran (50 mL). To the reaction mixture triphenyl phosphine on polymer (3.00 g, 8.84 mmol), oxadiazolinone (720 mg, 7.23 mmol), and azodicarboxylic acid dimethyl ester (1.17 g, 3.21 mmol) were added and stirred 6 hr at room temperature. The reaction mixture was filtered over celite and concentrated to give a cloudy yellow oil (2.81 g). The oil was chromatographed on silica, eluting with ethyl acetate in hexane, to give a clear colorless oil (1.66 g, 68%). Mass Spectrometry: M+H 456, M+N 473, - boc 356,-2 boc 256. (¹H)NMR (400 MHz, CDCl₃) 5.65 ppm (m, 1H), 5.45 ppm (m,1H), 4.79 ppm (m, 1H), 4.11l ppm (d, 2H), 3.68 ppm (s, 3H), 2.17 ppm (m, 4H), 1.47 ppm (s, 18 H).

### Ex-U-7

### Preparation of

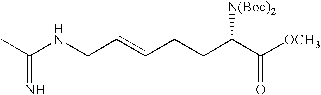 Product from Ex-U-6 (300 mg, 0.66 mmol) was dissolved in a solution of acetic acid and water (10 mL, 25/75) containing zinc metal and sonicated for 3 hr. The reaction mixture was filtered over celite and chromatographed on reverse phase HPLC to give a clear colorless residue (13 mg, 4%). (¹H)NMR (400 MHz, CDCl₃) 8.89 ppm (m, 1H), 5.68 ppm (m,1H), 5.47 ppm (m, 1H), 3.80 ppm (d, 2H), 3.71 ppm (s, 3H), 2.18 ppm (m, 4H), 1.41 ppm (s, 18 H).

### Ex-U-8

### Preparation of

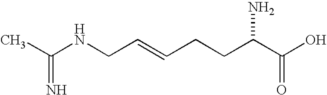 The product from Ex-U-7 (13.0 mg, 0.031 mmol) was dissolved in 2 N HCl (1.22 mL, 2.44 mmol) and refluxed 1 hr. The reaction mixture was cooled, concentrated, to give a clear colorless oil (6.6 mg, 95%). Mass Spectrometry: M+H 200, (¹H)NMR (400 MHz, D₂O) 5.65 ppm (m, 1H), 5.47 ppm (m,1H), 3.80ppm (t, 1H), 3.72 ppm (d, 2H), 2.0 ppm (m, 5H), 1.87 ppm (m, 2H).

### Example V

### Preparation of (αR,2S)-α-aminohexahydro-7-imino-1H-azepine-2-hexanoic acid, trihydrate hydrochloride

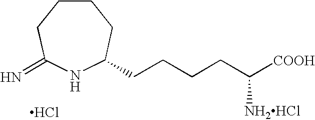

### Ex-V-1

### Preparation of

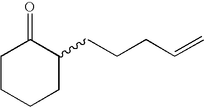 A 3-neck 3L flask was purged with N₂ before it was charged with cyclohexanone (1.27 mol, 132 mL) and 500 mL of toluene. This stirred mixture was cooled to 0° C. and 157.2 g (1.1 eq) of potassium t-butoxide was added. After stirring this mix for 1 hr, a color and texture change was noted before a solution of 5-pentenyl bromide (1.27 mol, 136 mL) in 100 mL toluene was added dropwise over 1 hr to the mechanically stirred reaction mixture. The reaction mixture was allowed to warm to 25° C. and stir overnight. It was then diluted with 800 mL of 1 N KHSO₄ and the organic phase was dried (MgSO₄), filtered and evaporated to dryness to yield 208.5 g of crude product. This material was then purified by vacuum distillation (under water aspirator pressure) to give the title product in 47% yield. ¹H NMR (CDCl₃, δ ppm): 1.0-2.4 (m, 13H), 4.9-5.1 (m, 2H), 5.7-5.9 (m, 1H).

### Ex-V-2

### Preparation of

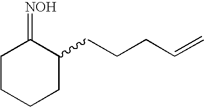 The product of Ex-V-1 (93.67 g, 0.563 mole) along with EtOH (600 mL), water (300 mL), NaOAc (101.67 g, 1.24 mole), and NH₂OH.HCl (78.31 g, 1.13 mole) were combined in a 3-neck 3 L flask. This stirred reaction mixture was refluxed for 16 hr and then stirred at 25° C. for another 24 hr. All solvent was removed under reduced pressure and the residue was partitioned between diethylether (Et₂O, 500 mL) and water (200 mL). The aqueous layer was extracted 3×200 mL ether. The combined organic layers were dried over MgSO₄, filtered, and stripped in vacuo to give the title oxime (121.3 g, 100% crude yield). ¹H NMR (CDCl₃, δ ppm): 1.2-2.6 (m, 13H), 4.9-5.1 (m, 2H), 5.7-5.9 (m, 1H).

### Ex-V-3

### Preparation of

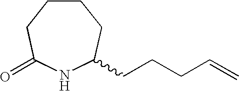 A 3-neck 3 L flask was purged with N₂ and then charged with hexamethydisiloxane (471.7 mL, 2.2 moles), toluene (500 mL), and phosphorous pentoxide (203.88 g, 1.4 moles). This heterogeneous mixture was refluxed until a clear solution was obtained (about 1.5 hr). After cooling this mixture to room temperature, the oxime product of Ex-V-2 (102.1 g, 0.563 moles) in 200 mL of toluene was added to the above reaction mixture over a 1 hr period at 25° C. The reaction mixture was stirred for another 4-6 hr (checked by TLC: 50% EA in Hex, I₂) before it was poured into ice water with thorough mixing. To this ice slurry mixture was added 250 g of NaCl and the resulting mixture was adjusted to pH 5 by adding solid potassium carbonate. This slurry was extracted with 3×500 mL of diethylether (Et₂O) and the combined organic fractions were dried over MgSO₄, filtered and stripped in vacuo to give the crude mixture of regioisomeric lactams (84.6 g).

### Ex-V-4

### Preparation of

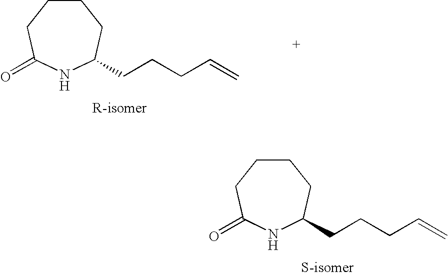 The product of Ex-V-3 was then subjected to chromatography (silica: acetonitrile) for purification and regioisomeric separation. From the crude sample, the 7-pentenyl regioisomer was isolated in 50% yield and after chiral chromatography, the desired single enantiomers were isolated in 43% yield each. R-isomer: Elemental analyses Calcd for C₁₁H₁₉NO: C, 71.99; H, 10.57; N, 7.63. Found: C, 71.97; H, 10.58; N, 7.52. ¹H NMR (CDCl₃, δ ppm): 1.3-1.6 (m, 7H), 1.75-1.9 (m, 2H), 1.95-2.15 (m, 3H), 2.4-2.5 (m, 2H), 3.25-3.35 (m, 1H), 4.95-5.05 (m, 2H), 5.7-5.85 (m, 1H). ¹³C NMR (CDCl₃, δ ppm): 23.166, 25.169, 29.601, 33.209, 35.475, 35.624, 36.783, 53.600, 114.976, 137.923, 177.703. [α]²⁵=+26.90 (CHCl₃) at 365 nm. S-isomer: Elemental analyses Calcd for C₁₁H₁₉NO: C, 71.99; H, 10.57; N, 7.63. Found: C, 72.02; H, 10.61; N, 7.57. ¹H NMR (CDCl₃, δ ppm): 1.3-1.6 (m, 7H), 1.75-1.9 (m, 2H), 1.95-2.15 (m, 3H), 2.4-2.5 (m, 2H), 3.25-3.35 (m, 1H), 4.95-5.05 (m, 2H), 5.7-5.85 (m, 1H). ¹³C NMR (CDCl₃, δ ppm): 23.187, 25.178, 29.630, 33.230, 35.526, 35.653, 36.778, 53.621, 115.032, 137.914, 177.703. [α]²⁵=−25.70 (CHCl₃) at 365 nm.

### Ex-V-5

### Preparation of

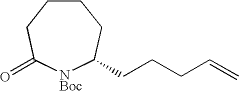 The R-isomer product of Ex-V-4 (102.1 g, 0.56 mol), dry THF (800 mL), DMAP (68.9 g, 0.56 mol), Di-t-butyl dicarbonate (Boc₂O, 99 g, 0.45 mol) were combined in a 3-neck 3L flask purged with argon. The reaction mixture was warmed to 70° C. within 30 min before an additional 52.8 g of Boc₂O and 200 mL of dry THF were added. After 30 min. another 32 g of Boc₂O was added and the mixture was stirred for 1 hr at 70° C. Another 36 g of Boc₂O was added and the mixture was stirred for 1 hr. The reaction mixture was cooled to room temperature and stripped of THF at 18° C. to 20° C. under reduced pressure. A precipitate was filtered and washed with 100 mL of ethylacetate (EA) and discarded ( 45 g). The EA filtrate was diluted with 500 mL of additional EA before it was washed with 500 mL of 1N KHSO₄, 500 mL of saturated aq. NaHCO₃, and 500 mL of brine and then dried over anhydrous Na₂SO₄ for 12 hr. This EA extract was then treated with 20 g of DARCO, filtered through celite topped with MgSO₄, and concentrated in vacuo to give 150 g of title product as a dark brown oil. ¹H NMR (CDCl₃, δ ppm): 1.3-1.6 (m, 4H), 1.5 (s, 9H), 1.6-1.9 (m, 6H), 1.95-2.05 (m, 2H), 2.5-2.7 (m, 2H), 4.2-4.25 (m, 1H), 4.95-5.05 (m, 2H), 5.7-5.85 (m, 1H).

### Ex-V-6

### Preparation of

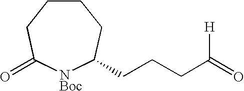 A 3-neck 3L flask containing the product of Ex-V-5 (150 g, 0.533) dissolved in 3 L of CH₂Cl₂ was cool to −78° C. A stream of O₃ was passed through the solution for 2.5 hr until the color of the reaction mixture turned blue. Argon was then bubbled through the solution maintained at −60° C. to −70° C. until the solution became clear and colorless (˜30 min.). Dimethylsulfide (DMS, 500 mL) was then added before the reaction was brought to reflux and this reflux was continued for 24 hr. Another 100 mL of DMS was added and reflux was continued for 12 hr. Another 100 mL of DMS was added and reflux continued for an additional 12 hr. The solvent and excess DMS were then stripped on a rotary evaporator at 20° C. The residual yellow oil obtained was diluted with 500 mL of DI water and extracted with 3×300 mL of EA. The EA layer was dried over anhydrous MgSO₄, treated with 20 g of DARCO, filtered through a thin layer of celite topped with anhydrous MgSO₄, and stripped of all solvent under reduced pressure to yield 156 g of the crude title product as orange yellow oil. ¹H NMR (CDCl₃, δ ppm): 1.3-1.6 (m, 4H), 1.5 (s, 9H), 1.6-1.9 (m, 6H), 2.45-2.75 (m, 4H), 4.2-4.25 (m, 1H), 9.75 (s, 1H).

### Ex-V-7

### Preparation of

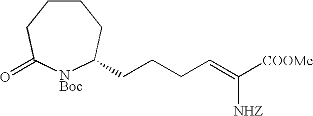 To a sample of N-(Benzyloxycarbonyl)-alpha-phosphonoglycine trimethyl ester (160 g, 0.48 mol) dissolved in 1 L of dichloromethane (CH₂Cl₂) and cooled to 0° C. was added a solution of DBU (110.29 g, 0.72 mol) in 100 mL of CH₂Cl₂. This clear colorless reaction mixture was stirred for 1 h at 0° C. to 6° C. before the Boc-aldehyde product of Ex-V-6 (150 g, 0.53 mol) in 600 mL of CH₂Cl₂ was added drop wise at −5° C. to −1° C. The reaction mixture was stirred for 30 min. at this temperature before it was slowly warmed to 10° C. in approximately 1 hr. The reaction mixture was washed with 1N KHSO₄ (500 mL), saturated aq. NaHCO₃ (200 mL) and 50 aq. NaCl (200 mL). The organic layer was then dried over anhydrous MgSO₄, treated with 40 g of DARCO, filtered through a thin layer of celite topped with anhydrous MgSO₄, and concentrated to give 258 g of the crude title product as an yellow oil. Chromatographic purification of this material gave 130 g (55%) of the pure title product. Elemental analyses Calcd for C₂₆H₃₆N₂O₇:C, 63.96; H,7.42; N, 5.77. Found: C, 63.42; H, 8.16; N, 5.31. ¹H NMR (CDCl₃, δ ppm): 1.25 (m, 2H), 1.5 (s, 9H), 1.51-1.9 (bm, 8H), 2.25 (m, 2H), 2.5 (m, 1H), 2.65 (m, 1H), 3.75 (s, 3H), 4.12 (m, 1H), 5.15 (s, 2H), 6.3 (bs, 1H), 6.55 (t, 1H), 7.45 (m,5H). ¹³C NMR (CDCl₃, δ ppm): 14.04, 22.62, 23.46, 24.08, 25.27, 27.89, 27.92, 28.34, 28.95, 31.81, 31.86, 32.05, 39.18, 52.31, 54.65, 67.27, 82.62, 128.07, 128.18, 128.46, 135.98, 136.82, 154.50, 164.92, 176.68. [α]²⁵=+10.90 (CHCl₃) at 365 nm.

### Ex-V-8

### Preparation of

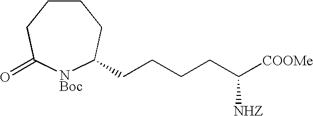 To a MeOH (1 L) solution of the product of Ex-V-7 (91.3 g, 0.19 mol) was added 2.5 g of S,S-Rh-DIPAMP catalyst followed by hydrogen. The hydrogenation was carried out at 25° C. in 1.5 hr in a Parr apparatus. The reaction mixture was filtered through celite before concentrating to provide the crude title product (90 g, 98%) as a brown oil. ¹H NMR (CDCl₃, δ ppm): 1.35 (m, 4H), 1.5 (s, 9H), 1.55-1.95 (m, 10H), 2.4-2.7 (m, 2H), 3.75 (s, 3H), 4.2 (m, 1H), 4.4 (m, 1H), 5.1 (m, 2H), 5.35 (d, 1H), 7.35 (m, 5H).

### Ex-V-9

### Preparation of

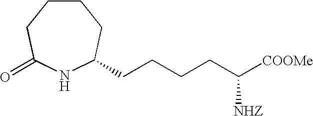 To a solution of the product of Ex-V-9 (90 g,) in 200 mL of glacial acetic acid was added 200 mL of 4N HCl in dioxane. The reaction mixture was stirred at 25° C. for 20 min. before it was stripped of all solvent under reduced pressure at 40° C. to give a red brown oil. This oily product was treated with 500 mL of water and extracted 2×300 mL of dichloromethane. The combined organic layer was washed with satd. sodium bicarbonate solution (100 mL), dried over magnesium sulfate, filtered and stripped of all solvent to give the crude title product. This material was chromatographed to provide 45 g (62%) of the pure title product. Elemental analyses Calcd for C₂₁H₃₀N₂O₅:C, 64.02; H, 7.68; N, 7.17. Found: C, 63.10; H, 7.88; N, 6.60. ¹H NMR (CDCl₃, δ ppm): 1.2-2.0 (m, 14H), 2.45 (t, 2H), 3.25 (m,1H), 3.75 (s, 3H), 4.38 (m, 1H), 5.1 (s, 2H), 5.3 (d, 1H), 5.45 (bs, 1H), 7.35 (m, 5H). ¹³C NMR (CDCl₃, δ ppm): 14.09, 23.11, 24.89, 25.41, 29.53, 32.33, 35.52, 35.79, 36.68, 52.26, 53.51, 53.55, 53.60, 60.26, 66.86, 127.97, 128.05, 128.40, 136.18, 155.85, 172.85, 177.80. [α]²⁵=−9.90 (CHCl₃) at 365 nm.

### Ex-V-10

### Preparation of

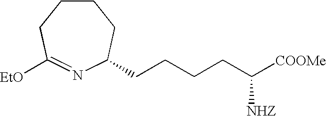 To a 45.0 g (0.115 mol) sample of the product of Ex-V-9 in 300 mL of dichloromethane purged with argon was added 23.0 g (0.121 mol) of triethyloxonium tetrafluoroborate. This mixture was stirred for 1 hr at 25° C. before 150 mL of satd. aq. sodium bicarbonate solution was added. The dichloromethane layer was separated, washed with 150 mL of 50% aq. NaCl solution, dried over sodium sulfate, filtered through celite and concentrated at 25° C. to give a clear yellow oil, 47.0 g (97%) of the title product. Elemental analyses Calcd for C₂₃H₃₄N₂O₅:C, 60.01; H, 8.19; N, 6.69. Found: C, 65.13; H, 8.45;N, 6.64. ¹H NMR (CDCl₃, δ ppm): 1.2 (t, 3H), 1.25-1.74 (m, 12H), 1.75-1.95 (m, 2H), 2.2-2.3 (m, 1H), 2.4-2.5 (m, 1H), 3.1 (m, 1H), 3.7 (s, 3H), 3.9-4.0 (m, 2H), 4.35 (m, 1H), 5.1 (s, 2H), 5.25 (d, 1H), 7.35 (m, 5H). ¹³C NMR (CDCl₃, δ ppm): 14.23, 23.38, 25.01, 25.21, 26.10, 30.24, 32.16, 32.77, 33.92, 39.15, 52.22, 53.91, 58.05, 60.19, 66.92, 128.11, 128.33, 128.48, 136.27, 155.83, 166.29, 173.11, 177.64.

### Ex-V-11

### Preparation of

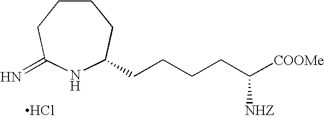 To 7.0 g (0.130 mol) of ammonium chloride in 500 mL methanol was added 31.2 g of the title material of Ex-V-10 (45.0 g, 0.107 mol). The reaction was refluxed at 65° C. for 5 hr before all solvent was removed under reduced pressure to yield 40 g (87%) of the crude product as a foamy viscous mass. This material was purified by column chromatography to provide 37 g (81%) of the title product. Elemental analyses Calcd for C₂₁H₃₁N₃O₄: C, 59.22; H, 7.57; N, 9.86; Cl, 8.32. Found for C₂₁H₃₁N₃O₄+1.2 HCl+0.5 H₂O: C, 57.20; H, 7.99; N, 9.66; Cl, 9.62. IR (Neat, δ max cm⁻¹): 2935, 1716, 1669. ¹H NMR (CDCl₃, δ ppm): 1.2-2.0 (m, 13H), 2.5 (t, 1H), 2.95 (m, 1H), 3.4 (bs, 1H), 3.7 (s, 3H), 4.3 (m, 1H), 5.1 (s, 2H), 5.55 (d, 1H), 7.3 (m, 5H), 8.75 (bs,1H), 8.9 (bs, 1H), 9.5 (s, 1H). ¹³C NMR (CDCl₃, δ ppm): 23.20, 24.95, 25.22, 28.94, 31.80, 32.05, 33.75, 34.89, 52.33, 53.76, 56.07, 66.83, 127.93, 128.04, 128.43, 136.26, 156.00, 172.24, 172.87. Mass (ESI): M/Z, 390. [α]²⁵=+31.50 at 365 nm.

### Ex-V-12

### Preparation of

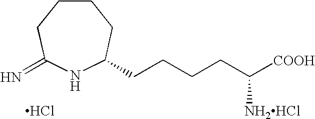 The title product of Ex-V-11 (36.0 g, 0.084 mol) in 1 L of 2.3 N HCl was refluxed for 3 hr. After cooling to room temperature, the solution was washed with 2×150 mL of CH₂Cl₂ and then stripped of all solvent in vacuo to give 25.6 g (96%) of the title amino acid product as a pale yellow foam. Elemental analyses Calcd for C₁₂H₂₃N₃O₂.2HCl: C, 46.02; H, 8.01; N, 13.39; Cl 22.45. Found for C₁₂H₂₃N₃O₂+2.2 HCl+0.1 H₂O: C, 42.76; H,8.02; N, 12.41; Cl, 22.79. IR (Neat, δ max, cm⁻¹): 2930, 2861, 1738,1665. ¹H NMR (CD₃OD, δ ppm): 1.3-2.5 (m, 16H), 2.6 (dd, 1H), 2.8 (t, 1H), 3.65 (m, 1H), 4.0 (t, 1H), 7.85 (s, 1H), 8.85 (s, 1H), 8.95 (s, 1H). ¹³C NMR (CD₃OD, δ ppm): 24.49, 25.67, 26.33, 29.71, 31.26, 32.45, 35.04, 35.87, 53.73, 57.21, 171.77, 173.96. UV, 282 nm, abs 0.015. Mass (M⁺¹)=242. [α]²⁵=−47.40 (MeOH) at 365 nm. ee=91% as determined by CE at δ=214 nm.

### Example W

### Preparation of (αS,2R)-α-aminohexahydro-7-imino-1H-azepine-2-hexanoic acid, trihydrate hydrochloride

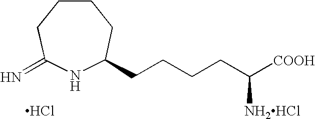

### Ex-W-1

### Preparation of

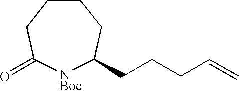 The S-isomer product of Ex-V-4 (5.45 g, 0.030 mol) was converted to its Boc derivative by the method of Ex-V-5. After chromatography, this reaction yielded 6.3 g (75%) of the desired title product. ¹H NMR (CDCl₃, δ ppm): 1.3-1.6 (m, 4H), 1.5 (s, 9H), 1.6-1.9 (m, 6H), 1.95-2.05 (m, 2H), 2.5-2.7 (m, 2H), 4.2-4.25 (m, 1H), 4.95-5.05 (m, 2H), 5.7-5.85 (m, 1H).

### Ex-W-2

### Preparation of

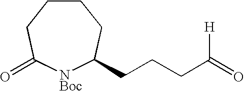 The product of Ex-W-1 (6.3 g, 0.025 mol) was ozonized by the method of Ex-V-6 to produce 8.03 g of the crude title aldehyde that was used without further purification. ¹H NMR (CDCl₃, δ ppm): 1.3-1.6 (m, 4H), 1.5 (s, 9H), 1.6-1.9 (m, 6H), 2.45-2.75 (m, 4H), 4.2-4.25 (m, 1H), 9.75 (s, 1H).

### Ex-W-3

### Preparation of

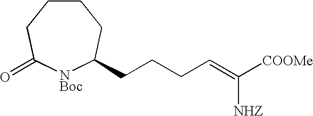 The product of Ex-W-2 (8.03 g, 0.024 mol) was condensed with N-(Benzyloxycarbonyl)-alpha-phosphonoglycine trimethyl ester (7.9 g, 0.024 mol) utilizing the procedure of Ex-V-7 to produce 4.9 g (44%) of the desired title product after chromatography. ¹H NMR (CDCl₃, δ ppm): 1.25 (m, 2H), 1.5 (s, 9H), 1.51-1.9 (bm, 8H), 2.25 (m, 2H), 2.5 (m, 1H), 2.65 (m, 1H), 3.75 (s, 3H), 4.15-4.25 (m, 1H), 5.15 (s, 2H), 6.3-6.4 (bs, 1H), 6.45-6.55 (t, 1H), 7.3-7.4 (m,5H).

### Ex-W-4

### Preparation of

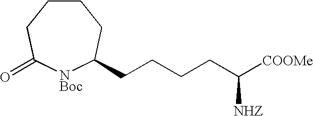 The product of Ex-W-3 (4.8 g, 0.010 mol) was reduced in the presence of R,R-Rh-DIPAMP catalyst by the method of Ex-V-8 to produce 2.9 g (60%) of the desired title product after chromatography.

### Ex-W-5

### Preparation of

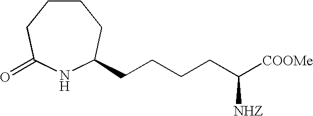 The product of Ex-W-4 (2.9 g, 0.006 mol) was deprotected by treatment with HCl using the method of Ex-V-9 to produce 2.3 g (100%) of the desired title product. ¹H NMR (CDCl₃, δ ppm): 1.3-2.0 (m, 14H), 2.45 (t, 2H), 3.25 (m,1H), 3.75 (s, 3H), 4.38 (m, 1H), 5.1 (s, 2H), 5.3 (d, 1H), 5.45 (bs, 1H), 7.35 (m, 5H).

### Ex-W-6

### Preparation of

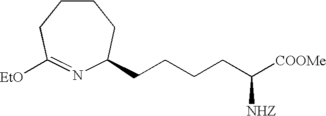 The product of Ex-W-5 (0.56 g, 0.0015 mol) was alkylated with triethyloxonium tetrafluoroborate using the method of Ex-V-10 to produce 0.62 g (98%) of the desired title product.

### Ex-W-7

### Preparation of

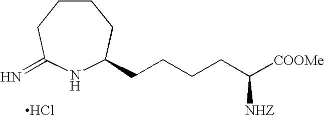 The product of Example W-6 (0.62 g, 0.0015 mol) was treated with ammonium chloride in methanol using the method of Ex-V-11 to produce 0.50 g (88%) of the desired title product after chromatographic purification.

### Ex-W-8

### Preparation of

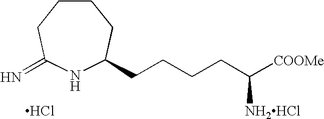 The product of Ex-W-7 (0.37 g, 0.0009 mol) dissolved in MeOH was added to a Parr hydrogenation apparatus. To this vessel was added a catalytic amount of 5%Pd/C. Hydrogen was introduced and the reaction was carried out at room temperature at pressure of 5 psi over a 7 hr period. The catalyst was removed by filtration and all solvent was removed under reduced pressure from the filtrate to produce 0.26 g (quantitative) of the desired title product.

### Ex-W-9

### Preparation of

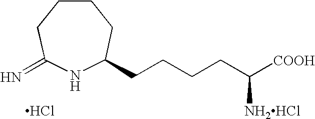 A solution of the product of Ex-W-8 dissolved in 2N HCl (30 mL) was maintained at reflux for 2 hr before it was cooled to room temperature. All solvent was removed under reduced pressure and the residue was dissolved in 50 mL of water. This solution was again stripped of all solvent under reduced pressure before it was again dissolved in 12 mL of water and then lyophilized to generated 0.245 g (71%) of the title compound. Elemental analyses Calcd for C₁₂H₂₃N₃O₂.2.3 HCl.1.9 H₂O: C, 40.10; H, 8.16; N, 11.69; Cl 22.69. Found for C₁₂H₂₃N₃O₂+2.1 HCl+0.7 H₂O: C, 40.27; H, 8.28; N, 11.62; Cl, 22.70. ¹H NMR (CD₃OD, δ ppm): 1.4-2.1 (m, 16H), 2.6 (dd, 1H), 2.8 (t, 1H), 3.65 (m, 1H), 4.0 (t, 1H), 7.85 (s, 1H), 8.45 (s, 1H), 8.9 (s, 1H). ¹³C NMR (CD₃OD, δ ppm): 24.46, 25.64, 26.31, 29.69, 31.24, 32.54, 35.00, 35.83, 53.75, 57.20, 171.85, 173.93. [α]²⁵=+25.70 (MeOH) at 365 nm.

### Example X

### Preparation of (αS,2S)-α-aminohexahydro-7-imino-1H-azepine-2-hexanoic acid, trihydrate hydrochloride

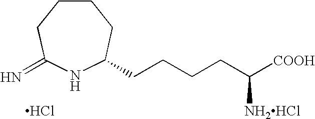

### Ex-X-1

### Preparation of

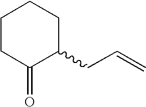 To a 22 L round bottom flask equipped with overhead stirrer, half moon shape paddle, heating mantle, thermocouple, and a silver vacuum jacketed distillation column (5 plates) was charged cyclohexanone (4500.0 g, 45.85 mol), acetone dimethyl acetal (5252.6 g, 50.43 mol), allyl alcohol (6390.87 g, 110.04 mol) and p-toluene sulfonic acid (PTSA) (0.256 g, 0.001 mol). After the stirring was started (137 rpm) the pot was heated slowly with the initial set point being 70° C. Heating was increased step wise to a final pot temperature of 150° C. The decision to increase the reactor set point was made based on distillation rate. If the rate of distillate slowed or stopped, additional heat was applied. The additional heating to 150° C. allowed the Claisen rearrangement to occur. After the pot temperature was raised to 150° C. and no distillate was observed, the heating mantle was lowered and the reaction mixture allowed to cool to 130° C. The PTSA was then neutralized with 3 drops of 2.5 N NaOH. The vacuum stripping was then started with the heating mantle lowered away from the flask. Evaporative cooling was used to lower the pot temperature, and the pressure was gradually lowered to 40 mm Hg. When the pot temperature had decreased to ˜100° C., the heating mantle was raised back into the proper position for heating. Unreacted cyclohexanone and low boiling impurities were distilled off. The pot temperature was slowly raised (the maximum temperature deferential between the pot and vapor was ˜12° C.). The product was isolated at 109-112° C.@40 mm Hg. Typical yields were 40-45%. Fractions which were <95% by area (GC) were combined and redistilled to afford the title product in a total yield of 55%. ¹H NMR (CDCl₃, δ ppm): 5.8-5.6 (m, 1H), 4.8-5.0 (m, 2H), 2.5-2.4 (m, 1H), 2.3-2.1 (m, 3H), 2.1-1.2 (m, 7H). ¹³C NMR (CDCl₃, δ ppm): 212.53, 136.62, 116.32, 50.39, 42.18, 33.91, 33.52, 28.09, 25.10. GC/MS m/z=138.

### Ex-X-2

### Preparation of

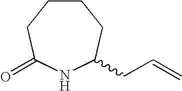 Hydroxyl amine-O-sulfonic acid (91.8 g) dissolved in acetic acid (470 g) was added to a 1 L Bayer flask equipped with a mechanical stirrer, thermocouple, condenser chilled to 0° C., and an addition funnel and heated to 70° C. The allyl cyclohexone (100 g) was added dropwise in approximately 40 min to the above solution while maintaining the temperature between 70 and 78° C. During the addition, the reaction appearance changed from a white slurry to a clear orange solution. After the addition, the reaction was heated and stirred for an additional 5 hr at 75° C. An IPC sample was taken each hour. After the reaction was complete, the acetic acid was stripped at 50° C. under reduced pressure on a rotary evaporator. Water (200 mL) was then added to the residue and the solution extracted with toluene (2×300 mL). The organic layers were combined, treated with water (150 ml) and stirred for 10 min. A sodium hydroxide solution (79.4 g of 50 solution) was added until the aqueous layer turned basic (pH 12). The neutralization was carried out in the reactor by controlling the temperature below 40° C. The layers were then separated and the toluene layer was passed through a filter to remove any solids or tarry material. The organic solution was then stripped at 50° C. under reduced pressure on a rotary evaporator. The residue was taken up in a mixture of toluene (510 mL) and heptanes (2040 mL) and heated to 60° C. in a 3 L reactor. A clear yellow-orange solution was obtained. The title product began to crystallize at 53° C. as the solution was slowly cooled to 5° C. while being stirred. The solid was filtered, washed with heptanes (50 mL) and dried over night at 40° C. under house vacuum to produce 66.3 g (60%) of title product as off-white crystals obtained. A portion of this material was recrystallized from toluene and heptane to generate the title product as a white crystalline solid. ¹H NMR (CDCl₃, δ ppm): 5.8-5.6 (m, 1H), 5.5 (bs, 1H), 4.8-5.0 (m, 2H), 3.4-3.3 (m, 1H), 2.5-2.3(m, 2H), 2.3-2.1 (m, 2H) 2.0-1.2 (m, 6H). ¹³C NMR (CDCl₃, δ ppm): 117.73, 133.83, 119.31, 52.88, 40.95, 37.20, 35.75, 29.96, 23.33. GC/MS (EI mode)=153. m.p.=97-99° C.

### Ex-X-3

### Preparation of

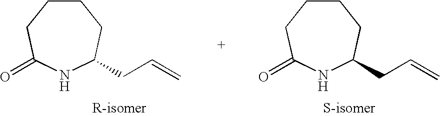 The racemic product mixture of Ex-X-2 was subjected to chiral chromatographic separation on a Chiralpac AS 20 μm column eluting with 100% acetonitrile. A 220 nM wavelength was employed in the detector. A sample loading of 0.08 g/mL of acetonitrile was used to obtain 90% recovery of separated isomers each with >95% ee. A portion of the R-isomer material was recrystallized from toluene and heptane to generate the R-isomer title product as a white crystalline solid. R-isomer: m.p.=81-82° C.

### Ex-X-4

### Preparation of

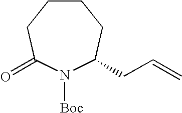 A 5-necked flat bottom flask equipped with dropping funnel, thermometer and mechanical overhead stirrer was evacuated and purged with N₂ 3 times. The R-isomer product lactam of Ex-X-3 (100.0 g, 0.653 mol), DMAP (7.98 g, 65 mmol) and N-diisopropylethyl amine (Hünigs base, 113.3 g, 0.876 mol) were dissolved in toluene (350 mL) and Di-tert-butyl dicarbonate (170.2 g, 0.78 mol) dissolved in toluene (100 mL) was added. (Note: the reaction works better, when 2.0 eq of Hünigs base were used). The mixture was heated to 65° C. (Note: Steady offgasing during the reaction was observed). After 1.5 hr another 86.25 g of Di-tert-butyl-dicarbonate (0.395 mol) dissolved in toluene (50 mL) were added. Heating was continued for 17 hr and IPC by HPLC showed 75% conversion. Another 42.78 g of di-tert-butyl dicarbonate (0.196 mol) in toluene (30 mL) were added and the brown mixture was heated 5.5 hr. After cooling to ambient temperature, the mixture was treated with 4M HCl (215 mL), and the aqueous layer was extracted with toluene (2×80 mL). The combined organic layers were washed with NaHCO₃ (170 mL) and 250 ml of water (Note: the internal temperature during the quench was controlled by external cooling with ice/water). Gas evolution was observed. The organic layer was evaporated to give 257.4 g brown liquid. This crude material was purified by plug filtration over SiO₂ (950 g) using toluene/EtOAc 9/1 (6 L) and toluene/AcOEt 1/1 (0.5 L) as eluent giving 139.5 g (51%) of the yellow liquid title product.

### Ex-X-5

### Preparation of

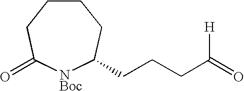 Into a 2-L stainless steel autoclave equipped with baffles and a 6-bladed gas dispersing axial impeller was charged Rh(CO)₂(acac) (0.248 g, 0.959 mmol), BIPHEPHOS (structure shown below and prepared as described in Example 13 of U.S. Pat. No. 4,769,498, 2.265 g, 2.879 mmol): 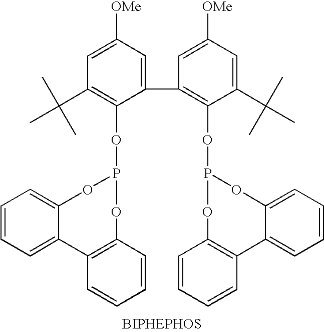
the product of Ex-X-4 (N-(tert-butoxycarbonyl)-S-7-allylcaprolactam (242.9 g, 0.959 mol), and toluene (965 g). The reactor was sealed and purged 100% carbon monoxide (8×515 kPa). The reactor was pressurized to 308 kPa (30 psig) with 100% carbon monoxide and then a 1:1 CO/H₂ gas mixture was added to achieve a total pressure of 515 kPa (60 psig). With vigorous mechanical agitation, the mixture was heated to 50° C. with a 1:1 CO/H₂ gas mixture added so as to maintain a total pressure of about 515 kPa (60 psig). After 22 hr, the mixture was cooled to about 25° C. and the pressure was carefully released. Vacuum filtration of the product mixture and evaporation of the filtrate under reduced pressure afforded a 267.7 g of a light yellow oil. Analysis by ¹H NMR was consistent with essentially quantitative conversion of the starting material with about 96% selectivity to the corresponding aldehyde product of Ex-X-5. This oil was used without further purification in the following example. ¹H NMR (CDCl₃) δ1.47 (s, 9H), 1.6-1.80 (m, 9H), 1.84-1.92(m, 1H), 2.41-2.58 (m, 3H), 2.61-2.71 (m, 1H), 4.2 (d, J=5.2 Hz, 1H), 9.74 (s, 1H).

### Ex-X-6

### Preparation of

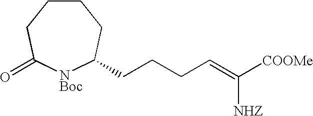 To a sample of N-(Benzyloxycarbonyl)-alpha-phosphonoglycine trimethyl ester (901.8 g, 2.7 mol) dissolved in CH₂Cl₂ and cooled to 0° C. was added a solution of DBU (597.7 g, 3.9 mol) in CH₂Cl₂. This clear colorless reaction mixture was stirred for 1 h at 0° C. to 6° C. before a sample of the Boc-aldehyde product Ex-X-5 (812.0 g, 2.9 mol) in CH₂Cl₂ was added drop wise at −5° C. to −1° C. The reaction, work up, and purification was completed as described in Ex-V-7 to give 1550 g of the title product of Ex-X-6 containing a small amount of CH₂Cl₂.

### Ex-X-7

### Preparation of

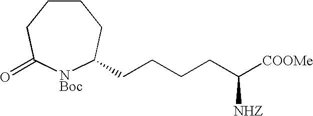 To a MeOH (1 L) solution of the product of Ex-X-6 (100 g, 0.20 mol) was added 3 g of RR-Rh-DIPAMP catalyst. The hydrogenation was carried out at 25° C. in 1.5 hr in a Parr apparatus. The reaction mixture was filtered through celite before concentrating to provide the crude Ex-X-7 title product as a brown oil (100 g). ¹H NMR (CDCl₃, δ ppm): 1.35 (m, 4H), 1.5 (s, 9H), 1.6-1.9(m, 10H), 2.5-2.8 (m, 2H), 3.75 (s, 3H), 4.25 (m, 1H), 4.45 (m, 1H), 5.1 (m, 2H), 5.65 (d, 1H), 7.35 (m, 5H).

### Ex-X-8

### Preparation of

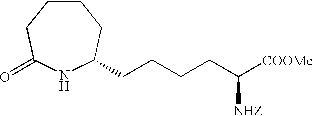 To a solution of the product of Ex-X-7 (100 g) in 200 mL glacial acetic acid was added 25 mL 4N HCl in dioxane. The reaction mixture was stirred at 25° C. for 20 min. before it was stripped of all solvent under reduced pressure at 40° C. to give 105 g of red brown oil. This oily product was treated with 500 mL of water and extracted 2×300 mL of dichloromethane. The combined organic layer was washed with satd. sodium bicarbonate solution (100 mL), dried over magnesium sulfate, filtered and stripped of all solvent to give 99.9 g of the title product as a red brown oil. ¹H NMR (CDCl₃, δ ppm): 1.25-2.0 (m, 14H), 2.45 (t, 2H), 3.25 (m,1H), 3.7 (s, 3H), 4.35 (m, 1H), 5.1 (s, 2H), 5.5 (d, 1H), 6.45 (bs, 1H), 7.35 (m, 5H). ee=95% as determined by chiral HPLC.

### Ex-X-9

### Preparation of

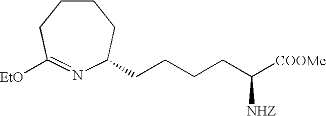 To a 30.0 g (0.077 mol) sample of the product of Ex-X-8 in 600 mL dichloromethane purged with argon was added 15.7 g (0.082mol) of triethyloxonium tetrafluoroborate. This mixture was stirred for 1 hr at 25° C. before 300 mL of satd. aq. sodium bicarbonate solution was added. The dichloromethane layer was separated, washed with 300 mL 50% aq. NaCl solution, dried over sodium sulfate, filtered through celite and concentrated at 25° C. to give a clear yellow oil, 31.2 g (97%) of the title product. Elemental analyses Calcd for C₂₃H₃₄N₂O₅: C, 60.01; H, 8.19; N, 6.69. Found for C₂₃H₃₄N₂O₅+0.5 H₂O: C, 64.66; H, 8.24;N,6.59. ¹H NMR (CDCl₃, δ ppm): 1.2 5(t, 3H), 1.28-1.75 (m, 12H), 1.8-1.98 (m, 2H), 2.2-2.3 (m, 1H), 2.4-2.5 (m, 1H), 3.1 (m, 1H), 3.78 (s, 3H), 3.9-4.0 (m, 2H), 4.35 (m, 1H), 5.1 (s, 2H), 5.25 (d, 1H), 7.35 (m, 5H). ¹³C NMR (CDCl₃, δ ppm): 14.27, 23.36, 25.21, 25.53, 26.09, 30.22, 32.15, 32.73, 33.90, 39.14, 52.21, 53.89, 58.04, 60.33, 66.89, 128.11, 128.35, 128.48, 136.29, 155.86, 166.30, 173.14, 177.69. IR (Neat, δ max, cm⁻¹): 3295, 2920, 1739, 1680. UV, 257 nm, abs 0.015. [α]²⁵=+39.80 (CHCl₃) at 365 nm.

### Ex-X-10

### Preparation of

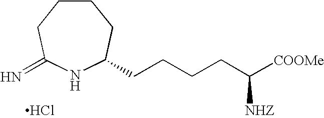 To 4.2 g (0.078 mol) of ammonium chloride in 500 mL methanol was added 31.2 g of the title material of Ex-X-9. The reaction was refluxed at 65° C. for 5 hr before all solvent was removed under reduced pressure to yield 29 g (92%) of the crude product as a foamy viscous mass. This material was purified by column chromatography to provide 23 g (70%) of the title product. Elemental analyses Calcd for C₂₁H₃₁N₃O₄.1HCl) C, 59.28; H, 7.57; N, 9.89; Cl, 8.39. Found (For C₂₁H₃₁N₃O₄+1HCl+1 H₂O): C, 56.73; H, 7.74; N, 9.40; Cl, 8.06. IR (Neat, δ max cm⁻¹): 3136, 30348, 2935, 1716, 1669. ¹H NMR (CDCl₃, δ ppm): 1.3-2.05 (m, 13H), 2.5 (t, 1H), 2.98 (m, 1H), 3.4 (bs, 1H), 3.75 (s, 3H), 4.35 (m, 1H), 5.1 (s, 2H), 5.5 (d, 1H), 7.35 (m, 5H), 8.75 (s,1H), 9.0 (s, 1H), 9.5 (s, 1H). ¹³C NMR (CDCl₃, δ ppm): 23.25, 25.01, 25.34, 29.01, 31.88, 32.26, 33.89, 35.06, 52.33, 53.73, 56.20, 66.89, 127.95, 128.06, 128.45, 136.27, 155.93, 172.27, 172.80. UV, 257 nm, abs 0.009. Mass (ESI): M/Z, 390. [α]²⁵=−42.80 (MeOH) at 365 nm. ee=96% as determined by chiral HPLC.

### Ex-X-11

### Preparation of

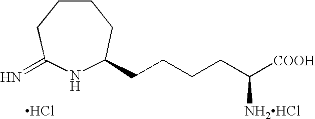 The title product of Ex-X-10 (23 g) in 500 mL 2N HCl was refluxed for 5 hr. All solvent was then removed in vacuo and the residue redissolved in water was washed with 2×300 mL of CH₂Cl₂. The aqueous was then concentrated in vacuo to give 17 g (100%) of the light brown hygroscopic solid title product. Elemental analyses Calcd for C₁₂H₂₃N₃O₂.2HCl: C, 45.86; H, 8.02; N, 13.37; Cl 22.56. Found for C₁₂H₂₃N₃O₂+2.1 HCl+0.7 H₂O: C, 43.94; H, 8.65; N, 12.52; Cl, 22.23. IR (Neat, δ max, cm⁻¹): 2936, 1742,1669. ¹H NMR (CD₃OD, δ ppm): 1.3-2.1 (m, 16H), 2.6 (dd, 1H), 2.8 (t, 1H), 3.65 (m, 1H), 4.0 (t, 1H), 7.85 (s, 1H), 8.4 (s, 1H), 8.95 (s, 1H). ¹³C NMR (CD₃OD, δ ppm): 24.49, 25.67, 26.33, 29.71, 31.26, 32.45, 35.04, 35.87, 53.73, 57.21, 171.77, 173.96. UV, 209 nm, abs 0.343. Mass (M⁺¹)=242. [α]²⁵=+60.00 (MeOH) at 365 nm. ee=92% as determined by CE at δ=210 nm.

### Example Y

### Preparation of (αR,2S)-α-aminohexahydro-7-imino-1H-azepine-2-hexanoic acid, trihydrate hydrochloride

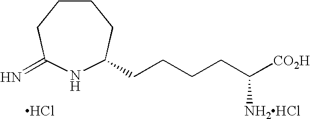

### Ex-Y-1

### Preparation of

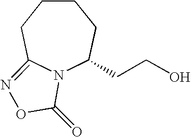 A solution of Ex-X-3 (3.0 g, 0.015 mol) in methylene chloride and methanol (75/45 mL) was cooled to −78° C. in a dry ice bath. The reaction stirred as ozone was bubble through the solution at a 3 ml/min flow rate. When the solution stayed a consistent deep blue, the ozone was removed and the reaction was purged with N₂. To the cold solution was added sodium borohydride (2.14 g, 0.061 mol) very slowly to minimize the evolution of gas at one time. To the reaction was added glacial acetic acid slowly to bring the pH to 3. The reaction was then neutralized with saturated sodium bicarbonate. The oraganics were then washed 3×50 mL with brine, dried over magnesium sulfate anhydrous, removed under reduced pressure. The pale oil was run through a plug of silica (15 g) to afford the alcohol 5.15 g, 0.026 mol (64%). C₉H₁₄N₂O₃. ¹H NMR (CDCl₃, δ ppm) 1.18-2.15(m, 8H), 3.59(m, 2H), 4.39(m, 1H). ¹³C NMR (CDCl₃, δ ppm) 24.45, 25.71, 26.47, 32.56, 34.67, 51.16, 58.85, 160.66, 160.89.

### Ex-Y-2

### Preparation of

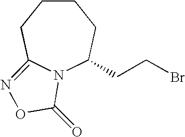 To a solution of Ex-Y-1 (5.15 g, 0.026 mol) in methylene chloride (100 mL) at 0° C. in an ice bath was added carbon tetrabromide(10.78 g, 0.033 mol). The solution was cooled to 0° C. in an ice bath. Then triphenylphosphine (10.23 g, 0.39 mol) was added portion wise as not to allow the temperature raise above 3° C. The reaction was stirred for 2 hr and the solvent was removed in vacuo. The crude was purified by flash chromatography to yield the bromide (5.9 g, 0.023 mol) in 87% yield. Elemental analysis calculated for C₁₀H₁₆N₂O₃: C, 41.40; H, 5.02; N, 10.73; Br, 30.60. Found: C, 41.59; H, 5.07; N, 10.60, Br, 30.86. ¹H NMR (CDCl₃, δ ppm) 1.50-2.60 (m, 9H), 2.99 (dd, 1H), 3.35 (m, 2H), 4.41 (m, 1H). ¹³C NMR (CDCl₃, δ ppm) 23.89, 25.33, 26.04, 28.06, 31.59, 35.05, 52.79, 159.3, 160.2.

### Ex-Y-3

### Preparation of

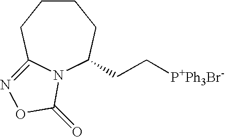 To a solution of Ex-Y-2 (5.71 g, 0.026 mol) in toluene (25 mL) was added triphenyl phosphine (7.17 g, 0.027 mol). The reaction refluxed in an oil bath for 16 hr. After cooling, the toluene was decanted from the glassy solid. The solid was triturated with diethyl ether overnight to afford the phosphonium bromide (10.21 g, 0.020 mol) in 90% yield. ¹H NMR (CDCl₃, δ ppm): 1.50-2.9 (m, 11H), 3.58 (m, 1H), 4.16 (m, 1H), 4.41 (m, 1H), 7.6-8.0 (m, 15H). ¹³C NMR (CDCl₃, δ ppm): 24.43, 24.97, 25.50, 55.08, 55.27, 116.9, 118.1, 130.4, 130.6, 133.5, 135.1, 135.2, 159.4, 160. ³¹P NMR (CDCl₃, δ ppm) 26.0.

### Ex-Y-4

### Preparation of

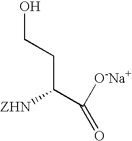 To a 1 L round bottom flask was added N-benzyloxycarbonyl-D-homoserine lactone (97 g, 0.442 mol) in ethanol (500 mL). To the reaction was added solution of sodium hydroxide (1M, 50mL). The reaction was monitored by thin layer chromatography for 12 hr until the starting material had been consumed. Toluene (60 mL) was added and then solvent was removed in vacuo. The residue was carried on with no further purification.

### Ex-Y-5

### Preparation of

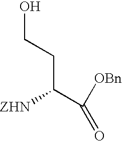 The residue from Ex-Y-4 was suspended in DMF in a 1 L round bottom flask. To the suspension was added benzyl bromide (76.9 g, 0.45 mol, 53.5 mL) and the mixture was stirred for 1 hr. A sample was quenched and analyzed by mass spec to indicate the consumption of the starting material and that there was no lactone reformation. To the reaction was added 1 L of ethyl acetate and 500 mL of brine. The aqueous layer was washed 2 additional times with 500 mL of ethyl acetate. The organics were combined, dried over MgSO₄ and concentrated. Silica gel chromatography provided N-benzyloxycarbonyl-S-homoserine benzyl ester as a white solid (80 g).

### Ex-Y-6

### Preparation of

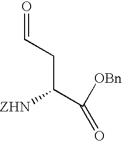 To a 2L Round Bottom Flask was added pyridinium chlorochromate (187 g, 0.867 mol) and silica gel (197 g) suspended in CH₂Cl₂ (600 mL). To the slurry was added a solution of the product of Ex-Y-5 (80 g, 0.233 mol) in CH₂Cl₂ (600 mL). The mixture was stirred for 4 hr. Thin layer chromatography indicated that the starting material was consumed. To the reaction was added 1 L of diethyl ether. The solution was then filtered through a pad of ceilite followed by a pad of silica gel. The solvent was removed in vacuo and the resulting oil was purified by silica gel chromatography to afford the aldehyde (58.8 g) in 38% overall yield. MH⁺342.5, MH+NH₄⁺359.5. ¹H NMR (CDCl₃, δ ppm) 3.15 (q, 2H), 4.12 (m, 1H), 5.15 (s, 2H), 5.20 (s, 2H), 7.31 (m, 10H), 9.72 (s,1H).

### Ex-Y-7

### Preparation of

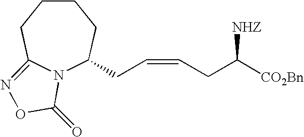 To a 3L 3-neck flask was added the phosphonium salt from Ex-Y-3 (56.86 g, 0.11 mol) that had been dried over P₂O₅ under a vacuum in THF (1 L). The slurry was cooled to −78° C. in a dry-ice bath. To the cold slurry was added KHMDS (220 mL, 0.22 mol) dropwise so that the temperature did not rise above −72° C. The reaction was stirred at −78° C. for 20 min and then −45° C. for 2 hr. The temperature was then dropped back to −78° C. and the aldehyde (15.9 g, 0.047 mol) from Ex-Y-6 was added in THF (50 mL) dropwise over 45 min. The reaction was stirred at −77° C. for 30 min then warmed to −50° C. for 1 hr before it was warmed to room temperature over 4 hr. To the reaction was added ethyl acetate (200 mL) and saturated ammonium chloride. The organics were collected, dried over MgSO₄ and concentrated in vacuo. The crude oil was purified on silica chromatography to afford the olefin compound (45.1 g) in 81% yield as a pale yellow viscous oil. ¹H NMR (CDCl₃, δ ppm) 1.4-2.6 (m,.10H), 2.92(d, 1H), 4.17(m, 1H), 4.38(m, 1H), 5.05(q, 2H), 5.40(m, 2H), 7.3(m,10H). ¹³C NMR (CDCl₃, δ ppm) 29.49, 29.64, 31.32, 39.60, 49.56, 53.98, 61.01, 65.25, 124.14, 127.81, 128.20, 128.55, 128.79, 129.30, 130.96, 135.68, 137.31, 152.59, 157.57, 171.61.

### Ex-Y-8

### Preparation of

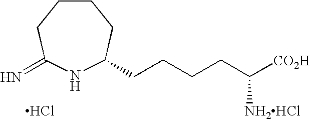 To a 20 mL vial was added the product from Ex-Y-7 (19.77 g, 0.039 mol) in Dioxane (50 mL) and 4N aqueous HCl (250 mL). This solution was added a cat. amount of 10% Pd on carbon in a hydrogenation flask. The flask was pressurized with H₂ (50 psi) for 5 hr. The reaction was monitored by mass spec and the starting material had been consumed. The solution was filtered through a pad of celite and washed with water. The solvent was removed by lyophollization to afford the title compound (7.52 g) in 81% yield. MH⁺242.2, MH+NH₄⁺259.2. ¹H NMR (CD₃OD δ ppm) 1.2-2.0 (m, 15H), 2.42 (d, 1H), 2.65 (dd, 1H), 3.49 (m, 1H), 3.98 (t, 1H), 7.26 (s), 8.05 (s), 8.35 (s). ¹³C NMR (CDCl₃, δ ppm) 24.43, 25.58, 26.00, 26.10, 32.75, 33.45, 35.31, 53.76, 54.55, 157.27, 175.13.

### Example Z

### Preparation of (αS,2S)-α-aminohexahydro-7-imino-1H-azepine-2-hexanoic acid, trihydrate hydrochloride

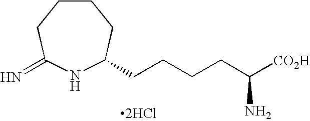

### Ex-Z-1

### Preparation of

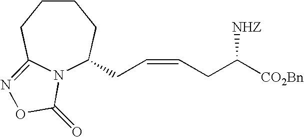 To a 1 L 3-neck flask was added the phosphonium salt from Ex-Y-3 (21.21 g, 0.041 mol) in THF (200 mL). The slurry was cooled to −78° C. in a dry-ice bath. To the cold slurry was added KHMDS (88 mL, 0.044 mol) dropwise so that the internal temperature did not rise above −72° C. The reaction stirred at −78° C. for 20 min then −45° C. for 1 hr. The temperature was then dropped back to −78° C. and the aldehyde (15.9 g, 0.047 mol) (prepared as in Ex-Y-(4-6) using N-benzyloxycarbonyl-L-homoserine lactone) was added in THF (50 mL) dropwise over 45 min. The reaction was stirred at −77° C. for 30 min then warmed to −50° C. for 30 min then warmed to room temperature over 4 hr. To the reaction was added ethyl acetate (100 mL) and saturated ammonium chloride. The organics were collected, dried over MgSO₄ and concentrated in vacuo. The crude oil was purified on silica chromatography to afford the olefin compound (9.0 g) in 45% yield as a pale yellow viscous oil. ¹H NMR (CDCl₃, δ ppm) 1.4-2.6 (m, 10H), 2.92 (d, 1H), 4.17 (m, 1H), 4.38 (m, 1H), 5.05 (q, 2H), 5.40 (m, 2H), 7.3 (m,10H). ¹³C NMR (CDCl₃, δ ppm) 29.49, 29.64, 31.32, 39.60, 49.56, 53.98, 61.01, 65.25, 124.14, 127.81, 128.20, 128.55, 128.79, 129.30, 130.96, 135.68, 137.31, 152.59, 157.57, 171.71.

### Ex-Z-2

### Preparation of

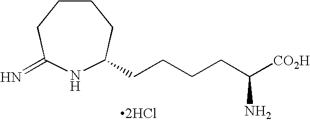 To a 20 mL vial was added the product from Ex-Z1 in dioxane (5 mL) and 4N aqueous HCl (16 mL). This solution was added a cat. amount of 10% Pd on carbon in a hydrogenation flask. The flask was pressurized with H₂ (50 psi) for 5 hr. The reaction was monitored by mass spec and the starting material had been consumed. The solution was filtered through a pad of ceilite and washed with water. The solvent was removed by lyophilization to afford the title compound (98.7 mg) in 79.4% yield. MH⁺242.2, MH+NH₄⁺259.2. ¹H NMR (CD₃OD, δ ppm) 1.2-2.0 (m, 15H), 2.42 (d, 1H), 2.6 (dd, 1H), 3.49 (m, 1H), 3.98 (t, 1H). ¹³C NMR (CDCl₃, δ ppm) 24.43, 25.58, 26.00, 26.10, 32.75, 33.45, 35.31, 53.76, 54.55, 157.27, 175.13.

### Example AA

### Preparation of (2S,4Z)-2-amino-6-[(2R)-hexahydro-7-imino-1H-azepin-2-yl]-4-hexenoic acid

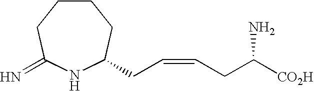

### Ex-AA-1

### Preparation of (2S,4Z)-6-[(2R)-hexahydro-7-imino-1H-azepin-2-yl]-2-[[(phenylmethoxy)carbonyl]amino]-4-hexenoic acid, phenylmethyl ester

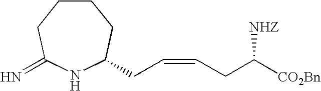 To a 50 mL flask was added a sample of Ex-Z-1 (1.5 g, 2.97 mmol) in methanol (25 mL). A 60% solution of glacial acetic acid (16 mL) was then added to the reaction mixture. A precipitate was observed. Additional methanol was added to dissolve the solid (1 mL). To the reaction was then added zinc dust (0.200 g). The reaction was sonicated for 4 hr during which the temperature was maintained at 37° C. The reaction was monitored by TLC and MS until the starting material was consumed and a mass corresponding to the product was observed. The solution was decanted from the zinc and a 30% solution of acetonitrile/water (100 mL) was added to the filtrate. The reaction was purified with 52% acetonitrile/water in 2 runs on the Waters Preparatory HPLC [a gradient of from 20% to 70% acetonitrile over 30 min]. Lyophilization of the resulting product afforded the title material of Ex-AA-1 (1.01 g) in 73% yield as a white solid. MH⁺464.4, MH+Na⁺486.4. ¹H NMR (CD₃OD, δ ppm): 1.2-2.0 (m, 8H), 2.42 (m, 2H), 2.6 (m, 5H), 3.49 (q, 1H), 4.31 (t, 1H), 5.15 (s, 2H), 5.22 (s, 2H), 5.43 (q, 1H), 5.59(q, 1H), 7.25 (bs, 10H). ¹³C NMR (CDCl₃, δ ppm): 24.37, 29.61, 30.76, 32.45, 33.73, 34.42, 55.40, 57.09, 68.06, 68.07, 122.3, 124.9, 128.76, 129.09, 129.28, 129.39, 129.51, 129.61, 155.71, 158.35, 173.90.

### Ex-AA-2

### Preparation of

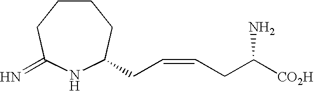 To a 250 mL flask was added the product of Ex-AA-1 (1.0 g, 2.2 mmol) in 4 M HCl (100 mL). The reaction was refluxed overnight, monitored by MS until the starting material had been consumed and the mass for the product was observed. The reaction, without further work up was purified in 2 runs on the Water's prep reverse phase column using 18% acetonitrile/water [0% to 30% acetonitrile/water over 30 min]. Lyophilization of the combined fractions afforded the title product (0.34 g) in 64% yield as a creamn colored foam. MH⁺240.3, MH+Na⁺486.4. ¹H NMR (CD₃OD, δ ppm): 1.2-2.0 (m, 6H), 2.35 (m, 2H), 2.45 (dd, 2H), 2.69 (m, 2H), 3.61 (dt, 1H), 3.98 (t, 1H), 5.59(m, 1H), 5.65 (m, 1H). ¹³C NMR (CDCl₃, δ ppm): 23.65, 24.66, 32.51, 32.84, 33.1, 33.25, 54.10, 56.1, 126.80, 129.33, 153.33, 172.52.

### Example BB

### Preparation of (2S,4E)-2-amino-6-[(2R)-hexahydro-7-imino-1H-azepin-2-yl]-4-hexenoic acid

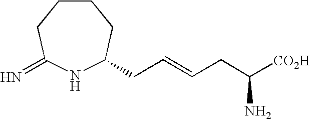

### Ex-BB-1

### Preparation of (2S,4E)-2-[[(phenylmethoxy)carbonyl]amino]-6-[(5R)-6,7,8,9-tetrahydro-3-oxo-3H,5H-[1,2,4]oxadiazolo[4,3-a]azepin-5-yl]-4-hexenoic acid, phenylmethyl ester

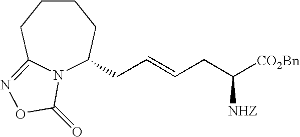 To a 250 mL flask was added Ex-Z1 (2.0 g, 3.9 mmol) and phenyl disulfide (0.860 g, 3.9 mmol) in a cyclohexane (70 mL)/benzene(40 mL) solution. Nitrogen was bubbled through the solution to purge the system of oxygen. The reaction was exposed to a short wave UV lamp for the weekend. The reaction was evaluated by normal phase HPLC (ethyl acetate/hexane). 71% of the trans isomer and 29% of the cis isomer was observed. The reaction was subjected to an additional 3 days of UV upon which 84% of the starting material converted to the trans isomer and 16% of the starting cis isomer remained. Purification by chromatography afforded Ex-BB-1 (0.956 g) in 48% yield. MH⁺506.1, MH+NH₄⁺523.2. ¹H NMR (CD₃OD, δ ppm): 1.2-2.0 (m, 8H), 2.42-2.6 (m, 6H), 2.91 (dd, 1H), 4.19 (m, 1H), 4.31 (dt, 1H), 5.09 (s, 2H), 5.11 (s, 2H), 5.18 (dt, 1H), 5.27(m, 1H), 7.25 (bs, 10H).

### Ex-BB-2

### Preparation of (2S,4E)-6-[(2R)-hexahydro-7-imino-1H-azepin-2-yl]-2-[[(phenylmethoxy)carbonyl]amino]-4-hexenoic acid, phenylmethyl ester, monohydrochloride

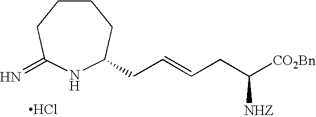 A sample of the product of Ex-BB-1 (0.956 g, 1.9 mmol) in MeOH (80 mL) was deprotected by method of Ex-AA-1 with Zn dust (1.5 g) and 60% HOAc/H₂O (40 mL). The resulting product was purified by reverse phase chromatography to afford the title material (0.248 g) in 28% yield.

### Ex-BB-3

### Preparation of

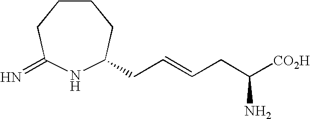 The product of Ex-BB-2 (0.248 g, 0.53 mmol) was transformed into the title product by the method of Example AA using HCl (2 mL), H₂O (2 mL), CH₃CN (4 mL). The crude product was purified by reverse phase chromatography to afford the title product of Example BB (0.073 g) in 57% yield. MH⁺240.3, MH+Na⁺486.4. ¹H NMR (CD₃OD, δ ppm) 1.2-2.0 (m, 6H), 2.35 (t, 2H), 2.55-2.82 (m, 4H), 3.68 (dt, 1H), 4.05 (t, 1H), 5.65 (m, 2H).

### Example CC

### Preparation of (E)-2-amino-2-methyl-6-[(1-iminoethyl)amino]-4-hexenoic acid, dihydrochloride

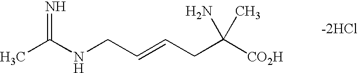

### Ex-CC-1

### Preparation of

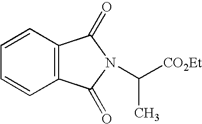 DL-Alanine ethyl ester hydrochloride (5 g, 32.5 mmol) was suspended in toluene (50 mL). Triethyl amine (4.5 mL, 32.5 mmol) was added followed by phthalic anhydride (4.8 g, 32.5 mL). The reaction flask was outfitted with a Dean-Stark trap and reflux condenser and the mixture was heated at reflux overnight. Approximately 10 mL of toluene/water was collected. The reaction mixture was cooled to room temperature and diluted with aqueous NH₄Cl and EtOAc. The layers were separated and the aqueous layer was extracted with EtOAc (3×). The ethyl acetate extract was washed with brine, dried over MgSO₄, filtered and concentrated in vacuo to give the title phthalyl-protected amino ester as a white crystalline solid in near quantitative yield. ¹H NMR (400 MHz, CDCl₃, δ ppm): 1.2 (t, 3H), 1.6 (d, 3H), 4.2 (m, 2H), 4.9 (q, 1H), 7.7 (m, 2H), 7.9 (m, 2H)

### Ex-CC-2

### Preparation of

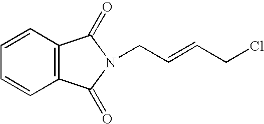 Potassium phthalimide (18.5 g, 0.1 mol) was added to a 250 mL round bottomed flask containing 1,4-butene dichloride (25 g, 0.2 mol). The reaction mixture was heated to 150° C. for 1.5 hr. The mixture was cooled to room temperature and was partitioned between brine and Et₂O. The organic layer was dried with MgSO₄, filtered and concentrated in vacuo. The residue was recrystallized from hot ethanol to give the title 1-chloro-4-phthalimidobutene (8.9 g, 39%) as orange crystals. HRMS calcd. For C₁₂H₁₀ClNO₂: m/z=236.0478 [M+H]. Found: 236.0449 ¹H NMR (300 MHz, CDCl₃, δ ppm 4.1 (d, 2H), 4.3 (d, 2H), 5.9 (m, 2H), 7.7 (m, 2H), 7.9 (m, 2H)

### Ex-CC-3

### Preparation of

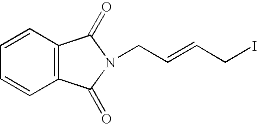 A sample of the product of Ex-CC-2 (2.3 g, 9.8 mmol) was dissolved in acetone (50 mL). NaI (3.2 g, 21 mmol) was added and the mixture was refluxed overnight. After cooling to room temperature, Et₂O was added and the mixture was washed sequentially with sodium thiosulfate and brine. The organic layer was dried with MgSO₄, filtered and concentrated in vacuo to give the title iodide (2.8 g, 87.5%) as a light yellow solid that was used without further purification. ¹H NMR (400 MHz, CDCl₃, δ ppm): 3.8 (d, 2H), 4.2 (d, 2H), 5.7 (m, 1H), 6.0 (m, 1H), 7.7 (m, 2H), 7.9 (m, 2H). Mass (M+1)=328

### Ex-CC-4

### Preparation of

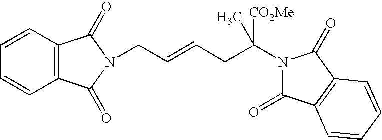 A solution of KHMDS (2.6 g, 13.3 mmol) in THF (50 mL) was cooled to −78° C. A solution of the product of Ex-CC-1 (2.2 g, 8.87 mmol) in THF (15 mL) was added and 1,3-dimethyl-3,4,5,6-tetrahydro-2(1H)-pyrimidinone (DMPU, 1.0 mL, 8.87 mL) was added immediately thereafter. After the solution was stirred at −78° C. for 40 min, a solution of the product of Ex-CC-3 (2.9 g, 8 87 mmol) in THF (15 mL) was added. The flask was removed from the cold bath and was stirred at room temperature for 3 h. The reaction mixture was partitioned between saturated aqueous NaHCO₃ and EtOAc. The organic extract was washed with brine, dried over MgSO₄, filtered and concentrated in vacuo to give the desired bis-pththalyl protected amino ester as a yellow solid. This residue was chromatographed on silica gel (1:1 hexanes:EtOAc) and gave 1.4 g (35%) of the title material as a white solid. ¹H NMR (300 MHz, CDCl₃, δ ppm: 1.2 (t, 3H), 1.6 (d, 3H), 2.8 (dd, 1H), 3.1 (dd, 1H), 4.2 (m, 4H), 5.6 (m, 1H), 5.8 (m, 1H), 7.6 (m, 4H), 7.7 (m, 2H), 7.9 (m, 2H). Mass (M+H)=447

### Ex-CC-5

### Preparation of

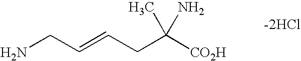 The product of Ex-CC-4 (0.78 g, 1.76 mmol) was dissolved in a mixture of formic acid (10 mL, 95%) and HCl (20 mL, concentrated HCl) and was refluxed for 3 days. The reaction mixture was cooled to 0° C. and filtered to remove phthalic anhydride. After concentrating in vacuo (T<40° C.), the title unsaturated alpha methyl lysine was obtained as a white solid (0.38 g, 95%), which was used without further purification. ¹H NMR (300 MHz, D₂O, δ ppm): 1.4 (s, 3H), 2.4 (dd, 1H), 2.6 (dd, 1H), 3.5 (d, 2H), 5.7 (m, 2H). Mass(M+H)=317

### Ex-CC-6

### Preparation of

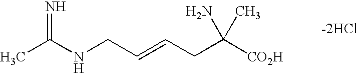 The product of Ex-CC-5 (0.2 g, 0.86 mmol) was dissolved in H₂O (8 mL) and was brought to pH 9 with 2.5 N NaOH. Ethyl acetimidate—HCl (0.42 g, 3.4 mmol) was added in 4 portions over 1 hr. After 1 h, the mixture was acidified to pH 4 with 10% HCl and was concentrated in vacuo. The residue was then passed through a water-washed DOWEX 50WX4-200 column (H form, 0.5 N NH₄OH eluent). The residue was concentrated in vacuo, acidified to pH 4 with 10% HCl, and concentrated to give the title product (17 mg, 6%) as an oil. HRMS calcd. For C₉H₁₇N₃O₂: m/z=200.1399 [M+H]. Found: 200.1417. ¹H NMR (400 MHz, D₂O, δ ppm): 1.4 (s, 3H), 2.1 (s, 3H), 2.5 (dd, 1H), 2.6 (dd, 1H), 3.8 (d, 2H), 5.6 (m, 2H)

### Example DD

### Preparation of (R, E)-2-amino-2-methyl-6-[(1-iminoethyl)amino]-4-hexenoic acid, dihydrochloride

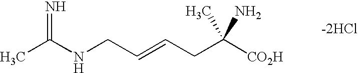

### Ex-DD-1

### Preparation of (2S, 4S)-3-Benzoyl-2-(tert-butyl)-4-methyl-1,3-oxazolidin-5-one

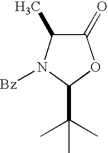 (2S, 4S)-3-Benzoyl-2-(tert-butyl)-4-methyl-1,3-oxazolidin-5-one was prepared according to Seebach's procedure. Seebach et al., Helvetica Chimica Acta, 68, 1243 (1985).

### Ex-DD-2

### Preparation of

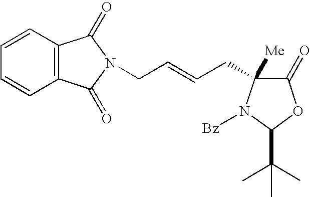 A solution of KHMDS (0.65 g, 3.24 mmol), DMPU (0.33 mL, 2.7 mmol) and THF (40 mL) was cooled to −78° C. A solution of (2S, 4S)-3-benzoyl-2-(tert-butyl)-4-methyl-1,3-oxazolidin-5-one (Ex-DD-1) (0.70 g, 2.7 mmol) in THF (10 mL) was added dropwise. After 45 min, a solution of the product of Ex-CC-3 (0.88 g, 2.7 mmol) in THF (10 mL) was added. The reaction mixture was stirred at room temperature for 2 hr and quenched with saturated aqueous NaHCO₃. The layers were separated and the aqueous layer was extracted with EtOAc. The organic layers were combined and washed with brine, dried over MgSO₄, filtered and concentrated in vacuo. The resulting yellow oil was chromatographed on silica gel (9:1 then 4:1 hexanes/ethyl acetate) to give the title protected unsaturated alpha methyl D-lysine (0.26 g, 20%) as a colorless oil. HRMS calcd. For C₂₇H₂₈N₂O₅: m/z=461.2076[M+H]. Found: 461.2033 ¹H NMR (400 MHz, CDCl₃, δ ppm: 0.9 (s, 9H), 1.5 (s, 3H), 4.3 (m, 2H), 5.5 (m, 2H), 5.6 (m, 2H), 6.1 (m, 1H), 7.5 (m, 5H), 7.7 (m, 2H), 7.9 (m, 2H)

### Ex-DD-3

### Preparation of

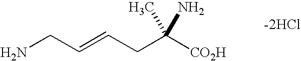 The product of Ex-DD-2 (0.255 mg, 0.55 mmol) was dissolved in 6N HCl (6 mL) and formic acid (6 mL) and was heated to reflux for 24 hr. The reaction mixture was cooled to room temperature and concentrated in vacuo. The residue was suspended in water and washed with CH₂Cl₂. The aqueous layer was concentrated and passed through a water-washed DOWEX 50WX4-200 column (H form, 0.5 N NH₄OH eluent). The residue was concentrated in vacuo, acidified to pH 4 with 10% HCl, and concentrated to give the title unsaturated D-lysine (71 mg, 55%) as an oil which was used without further purification. ¹H NMR (400 MHz, D₂O, δ ppm: 1.4 (s, 3H), 2.5 (dd, 1H), 2.6 (dd, 1H), 3.4 (d, 2H), 5.6 (m, 2H), 5.7 (m, 2H)

### Ex-DD-4

### Preparation of

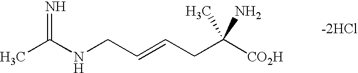 The product of Ex-DD-3 (13 mg, 0.056 mmol) was dissolved in H₂O (5 mL) and was brought to pH 9 with 2.5 N NaOH. Ethyl acetimidate—HCl (27 mg, 0.2 mmol) was added in 4 portions over 2 hr. After 2 h, the mixture was acidified to pH 4 with 10% HCl and was concentrated in vacuo. The residue was passed through a water-washed DOWEX 50WX4-200 column (H form, 0.5 N NH₄OH eluent). The residue was concentrated in vacuo, acidified to pH 4 with 10% HCl, and concentrated to give the title product (45 mg) as an oil. HRMS calcd. For C₉H₁₇N₃O₂: m/z=200.1399 [M+H]. Found: 200.1386. ¹H NMR (400 MHz, D₂O, δ ppm): 1.4 (s, 3H), 2.1 (s, 3H), 2.5 (dd, 1H), 2.6 (dd, 1H), 3.8 (d, 2H), 5.6 (m, 2H)

### Example EE

### Preparation of (S, E)-2-amino-2-methyl-6-[(1-iminoethyl)amino]-4-hexenoic acid, dihydrochloride

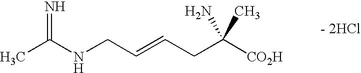

### Ex-EE-1

### Preparation of (2R, 4R)-3-Benzoyl-2-(tert-butyl)-4-methyl-1,3-oxazolidin-5-one

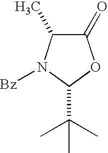 (2R, 4R)-3-Benzoyl-2-(tert-butyl)-4-methyl-1,3-oxazolidin-5-one was prepared according to Seebach's procedure. Seebach et al., Helvetica Chimica Acta, 68, 1243 (1985).

### Ex-EE-2

### Preparation of

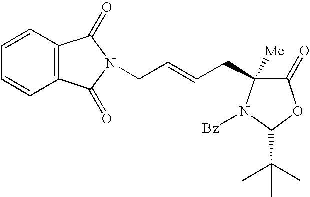 A solution of the (2R, 4R)-3-benzoyl-2-(tert-butyl)-4-methyl-1,3-oxazolidin-5-one product of Ex-EE-1 (2.0 g, 7.6 mmol) in THF (50 mL) was cooled to −78° C. A −78° C. solution of KHMDS (0.65 g, 3.24 mmol) in THF (25 mL) was added dropwise. After 30 min, a solution of the product of Ex-CC-3 (2.8 g, 8.6 mmol) in THF (25 mL) was added. The reaction mixture was stirred at room temperature for 1 hr and quenched with saturated aqueous NaHCO₃. The layers were separated and the aqueous layer was extracted with EtOAc. The organic layers were combined and washed with brine, dried with MgSO₄, filtered and concentrated in vacuo. The resulting orange oil was chromatographed on silica gel (9:1 then 4:1 hexanes/ethyl acetate) to give the protected title unsaturated alpha methyl L-lysine (0.5 g, 15%) as a white solid. HRMS calcd. For C₂₇H₂8N₂O₅: m/z=461.2076[M+H]. Found: 461.2043. ¹H NMR (400 MHz, CDCl₃, δ ppm): 0.9 (s, 9H), 1.5 (s, 3H), 4.3 (m, 2H), 5.5 (m, 2H), 5.6 (m, 2H), 6.1 (m, 1H), 7.5 (m, 5H), 7.7 (m, 2H), 7.9 (m, 2H).

### Ex-EE-3

### Preparation of

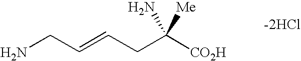 The product of Ex-EE-2 (0.5 g, 1 mmol) was dissolved in 12N HCl (10 mL) and formic acid (5 mL) and this mixture was heated to reflux for 12 hr. The reaction mixture was cooled in the freezer for 3 h and the solids were removed by filtration. The residue was washed with CH₂Cl₂ and EtOAc. The aqueous layer was concentrated in vacuo and gave the title unsaturated alpha methyl L-lysine (0.26 g, 99%) as an oil which was used without further purification. ¹H NMR (300 MHz, D₂O, δ ppm): 1.4 (s, 3H), 2.5 (dd, 1H), 2.6 (dd, 1H), 3.4 (d, 2H), 5.7 (m, 2H)

### Ex-EE-4

### Preparation of

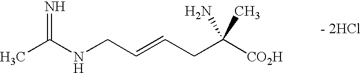 The product of Ex-EE-3 (0.13 g, 0.56 mmol) was dissolved in H₂O (1 mL) and was brought to pH 9 with 2.5 N NaOH. Ethyl acetimidate—HCl (0.28 g, 2.2 mmol) was added in 4 portions over 1 hr. After 1 h, the mixture was acidified to pH 4 with 10% HCl and was concentrated in vacuo. The residue was and passed through a water-washed DOWEX 50WX4-200 column (0.5 N NH₄OH eluent). The residue was concentrated in vacuo, acidified to pH 4 with 10% HCl, and concentrated to give the title product as an oil (40 mg). HRMS calcd. For C₉H₁₇N₃O₂: m/z=222.1218 [M+Na]. Found: 222.1213 ¹H NMR (300 MHz, D₂O, δ ppm): 1.4 (s, 3H), 2.1 (s, 3H), 2.4 (dd, 1H), 2.6 (dd, 1H), 3.8 (d, 2H), 5.6 (m, 2H)

### Example FF

### Preparation of 2-amino-2-methyl-6-[(1-iminoethyl)amino]-4-hexynoic acid, dihydrochloride

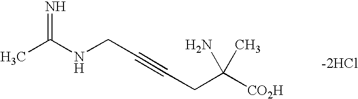

### Ex-FF-1

### Preparation of N-boc-1-amino-4-chlorobut-2-yne

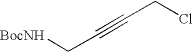 The N-boc-1-amino-4-chlorobut-2-yne was prepared following the procedure described in Tetrahedron Lett. 21, 4263 (1980).

### Ex-FF-2

### Preparation of methyl N-(diphenylmethylene)-L-alaninate

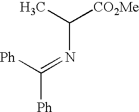 Methyl N-(diphenylmethylene)-L-alaninate was prepared by following the procedure described in J. Org. Chem., 47, 2663 (1982).

### Ex-FF-3

### Preparation of

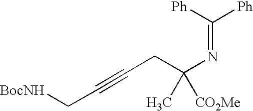 Dry TBF (1000 mL) was placed in a flask purged with argon and 60% NaH dispersed in mineral oil (9.04 g, 0.227 mol) was added. To this mixture was added the product of Ex-FF-2 (30.7 g, 0.114 mol). The reaction mixture was then stirred at 10° C.-15° C. for 30 min. Potassium iodide (4 g) and iodine (2 g) were added and immediately followed by the addition of the product of Ex-FF-2 (23 g, 0.113 mol in 200 mL THF) in 30 min. The reaction mixture was then stirred at 55° C. until the starting material disappeared (2 hr). The reaction mixture was then cooled to room temperature and the solvent was evaporated. Ethyl acetate (500 mL) was added and the mixture was carefully washed with 2×200 mL deionized water. The organic layer was dried over anhydrous MgSO₄, filtered and evaporated to give 44 g of crude product. Purification by chromatography using 20% ethyl acetate in hexane afforded the title protected unsaturated alpha-methyl lysine (28 g; 57%). Anal.Calcd for C₂₆H₃₀N₂O₄ and 0.5 ethylacetate: C,70.42; H, 7.14; N, 5.91. Found: C, 70.95; H, 7.73; N, 6.09. IR (Neat, δ max, cm⁻¹): 2981, 1714,1631. ¹H NMR (CDCl₃, δ ppm): 1.28 (s, 9H), 1.4 (s, 3H), 2.65-2.76(m, 2H), 3.15 (s, 3H), 3.7 (bs, 2H), 4.6 (bs, 1H), 6.95-7.4 (m, 10H). ¹³C NMR (CDCl₃, δ ppm): 24.29, 28.33, 28.39, 33.24, 51.60, 53.55, 127.79, 127.97, 128.26, 128.36, 128.43, 128.54, 128.66, 130.05, 130.22, 132.39. Mass (M+1)=435. DSC purity: 261.95° C.

### Ex-FF-4

### Preparation of

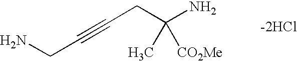 The product of Ex-FF-3 (16 g, 0.0368 mol) was dissolved in 1N HCl (300 mL) and stirred at 25° C. for 2 hr. The reaction mixture was washed with ether (2×150 mL) and the aqueous layer separated and decolorized with charcoal. Concentration afforded 9 g (100% yield) of the deprotected unsaturated alpha-methyl lysine ester Ex-FF-4 as white foamy solid. Anal.Calcd for C₈H₁₄N₂O₂ containing 2.26 HCl and 1.19 H₂O: C,35.06; H, 6.86; N, 10.22; Cl, 29.24. Found: C, 35.31; H, 7.38; N, 10.70; Cl, 29.77. ¹H NMR (D₂O, δ ppm): 1.56 (s, 3H), 2.8-3.0 (2 dt, 2H), 3.75(s, 2H), 3.79 (s, 3H). ¹³C NMR (D₂O, δ ppm) 29.81, 32.05, 57.08, 61.90, 79.57, 82.43, 173.92. Mass (M+1)=171. DSC purity: 114.22° C. UV=206 nm,abs 0.013. [α]₂₅ in methanol=0 at 365 nm.

### Ex-FF-5

### Preparation of

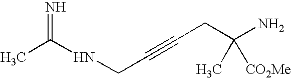 The product of Ex-FF-4 (2.43 g, 0.01 mol) was dissolved in deionized water (25 mL). A solution of NaOH (400 mg, 0.01 mol) in deionized water (25 mL) was added at 25° C. to bring the pH to 7.95 and stirring was continued another 10 min. Ethylacetimidate hydrochloride (988 mg, 0.008 mol) was added to the reaction mixture with simultaneous adjustment of the pH to 8.5 by adding 1N NaOH. The reaction mixture was stirred at pH 8 to 8.5 for 3 hr following acetimidate addition. 1N HCl was added to the reaction mixture (4.1 pH). The solvent was evaporated at 50° C. to afford a yellow crude hygroscopic residue (4 g, >100% yield). Purification was carried out on the Gilson chromatography system using 0.1% AcOH/CH₃CN/H₂O. Anal.Calcd for C₁₀H₁₇N₃O₂ containing 2.25 HCl and 1.7 H₂O: C, 37.08; H, 7.05; N, 12.97; Cl, 24.63. Found: C, 37.01; H, 6.79; N, 12.76; Cl, 24.87. IR (Neat, δ max, cm⁻¹): 2953, 2569, 1747, 1681, 1631. ¹H NMR (D₂O, δ ppm): 1.52 (s, 3H), 2.12 (s, 3H), 2.74-2.96 (2 dt, 2H), 3.75 (s, 3H), 3.95 (t, 2H). ¹³C NMR (D₂O, δ ppm): 23.89, 29.81, 32.05, 57.08, 61.90, 79.57, 82.43, 173.92. Mass (M+1)=212

### Ex-FF-6

### Preparation of

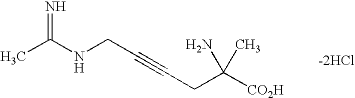 The product of Ex-FF-5 (100 mg, 0.0005 mol) was dissolved in 8N HCl (20 mL) and stirred for 10 hr at reflux. The reaction mixture was cooled to room temperature and the aq. HCl was evaporated on rotavap. The residue was dissolved in deionized water (10 mL) and water and reconcentrated under vacuum to afford the title product as a yellow glassy solid in almost quantitative yield (88 mg). Anal.Calcd for C₉H₁₅N₃O₂ containing 2.4 HCl and 1.8 H₂O: C, 34.08; H, 6.67; N, 13.25; Cl, 26.83. Found: C, 34.32; H, 6.75; N, 13.63; Cl, 26.47. IR (Neat, δ max, cm⁻¹): 1738, 1677, 1628, 1587. ¹H NMR (D₂O, δ ppm): 1.6 (s, 3H), 2.24 (s, 3H), 2.8-3.0 (2 dt, 2H), 4.1 (s, 2H). ¹³C NMR (D₂O, δ ppm): 21.22, 24.10, 29.88, 34.58, 80.04, 80.99, 128.39, 168.07, 176.13. Mass (M+1)=198.

### Example GG

### Preparation of (2R/S,4Z)-2-amino-2-methyl-7-[(1-iminoethyl)amino]-4-heptenoic acid, dihydrochloride

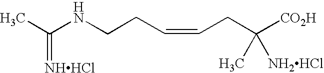

### Ex-GG-1

### Preparation of

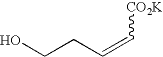 5,6 dihydropyran-2-one (49.05 g, 0.5mol) was dissolved in 200 mL of water. Potassium hydroxide (35 g, 0.625 mol) was added and the reaction mixture stirred at ambient temperature for 5 hr. The solvent was removed in vacuo to yield a colorless glassy solid (65 g, 84%) that was characterized by NMR to be predominantly the cis isomer of the title compound. ¹H NMR (CDCl₃) δ: 2.7 (m, 2H), 3.6 (t, 2H), 5.8-5.85(m, 1H), 5.9-5.97 (m, 1H).

### Ex-GG-2

### Preparation of

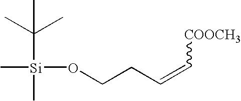 The product of Ex-GG-1 was dissolved in 100 mL of dimethyl formamide. Methyl Iodide (52 mL, 0.84 mol) was then added resulting in an exotherm to 40° C. The reaction mixture was stirred at room temperature for 10 hr and partitioned between 150 mL of ethylacetate/diethylether in a 20/80 ratio and ice water. The aqueous layer was separated and re-extracted with 100 mL of diethyl ether. The organic layers were combined, dried (Na₂SO₄), filtered and stripped of all solvent to yield the desired methyl ester product (40 g, 71%). This material was dissolved in 200 mL of methylene chloride and the solution cooled to 0° C. Tertiarybutyl dimethylsilylchloride, triethylamine and dimethylaminopyridine were added. The reaction mixture was slowly warmed to room temperature and stirred for 10 hr under N₂. The reaction was extracted with 100 mL of 1N aqueous potassium bisulfate solution. The organic layer was washed with 2×100 mL of brine and then with 3×150 mL of water. The organic layer was dried (Na₂SO₄), filtered and stripped to yield 42 g (56%) of the title material. ¹H NMR (CDCl₃) δ: 0.02 (s, 6H), 0.085 (s, 9H), 2.8-2.85 (m, 2H), 3.65 (s, 3H), 3.66-3.7 (m 2H), 5.8 (m, 1H), 6.3 (m, 1H)

### Ex-GG3

### Preparation of

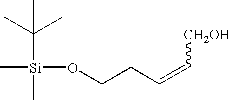 The material from Ex-GG-2 was dissolved in 25 mL of toluene and cooled to 0° C. Diisobutylaluminum hydride (1.0 M in toluene, 32 mL, 48 mmol) was added dropwise maintaining the temperature between 5 and −10° C. The reaction mixture was stirred for 1.5 hr between 6 and −8° C. before it was cooled to −25° C. To this mixture was added 100 mL of 0.5N sodium potassium tartarate. The reaction mixture was allowed to warm up to room temperature and stirr for 1 hr. A gelatinous precipitate was formed which was filtered. The aqueous was extracted with 2×100 mL EtOAc. The combined organic layers were dried (sodium sulfate), filtered and concentrated in vacuo to yield title product (3.45 g, 66%) as a colorless oil. ¹H NMR (CDCl₃) δ: 0.02 (s, 6H), 0.085 (s, 9H), 2.25-2.32 (m, 2H), 2.6 (bs, 1H), 3.6 (t, 2H), 4.08 (d, 2H), 5.45-5.55 (m, 1H), 5.7-5.75 (m, 1H)

### Ex-GG-4

### Preparation of

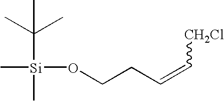 The product (8 g, 37 mmol) from Ex-GG3 was dissolved in 100 mL methylene chloride and this solution was cooled to 0° C. Methanesulfonyl chloride was then added and this mixture was stirred for 5 min. Triethylamine was then added. The temperature maintained between 0 and −10° C. during the addition of the aforementioned reagents. The reaction mixture was subsequently warmed up to room temperature and stirred for 24 hr. It was then extracted with 100 mL of 50% aqueous sodium bicarbonate solution. The organic layer was washed with 100 mL of saturated aqueous brine solution, dried (sodium sulfate), filtered and stripped in vacuo to yield the title material (8.2 g, 94%). ¹H NMR (CDCl₃) 6: 0.02 (s, 6H), 0.085 (s, 9H), 2.25-2.32 (m, 2H), 3.6 (t, 2H), 4.08 (d, 2H), 5.6-5.7 (m, 2H)

### Ex-GG-5

### Preparation of

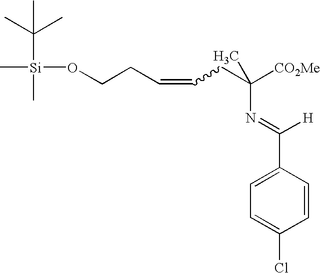

A solution of N-p-chloro phenylimine alanine methyl ester (8.85 g, 34 mmol) dissolved in 59 mL of tetrahydrofuran was purged with Argon. NaH (1.64 g, 41mmol) was added whereupon the solution turned bright orange and subsequently a deep red. A solution of the title material from Ex-GG-4 (8 g, 34 mmol) in 40 mL of tetrahydrofuran was added to the above anionic solution. An exotherm was observed raising the temperature to almost 40° C. The reaction mixture was maintained between 48 and −52° C. for 2 hr. It was then cooled to room temperature and filtered. Filtrate was stripped in vacuo to yield the title material (8.4 g, 50% crude yield) as a yellow oil. ¹H NMR (CDCl₃) δ: 0.02 (s, 6H), 0.085 (s, 9H), 1.45 (s, 3H), 1.6 (s, 1H), 2.2-2.25(m, 2H), 2.65 (d, 2H), 3.55 (m, 2H), 3.7 (s, 3H), 5.45-5.55 (m, 2H), 7.35-7.7 (m, 4H).

### Ex-GG-6

### Preparation of

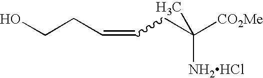

The title material from Ex-GG-5 (8.4 g, 18.2 mmol) was treated with 125 mL 1N hydrochloric acid and the reaction was stirred for 1 hr at room temperature. After the reaction mixture had been extracted 2×75 mL of ethylacetate the aqueous layer was stripped in vacuo at 56° C. to yield 4 g of the title material (100% crude yield). ¹H NMR (CD₃OD) δ: 1.6 (s, 3H), 2.3-2.4 (m, 2H), 2.65-2.8 (m, 2H), 3.6-3.65 (m, 2H), 3.87 (s, 3H), 5.4-5.5 (m, 1H), 5.75-5.85 (m, 1H).

### Ex-GG-7

### Preparation of

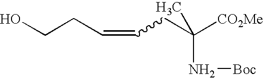

The title product of Ex-GG-6 (1.9 g, 8.5 mmol) was dissolved in a mixture of 15 mL dioxane and 8 mL of water. Solid potassium bicarbonate was then carefully added to avoid foaming. The reaction mixture was stirred for 10 min before tertiarybutyloxycarbonyl anhydride was added portion-wise and reaction mixture was stirred at ambient temperature for 24 hr. The reaction mixture was diluted with 100 mL of ethylacetate and 50 mL of water before it was poured into a separatory funnel. The organic layer was separated, dried (Na₂SO₄), filtered and stripped to yield the title material as a colorless oil (1.9 g, 78% crude yield). ¹H NMR (CDCl₃) δ: 1.42 (s, 9H), 1.55 (s, 3H), 2.3-2.36 (m, 2H), 2.58-2.65 (m, 2H), 3.65-3.7 (t, 2H), 3.75 (s, 3H), 5.42-5.5 (m, 1H), 5.55-5.62 (m, 1H)

Ex-GG-8. Another 1.9 g sample of the title material from Ex-GG-6 was converted by the methods of Ex-GG-7 to the crude Z/E mixture of the title product of Ex-GG-7. This material further purified on silica with a solvent system of ethylacetate/hexane in a 20/80 ratio to obtain the minor E-isomer as well as the major Z-isomer.

### Ex-GG-9

### Preparation of

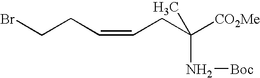

The title Z-isomer from Ex-GG-8 (1.8 g, 6.25 mmol) was dissolved in 20 mL of acetonitrile and this solution was cooled to 0° C. Pyridine (0.76 g, 9.4 mmol) was then added followed by the portion-wise addition of solid dibromotriphenylphosphorane (3.46 g, 8.2 mmol) over 10 min. The reaction mixture was stirred under Argon for 24 hr at room temperature. The precipitate that formed was filtered off. The filtrate was concentrated in vacuo to give 2.8 g of an oil that was purified on silica gel using a solvent system of ethylacetate/hexane in a 60/40 ratio. The 1.1 g of title material (50%) was characterized by NMR. ¹H NMR (CDCl₃) δ: 1.44 (s, 9H), 1.55 (s, 3H), 2.6-2.65 (m, 4H), 3.35-3.4 (m, 2H), 3.75 (s, 3H), 5.4-5.45 (m, 1H), 5.55-5.6 (m, 1H)

### Ex-GG-10

### Preparation of

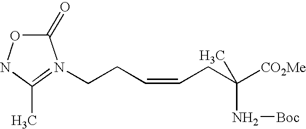

The title material from Ex-GG-8 (300 mg, 0.86 mmol) was dissolved in 25 mL of dimethylformamide (DMF). The potassium salt of 3-methyl-1,2,4-oxadiazolin-5-one (130 mg, 0.94 mmol) was added and the reaction mixture was heated to 52° C. and maintained there for 18 hr with stirring. It was then cooled to room temperature before the DMF was stripped in vacuo at 60° C. The residue was purified on silica gel with a gradient of 60/40 to 90/10 ethyl acetate/hexane to yield 300 mg (95%) of the title material. ¹H NMR (CD₃OD) δ: 1.35 (s, 3H), 1.43 (s, 9H), 2.32 (s, 3H), 2.45-2.55 (m, 4H), 3.65-3.7 (m, 2H), 3.72 (t, 3H), 5.5-5.6 (m, 2H)

### Ex-GG-11

### Preparation of

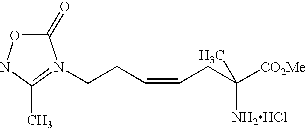

The product of Ex-GG-10 (300 mg) was treated with 0.05 N of aqueous HCl and this solution was stirred for 30 min. The solvent was removed in vacuo to afford the desired material in nearly quantitative yield. ¹H NMR (CD₃OD) δ: 1.6 (s, 3H), 2.25 (s, 3H), 2.45-2.55 (m, 2H), 2.7-2.8 (m, 2H), 3.3-3.4(m, 5H), 5.5-5.6 (m, 1H), 5.7-5.8 (m, 1H)

### Ex-GG-12

### Preparation of

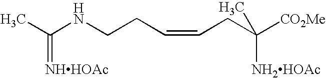

The title material from Ex-GG-11 (198 mg, 0.54 mmol) was dissolved in 50 mL of MeOH. Formic acid (40 mg) was then added followed by Palladium on Calcium carbonate (400 mg). The reaction mixture was heated to 65° C. with stirring in a sealed tube for 24 hr. It was then cooled to room temperature and filtered. The filtrate was concentrated in vacuo and the residue purified by reverse phase HPLC to yield 115 mg (75%) of the title material. ¹H NMR (CD₃OD) δ: 1.4 (s, 3H), 1.95 (s, 3H), 2.25 (s, 3H), 2.4-2.52 (m, 4H), 3.25-3.35 (m, 2H), 3.75 (t, 3H), 5:54-5.62 (m, 2H)

### Ex-GG-13

### Preparation of

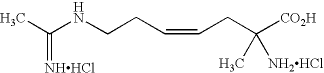

The title material (75 mg) from Ex-GG-12 was dissolved in 15 mL of 2N hydrochloric acid. The reaction mixture was heated to a reflux and stirred for 6 hr before ot was cooled to room temperature. The solvent was removed in vacuo. The residue was dissolved in 25 mL of water and stripped on the rotary evaporator to remove excess hydrochloric acid. The residue was dissolved in water and lyophilized to give 76 mg (˜100%) of the title material. Elemental analyses Calcd for C₁₀H₁₉N₃O₂+2.2HCl+2.2 H₂O: C, 36.06; H, 7.75; N, 12.61. Found for C₁₀H₁₉N₃O₂+2.2HCl+2.2 H₂O: C, 35.91; H, 7.61; N, 12.31. ¹H NMR (CD₃OD) δ: 1.47 (s, 3H), 2.32 (s, 3H), 2.45-2.64 (m, 4H), 2.58-2.65 (m, 2H), 3.65-3.7 (t, 2H), 5.55-5.65 (m, 2H)

### Example HH

### Preparation of (2S,5E)-2-amino-2-methyl-6-fluoro-7-[(1-iminoethyl)amino]-5-heptenoic acid, dihydrochloride

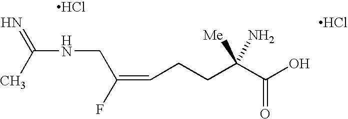

### Ex-HH-1

### Preparation of

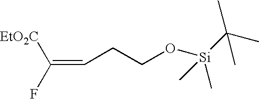

To a cold (−78° C.) solution of triethyl 2-fluorophosphonoacetate (25.4 g, 105 mmol) in 100 mL of THF was added n-butyl lithium (63 mL of 1.6 M in hexane, 101 mmol). This mixture was stirred at −78° C. for 20 min producing a bright yellow solution. A solution of crude 3-[(tert-butyldimethylsilyl)oxy]propanal (J. Org. Chem., 59, 1139-1148 (1994)) (20.0 g, 105 mmol) in 120 mL of THF was then added dropwise over 10 min, and the resulting mixture was stirred for 1.5 hr at −78° C., at which time analysis by thin layer chromatography (5% ethyl acetate in hexane) showed that no starting material remained. The reaction was quenched at −78° C. with sat. aqueous NH₄Cl (150 mL). The organic layer was collected, and the aqueous layer was extracted with diethyl ether (300 mL). The combined organics were washed with brine (200 mL), dried over MgSO₄, filtered and concentrated. The crude material was filtered through a plug of silica gel (150 g) eluting with hexane (2 L) to give 14.38 g (52%) of the desired (2E)-5-[[(1,1-dimethylethyl)di-methylsilyl]oxy]-2-fluoro-2-pentenoic acid ethyl ester product as a clear oil. ¹H NMR and ¹⁹F NMR indicated that the isolated product had an approximate E:Z ratio of 95:5. HRMS calcd. for C₁₃H₂₆FO₃Si: m/z=277.1635 [M+H]⁺, found: 277.1645. ¹H NMR (CDCl₃) δ 0.06 (s, 6H), 0.94 (s, 9H), 1.38 (t, 3H), 2.74 (m, 2H), 3.70 (m, 2H), 4.31 (q, 2H), 6.0 (dt, vinyl, 1H). ¹⁹F NMR (CDCl₃) δ −129.78 (d, 0.05 F, J=35 Hz, 5% Z-isomer), −121.65 (d, 0.95 F, J=23 Hz, 95% E-isomer).

### Ex-HH-2

### Preparation of

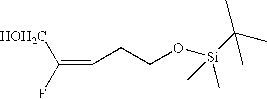

To a solution of Ex-HH-1 (6.76 g, 24.5 mmol) in 100 mL of methanol at room temperature was added solid NaBH4 (4.2 g, 220 mmol) in 1.4 g portions over 3 hr. After 3.5 hr water was added (10 mL). Additional solid NaBH4 (4.2 g, 220 mmol) was added in 1.4 g portions over 3 hr. The reaction was quenched with 150 mL of sat. aqueous NH₄Cl and extracted with diethyl ether (2×250 mL). The organic layers were combined, dried over MgSO₄, filtered and concentrated. The crude material, 4.81 g of clear oil, was purified by flash column chromatography on silica gel eluting with 10% ethyl acetate in hexane to give 2.39 g (42%) of the desired (2E)-5-[[(1,1-dimethylethyl)dimethylsilyl]oxy]-2-fluoro-2-penten-1-ol product as a clear oil, that contained an approximate E:Z ratio of 93:7 by ¹⁹F NMR. HRMS calcd. for C₁₁H₂₄FO₂Si: m/z=235.1530 [M+H]⁺, found: 235.1536. ¹H NMR (CDCl₃) δ 0.06 (s, 6H), 0.88 (s, 9H), 2.35 (m, 2H), 3.62 (t, 2H), 4.19 (dd, 2H), 5.2 (dt, vinyl, 1H). ¹⁹F NMR (CDCl₃) δ −120.0 (dt, 0.07F, 7% Z-isomer), −109.82 (q, 0.93 F, J=21 Hz, 93% E-isomer).

### Ex-HH-3

### Preparation of

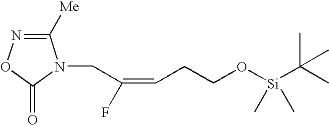

To a mixture of Ex-HH-2 (2.25 g, 9.58 mmol), polymer-supported triphenylphosphine (3 mmol/g, 1.86 g, 15 mmol) and 3-methyl-1,2,4-oxadiazolin-5-one (1.25 g, 12.5 mmol) in 60 mL of THF was added dropwise diethylazodicarboxylate (2.35 mL, 14.7 mmol). The reaction mixture was stirred for 1 hr at room temperature, and additional 3-methyl-1,2,4-oxadiazolin-5-one (0.30 g, 3.0 mmol) was added. After 30 min, the mixture was filtered through celite, and the filtrate was concentrated. The resulting yellow oil was triturated with diethyl ether (30 mL) and the solid removed by filtration. The filtrate was concentrated, triturated with hexane (30 mL) and filtered. The filtrates was concentrated to an oil which was purified by flash column chromatography on silica gel eluting with 15% ethyl acetate in hexane to give 1.83 g (60%) of the desired 4-[(2E)-5-[[(1,1-dimethylethyl)dimethylsilyl]oxy]-2-fluoro-2-pentenyl]-3-methyl-1,2,4-oxadi-azol-5(4H)-one product as a clear oil, that contained only the desired E-isomer by ¹⁹F NMR. HRMS calcd. for C₁₄H₂₆FN₂O₃Si: m/z=317.1697 [M+H]⁺, found: 317.1699. ¹H NMR (CDCl₃) δ 0.04 (s, 6H), 0.85 (s, 9H), 2.28 (s, 3H), 2.37 (m, 2H), 3.64 (t, 2H), 4.32 (d, 2H), 5.4 (dt, vinyl, 1H). ¹⁹F NMR (CDCl₃) δ −110.20 (q, 1 F, J=21 Hz).

### Ex-HH-4

### Preparation of

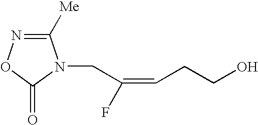

A solution of Ex-HH-3 (1.83 g, 5.78 mmol) in a mixture of acetic acid (6 mL), THF (2 mL) and water (2 mL) was stirred at room temperature for 2.5 hr. The resulting solution was concentrated in vacuo to an oil which was dissolved in diethyl ether (50 mL). The organic layer was washed with saturated NaHCO₃, and the aqueous layer was extracted with diethyl ether (2×50 mL) and ethyl acetate (2×50 mL). The combined organic layers were dried (MgSO₄), filtered and evaporated to give 1.15 g (98%) of the desired 4-[(2E)-2-fluoro-5-hydroxy-2-pentenyl]-3-methyl-1,2,4-oxadiazol-5(4H)-one product as a clear colorless oil. HRMS calcd. for C₈H₁₂FN₂O₃: m/z=203.0832 [M+H]⁺, found: 203.0822. ¹H NMR (CDCl₃) δ 2.31 (3H), 2.4 (m, 2H), 3.66 (t, 2H), 4.37 (d, 2H), 5.42 (dt, vinyl, 1H). ¹⁹F NMR (CDCl₃) δ −110.20 (q, 1 F, J=21 Hz).

### Ex-HH-5

### Preparation of

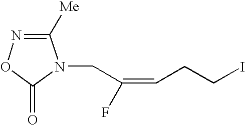

To a CH₂Cl₂ (2 mL) solution of triphenylphosphine (238 mg, 0.91 mmol) and imidazole (92 mg) at 0° C. was added solid iodine (230 mg, 0.91 mmol), and the mixture was stirred for 5 min. To the resulting yellow slurry was added a CH₂Cl₂ (1.5 mL) solution of Ex-HH-4 (0.15 g, 0.74 mmol). The slurry was allowed to warm to room temperature and stirred 30 min. The reaction mixture was diluted with CH₂Cl₂ (10 mL), washed with saturated Na₂S₂O₃ (5 mL) and brine (5 mL), dried (MgSO₄), filtered and evaporated to an oil. Addition of diethyl ether (10 mL) to the oil gave a white precipitate that was removed by filtration and the filtrate was concentrated to an oil. The crude material was purified by flash column chromatography on silica gel eluting with 30% ethyl acetate in hexane to give 0.18 g (78%) of the desired 4-[(2E)-2-fluoro-5-iodo-2-pentenyl]-3-methyl-1,2,4-oxadiazol-5(4H)-one product as a clear oil, which solidified upon standing, mp=58.1-58.6° C. Anal. calcd. for C₈H₁₀FIN₂O₂: C, 30.79; H, 3.23; N, 8.98. Found: C, 30.83; H, 3.11; N, 8.85. HRMS calcd. for C₈H₁₁FIN₂O₂: m/z=330.0115 [M+H]⁺, found: 330.0104. ¹H NMR (CDCl₃) δ 2.31 (s, 3H), 2.75 (q, 2H), 3.21 (t, 2H), 4.31 (d, 2H), 5.39 (dt, vinyl, 1H). ¹⁹F NMR (CDCl₃) δ −108.21 (q, 1F, J=21 Hz).

### Ex-HH-6

### Preparation of

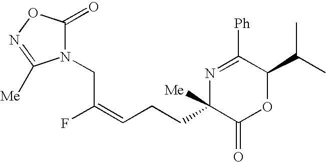

To a 1-methyl-2-pyrrolidinone (12 mL) solution of (3S, 6R)-6-isopropyl-3-methyl-5-phenyl-3,6-dihydro-2H-1,4-oxazin-2-one (Synthesis, 4, 704-717 (1999)) (1.10 g, 4.76 mmol), LiI (0.63 g, 4.76 mmol) and Ex-HH-5 (0.85 g, 2.72 mmol) in an ice bath was added 2-tert-butylimino-2-diethylamino-1,3-dimethylperhydro-1,3,2-diazaphosphorine (1.38 mL, 4.76 mmol). The yellow solution became orange upon addition of the base, and the resulting solution was allowed to stir at room temperature for 1 hr. The reaction mixture was diluted with ethyl acetate (100 mL), washed with water (2×30 mL), dried (MgSO₄), filtered and evaporated to a yellow oil. The crude material was purified by flash column chromatography on silica gel eluting with 30% ethyl acetate in hexane to give 0.64 g (57%) of the desired alkylated product as a clear oil. ¹H NMR (C₆D₆) δ 0.57 (d, 3H), 0.89 (d, 3H), 1.30 (s, 3H), 1.65 (s, 3H), 1.8 (m, 2H), 2.0 (m, 2H), 2.1 (m, 1H), 3.22 (m, 2H), 4.88 (dt, vinyl, 1H), 5.49 (d, 1H), 7.1 (m, 3H), 7.6 (m, 2H). ¹⁹F NMR (CDCl₃) δ −110.37 (q, 1 F, J=21 Hz).

### Ex-HH-7

### Preparation of

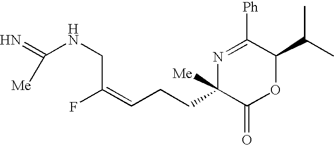

To a methanol (20 mL) solution of Ex-HH-6 (0.13 g, 0.31 mmol) was added Lindlar catalyst (1.0 g). The stirred slurry was heated to 60° C. for 1 hr, and additional Lindlar catalyst (0.30 g) was added. The slurry was stirred an additional 1 hr at 60° C., then cooled to room temperature. The catalyst was removed by filtration through celite, and the filtrate was stripped to give 0.58 g (100%) of the desired deprotected amidine product as a pale yellow oil. MS: m/z=374.2 [M+H]⁺. ¹H NMR (CD₃OD) δ 0.77 (d, 3H), 1.07 (d, 3H), 1.58 (s, 3H), 2.02 (s, 3H), 1.8-2.2 (m, 5H), 3.83 (d, 2H), 5.20 (dt, vinyl, 1H), 5.69 (d, 1H), 7.4 (m, 3H), 7.7 m, 2H). ¹⁹F NMR (CDCl₃) δ −109.4 (m, 1F, J=21 Hz)

### Ex-HH-8

### Preparation of

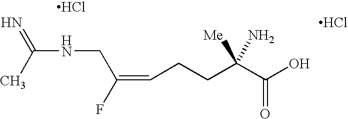

A solution of the product from Ex-HH-7 (0.58 g, 1.54 mmol) in 1.5 N HCl (25 mL) was washed with diethyl ether (2×20 mL) and refluxed for 1 hr. The solvent was stripped and the crude amino acid ester was dissolved in 6 N HCl (15 mL) and heated to reflux. After 6 hr, the solvent was removed in vacuo, and the resulting foam was purified by reverse-phase HPLC eluting with a 30 min gradient of 0-40% CH₃CN/H₂O(0.25% acetic acid). Fractions containing product were combined and concentrated to a foam. The product was dissolved in 1 N HCl and the solvent removed in vacuo (2×) to give 0.15 g (29%) of the desired (2S,5E)-2-amino-2-methyl-6-fluoro-7-[(1-iminoethyl)amino]-5-heptenoic acid, dihydrochloride product. HRMS calcd. for C₁₀H₁₉FN₃O₂: m/z=232.1461 [M+H]⁺, found: 232.1485. ¹H NMR (D₂O) δ 1.43 (s, 3H), 2.10 (s, 3H), 1.8-2.1 (m, 4H), 3.98 (d, 2H) 5.29 (dt, vinyl, 1H). ¹⁹F NMR (CDCl₃) δ −109.97 (q, 1 F, J=21 Hz).

### Example II

### Preparation of (2S,5E)-2-amino-2-methyl-6-fluoro-7-[(1-iminoethyl)amino]-5-heptenoic acid, dihydrochloride

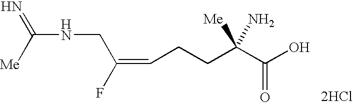

### Ex-II-1

### Preparation of

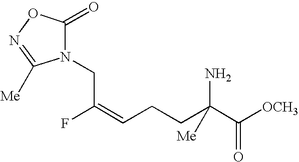

To a 1-methyl-2-pyrrolidinone (7500 mL) solution of methyl N-[(3,4-dichlorophenyl)-methylene]-alaninate (748.5 g, 2.88 mol) under N₂ was added LiI (385.5 g, 2.88 mol) and the resulting slurry stirred approximately 20 min to give a clear solution. The solid from Ex-HH-5 (750 g, 2.40 mol) was then added and the resulting solution cooled in an ice bath to ˜0° C. Neat BTPP (900 g, 2.88 mol) was added dropwise over 25 min maintaining the internal temperature below 5° C. After stirring for an additional 1.5 hr at 5° C., the reaction was determined to be complete by HPLC. At this time, 7500 mL of methyl t-butyl ether (MTBE) was added followed by addition of 9750 mL of a water/crushed ice mixture. The temperature rose to 20° C. during this operation. After stirring vigorously for 5-10 min, the layers were separated and the aqueous layer washed with twice with 6000 mL of MTBE. The MTBE layers were combined and washed 2 times with 7500 mL of water. The resulting MTBE solution was then concentrated to ˜5000 mL, treated with 11625 mL of 1.0 N HCl, and stirred vigorously at room temperature for 1 hr. The layers were separated and the aqueous layer washed with 7500 ml of MTBE. About 1 kg of sodium chloride was added to the aqueous layer and the resulting mixture stirred until all the salt had dissolved. At this point, 7500 mL of ethyl acetate was added, the resulting mixture cooled to 10° C., and 2025 mL of 6.0 N sodium hydroxide added with good agitation. The resulting pH should be about 9. The layers were separated and the aqueous layer was saturated with sodium chloride and extracted again with 7500 mL of ethyl acetate. The combined ethyl acetate extracts were dried (MgSO₄) and concentrated to a light oil. It should be noted that the ethyl acetate was not complete removed. With agitation, 3000 ml of hexane then is added to generate a slurry that was cooled to 10° C. The granular solid was collected by filtration and washed with 1500 mL of hexane. About 564 g (82% yield) of the desired pure aminoester (>95% pure by HPLC) was obtained as a white solid, m.p. 82.9-83.0° C. LCMS: m/z=288.2 [M+H]⁺. Chiral HPLC (Chiralpak-AD normal phase column, 100% acetonitrile, 210 nm, 1 mL/min): Two major peaks at 4.71 and 5.36 min (1:1). ¹H NMR (CDCl₃): δ 1.40 (s, 3H), 1.7-1.8 (m, 2H), 2.0 (br s, 2H), 2.2 (m, 2H), 2.29 (s, 3H), 3.73 (s, 3H), 4.34 (dd, 2H), 5.33 (dt, 1H).

### Ex-II-2

### Preparation of

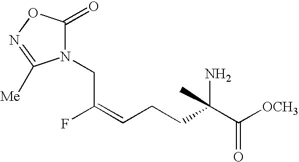

Separation of the individual enantiomers of the product from Ex-II-1 was accomplished on preparative scale using chiral HPLC chromatography (ChiralPak-AD, normal phase column, 100% acetonitrile) to give the desired pure (2S)-2-methyl amino ester product title product. ChiralPak-AD, normal phase column, 100% acetonitrile, 210 nm, 1 mL/min): 5.14 min (99%).

### Ex-II-3

### Preparation of

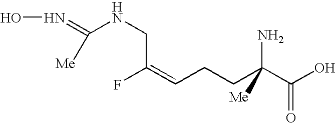

A slurry of the product of Ex-II-2 (2.30 g, 8.01 mmol) in 0.993 M NaOH (30.0 ml, 29.79 mmol) was stirred 2 hr at room temperature. To the resulting clear colorless solution was added 1.023 M HCl (29.10 mL, 29.76 mmol). The resulting clear solution was concentrated until a precipitate began to form (approx. 30 mL). The slurry was warmed to give a clear solution that was allowed to stand at room temperature overnight. The precipitate was isolated by filtration. The solid was washed with cold water (2×10 mL), cold methanol (2×10 mL) and Et₂O (2×20 mL). The white solid was dried in vacuo at 40° C. 4 hr to give 1.04 g (53%) of the desired N-hydroxy illustrated product. mp=247.2° C. Anal. calcd. for C₁₀H₁₈FN₃O₃: C, 48.57; H, 7.34; N, 16.99; Cl, 0.0. Found: C, 48.49; H, 7.37; N, 16.91; Cl, 0.0. HRMS calcd. for C₁₀H₁₉FN₃O₃: m/z=248.1410 [M+H]⁺, found: 248.1390. ¹H NMR (D₂O) δ 1.35 (s, 3H), 1.81 (s, 3H), 1.7-2.0 (m, 4H), 3.87 (d, 2H) 5.29 (dt, vinyl, 1H). ¹⁹F NMR (CDCl₃) δ −112.51 (q, 1 F, J=21 Hz).

Ex-II-4. To a solution of Ex-II-3 in methanol is added Lindlar catalyst. The stirred slurry is refluxed for 2 hr, then cooled to room temperature. The catalyst is removed by filtration through celite, and the filtrate is stripped. The resulting solid is dissolved in water and concentrated repeatedly from 1.0 N HCl to give the desired (2R,5E)-2-amino-2-methyl-6-fluoro-7-[(1-iminoethyl)amino)-5-heptenoic acid, dihydrochloride product.

### Ex-II-5

### Preparation of

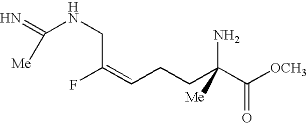

Method A. A solution of 73.5 g (0.3 mol).of the product from Ex-II-2 was dissolved in 300 mL of methanol and added dropwise to a preformed mixture of 13.7 g of Lindlar catalyst and 73.5 g of formic acid (1.53 mol) in 312 mL of methanol while maintaining the reaction temperature between 22° C. and 26° C. After stirring at room temperature for an additional 15 hr, the reaction was determined to be complete by F¹⁹ NMR. The resulting reaction mixture was filtered through celite and the celite washed 3 times with 125 mL of methanol. The methanol filtrates were combined and concentrated to generate 115 g of the desired amidine title product as a viscous oil. MS: m/z=246 (M+H)⁺. ¹H NMR (CD₃OD) δ ppm, 4H) 2.3 (s, 3H), 3.9 (s, 3H), 4.2 (d, 2H), 5.4 (dt,vinyl), 8.4 (s, 3H). F¹⁹NMR (CD₃OD) δ ppm, J=21 Hz) −111.7 (q, J=21 Hz). To remove trace levels of lead, the crude product was dissolved in 750 mL of methanol and 150 g of a thiol-based resin (Deloxan THP 11) was added. After stirring 3 hrs at room temperature, the resin was filtered off and washed 2 times with 500 mL methanol. The filtrates were collected and concentrated to 99 g of the desired amidine title product as a viscous oil.

Method B. A total of 5.0 g of the product from Ex-II-2 (0.0174 mole, 1.0 equiv) was mixed with 5.0 g of zinc dust (0.0765 moles, 4.39 equiv) in 40 mL of 1-butanol and 10 mL of acetic acid. After stirring for 5 hrs at 50° C., LC analyses indicated the reaction to be complete. The solids were readily filtered off. The filtrate, after cooling in ice water to 7° C., was treated with 30 mL of 6 N NaOH (0.180 moles) in 1 portion with vigorous stirring. After cooling the reaction mixture from 33° C. to 20° C., the clear butanol layer was separated off and the aqueous layer extracted again with 40 mL of 1-butanol. The butanol extracts were combined, and washed with 30 mL of brine, followed by approx 10 mL of 6N HCl. After concentration at 70° C., a clear glass resulted which was identified as the desired amidine title product.

### Ex-II-6

### Preparation of

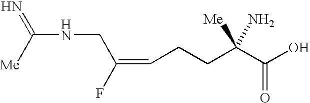

A solution of 99 g of the product from Ex-II-5 in 6 N HCl was refluxed for 1 hr at which time LC analyses indicated the reaction to be complete. The solvent was removed in vacuo to yield 89.2 g of a glassy oil which was dissolved in a mixture of 1466 mL ethanol and 7.5 ml of deionized water. THF was added to this agitated solution at ambient temperature until the cloud point was reached (5.5 liters). An additional 30 ml of deionized water was added and the solution agitated overnight at room temperature. The resulting slurry was filtered and washed with 200 mL of THF to yield 65 g of a white solid identified as the desired title product. [□]_{D}²⁵=+7.2 (c=0.9, H₂O). mp=126-130° C. MS: m/z=232 (M+H)⁺. Anal. Calcd for C₁₀H₂₂N₃F₁O₃Cl₂: C, 37.28; H, 6.88; N, 13.04; Cl, 22.01. Found: C, 37.52, H, 6.84, N, 13.21, Cl, 21.81. ¹H NMR (D₂O) δ ppm, 3H), 1.8-2.1 (m, 4H), 1.9 (s,3H), 4.0(d, 2H), 5.3(dt, vinyl, 1H). F¹⁹NMR (D₂O) δ, J=21 Hz) −112.1 (q, J-21 Hz).

### Example JJ

### Preparation of (2R,5E)-2-amino-2-methyl-6-fluoro-7-[(1-iminoethyl)amino]-5-heptenoic acid, dihydrochloride

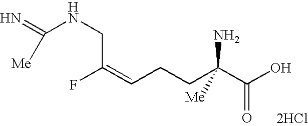

### Ex-JJ-1

### Preparation of

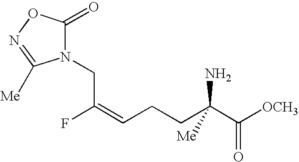

Separation of the individual enantiomers of the product from Ex-II-1 was accomplished on preparative scale using chiral HPLC chromatography to give the desired pure (2R)-2-methyl amino ester product.

### Ex-JJ-2

### Preparation of

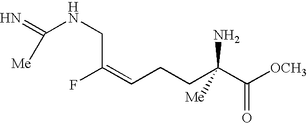

The product from Ex-JJ-1 is dissolved in water and acetic acid. Zinc dust is added, and the mixture is heated at 60° C. until HPLC analysis shows that little of the starting material 10 remains. The Zn is filtered through celite from the reaction mixture, and the filtrate is concentrated. The crude material is purified by reverse-phase HPLC column chromatography. Fractions containing product are combined and concentrated affording the desired (2R)-2-methyl acetamidine product.

### Ex-JJ-3

### Preparation of

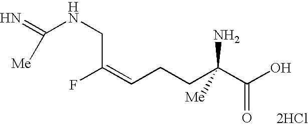

A solution of Ex-JJ-2 in 2.0 N HCl is refluxed for 2 hr. The solvent is removed in vacuo. The resulting solid is dissolved in water and concentrated repeatedly from 1.0 N HCl to give the desired (2R,5E)-2-amino-2-methyl-6-fluoro-7-[(1-iminoethyl)amino]-5-heptenoic acid, dihydrochloride product.

### Example KK

### Preparation of (2R/S,5E)-2-amino-2-methyl-6-fluoro-7-[(1-iminoethyl)amino]-5-heptenoic acid, dihydrochloride

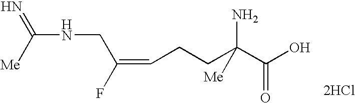

### Ex-KK-1

### Preparation of

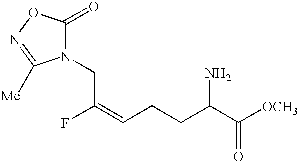

To an 1-methyl-2-pyrrolidinone (5 mL) solution of methyl N-[(4-chlorophenyl)methylene]-glycinate (0.33 g, 1.6 mmol), LiI (0.20 g, 1.0 mmol) and a sample of the product of Ex-HH-5 (0.30 g, 0.96 mmol) in an ice bath was added 2-tert-butylimino-2-diethylamino-1,3-dimethylperhydro-1,3,2-diazaphosphorine (0.433 mL, 1.5 mmol). The solution was allowed to stir at room temperature for 1.5 hr. The reaction mixture was diluted with ethyl acetate (30 mL), washed with water (2×20 mL), dried (MgSO₄), filtered, and evaporated to give the crude desired racemic alkylated imine as a yellow oil. The crude material was dissolved in ethyl acetate (10 mL) and 1N HCl (10 mL) was added. The mixture was stirred for 2 hr at room temperature, and the organic layer was separated. The aqueous layer was neutralized with solid NaHCO₃ and extracted with ethyl acetate (2×30 mL). The organic layer was dried (MgSO₄), filtered and evaporated to give 0.13 g of the desired title racemic amino ester product as a yellow oil. This product was used in the next step without further purification. LCMS: m/z=288.2 [M+H]⁺.

### Ex-KK-2

### Preparation of

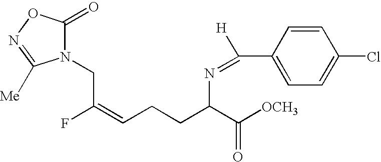

To a CH₂Cl₂ (15 mL) solution of Ex-KK-1 (1.36 g, 4.98 mmol) was added 4-chlorobenzaldehyde (0.70 g, 5.0 mmol) and MgSO₄ (˜5 g). The slurry was stirred at room temperature for 18 hr. The slurry was filtered, and the filtrate stripped to give 1.98 g (100%) of the desired title imine product as a pale yellow oil. This product was used in the next step without further purification. ¹H NMR (C₆D₆) δ 1.34 (s, 3H), 2.0 (br m, 4H), 3.32 (s, 3H), 3.42 (m, 2H), 3.83 (t, 1H), 4.98 (dt, vinyl, 1H).

### Ex-KK-3

### Preparation of

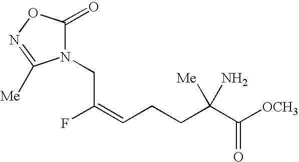

To a CH₂Cl₂ (2 mL) solution of the product of Ex-KK-2 (0.25 g, 0.63 mmol) was added methyl iodide (0.200 mL, 3.23 mmol) and O(9)-allyl-N-(9-anthracenylmethyl)-cinchonidinium bromide (40 mg, 0.066 mmol). The solution was cooled to −78° C. and neat BTPP (0.289 mL, 0.95 mmol) was added. The resulting orange solution was stirred at −78° C. for 2 hr and allowed to reach −50° C. After 2 hr at −50° C., the solution was diluted with CH₂Cl₂ (10 mL), washed with water (10 mL), dried (MgSO₄), filtered, and evaporated to give the crude desired racemic alkylated imine as a yellow oil. The crude material was dissolved in ethyl acetate (10 mL) and 1N HCl (10 mL) was added. The mixture was stirred for 1 hr at room temperature, and the organic layer was separated. The aqueous layer was neutralized with solid NaHCO₃ and extracted with ethyl acetate (2×30 mL). The organic layer was dried (MgSO₄), filtered and evaporated to give 0.16 g of the desired racemic 2-methylamino ester product as a yellow oil. The product was used in the next step without further purification. LCMS: m/z=288.2 [M+H]⁺.

### Ex-KK-4

### Preparation of

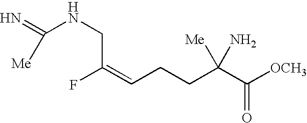

The racemic product from Ex-KK-3 is dissolved in water and acetic acid. Zinc dust is added, and the mixture is heated at 60° C. until HPLC analysis shows that little of the starting material remains. The Zn dust is filtered through celite from the reaction mixture, and the filtrate is concentrated. The crude material is purified by reverse-phase HPLC column chromatography. Fractions containing product are combined and concentrated affording the desired acetamidine product.

### Ex-KK-5

### Preparation of

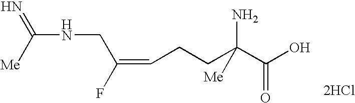

A solution of racemic Ex-KK-4 in 2.0 N HCl is refluxed for 1 hr. The solvent is removed in vacuo. The resulting solid is dissolved in water and concentrated repeatedly from 1.0 N HCl to give the desired title (2R/S,5E)-2-amino-2-methyl-6-fluoro-7-[(1-iminoethyl)amino]-5-heptenoic acid, dihydrochloride product.

### Example LL

### Preparation of (2S,5Z)-2-amino-2-methyl-7-[(1-iminoethyl)amino]-5-heptenoic acid, dihydrochloride

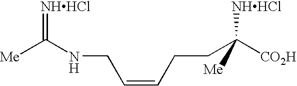

### Ex-LL-1

### Preparation of 4-[(tetrahydropyranyl)oxy]butyne

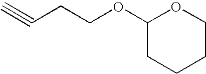

A mixture of 4-dihydro-2H-pyridine (293.2 g 3.5 mol) and concentrated HCl (1.1 mL) was cooled to 5° C. While continuing to cool externally, 3-butyn-1-ol (231.5 g, 3.3 mol) was added over a period of 30 min allowing the temperature to reach 50° C. Reaction was held with mixing at room temperature for 2.5 hr before it was diluted with MTBE (1.0 L). The resulting mixture was washed with saturated sodium bicarbonate (2×150 mL). The organic phase was dried over sodium sulfate and concentrated under reduced pressure to afford 500 g (98% crude yield) of product; GC area % of 96%.

### Ex-LL-2

### Preparation of 5-(tetrahydro-pyran-2-yloxy)-pent-2-yn-1-ol

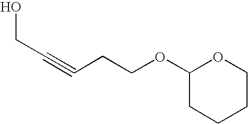

To a solution of the 4-[(tetrahydropyranyl)oxy]butyne product of Ex-LL-1 (50.0 g, 0.33 mol) in THF (125 mL) was added a solution of 2N EtMgCl in THF (242 mL, 0.48 mol) under an N₂ atmosphere over a 30 min period, allowing the temperature to rise to 48° C. Mixture was further heated to 66° C. and was held at this temperature for 2 hr before cooling to ambient temperature. Paraformaldehyde (14.5 g, 0.48 mol) was added (small exotherm was observed) and the resulting mixture was heated to 45° C. After 1 hr of controlling the temperature between 45-55° C., the mixture turned clear. At this point, the mixture was heated up to 66° C. and stirred for 2.5 hr. Mixture was cooled to room temperature and saturated ammonium chloride (125 mL) was added slowly over 30 min (strong exotherm was observed) keeping the temperature below 40° C. The liquid phase was separated by decantation; ethyl acetate (250 mL) and brine (50 mL) were added. The organic phase was separated and washed with brine (2×50 mL) and water (1×50 mL). The organic layer was dried over sodium sulfate and concentrated under reduced pressure to afford 51 g of a lightly yellow colored oil (85% crude yield); GC area %=88% title product, 6% starting material.

### Ex-LL-3

### Preparation of 5-(tetrahydro-pyran-2-yloxy)-pent-2-en-1-ol

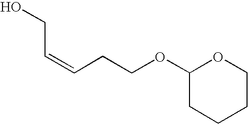

To a 500 mL Parr bottle, under an N₂ atmosphere, was charged the 5-(tetrahydro-pyran-2-yloxy)-pent-2-yn-1-ol product of Ex-LL-2 (40.2 g, 0.22 mol), Lindlar catalyst (2.0 g), ethanol (120 mL), hexane (120 mL), and 2,6-lutidine (457 mg). Reaction mixture was f purged 5 times each with N₂ and H₂. Parr bottle was pressurized with hydrogen to 5 psi and shaken until 98% of the theoretical hydrogen was consumed. Hydrogen was released from the vessel and the reaction was purged with N₂ 5 times. Mixture was filtered through a pad of Solka Floc and the catalyst was rinsed with ethanol (2×50 mL). The filtrate and rinses were combined and concentrated under reduced pressure to afford 40.3 g (99% yield) of the title material as a yellow colored oil (GC area %=96%).

### Ex-LL-4

### Preparation of 3-methyl4-[5-(tetrahydro-pyran-2-yloxy)-pent-2-enyl]-4H-[1,2,4]oxadiazol-5-one

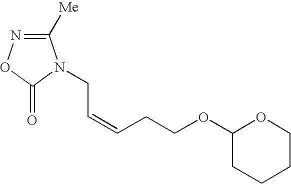

To a solution of the 5-(tetrahydro-pyran-2-yloxy)-pent-2-en-1-ol product of Ex-LL-3 (11.8 g, 0.063 mol) in toluene (42 mL) was added) triethylamine (6.4 g, 0.063 mol). The mixture was cooled to −5° C. and methanesulfonyl chloride (7.3 g, 0.63 mol) was added via syringe at such rate as to keep the pot temperature below 10° C. The mixture was allowed to warm to room temperature and stirred for 2 hr. The mixture was filtered by suction and rinsed on the filter with toluene (2×20 mL). The filtrate and washes were added to a mixture of the sodium salt of 3-methyl-1,2,4-oxadiazolin-5-one (8.6 g, 0.063 mol) in DMF (10 mL). The mixture was stirred with a mechanical stirrer and heated at 45° C. for 5 hr. Water (40 mL) was added and the mixture was stirred for 5 min and then the layers were separated. The toluene layer was washed with water (3×20 mL), dried over MgSO₄, and concentrated to afford 16.5 g (97.3%) of an orange colored crude product (area % GC consisted of 71% title product, 18% toluene, and 4% of an impurity).

### Ex-LL-5

### Preparation of 4-(5-hydroxy-pent-2-enyl)-3-methyl-4H-[1,2,4]oxadiazol-5-one

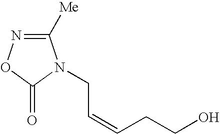

To a solution the 3-methyl-4-[5-(tetrahydro-pyran-2-yloxy)-pent-2-enyl]-4H-[1,2,4]oxadi-az-ol-5-one product of Ex-LL-4 (16 g, 0.06 mol) in methanol (48 mL) was added p-toluenesulfonic acid (0.34 g, 2.0 mmol). The mixture was stirred at room temperature for 4 hr. Sodium bicarbonate (0.27 g, 3.0 mmol) was added and the mixture was concentrated on a rotary evaporator. The residue was diluted with saturated NaHCO₃ (20 mL) and the resulting mixture was extracted with ethyl acetate (2×60 mL). Extracts were combined and washed with water (2×25 mL), dried over MgSO₄, and concentrated to afford 8.4 g of the crude, orange colored oil title product (area % GC=80%).

### Ex-LL=6

### Preparation of methanesulfonic acid 5-(3-methyl-5-oxo-[1,2,4]oxadiazol-4-yl)-pent-3-enyl ester

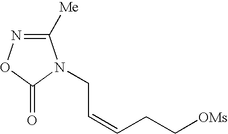

To a solution of the 4-(5-hydroxy-pent-2-enyl)-3-methyl-4H-[1,2,4]oxadiazol-5-one product of Ex-LL-5 (8.27 g, 0.045 mol) in methylene chloride (33 mL) was added triethylamine (5.0 g, 0.49 mol). The mixture was cooled to −5° C. and methanesulfonyl chloride (5.5 g, 0.048 mol) was added at such rate as to keep the temperature below 8° C. The cooling bath was removed and the mixture was stirred for 3 hr as it warmed up to room temperature. Water (15 mL) was added and the mixture was stirred for 5 min and then the layers were separated. The organic phase was washed with water (10 mL), dried over MgSO₄, and concentrated to give a light amber colored residue. The residue was dissolved in ethyl acetate (8 mL) and kept at 5° C. overnight. Precipitated solids were filtered off by suction and rinsed on the filter with minimum volume of ethyl acetate and then air-dried on the filter to afford 6.8 g (58% yield) of the title product. ¹H NMR (CDCl₃) δ 5.76 (dtt, J=10.9, 7.5, 1.5 Hz, 1H), δ 5.59 (dtt, J=10.9, 7.0, 1.5 Hz, 1H), δ 4.31 (t, J=6.3 Hz, 2H), δ 4.27 (dd, J=7.0, 1.5 Hz, 2H), δ 3.04 (s, 3H), δ 2.67 (q, J=6.7 Hz, 2H), δ 2.28 (s, 3H). ¹³C (CDCl₃) δ 159.0, 156.3, 129.9, 125.1, 68.4, 38.9, 37.2, 27.5, 10.2. IR (cm⁻¹) 1758, 1605, 1342,1320,1170. Anal. Calcd. for C₉H₁₄N₂O₅S: C, 41.21; H, 5.38; N, 10.68. Found: C, 41.15; H, 5.41; N, 10.51.

### Ex-LL-7

### Preparation of 4-(5-iodo-pent-2-enyl)-3-methyl-4H-[1,2,4]oxadiazol-5-one

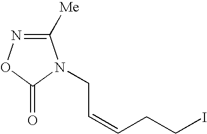

To a solution of the methanesulfonic acid 5-(3-methyl-5-oxo-[1,2,4]oxadiazol-4-yl)-pent-3-enyl ester product of Ex-LL-6 (20.0 g, 0.076 mol) in acetone (160 ml) was added sodium iodide (17.15 g, 0.114 mol). The mixture was heated to reflux and was stirred for 3 hr. External heating was stopped and the mixture was held at room temperature overnight. Solids were removed by filtration and rinsed on the filter. The filtrate and washes were combined and concentrated and the heterogeneous residue was extracted with ethyl acetate (120 mL). The organic layer was washed with water (60 mL), 15% aqueous solution of sodium thiosulfate (60 mL) and water (60 mL); dried over MgSO₄ and concentrated under reduced pressure to afford 22.1 g (98% yield) of the title oil product.

### Ex-LL-8

### Preparation of 2-[(3,4-dichloro-benzylidene)-amino]-propionic acid methyl ester

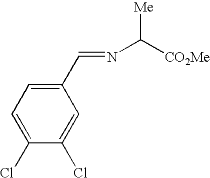

To a mechanically stirred slurry of L-alanine methyl ester hydrochloride (200.0 g, 1.43 mol) in methylene chloride (2.1 L) under an N₂ atmosphere was added triethylamine (199.7 mL, 1.43 mol) over 12 min (during the addition solids partially dissolved and then reprecipitated). After 10 min, 3,4-dichlorobenzaldehyde (227.5 g, 1.30 mol) and magnesium sulfate (173.0 g, 1.43 mol) were added (temperature increased 6° C. over 30 min). After 2.5 hr, the mixture was filtered. The filtrate was washed with water (1×1 L) and brine (1×500 mL), dried over sodium sulfate, filtered and concentrated to give 313.3 g, 92.4% yield of oil product. ¹H NMR (400 MHz, CDCl3) δ 8.25 (s, 1H), 7.91 (d, 1H), 7.58 (dd, 1H), 7.49 (d, 1H), 4.17 (t, 1H), 3.76 (s, 3H), 1.53 (d, 3H). Anal. Calcd for C₁₁H₁₁Cl₂NO₂: C, 50.79; H, 4.26; Cl, 27.26; N, 5.38. Found: C, 50.37; H, 4.10; Cl, 26.87; N, 5.38.

### Ex-LL-9

### Preparation of Rac-2-amino-2-methyl-7-(3-methyl-5-oxo-[1,2,4]oxadiazol-4-yl)-hept-5-enoic acid methyl ester

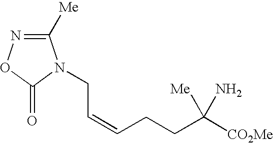

Method A. A solution of the product of Ex-LL-7 (114.2 g, 0.39 mol) and the product of Ex-LL-8 (151.5 g, 0.58 mol) in dimethylformamide (1.4 L) under N₂ atmosphere was cooled to −8° C. Lithium iodide (78.1 g, 0.58 mol) was then added in 3 equal portions over 19 min. The mixture was stirred for 20 min at −7° C. and then (tert-butylimino)-tris(pyr-rolidino)phosphorane (194.0 mL, 0.62) was added over 36 min (maximum temperature=−2.6° C.). After 10 min, the cooling bath was removed and the solution was stirred at ambient temperature for 1 h. The mixture was then poured into cold water (1.4 L) and extracted with ethyl acetate (2×1.0 L). The combined organic layers were washed with water (2×400 mL) and brine. The ethyl acetate layer was treated with 1 N HCl (780 mL) and stirred for 1 hr. The aqueous layer was separated and extracted with ethyl acetate (2×400 mL) and then neutralized with sodium bicarbonate (110 g). The mixture was extracted with ethyl acetate (1×500 mL). The organic layer was dried over sodium sulfate, filtered, concentrated and then treated with methyl t-butyl ether to give a crystalline product: first crop 14.4 g; second crop 6.6 g (GC purity=96.2 and 91.9%, respectively). The aqueous phase was saturated with sodium chloride and extracted with ethyl acetate (4×500 mL). The combined organic layers were dried over sodium sulfate, filtered, concentrated and then treated with methyl t-butyl ether to give a crystalline product: first crop 33.4 g; second crop 10.8 g (GC purity=89.6 and 88.8%, respectively. Total crude yield 65.2 g, 62.4%.

Method B. To a solution of the product of Ex-LL-7 (20.7 g, 0.070 mol) and the product of Ex-LL-8 (22.9 g, 0.088 mol) in dimethylformamide (207 mL) under an N₂ atmosphere was added cesium carbonate (29.8 g, 0.092). The mixture was stirred at room temperature for 16 hr and then diluted with water (300 mL) and extracted with ethyl acetate (2×200 mL). The combined ethyl acetate layers were washed with water (3×100 mL) and brine and then treated with 1 N HCl (184 mL). After 1 hr, the layers were separated and the aqueous layer was extracted with ethyl acetate (3×100 mL) and then neutralized with sodium bicarbonate (15.5 g). The mixture was extracted with ethyl acetate (1×150 mL). The aqueous layer was saturated with sodium chloride and extracted with ethyl acetate (3×100 mL). The combined organic layers were dried over sodium sulfate, filtered and concentrated to give a yellow solid, 11.9 g, 62.9%; GC purity=96.6%. The crude product was recrystallized from warm methyl t-butyl ether or ethyl acetate.

¹H NMR (400 MHz, CDCl₃) δ 5.68 (m, 1H), 5.36 (m, 1H), 4.23 (d, 2H), 3.73 (s, 3H), 2.43 (s, 3H), 2.18 (m, 2H), 1.81 (m, 1H), 1.69 (s, br, 2H), 1.66 (m, 1H), (1.36, 3H). ¹³C NMR (400 MHz, CDCl₃) δ 177.60, 159.01, 156.10, 135.12, 121.82, 57.48, 52.29, 40.12, 39.00, 26.62, 22.56, 10.41

### Ex-LL-10

### Preparation of Rac-2-amino-2-methyl-7-(3-methyl-5-oxo-[1,2,4]oxadiazol-4-yl)-hept-5-enoic acid

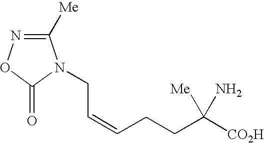

The product of Ex-LL-9 (0.269 g, 1 mmol) was dissolved in 5 mL 2 N HCl and heated to reflux under argon. After refluxing for 6 hrs followed by stirring at room temperature for 72 hr, an aliquot was removed and checked by ¹H NMR. Approximately 6% of unreacted starting ester remained along with the desired product (verified by LC-MS). The aqueous portion was removed in vacuo, leaving 0.38 g of a thick, amber oil. After purification via reverse phase chromatography, followed by lyophilization, one obtained 0.23 g, 90.2% of the title compound as white, non-deliquescent solids. Anal. Calcd. for C₁₁H₁₇N₃O₄.0.77H₂O: C, 49.09; H, 6.94; N, 15.61. Found: C, 48.71; H, 6.94; N, 15.98. Mass spec: M+1=256.

### Ex-LL-11

### Preparation of (2S,5Z)-2-amino-2-methyl-7-(3-methyl-5-oxo-[1,2,4]oxadiazol-4-yl)-hept-5-enoic acid methyl ester

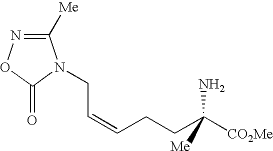

The title compound (827.3 g) was separated from its R enantiomer by preparative chiral chromatography using Novaprep 200 instrument with steady state recycling option. The material was dissolved in absolute ethanol at a concentration of 40 mg/ml and loaded on a 50×500 mm prepacked Chiral Technologies stainless steel column. The adsorbent was 20μ ChiralPak AD. The mobile phase was ethanol/triethylamine 100/0.1; the flow rate equaled 125 ml per min. The crude solution (25 mL) was loaded on the column every 12 mins. A steady state recycling technique was used. Solvent was removed using a rotovap. The final product was isolated as gold oil which solidified on standing; 399.0 g (96.4% recovery). ¹H (400 MHz, CD₃OD) δ 5.68 (dtt, 1H, J_{olefinic}=10.7 Hz), 5.43 (dtt, 1H, J_{olefinic}=10.7 Hz), 4.82 (s, br, 2H), 4.28 (d, 2H, J=5.5 Hz), 3.73 (s, 3H), 2.27 (s, 3H), 2.26 (m, 1H), 2.14 (m,1H), 1.82 (ddd, 1H, J=13.6,11.3, 5.4 Hz), 1.67 (ddd, 1H, J=13.6, 11.2 5.5 Hz), 1.34 (s, 3H). ¹³C NMR (400 MHz, CD₃OD) δ 178.49, 161.13, 158.70, 135.92, 123.47, 58.55, 52.77, 41.38, 39.96, 26.23, 23.47, 10.23. Anal. Calcd for C₁₂H₁₉N₃O₄: C, 53.52; H, 7.11; N, 15.60. Found: C 52.35; H, 7.20; N, 15.60.

### Ex-LL-12

### Preparation of (2S,5Z)-7-acetimidoylamino-2-amino-2-methyl-hept-5-enoic acid methyl ester, dihydrochloride hydrate

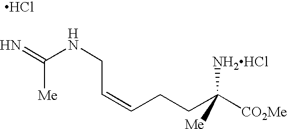

To a solution of the product of Ex-LL-11 (114.5 g, 0.425 mol) in methanol (2.4 L) was added the solid dibenzoyl-L-tartaric acid (152.5 g, 0.425 mol) and 88% formic acid (147 mL, 3.428 mol) at ambient temperature. A slurry of Lindlar catalyst, 5 wt % palladium on calcium carbonate poisoned with lead acetate (37.9 g), in methanol (200 mL) was prepared under N₂. The solution of starting material was then added at ambient temperature to the light grey catalyst slurry followed by a methanol rinse (200 mL). The heterogeneous reaction mixture was heated at 45° C. for 1½ hr. Steady gas evolution was observed starting at about 40° C., which indicated the ongoing reaction. The mixture was cooled in an ice/water bath and then filtered through a plug of Supercell HyFlo. The yellow solution was concentrated in vacuo to give a viscous oil, which was dissolved and partitioned between 2 N aqueous HCl (2 L) and ethyl acetate (0.8 L). Layers were separated and the aqueous layer was washed once with ethyl acetate (0.8 L). Solvent and volatiles were removed in vacuo at elevated temperatures (=70° C.). The intermediate product was used in next the step without further purification or characterization. LC-MS [M+H]⁺=228.

### Ex-LL-13

### Preparation of

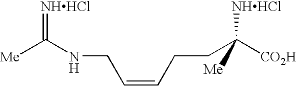

The crude product of Ex-LL-12 (170 g) was dissolved in 2 N aqueous HCl (1 L). The resulting orange solution was refluxed overnight before it was allowed to cool back to ambient temperature. The reaction mixture was concentrated to about ⅓ of its volume, and the acidic solution was passed through a solid phase extraction cartridge (25 g of C18 silica) to remove color and other impurities. Solvent was removed in vacuo (=70° C.) to give 208 g of crude product as yellowish gum. The crude gum (31.3 g) was taken up in water (250 mL) and the material was loaded onto a pretreated ion exchange column packed with the acidic resin Dowex 50WX4-400 (about 600 g). The resin was first washed with water (1 L), then with dilute aqueous HCl (1 L of 10/90 v/v conc. HCl/water). The product was eluted off the resin with higher ion strength aqueous HCl (1.5 L of 20/90 v/v to 25/75 v/v conc. HCl/water). The aqueous solvent was removed in vacuo (=70° C.), and the gummy residue was taken up in 4 vol % aqueous trifluoroacetic acid (100 mL). The aqueous solvent was removed in vacuo (=70° C.), and the procedure was repeated once more. The residue was then dried under high vacuum to give 32.2 g of gum as the trifluoroacetic acid salt. Crude (2S,5Z)-7-acetimidoylamino-2-amino-2-methyl-hept-5-enoic acid, ditrifluoroace-tic acid salt hydrate (32.2 g) was purified by reverse-phase preparative chromatography. The crude was dissolved in 0.1% aqueous TFA (50 ml) and loaded onto a 2-inch ID×1 meter stainless steel column packed with adsorbent (BHK polar W/S, 50 □, 1.16 kg). The product was eluted at a flow rate of 120 mL/min with a step gradient from 0.1% aqueous TFA to 25/75/0.1 acetonitrile/water/TFA. The loading ratio was 36:1 w/w silica to sample. Solvent was removed in vacuo, and the material was converted into the HCl salt by repeated rinses with dilute aqueous HCl and solvent removals in vacuo. Drying under high vacuum gave 27.4 g of the title dihydrochloride hydrate as yellowish gum. LC-MS [M+H]⁺=214.16 Da. ¹H NMR (D₂O, δ: 1.48 (s, 3H), 1.8-1.9 (AB, 2H), 2.10 (s, 3H), 2.01/2.12 (AB, 2H), 3.78 (d, 2H), rotamere 3.87 (d, 2H), 5.6/5.5 (dt, 2H, 11 Hz). ¹³C NMR (D₂O) δ: 18.7, 21.5, 21.6, 36.4, 39.1, 59.8, 122.6, 134.3, 164.5, 173.7. Elemental Anal. Calcd. for C₁₀H₁₉N₃O₂.2.2HCl.2H₂O: C, 36.21; H, 8.33; N, 12.67; Cl 23.51. Found: C, 36.03; H, 7.72; N, 12.67; Cl, 23.60.

### Example MM

### Preparation of (2R,5Z)-2-amino-2-methyl-7-[(1-iminoethyl)amino]-5-heptenoic acid, dihydrochloride

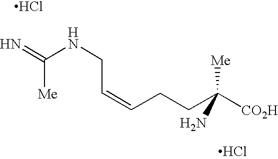

The R-enantiomer isolated during the separation described in Ex-LL-11 (1.13 g, 4.2 mmol) was dissolved in 11 mL 25% aqueous acetic acid and heated to 60° C. Zinc dust (1.10 g) was then added in 4 equal portions at 30-min intervals. After heating for a total of 3 hr, an aliquot was removed and checked by LC-MS, which indicated only a trace of unreacted starting material remaining, along with desired product. The mixture was cooled to room temperature, filtered and stripped in vacuo, leaving 2.31 g of a slushy white solid. The methyl ester was hydrolysed with dilute hot HCl to the title compound. After purification by reverse phase chromatography followed by lyophilization, 0.31 g of the title compound as a glassy solid was obtained. Anal. Calcd. for C₁₀H₁₉N₃O₂.1.22HCl.1.15H₂O: C, 46.13; H, 8.15; N, 15.09; Cl, 15.53. Found: C, 46.38; H, 8.51; N, 15.13; Cl, 15.80. Mass spec: M+1=214

### Example NN

### Preparation of 2S-amino-6-[(1-iminoethyl)amino]-N-(1H-tetrazol-5-yl)-hexanamide, hydrate, dihydrochloride

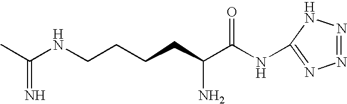

Ex-NN-1. To a stirring solution of Boc-L-Lys(Cbz)-OH (5 g, 13.18 mmol), 5-aminotetrazole monohydrate (1.36 g, 13.18 mmol ) and N,N-diisopropylethylamine (DIPEA) (5.1 g, 6.9 mL, 39.54 mmol) in 20 mL of dimethylformamide (DMF) at ambient temperature was added benzotriazol- 1-yl-oxy-tris-(dimethylamino)phosphonium hexafluorophosphate (BOP) (6.4 g, 14.49 mmol). After being stirred for 1 hr, the reaction mixture was concentrated under vacuum. The residue was distributed between 60 mL of ethyl acetate (EtOAc) and 50 mL of water. The layers were separated. The organic layer was washed with 50 mL of 1M KHSO₄ solution and 2 times with 50 mL of water. The product started to precipitate and the suspension was concentrated in vacuum giving 9 g of crude compound. After drying, the product was purified by boiling in methylene chloride followed by filtration, giving 3.7 g of 1A (62.7%). The compound was characterized by ¹H NMR.

Ex-NN-2. (2 g, 4.5 mmol) was reduced under catalytic hydrogenation conditions using Pd black at 5 psi in 50% EtOH/AcOH solution for 12 hr, giving 1.55 g (100%) of NN-2. The compound was characterized by ¹H NMR.

Ex-NN-3. To a stirring solution of NN-2 (1.55 g, 4.15 mmol) and methyl acetimidate hydrochloride (0.91 g, 8.31 mmol) in 25 mL of DMF was added triethylamine (TEA) (1.26 g, 1.74 mL, 12.45 mmol). After being stirred 16 hr at ambient temperature, the reaction mixture was filtered from triethylamine hydrochloride and the filtrate was concentrated in vacuum. The residue was dissolved in 50% AcOH and lyophilized. The crude product (2 g) was purified using reverse-phase chromatography on a C-18 column giving 0.9 g (52.3%) of 1C. The product was characterized by ¹H NMR.

Ex-NN-4. (0.9 g, 2.17 mmol) was dissolved in 30 mL of acetic acid and 3 mL of 4 N HCl/dioxane were added. The reaction was stirred for 20 min. at ambient temperature then 150 mL of ethyl ether were added. After 2 hr, the precipitate was filtered, washed with ethyl ether, and dried giving 0.78 g of 1 (96%). Anal. Calcd. for C₉H₁₈N₈O,2HCl, 1.25H₂O: C,30.91; H, 6.48; N, 32.04; Cl, 20.27. Found: C, 31.64; H, 6.43; N, 32.19; Cl, 20.19. DSC mp 144.9° C.

Example NN is a desirable crystalline product, as are all its intermediates. In contrast, NIL is a glass, which makes it difficult to handle.

### Biological Data

Some (or all) of the following assays may be used to demonstrate the nitric oxide synthase inhibitory activity of the invention's compounds, as well as demonstrate the useful pharmacological properties.

### A. Citrulline Assay for Nitric Oxide Synthase

Nitric oxide synthase (NOS) activity can be measured by monitoring the conversion of L-[2,3-³H]-arginine to L-[2,3-³H]-citrulline (Bredt and Snyder, Proc. Natl. Acad. Sci. U.S.A., 87, 682-685, 1990 and Moore et al, J. Med. Chem., 39, 669-672, 1996). Human inducible NOS (hiNOS), human endothelial constitutive NOS (hecNOS) and human neuronal constitutive NOS (hncNOS) are each cloned from RNA extracted from human tissue. The cDNA for human inducible NOS (hiNOS) is isolated from a λcDNA library made from RNA extracted from a colon sample from a patient with ulcerative colitis. The cDNA for human endothelial constitutive NOS (hecNOS) is isolated from a λcDNA library made from RNA extracted from human umbilical vein endothelial cells (HUVEC) and the cDNA for human neuronal constitutive NOS (hncNOS) is isolated from a λcDNA library made from RNA extracted from human cerebellum obtained from a cadaver. The recombinant enzymes are expressed in Sf9 insect cells using a baculovirus vector (Rodi et al, in The Biology of Nitric Oxide, Pt. 4: Enzymology, Biochemistry and Immunology; Moncada, S., Feelisch, M., Busse, R., Higgs, E., Eds.; Portland Press Ltd.: London, 1995; pp 447-450). Enzyme activity is isolated from soluble cell extracts and partially purified by DEAE-Sepharose chromatography. To measure NOS activity, 10 μL of enzyme is added to 40 μL of 50 mM Tris (pH 7.6) in the presence or absence of test compounds and the reaction initiated by the addition of 50 μL of a reaction mixture containing 50 mM Tris (pH 7.6), 2.0 mg/mL bovine serum albumin, 2.0 mM DTT, 4.0 mM CaCl₂, 20 μM FAD, 100 μM tetrahydrobiopterin, 0.4 mM NADPH and 60 μM L-arginine containing 0.9 μCi of L-[2,3-³H]-arginine. The final concentration of L-arginine in the assay is 30 μM. For hecNOS or hncNOS, calmodulin is included at a final concentration of 40-100 nM. Following incubation at 37° C. for 15 min, the reaction is terminated by addition of 400 μL of a suspension (1 part resin, 3 parts buffer) of Dowex 50W X-8 cation exchange resin in a stop buffer containing 10 mM EGTA, 100 mM HEPES, pH 5.5 and 1 mM L-citrulline. After mixing the resin is allowed to settle and L-[2,3-³H]-Citrulline formation is determined by counting aliquots of the supernatant with a liquid scintillation counter. Results are reported in Table I as the IC₅₀ values of compounds for hiNOS, hecNOS and hncNOS.

### B. Raw Cell Nitrite Assay

RAW 264.7 cells can be plated to confluency on a 96-well tissue culture plate grown overnight (17 h) in the presence of LPS to induce NOS. A row of 3-6 wells can be left untreated and served as controls for subtraction of nonspecific background. The media can be removed from each well and the cells washed twice with Kreb-Ringers-Hepes (25 mM, pH 7.4) with 2 mg/ml glucose. The cells are then placed on ice and incubated with 50 μL of buffer containing L-arginine (30 μM)±inhibitors for 1 h. The assay can be initiated by warming the plate to 37° C. in a water bath for 1 h. Production of nitrite by intracellular iNOS will be linear with time. To terminate the cellular assay, the plate of cells can be placed on ice and the nitrite-containing buffer removed and analyzed for nitrite using a previously published fluorescent determination for nitrite. T. P. Misko et al, Analytical Biochemistry, 214, 11-16 (1993).

### C. Human Cartilage Explant Assay

Bone pieces are rinsed twice with Dulbecco's Phosphate Buffered Saline (GibcoBRL) and once with Dulbecco's Modified Eagles Medium (GibcoBRL) and placed into a petri dish with phenol red free Minimum Essential Medium (MEM) (GibcoBRL). Cartilage was cut into small explants of approximately 15-45 mg in weight and 1 or 2 explants per well are placed into either 96 or 48 well culture plates with 200-500 μL of culture media per well. The culture media was either a custom modification of Minimum Essential Medium(Eagle) with Earle's salts (GibcoBRL) prepared without L-Arginine, without L-Glutamine and without phenol red or a custom modification of serumless Neuman and Tytell (GibcoBRL) medium prepared without L-arginine, without insulin, without ascorbic acid, without L-glutamine and without phenol red. Both are supplemented before use with 100 μM L-Arginine (Sigma), 2 mM L-glutamine, 1× HL-1 supplement (BioWhittaker), 50 mg/ml ascorbic acid (Sigma) and 150 pg/ml recombinant human IL-1□ (RD Systems) to induce nitric oxide synthase. Compounds are then added in 10 μL aliquots and the explants incubated at 37° C. with 5% CO₂ for 18-24 hr. The day old supernatant is then discarded and replaced with fresh culture media containing recombinant human IL-1β and compound and incubated for another 20-24 hr. This supernatant is analyzed for nitrite with a fluorometric assay (Misko et al, Anal. Biochem., 214, 11-16 (1993)). All samples are done in quadruplicate. Unstimulated controls are cultured in media in the absence of recombinant human IL-1\tilde{□} IC₅₀ values (Table I) are determined from plotting the percent inhibition of nitrite production at 6 different concentrations of inhibitor.

Table I shows examples of biological activity for some compounds of the present invention.

TABLE I
Biological Activity
(values represent averages across all experiments and all lots studied)

| | | | | Human |
|---|---|---|---|---|
| Example | hiNOS | hecNOS | hncNOS | Cartilage |
| Number of | IC₅₀ | IC₅₀ | IC₅₀ | IC₅₀ |
| Compound | (μM) | (μM) | (μM) | (μM) |
| Example A | 0.36 | 68 | 3.6 | 0.1 |
| Example B | 2.2 | 195 | 21 | 0.2 |
| Example C | 12 | 303 | 105 | |
| Example D | 8.6 | 112 | 65 | 2.5 |
| Example E | <5 | 279 | 29 | |
| Example I | 3.1 | 77 | 15 | 0.7 |
| Example J | 4.4 | 302 | 58 | 8.2 |
| Example K | 74 | 266 | 86 | |
| Example L | 197 | 1100 | 539 | |
| Example M | 3.4 | 78 | 17 | |
| Example N | 0.9 | 26 | 6.0 | |
| Example O | 7.2 | >100 | 36 | 0.7 |
| Example P | 12 | >100 | 181 | |
| Example Q | 12 | 1080 | 220 | |
| Example S | 172 | 1490 | 523 | |
| Example T | 0.9 | 89 | 8 | 0.1 |
| Example U | 20 | 418 | 150 | |
| Example V | <3 | >30 | >3 | <10 |
| Example W | <5 | >150 | >10 | >30 |
| Example X | <3 | >15 | >3 | <10 |
| Example Y | <3 | >30 | >3 | <10 |
| Example Z | <3 | >15 | >3 | <10 |
| Example AA | <3 | >5 | <3 | <3 |
| Example BB | <10 | >25 | <10 | |
| Example CC | 2.9 | 29 | 9.9 | 0.5 |
| Example DD | 10 | 74 | 31 | 1.8 |
| Example EE | 1.4 | 18 | 5.8 | 0.5 |
| Example FF | 16 | 86 | 45 | |
| Example GG | 34 | 386 | 122 | |
| Example HH | 0.4 | 37 | 7.6 | 0.4 |
| Example JJ | 56 | 352 | 584 | |
| Example KK | 0.57 | 52 | 13 | |
| Example LL | 0.7 | 31 | 12 | 0.8 |
| Example MM | 121 | 1930 | 1480 | |
| Example NN | 21.4 | 2425 | | |

### D. In Vivo Assay

Rats can be treated with an intraperitoneal injection of 1-12.5 mg/kg of endotoxin (LPS) with or without oral administration of the nitric oxide synthase inhibitors. Plasma nitrite/nitrate levels can be determined 5 hr post-treatment. The results can be used to show that the administration of the nitric oxide synthase inhibitors decreases the rise in plasma nitrite/nitrate levels, a reliable indicator of the production of nitric oxide induced by endotoxin. As shown in Table II, Example A ((2S,5E)-2-amino-6-fluoro-7-[(1-iminoethyl)amino]-5-heptenoic acid, dihydrochloride) inhibited the LPS-induced increase in plasma nitrite/nitrate levels with an observed ED₅₀ value of <0.1 mg/kg, demonstrating the ability to inhibit inducible nitric oxide synthase activity in vivo.

TABLE II
ED₅₀'s for Compounds Determined in Endotoxin-Treated Rats
(all compounds administered orally unless otherwise noted)

| Compound | ED₅₀ (mg/kg) |
|---|---|
| Example A | <0.1 |
| Example D | >10 |
| Example G | <0.1 |
| Example H | <0.3 |
| Example V | <3 |
| Example W | >10 |
| Example X | <5 |
| Example Y | <3 |
| Example Z | <5 |
| Example AA | <10 |
| Example CC | <3 |
| Example EE | 0.2 |
| Example HH | 0.4 |
| Example KK | 0.3 |
| Example LL | 0.3 |

### E. Assay for Time Dependent Inhibition

Compounds are evaluated for time dependent inhibition of human NOS isoforms by preincubation of the compound with the enzyme at 37° C. in the presence of the citrulline enzyme assay components, minus L-arginine, for times ranging from 0-60 min. Aliquots (10 μL) are removed at 0, 10, 21, and 60 min and immediately added to a citrulline assay enzyme reaction mixture containing L-[2,3-³H]-arginine and a final L-arginine concentration of 30 μM in a final volume of 100 μL. The reaction is allowed to proceed for 15 min at 37° C. and terminated by addition of stop buffer and chromatography with Dowex 50W X-8 cation exchange ion exchange resin as described for the citrulline NOS assay. The % inhibition of NOS activity by an inhibitor was taken as the per cent inhibition in activity compared to control enzyme preincubated for the same time in the absence of inhibitor. Data shown in Table III is the % inhibition after 21 and 60 min preincubation of inhibitor with enzyme.

TABLE III

| Example | | | |
|---|---|---|---|
| No. | hiNOS | hecNOS | hncNOS |
| V | 75%@2.8 μM@21 min | 11%@33 μM@21 min | 0%@5 μM@21 min |
| | 76%@2.8 μM@60 min | 11%@33 μM@60 min | 0%@5 μM@60 min |
| W | 34%@4.2 μM@21 min | 9%@173 μM@21 min | 0%@13 μM@21 min |
| | 38%@4.2 μM@60 min | 0%@173 μM@60 min | 0%@13 μM@60 min |
| X | 86%@2.2 μM@21 min | 18%@15 μM@21 min | 0%@3 μM@21 min |
| | 85%@2.2 μM@60 min | 16%@15 μM@60 min | 0%@3 μM@60 min |
| Y | 75%@2.8 μM@21 min | 11%@33 μM@21 min | 0%@5 μM@21 min |
| | 76%@2.8 μM@60 min | 11%@33 μM@60 min | 0%@5 μM@60 min |
| Z | 86%@2.2 μM@21 min | 18%@15 μM@21 min | 0%@3 μM@21 min |
| | 85%@2.2 μM@60 min | 16%@15 μM@60 min | 0%@3 μM@60 min |
| AA | 96%@2.2 μM@21 min | 58%@5.7 μM@21 min | 34%@0.9 μM@21 min |
| | 97%@2.2 μM@60 min | 55%@2.2 μM@60 min | 0%@0.9 μM@60 min |

### Dosages, Formulations and Routes of Administration

Many of the iNOS selective inhibitor compounds useful in the methods of the present invention can have at least 2 asymmetric carbon atoms, and therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention. Isomers may include geometric isomers, for example cis-isomers or trans-isomers across a double bond. All such isomers are contemplated among the compounds useful in the methods of the present invention. The methods also contemplate use of tautomers, salts, solvates and prodrugs of iNOS selective inhibitor compounds.

For the methods of the present invention, suitable routes of administration of the selective iNOS inhibitors include any means that produce contact of these compounds with their site of action in the subject's body, for example especially in the brain. More specifically, suitable routes of administration include inhalation, including oral inhalation or nasal inhalation, intranasal mucosal administration, oral, intravenous, subcutaneous, rectal, topical, buccal (i.e. sublingual), intramuscular, and intradermal.

For the treatment of cancer, the methods include use of an iNOS selective inhibitor as the compound per se, or as pharmaceutically acceptable salts thereof. The term “pharmaceutically-acceptable salt” embraces, for example, any salts commonly used to form alkali metal salts and to form addition salts of free acids or free bases. The nature of the salt is not critical, provided that it is pharmaceutically acceptable. Pharmaceutically acceptable salts are particularly useful as products of the methods of the present invention because of their greater aqueous solubility relative to a corresponding parent or neutral compound. Such salts must have a pharmaceutically acceptable anion or cation. Suitable pharmaceutically acceptable acid addition salts of compounds of the present invention may be prepared from inorganic acid or from an organic acid. Examples of such inorganic acids are hydrochloric, hydrobromic, hydroiodic, nitric, carbonic, sulfuric and phosphoric acid. Appropriate organic acids include from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, galacturonic acid. Suitable pharmaceutically-acceptable base addition salts of compounds of the present invention include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic (including carbonate and hydrogen carbonate anions), sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. The chloride salt is particularly preferred for medical purposes. Suitable pharmaceutically acceptable base salts include ammonium salts, alkali metal salts such as sodium and potassium salts, and alkaline earth salts such as magnesium and calcium salts. All of these salts may be prepared by conventional means from the corresponding conjugate base or conjugate acid of the compounds of the present invention by reacting, respectively, the appropriate acid or base with the conjugate base or conjugate acid of the compound.

In one embodiment, the iNOS selective inhibitors useful in the methods of the present invention are presented with an acceptable carrier in the form of a pharmaceutical combination or medicament. The carrier must be acceptable in the sense of being compatible with the other ingredients of the pharmaceutical combination and must not be deleterious to the subject. Suitable forms for the carrier include solid or liquid or both, and in an exemplary embodiment the carrier is formulated with the therapeutic compound as a unit-dose combination, for example as a tablet that contains from about 0.05% to about 95% by weight of the active compound. In alternative embodiments, other pharmacologically active substances are also present, including other compounds of the present invention. The pharmaceutical compounds of the present invention are prepared by any of the well-known techniques of pharmacy, consisting essentially of admixing the ingredients.

Preferred unit dosage formulations are those containing an effective dose, as herein below described, or an appropriate fraction thereof, of one or more of the therapeutic compounds of the combinations.

In general, a total daily dose of an iNOS selective inhibitor is in the range of about 0.001 mg/kg body weight/day to about 2500 mg/kg body weight/day. The dose range for adult humans is generally from about 0.005 mg to about 10 g per day. Tablets or other forms of presentation provided in discrete units may conveniently contain an amount of a therapeutic compound that is effective at such dosage, or at a multiple of the same. For instance, selective iNOS inhibitory compounds used in the present invention can be presented in units containing 5 mg to 500 mg, and typically around 10 mg to about 200 mg.

In the case of pharmaceutically acceptable salts of the therapeutic compounds, the weights indicated above refer to the weight of the acid equivalent or the base equivalent of the therapeutic compound derived from the salt.

For the methods herein described, it should be understood that the amount of a selective iNOS inhibitory compound that is required to achieve the desired biological effect depends on a number of factors, including the specific individual compound or compounds chosen, the specific use, the route of administration, the clinical condition of the subject, and the age, weight, gender, and diet of the subject.

The daily doses described in the preceding paragraphs for the various therapeutic compounds are administered in a single dose, or in proportionate multiple subdoses. Subdoses are administered from 2 to 6 times per day. In one embodiment, doses are administered in sustained release form effective to obtain the desired biological effect.

Delivery by inhalation, whether oral or nasal inhalation, according to the methods of the present invention can include formulations as are well known in the art, that are capable of being aerosolized for delivery by inhalation. A metered dose inhaler or a nebulizer provides aerosol delivery. Both devices are capable of providing delivery of a range of particle sizes including particles in the preferred range of about 1 micron to about 5 microns. Particles larger than about 10 microns are deposited primarily in the mouth and oropharynx, while particles smaller than about 0.5 microns are inhaled to the alveolac and then exhaled without significant deposition in the lungs. An alternative device for inhalant therapy is a dry powder inhaler using, for example, lactose or glucose powder to carry the therapeutic compound. For all forms of inhalant therapy, factors other than particle size and type of device also influence the amount of deposition in the lungs, including tidal volume, rate of breathing and breath holding. Therefore, an individual being instructed in inhalation therapy according to the methods of current invention should also be instructed to take slow deep breaths and hold each breath for several seconds, and preferably for about 5-10 seconds. Typically, the total daily dose of the therapeutic compounds according to the methods of the present invention will be administered as 1-4 puffs on a b.i.d-q.i.d. basis (i.e. twice-a-day, 3 times per day or 4 times a day), and as needed, or solely on an as-needed basis.

Oral delivery according to the methods of the present invention can include formulations, as are well known in the art, to provide prolonged or sustained delivery of the drug by any number of mechanisms. These include, but are not limited to, pH sensitive release from the dosage form based on the changing pH of the small intestine, slow erosion of a tablet or capsule, retention in the stomach based on physical properties of the formulation, bioadhesion of the dosage form to the mucosal lining of the intestinal tract, or enzymatic release of the active drug from the dosage form.

Oral delivery according to the methods of the present invention can be achieved using a solid, semi-solid or liquid dosage form. Suitable semi-solid and liquid forms include, for example, a syrup or liquid contained in a gel capsule.

To practice the methods of the present invention, pharmaceutical compositions suitable for oral administration can be presented in discrete units, such as capsules, cachets, lozenges, or tablets, each containing a predetermined amount of at least one of the therapeutic compounds useful in the methods of the present invention; as a powder or in granules; as a solution or a suspension in an aqueous or non-aqueous liquid; or as an oil-in-water or water-in-oil emulsion.

### Examples of Embodiments

The following examples are illustrate various pharmaceutical compositions suitable for practicing the treatment methods of the present invention. These examples are merely illustrative, and not limiting to this disclosure in any way.

Pharmaceutical Composition Example 1. 100 mg tablets of the composition set forth in Table IV can be prepared for oral administration using wet granulation techniques:

TABLE IV

| Ingredient | Weight (mg) |
|---|---|
| Compound LL | 25 |
| Lactose | 54 |
| Microcrystalline Cellulose | 15 |
| Hydroxypropyl Methylcellulose | 3 |
| Croscarmelose Sodium | 2 |
| Magnesium Stearate | 1 |
| Total Tablet Weight | 100 |

Pharmaceutical Composition Example 1. 100 mg tablets of the composition set forth in Table V can be prepared using direct compression techniques:

TABLE V

| Ingredient | Weight (mg) |
|---|---|
| Compound LL | 25 |
| Microcrystalline Cellulose | 69.5 |
| Colloidal Silicon Dioxide | 0.5 |
| Talc | 2.5 |
| Croscarmelose Sodium | 0.5 |
| Magnesium Stearate | 1 |
| Total Tablet Weight | 100 |

The examples described herein can be performed by substituting the generically or specifically described therapeutic compounds or inert ingredients for those used in the preceding examples.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

1. A method of treating cancer in a subject in need of such treatment, wherein:
the method comprises administering to the subject a carbamoylating chemotherapeutic agent in combination with a selective iNOS inhibitor; and
the selective iNOS inhibitor and carbomoylating chemotherapeutic agent are administered in amounts that together are treatment-effective.

2. The method of claim 1, wherein the chemotherapeutic agent is an alkylating agent.

3. The method of claim 1, wherein the carbamoylating chemotherapeutic agent is selected from the group consisting of:

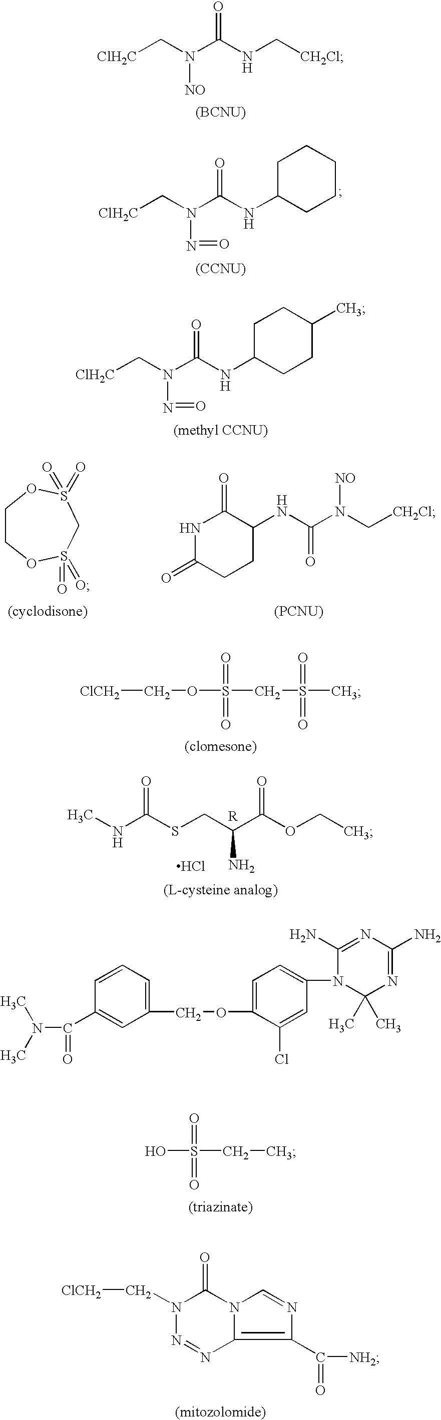

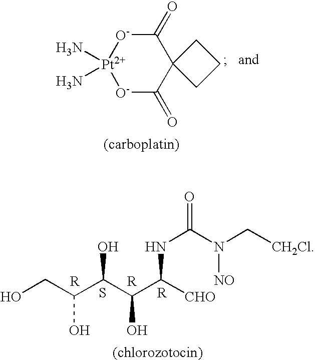

4. The method of claim 3, wherein the chemotherapeutic agent is BCNU.

5. The method of claim 3, wherein the chemotherapeutic agent is CCNU.

6. The method of claim 1, wherein:
the iNOS selective inhibitor is:
a compound corresponding in structure to a formula selected from the group consisting of Formula I, Formula II, Formula III, Formula IV, Formula V, Formula VI, Formula VII, Formula VIII, Formula IX, Formula X, Formula XI, Formula XII, Formula XIII, Formula XIV, Formula XV, and 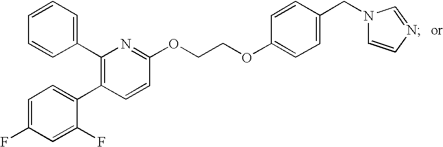
a stereoisomer of the compound, a pharmaceutically acceptable salt of the compound or stereoisomer, or a prodrug of the compound or stereoisomer;
Formula I is: 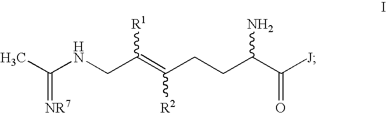
R¹ is selected from the group consisting of H, halo, and alkyl, wherein:
the alkyl is optionally substituted by one or more halo;
R² is selected from the group consisting of H, halo, and alkyl, wherein:
the alkyl is optionally substituted by one or more halo;
at least one of R¹ or R² contains a halo;
R⁷ is selected from the group consisting of H and hydroxy;
J is selected from the group consisting of hydroxy, alkoxy, and —NR³R⁴;
R³ is selected from the group consisting of H, lower alkyl, lower alkenyl, and lower alkynyl;
R⁴ is selected from the group consisting of H and a heterocyclic ring, wherein:
at least one member of the heterocyclic ring is carbon,
from 1 to about 4 heteroatoms of the heterocyclic ring are independently selected from the group consisting of oxygen, nitrogen, and sulfur, and
the heterocyclic ring is optionally substituted with heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, guanidino, amidino, or acylamino;
Formula II is: 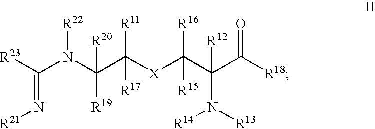
X is selected from the group consisting of —S—, —S(O)—, and —S(O)₂;
R¹² is selected from the group consisting of C₁-C₆ alkyl, C₂-C₆ alkenyl, C₂-C₆ alkynyl, C₁-C₅ alkoxy-methyl, and C₁-C₅ alkylthio-methyl, wherein:
each of these groups is optionally substituted by one or more substituents selected from the group consisting of —OH, alkoxy, and halogen;
as to R¹⁸:
R¹⁸ is selected from the group consisting of —OR²⁴ and —N(R²⁵)(R²⁶);
R¹⁸ is —N(R³⁰)— and R¹³ is —C(O)— such that, R¹⁸ and R¹³, together with the atoms to which they are attached, form a ring; or
R¹⁸ is —O— and R¹³ is —C(R³¹)(R³²)— such that, R¹⁸ and R¹³, together with the atoms to which they are attached, form a ring;
as to R¹³:
R¹³ is selected from the group consisting of —H, —OH, —C(O)—R²⁷, —C(O)—O—R²⁸, and —C(O)—S—R²⁹:
R¹³ is —C(O)— and R¹⁸ is —N(R³⁰)— such that R¹³ and R¹⁸, together with the atoms to which they are attached, form a ring; or
R¹³ is —C(R³¹)(R³²)— and R¹⁸ is —O— such that R¹³ and R¹⁸, together with the atoms to which they are attached, form a ring;
R¹⁴ is —H, except that:
R¹⁴ is —C(O)—O—R³³ when R¹³ is —(R³¹)(R³²)—;
R¹¹, R¹⁵, R¹⁶, and R¹⁷ independently are selected from the group consisting of —H, halogen, C₁-C₆ alkyl, C₂-C₆ alkenyl, C₂-C₆ alkynyl, and C₁-C₅ alkoxy-methyl, wherein:
the C₁-C₆ alkyl, C₂-C₆ alkenyl, or C₂-C₆ alkynyl is optionally substituted with one or more substituents selected from the group consisting of —OH, alkoxy, and halogen;
R¹⁹ and R²⁰ independently are selected from the group consisting of —H, C₁-C₆ alkyl, C₂-C₆ alkenyl, C₂-C₆ alkynyl, and C₁-C₅ alkoxy-methyl, wherein:
the C₁-C₆ alkyl, C₂-C₆ alkenyl, or C₂-C₆ alkynyl is optionally substituted with one or more substituents selected from the group consisting of —OH, alkoxy, and halogen;
as to R²¹:
R²¹is selected from the group consisting of —H, —OH, —C(O)—O—R³⁴, and —C(O)—S—R³⁵;
R²¹ is —O— and R²² is —C(O)— such that, R²¹ and R²², together with the atoms to which they are attached, form a ring; or
R²¹ is —C(O)— and R²² is —O— such that, R²¹ and R²², together with the atoms to which they are attached, form a ring;
as to R²²:
R²² is selected from the group consisting of —H, —OH, —C(O)—O—R³⁶, and —C(O)—S—R³⁷;
R²² is —C(O)— and R²¹ is —O— such that R²² and R²¹, together with the atoms to which they are attached, form a ring; or
R²² is —O— and R²¹ is —C(O)— such that R²² and R²¹, together with the atoms to which they are attached, form a ring;
R²³ is methyl optionally substituted with one or more substituents selected from the group consisting of —OH, alkoxy, and halogen;
R²⁴ is selected from the group consisting of —H and C₁-C₆ alkyl, wherein:
the C₁-C₆ alkyl is optionally substituted by one or more substituents selected from the group consisting of cycloalkyl, heterocyclyl, aryl, heteroaryl, —OH, alkoxy, and halogen;
as to R²⁵ and R²⁶:
R²⁵ is selected from the group consisting of —H, alkyl, and alkoxy; and R²⁶ is selected from the group consisting of —H, —OH, alkyl, alkoxy, —C(O)—R³⁸, —C(O)—O—R³⁹, and —C(O)—S—R⁴⁰, wherein:
each alkyl or alkoxy is, optionally substituted with one or more substituents selected from the group consisting of cycloalkyl, heterocyclyl, aryl, heteroaryl, —OH, alkoxy, and halogen; or
R²⁵ is —H; and R²⁶ is selected from the group consisting of cycloalkyl, heterocyclyl, aryl, and heteroaryl, wherein:
the cycloalkyl, heterocyclyl aryl, or heteroaryl optionally is substituted with one or more substituents selected from the group consisting of —OH, alkoxy, and halogen;
R²⁷, R²⁸, R²⁹, R³⁰, R³¹, R³², R³³, R³⁴, R³⁵, R³⁶, R³⁷, R³⁸, R³⁹, and R⁴⁰ independently are selected from the group consisting of —H and alkyl, wherein:
the alkyl is optionally substituted by one or more substituents selected from the group consisting of cycloalkyl, heterocyclyl, aryl, heteroaryl, —OH, alkoxy, and halogen;
Formula III is: 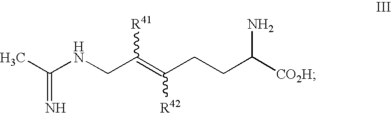
R⁴¹ is selected from the group consisting of H and methyl;
R⁴² is selected from the group consisting of H and methyl;
formula IV is: 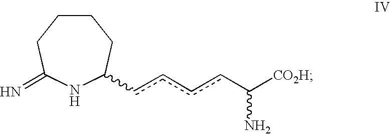
Formula V is: 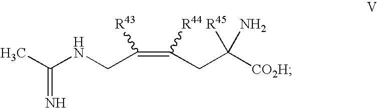
R⁴³ is selected from the group consisting of hydrogen, halo, and C₁-C₅ alkyl, wherein:
the C₁-C₅ alkyl optionally is substituted by one or more substituents selected from the group consisting of alkoxy and halo;
R⁴⁴ is selected from the group consisting of hydrogen, halo, and C₁-C₅ alkyl, wherein:
the C₁-C₅ alkyl optionally is substituted by one or more substituents selected from the group consisting of alkoxy and halo;
R⁴⁵ is C₁-C₅ alkyl optionally substituted by one or more substituents selected from the group consisting of alkoxy and halo;
Formula VI is: 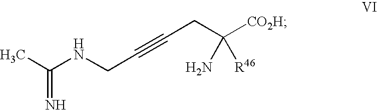
R⁴⁶ is C₁-C₅ alkyl, optionally substituted by halo or alkoxy, wherein:
the alkoxy optionally is substituted by one or more halo;
Formula VII is: 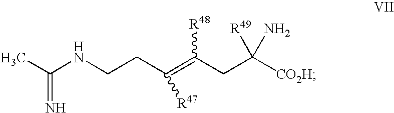
R⁴⁷ is selected from the group consisting of hydrogen, halo, and C₁-C₅ alkyl, wherein:
the C₁-C₅ alkyl optionally is substituted by one or more substituents selected from the group consisting of alkoxy and halo;
R⁴⁸ is selected from the group consisting of hydrogen, halo, and C₁-C₅ alkyl, wherein:
the C₁-C₅ alkyl optionally is substituted by one or more substituents selected from the group consisting of alkoxy and halo;
R⁴⁹ is C₁-C₅ alkyl optionally substituted by one or more substituents selected from the group consisting of alkoxy and halo;
Formula VIII is: 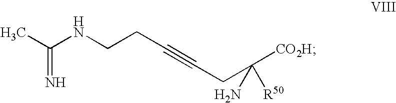
R⁵⁰ is C₁-C₅ alkyl, optionally substituted by halo or alkoxy, wherein:
the alkoxy optionally substituted by one or more halo;
formula IX is: 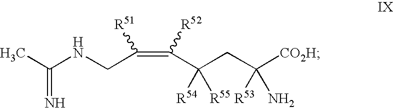
R⁵¹ is selected from the group consisting of hydrogen, halo, and C₁-C₅ alkyl, wherein:
the C₁-C₅ alkyl optionally is substituted by halo or alkoxy, wherein:
the alkoxy optionally is substituted by one or more halo;
R⁵² is selected from the group consisting of hydrogen, halo, and C₁-C₅ alkyl, wherein:
the C₁-C₅ alkyl optionally is substituted by halo or alkoxy, wherein:
the alkoxy optionally is substituted by one or more halo;
R⁵³ is C₁-C₅ alkyl, optionally substituted by halo or alkoxy, wherein:
the alkoxy optionally is substituted by one or more halo;
R⁵⁴ is selected from the group consisting of hydrogen, halo, and C₁-C₅ alkyl, wherein:
the C₁-C₅ alkyl optionally is substituted by halo or alkoxy, wherein:
the alkoxy optionally is substituted by one or more halo;
R⁵⁵ is selected from the group consisting of hydrogen, halo, and C₁-C₅ alkyl, wherein:
the C₁-C₅ alkyl optionally is substituted by halo or alkoxy, wherein:
the alkoxy optionally is substituted by one or more halo;
formula X is: 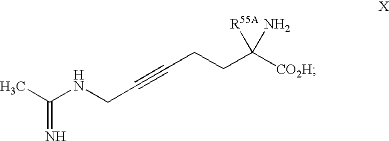
R^{56A} is C₁-C₅ alkyl, optionally substituted by halo or alkoxy, wherein:
the alkoxy optionally is substituted by one or more halo;
formula XI is: 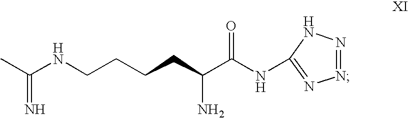
formula XII is: 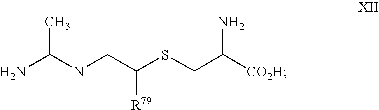
R⁷⁹ is selected from C₁₋₄ alkyl, C₃₋₄ cycloalkyl, C₁₋₄ hydroxyalkyl, and C₁₋₄ haloalkyl;
Formula XIII is: 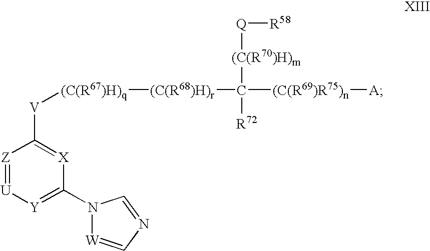
Formula XIV is: 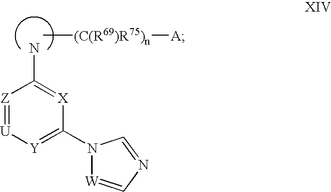
Formula XV is: 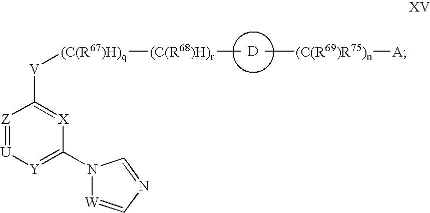
A is selected from the group consisting of —R⁵⁶, —OR⁵⁶, —C(O)N(R⁵⁶)R⁵⁷, —P(O)[N(R⁵⁶)R⁵⁷]₂, —N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —S(O)ₜR⁵⁶, —SO₂NHC(O)R⁵⁶, —NHSO₂R⁷⁷, —SO₂NH(R⁵⁶)H, —C(O)NHSO₂R⁷⁷, and —CH═NOR⁵⁶;
each X, Y, and Z is independently selected from the group consisting of N and C(R⁷⁴);
each U is C(R⁶⁰), except that:
U is selected from the group consisting of C(R⁶⁰) and N when X is N, and Z and Y are each CR⁷⁴;
V is selected from the group consisting of N(R⁵⁹), S, O, and C(R⁵⁹)H;
each W is N or CH;
as to Q:
Q is selected from the group consisting of a bond, —C(O)—, —O—, —C(═N—R⁵⁶)—, —S(O)ₜ—, and —N(R⁶¹); or
Q and R⁵⁸, taken together, form a substituent selected from the group consisting of —C(O)OH, —C(O)N(R⁵⁶)R⁵⁷, and 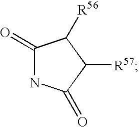
m is selected from the group consisting of zero, 1, 2, 3, and 4;
n is selected from the group consisting of zero, 1, 2, and 3;
q is selected from the group consisting of zero and one;
r is selected from the group consisting of zero and one;
at least one of m, q, and r is other than zero when Q and V are heteroatoms;
at least one of n, q, and r is other than zero when A is —OR⁵⁶, —N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —SR⁵⁶, or —NHSO₂R⁷⁷;
at least one of m and n is other than zero when Q is a heteroatom and A is —OR⁵⁶, —N(R⁵⁶)C(O)R⁵⁷, —N(R⁷¹)C(O)OR⁵⁷, —N(R⁵⁶)R⁷⁶, —N(R⁷¹)C(O)N(R⁵⁶)R⁷¹, —SR⁵⁶, or —NHSO₂R⁷⁷;
t is selected from the group consisting of zero, one, and two; 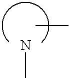
is optionally substituted N-heterocyclyl; 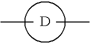
is selected from the group consisting of optionally substituted carbocyclyl and optionally substituted N-heterocyclyl;
as to each R⁵⁶ independently:
R⁵⁶ is selected from the group consisting of hydrogen, optionally substituted C₁-C₂₀ alkyl, optionally substituted cycloalkyl, —[C₀-C₈ alkyl]-R⁶⁴, —[C₂-C₈ alkenyl]-R⁶⁴, —[C₂-C₈ alkynyl]-R⁶⁴, —[C₂-C₈ alkyl]-R⁶⁵ (optionally substituted by hydroxy), —[C₁-C₈]-R⁶⁶ (optionally substituted by hydroxy), and optionally substituted heterocyclyl;
R⁵⁶ and an R⁵⁷, together with the nitrogen atom to which they are attached, is optionally substituted N-heterocyclyl; or
R⁵⁶ and R⁷⁶, taken together with the nitrogen to which they are attached, form optionally substituted N-heterocyclyl;
as to each R⁵⁷ independently:
R⁵⁷ is selected from the group consisting of hydrogen, optionally substituted C₁-C₂₀ alkyl, optionally substituted cycloalkyl, —[C₀-C₈ alkyl]-R⁶⁴, —[C₂-C₈ alkenyl]-R⁶⁴, —[C₂-C₈ alkynyl]-R⁶⁴, —[C₂-C₈ alkyl]-R⁶⁵ (optionally substituted by hydroxy), —[C₁-C₈]—R⁶⁶ (optionally substituted by hydroxy), optionally substituted heterocyclyl; or
R⁵⁷ and an R⁵⁶, together with the nitrogen atom to which they are attached, is optionally substituted N-heterocyclyl;
as to R⁵⁸:
R⁵⁸ is selected from the group consisting of hydrogen, alkyl, cycloalkyl, optionally substituted aryl, haloalkyl, —[C₁-C₈ alkyl]-C(O)N(R⁵⁶)R⁵⁷, —[C₁-C₈ alkyl]-N(R⁵⁶)R⁵⁷, —[C₁-C₈ alkyl]-R⁶³, —[C₂-C₈ alkyl]-R⁶⁵, —[C₁-C₈ alkyl]-R⁶⁶, and heterocyclyl, wherein:
the heterocyclyl optionally is substituted by one or more substituents selected from the group consisting of halo, alkyl, alkoxy, and imidazolyl; or
when Q is —N(R⁶¹)— or a to R⁵⁸, R⁵⁸ may additionally be selected from the group consisting of aminocarbonyl, alkoxycarbonyl, alkylsulfonyl, monoalkylaminocarbonyl, dialkylaminocarbonyl, and —C(═NR⁷³)—NH₂; or
R⁵⁸ and Q, taken together, form a substituent selected from the group consisting of —C(O)OH, —C(O)N(R⁵⁶)R⁵⁷, and 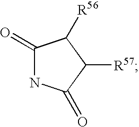
R⁵⁹ is selected from the group consisting of hydrogen, alkyl, aryl, aralkyl and cycloalkyl, except that:
when A is —R⁵⁶ or —OR⁵⁶, R⁵⁹ is selected from the group consisting of alkyl, aryl, aralkyl, and cycloalkyl;
when V is C(R⁵⁹)H, R⁵⁹ is selected from the group consisting of hydrogen, alkyl, aryl, aralkyl, cycloalkyl, and hydroxy;
R⁶⁰ is selected from the group consisting of hydrogen, alkyl, aryl, aralkyl, haloalkyl, optionally substituted aralkyl, optionally substituted aryl, —OR⁷¹, —S(O)ₜ—R⁷¹, —N(R⁷¹)R⁷⁶, —N(R⁷¹)C(O)N(R⁵⁶)R⁷¹, —N(R⁷¹)C(O)OR⁷¹, —N(R⁷¹)C(O)R⁷¹, —[C₀-C₈ alkyl]-C(H)[C(O)R⁷¹]₂, and —[C₀-C₈ alkyl]-C(O)N(R⁵⁶)R⁷¹;
R⁶¹ is selected from the group consisting of hydrogen, alkyl, cycloalkyl, —[C₁-C₈ alkyl]-R⁶³, —[C₂-C₈]alkyl]-R⁶⁵, —[C₁-C₈ alkyl]-R⁶⁶acyl, —C(O)R⁶³, —C(O)—[C₁-C₈ alkyl]-R⁶³, alkoxycarbonyl, optionally substituted aryloxycarbonyl, optionally substituted aralkoxycarbonyl, alkylsulfonyl, optionally substituted aryl, optionally substituted heterocyclyl, alkoxycarbonylalkyl, carboxyalkyl, optionally substituted arylsulfonyl, aminocarbonyl, monoalkylaminocarbonyl, dialkylaminocarbonyl, optionally substituted arylaminocarbonyl, aminosulfonyl, monoalkylaminosulfonyl dialkylaminosulfonyl, arylaminosulfonyl, arylsulfonylaminocarbonyl, optionally substituted N-heterocyclyl, —C(═NH)—N(CN)R⁵⁶, —C(O)R⁷⁸—N(R⁵⁶)R⁵⁷, and —C(O)—N(R⁵⁶)R⁷⁸—C(O)OR⁵⁶;
each R⁶³ and R⁶⁴ are independently selected from the group consisting of haloalkyl, cycloalkyl (optionally substituted with halo, cyano, alkyl, or alkoxy), carbocyclyl (optionally substituted with one or more substituents selected from the group consisting of halo, alkyl, and alkoxy), and heterocyclyl (optionally substituted with alkyl, aralkyl, or alkoxy);
each R⁶⁵ is independently selected from the group consisting of halo, alkoxy, optionally substituted aryloxy, optionally substituted aralkoxy, optionally substituted —S(O)ₜ—R⁷⁷, acylamino, amino, monoalkylamino, dialkylamino, (triphenylmethyl)amino, hydroxy, mercapto, and alkylsulfonamido;
each R⁶⁶ is independently selected from the group consisting of cyano, di(alkoxy)alkyl, carboxy, alkoxycarbonyl, aminocarbonyl, monoalkylaminocarbonyl, and dialkylaminocarbonyl;
each R⁶⁷, R⁶⁸, R⁶⁹, R⁷⁰, R⁷², and R⁷⁵ is independently selected from the group consisting of hydrogen and alkyl;
as to each R⁷¹:
each R⁷¹ is independently selected from the group consisting of hydrogen, alkyl, optionally substituted aryl, optionally substituted aralkyl, and cycloalkyl; or
R⁷¹ and R⁷⁶, taken together with the nitrogen to which they are attached, form optionally substituted N-heterocyclyl;
R⁷³ is selected from the group consisting of hydrogen, NO₂, and toluenesulfonyl;
each R⁷⁴ is independently selected from the group consisting of hydrogen, alkyl (optionally substituted with hydroxy), cyclopropyl, halo, and haloalkyl;
as to each R⁷⁶:
each R⁷⁶ is independently selected from the group consisting of hydrogen, alkyl, cycloalkyl, optionally substituted aryl, optionally substituted aralkyl, —C(O)R⁷⁷, and —SO₂R⁷⁷;
R⁷⁶ and R⁵⁶, taken together with the nitrogen to which they are attached, form optionally substituted N-heterocyclyl; or
R⁷⁶ and R⁷¹, taken together with the nitrogen to which they are attached, form optionally substituted N-heterocyclyl;
each R⁷⁷ is independently selected from the group consisting of alkyl, cycloalkyl, optionally substituted aryl, and optionally substituted aralkyl; and
R⁷⁸ is an amino acid residue

7. The method of claim 6, wherein the selective iNOS inhibitor is a selective iNOS substrate inhibitor.

8. The method of claim 7, wherein the selective iNOS inhibitor is a compound (or a pharmaceutically acceptable salt thereof) selected from the group consisting of:

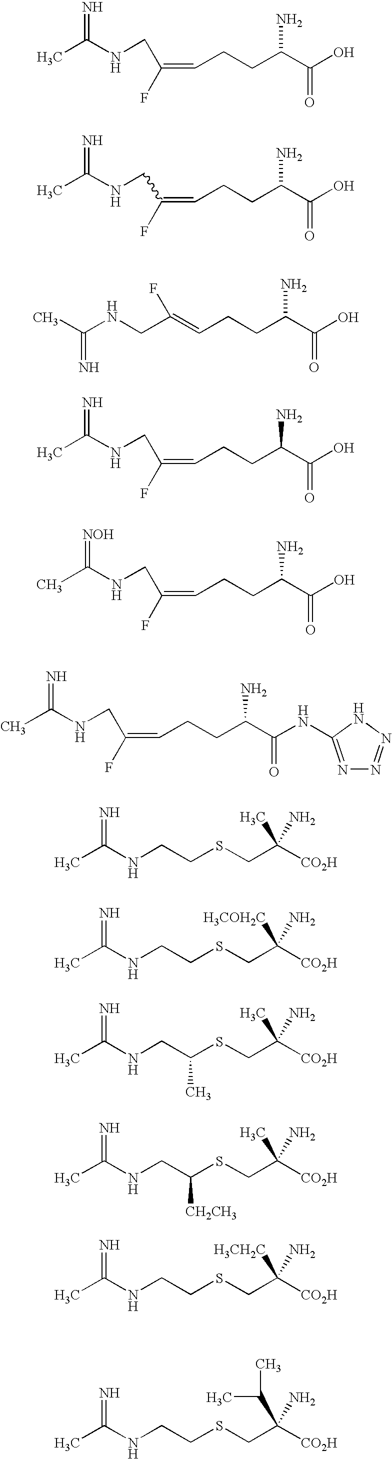

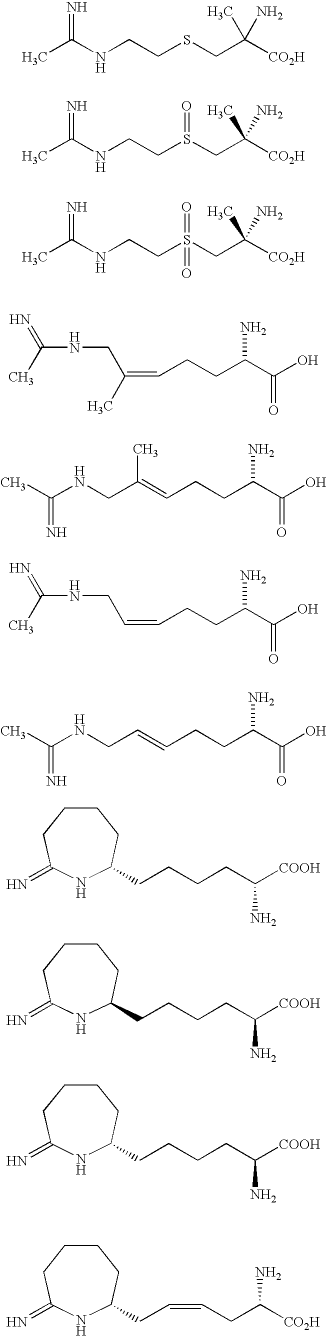

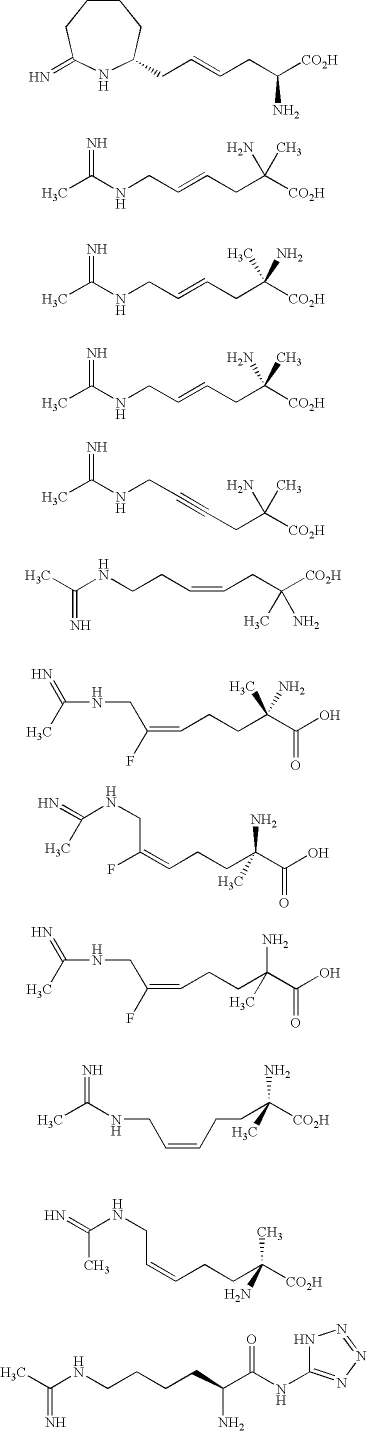

9. The method of claim 8, wherein the selective iNOS inhibitor is: 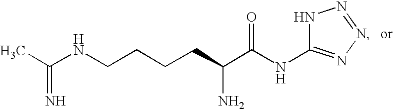
a pharmaceutically acceptable salt thereof

10. The method of claim 8, wherein the selective iNOS inhibitor is

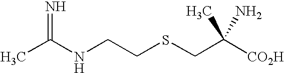

11. The method of claim 8, wherein the selective iNOS inhibitor is

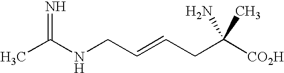

12. The method of claim 8, wherein the selective iNOS inhibitor is

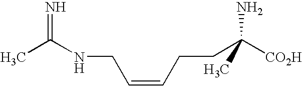

13. The method of claim 8, wherein the selective iNOS inhibitor is 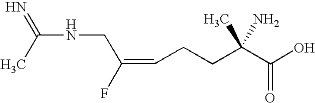
or a pharmaceutically acceptable salt thereof.

14. The method of claim 7, wherein the selective iNOS inhibitor is 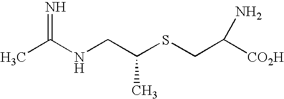
or a pharmaceutically acceptable salt thereof.

15. The method of claim 1, wherein the cancer is selected from the group consisting of adrenocortical carcinoma, cerebellar astrocytoma, brain stem glioma, ependymoma, medulloblastoma, supratentorial primitive neuroectodermal and pineal tumors, visual pathway and hypothalamic gliomas, astrocytomas, primary central nervous system lymphoma, eye cancers, head and neck cancer, neuroblastoma, pituitary tumor, meningioma, primitive neuroectodermal tumor, and secondary brain tumor.

16. The method of claim 15, wherein the cancer is glioblastoma multiforme.

17. A medicament, wherein the medicament:
is characterizeable by its ability to treat neoplasias resistant to carbamoylating chemotherapeutic agents; and
comprises:
a selective iNOS inhibiting compound or a selective iNOS inhibiting pharmaceutically acceptable salt,
a carbamoylating chemotherapeutic agent, and
a pharmaceutically acceptable carrier; and
the selective iNOS inhibiting compound or salt and carbomoylating chemotherapeutic agent are present in amounts that together are treatment-effective.

18. A kit, wherein:
the kit comprises:
a selective iNOS inhibiting compound or a selective iNOS inhibiting pharmaceutically acceptable salt, and
a carbamoylating chemotherapeutic agent; and
the selective iNOS inhibiting compound or salt and carbomoylating chemotherapeutic agent are present in amounts that together are treatment-effective.

19. The method of claim 7, wherein the selective iNOS inhibitor is selected from the group consisting of:

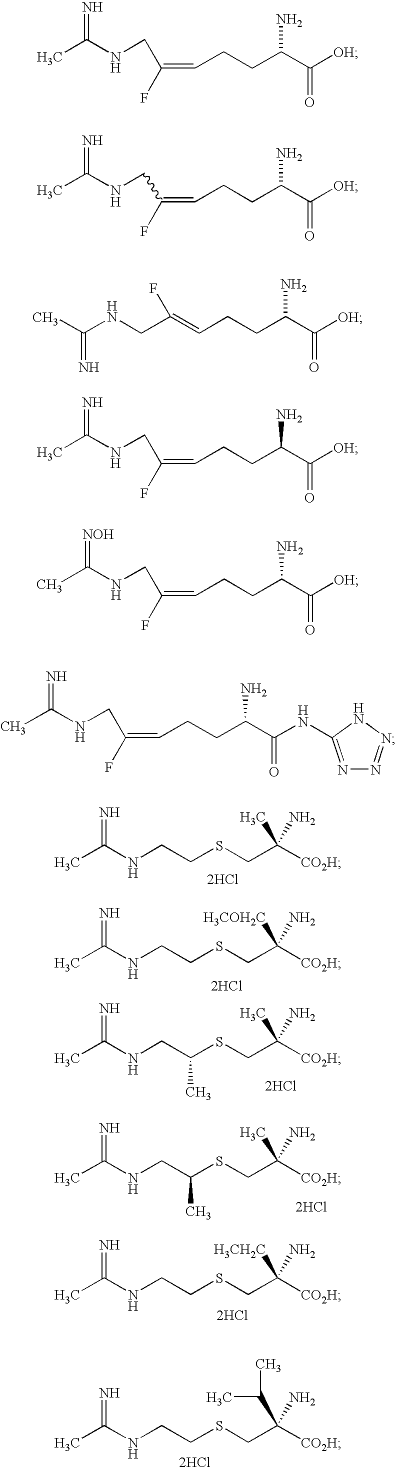

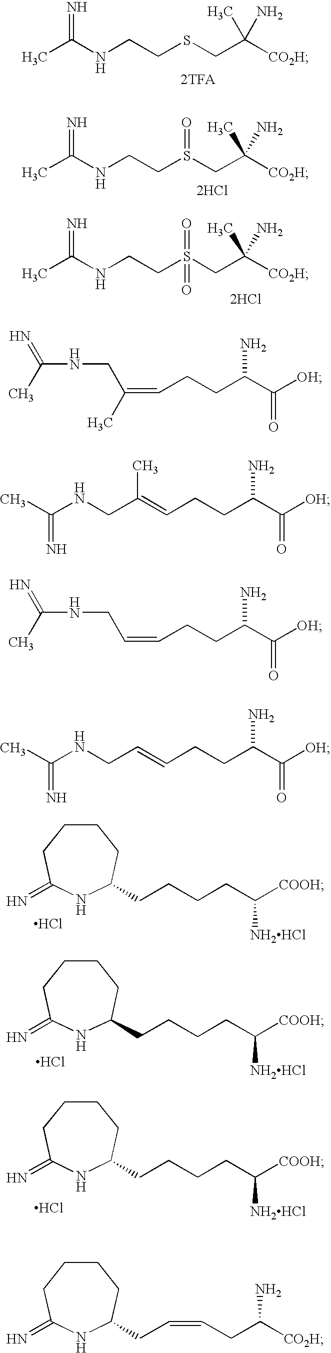

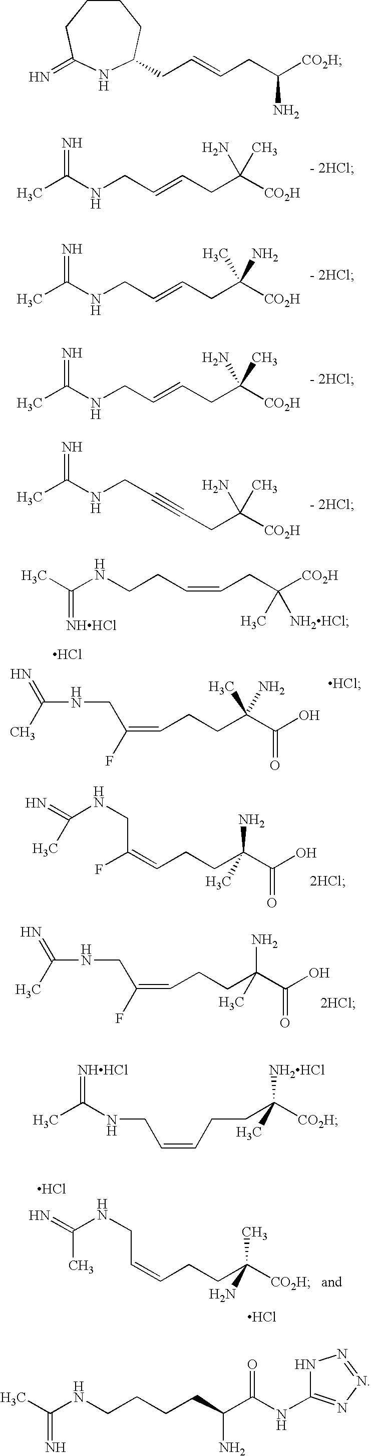

20. The method of claim 7, wherein the selective iNOS inhibitor is selected from the group consisting of:
(2S,5E)-2-amino-6-fluoro-7-[(1-iminoethyl)amino]-5-heptenoic acid, dihydrochloride, monohydrate;
(2S,5E/Z)-2-amino-6-fluoro-7-[(1-iminoethyl)amino]-5-heptenoic acid, dihydrochloride;
(2S,5Z)-2-amino-6-fluoro-7-[(1-iminoethyl)amino]-5-heptenoic acid, dihydrochloride;
(2S,5Z)-2-amino-6-fluoro-7-[(1-iminoethyl)amino]-5-heptenoic acid, trihydrochloride, dehydrate;
(2R,5E)-2-amino-6-fluoro-7-[(1-iminoethyl)amino]-5-heptenoic acid, dihydrochloride, monohydrate;
(2S,5E)-2-amino-6-fluoro-7-[(1-iminoethyl)amino]-5-heptenoic acid, dihydrochloride, monohydrate;
(2S,5E)-2-amino-6-fluoro-7-[(1-hydroximinoethyl)amino]-5-heptenoic acid;
(2S, 5E)-2-amino-6-fluoro-7-[(1-iminoethyl)amino]-N-(1H-tetrazol-5-yl) 5-heptenamide, dihydrochloride;
S-[2-[(1-iminoethyl)amino]ethyl]-2-methyl-L-cysteine, dihydrochloride;
2-[[[2-[(1-iminoethyl)amino]ethyl]thio]methyl]-O-methyl-D-serine, dihydrochloride;
S-[(1R)-2-[(1-iminoethyl)amino]-1-methylethyl]-2-methyl-L-cysteine, dihydrochloride;
S-[(1S)-2-[(1-iminoethyl)amino]-1-methylethyl]-2-methyl-L-cysteine, dihydrochloride;
S-[2-[(1-iminoethyl)amino]ethyl]-2-ethyl-L-cysteine, dihydrochloride;
2-[[[[2-(1-iminoethyl)amino]ethyl]thio]methyl]-D-valine, dihydrochloride;
S-[2-(1-iminoethylamino)ethyl]-2-methyl-(D/L)-cysteine, bistrifluoroacetate;
(2R)-2-amino-3[[2-[(1-iminoethyl)amino]ethyl]sulfinyl]-2-methylpropanoic acid, dihydrochloride;
(2R)-2-amino-3[[2-[(1-iminoethyl)amino]ethyl]sulfonyl]-2-methylpropanoic acid dihydrochloride;
(2S,5Z)-2-amino-6-methyl-7-[(1-iminoethyl)amino]-5-heptenoic acid, dihydrochloride;
(2S,5E)-2-amino-6-methyl-7-[(1-iminoethyl)amino]-5-heptenoic acid, dihydrochloride;
(2S,5Z)-2-amino-7-[(1-iminoethyl)amino]-5-heptenoic acid, dihydrochloride;
(2S,5E)-2-amino-7-[(1-iminoethyl)amino]-5-heptenoic acid, dihydrochloride;
(αR,2S)-α-aminohexahydro-7-imino-1H-azepine-2-hexanoic acid, trihydrate hydrochloride;
(αS,2R)-α-aminohexahydro-7-imino-1H-azepine-2-hexanoic acid, trihydrate hydrochloride;
(αS,2S)-α-aminohexahydro-7-imino-1H-azepine-2-hexanoic acid, trihydrate hydrochloride;
(2S,4Z)-2-amino-6-[(2R)-hexahydro-7-imino-1H-azepin-2-yl]-4-hexenoic acid;
(2S,4E)-2-amino-6-[(2R)-hexahydro-7-imino-1H-azepin-2-yl]-4-hexenoic acid;
(E)-2-amino-2-methyl-6-[(1-iminoethyl)amino]-4-hexenoic acid, dihydrochloride;
(R, E)-2-amino-2-methyl-6-[(1-iminoethyl)amino]-4-hexenoic acid, dihydrochloride;
(S, E)-2-amino-2-methyl-6-[(1-iminoethyl)amino]-4-hexenoic acid, dihydrochloride;
2-amino-2-methyl-6-[(1-iminoethyl)amino]-4-hexynoic acid, dihydrochloride;
(2R/S,4Z)-2-amino-2-methyl-7-[(1-iminoethyl)amino]-4-heptenoic acid, dihydrochloride;
(2S,5E)-2-amino-2-methyl-6-fluoro-7-[(1-iminoethyl)amino]-5-heptenoic acid, dihydrochloride;
(2R,5E)-2-amino-2-methyl-6-fluoro-7-[(1-iminoethyl)amino]-5-heptenoic acid, dihydrochloride;
(2R/S,5E)-2-amino-2-methyl-6-fluoro-7-[(1-iminoethyl)amino]-5-heptenoic acid, dihydrochloride;
(2S,5Z)-2-amino-2-methyl-7-[(1-iminoethyl)amino]-5-heptenoic acid, dihydrochloride;
(2R,5Z)-2-amino-2-methyl-7-[(1-iminoethyl)amino]-5-heptenoic acid, dihydrochloride; and
(2S-amino-6-[(1-iminoethyl)amino]-N-(1H-tetrazol-5-yl)hexanamide).

21. The method of claim 9, wherein the selective iNOS inhibitor is 2S-amino-6-[(1-iminoethyl)amino]-N-(1H-tetrazol-5-yl)hexanamide, hydrate, dihydrochloride.

22. The method of claim 12, wherein the selective iNOS inhibitor is

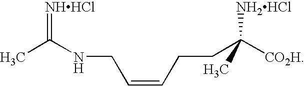

23. The method of claim 14, wherein the selective iNOS inhibitor is

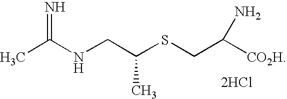

24. The method of claim 15, wherein the cancer is selected from the group consisting of intraocular melanoma and retinoblastoma.
